SEQUENCE LISTING (10 sequences)

SEQ ID NO: 1 - protein, 209 aa, Artificial Sequence
  sequence of Fc/3M
GGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLS

SEQ ID NO: 2 - protein, 225 aa, Homo sapiens
  sequence of Fc wild type
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 120 aa, Artificial Sequence
  variable region of heavy chain of antibody 3F2
EVQLVESGGGVVRPGGSLRLSCAASGFTVSDYSMNWVRQAPGKGLEWIGFIRNKANAYTTEYSASVKGRFTISRDDSKNTLYLQMNSLKTEDTAVYYCTTYPRYHAMDSWGQGTMVTVSS

SEQ ID NO: 4 - protein, 107 aa, Artificial Sequence
  variable region of light chain of antibody 3F2
AIQLTQSPSSLSASVGDRVTITCRASQSISNNLHWYLQKPGQSPQLLIYYGFQSISGVPSRFSGSGSGTDFTLTISSLQPEDFATYYCQQANSWPLTFGGGTKLEIK

SEQ ID NO: 5 - DNA, 57 bp, Artificial Sequence
  synthesized oligo
tatatatatctagacatatatatgggtgacaatgacatccactttgcctttctctcc

SEQ ID NO: 6 - DNA, 58 bp, Artificial Sequence
  synthesized oligo
tccactttgcctttctctccacaggtgtccactccactcacacatgcccaccgtgccc

SEQ ID NO: 7 - DNA, 40 bp, Artificial Sequence
  synthesized oligo
gatcaatgaattcgcggccgctcatttacccggagacagg

SEQ ID NO: 8 - protein, 223 aa, Artificial Sequence
  sequence of Fc/Mut1
THTCPPCPAPELLGGPSVFLFPPKPKDTLMISRTPEVTCVVVDVSHEDPEVKFNWYVDGVEVHNAKTKPREQNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 222 aa, Artificial Sequence
  sequence of Fc/Mut2
THTCPPCPAPELLGGPSVFLFPPKPKDTLMISRTPEVTCVVVDVSHEDPEVKFNWYVDGVEVHNAKTKPRENSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 220 aa, Artificial Sequence
  sequence of Fc/Mut3
THTCPPCPAPELLGGPSVFLFPPKPKDTLMISRTPEVTCVVVDVSHEDPEVKFNWYVDGVEVHNAKTKPRENSTVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2011/0091992 A1
(43) Pub. Date: Apr. 21, 2011

(54) CRYSTALS AND STRUCTURE OF HUMAN IgG Fc VARIANT

(75) Inventors: William Dall'Acqua, Gaithersburg, MD (US); Vaheh Oganesyan, North Potomac, MD (US); Jose Casas-Finet, Gaithersburg, MD (US); Bradford Braden, Baltimore, MD (US); Herren Wu, Boyds, MD (US)

(73) Assignee: MEDIMMUNE, LLC, Gaithersburg, MD (US)

(21) Appl. No.: 12/666,345

(22) PCT Filed: Jul. 10, 2008
(86) PCT No.: PCT/US08/08482

§ 371 Date: Nov. 12, 2010

Related U.S. Application Data
(60) Provisional application No. 60/959,048, filed on Jul. 10, 2007.
(60) Provisional application No. 60/959,126, filed on Jul. 11, 2007.
(60) Provisional application No. 60/966,050, filed on Aug. 23, 2007.
(60) Provisional application No. 60/981,441, filed on Oct. 19, 2007.
(60) Provisional application No. 61/064,361, filed on Feb. 29, 2008.
(60) Provisional application No. 61/064,460, filed on Mar. 6, 2008.

(51) Int. Cl. C07K 16/28; G01N 33/566; G06G 7/60

(52) U.S. Cl. 436/501; 530/387.3; 703/11

(57) ABSTRACT

The present invention provides crystalline forms of a human IgG Fc variant comprising one or more amino acid residues that provides for enhanced effector function, methods of obtaining such crystals and high-resolution X-ray diffraction structures and atomic structure coordinates. The present invention also provides machine readable media embedded with the three-dimensional atomic structure coordinates of the human IgG Fc variant and methods of using them. The present invention also provides human IgG Gc variants with reduced binding to at least one FcγR.

1. RELATED APPLICATIONS

This application claims the benefit of priority of U.S. provisional application No. 60/959,048, filed Jul. 10, 2007, 60/959,126, filed Jul. 11, 2007, 60/966,050, filed Aug. 23, 2007, 60/981,441, filed Oct. 19, 2007, 61/064,361, filed Feb. 29, 2008, and 61/064,460, filed Mar. 6, 2008, the contents of which are hereby incorporated by reference in their entireties.

2. FIELD OF THE INVENTION

The present invention provides crystalline forms of a human IgG Fc variant comprising one or more amino acid residues that provides for enhanced effector function, methods of obtaining such crystals and high-resolution X-ray diffraction structures and atomic structure coordinates. The crystals of the invention and the atomic structural information are useful for solving crystal and solution structures of related and unrelated proteins, and for screening for, identifying or designing compounds or antibodies that have altered, e.g., enhanced antibody dependent cell mediated cytotoxicity (ADCC). The invention further provides human IgG Fc variants having altered effector function. In particular, human IgG Fc variants are provided having reduced binding to one or more FcγRs.

### 3. BACKGROUND OF THE INVENTION

Antibodies are immunological proteins that bind a specific antigen. In most mammals, including humans and mice, antibodies are constructed from paired heavy and light polypeptide chains. Antibodies are made up of two distinct regions, referred to as the variable (Fv) and constant (Fc) regions. The light and heavy chain Fv regions contain the antigen binding determinants of the molecule and are responsible for binding the target antigen. The Fc regions define the class (or isotype) of antibody (IgG for example) and are responsible for binding a number of natural proteins to elicit important biochemical events.

The Fc region of an antibody interacts with a number of ligands including Fc receptors and other ligands, imparting an array of important functional capabilities referred to as effector functions. An important family of Fc receptors for the IgG class are the Fc gamma receptors (FcγRs). These receptors mediate communication between antibodies and the cellular arm of the immune system (Raghavan et al., 1996, Annu Rev Cell Dev Biol 12:181-220; Ravetch et al., 2001, Annu Rev Immunol 19:275-290). In humans this protein family includes FcγRI (CID64), including isoforms FcγRIA, FcγRIB, and FcγRIC; FcγRII (CD32), including isoforms FcγRIIA, FcγRIIB, and FcγRIIC; and FcγRII (CID16), including isoforms FcγRIIIA and FcγRIIB (Jefferis et al., 2002, Immunol Lett 82:57-65). These receptors typically have an extracellular domain that mediates binding to Fc, a membrane spanning region, and an intracellular domain that may mediate some signaling event within the cell. These different FcγR subtypes are expressed on different cell types (reviewed in Ravetch et al., 1991, Annu Rev Immunol 9:457-492). For example, in humans, FcγRIIIB is found only on neutrophils, whereas FcγRIIIA is found on macrophages, monocytes, natural killer (NK) cells, and a subpopulation of T-cells.

Formation of the Fc/FcγR complex recruits effector cells to sites of bound antigen, typically resulting in signaling events within the cells and important subsequent immune responses such as release of inflammation mediators, B cell activation, endocytosis, phagocytosis, and cytotoxic attack. The ability to mediate cytotoxic and phagocytic effector functions is a potential mechanism by which antibodies destroy targeted cells. The cell-mediated reaction wherein nonspecific cytotoxic cells that express FcγRs recognize bound antibody on a target cell and subsequently cause lysis of the target cell is referred to as antibody dependent cell-mediated cytotoxicity (ADCC) (Raghavan et al., 1996, Annu Rev Cell Dev Biol 12:181-220; Ghetie et al., 2000, Annu Rev Immunol 18:739-766; Ravetch et al., 2001, Annu Rev Immunol 19:275-290). Notably, the primary cells for mediating ADCC, NK cells, express only FcγRIIIA, whereas monocytes express FcγRI, FcγRII and FcγRIII (Ravetch et al., 1991, supra).

Several key features of antibodies including but not limited to, specificity for target, ability to mediate immune effector mechanisms, and long half-life in serum, make antibodies and related immunoglobulin molecules powerful therapeutics. Numerous monoclonal antibodies are currently in development or are being used therapeutically for the treatment of a variety of conditions including cancer. Examples of these include Vitaxin™ (MedImmune), a humanized Integrin αvβ3 antibody (e.g., PCT publication WO 2003/075957), Herceptin® (Genentech), a humanized anti-Her2/neu antibody approved to treat breast cancer (e.g., U.S. Pat. No. 5,677,171), CNTO 95 (Centocor), a human Integrin av antibody (PCT publication WO 02/12501), Rituxan™ (IDEC/Genentech/Roche), a chimeric anti-CD20 antibody approved to treat Non-Hodgkin's lymphoma (e.g., U.S. Pat. No. 5,736,137) and Erbitux® (ImClone), a chimeric anti-EGFR antibody (e.g., U.S. Pat. No. 4,943,533).

There are a number of possible mechanisms by which antibodies destroy tumor cells, including anti-proliferation via blockage of needed growth pathways, intracellular signaling leading to apoptosis, enhanced down regulation and/or turnover of receptors, ADCC, CDC, and promotion of an adaptive immune response (Cragg et al., 1999, Curr Opin Immunol 11:541-547; Glennie et al., 2000, Immunol Today 21:403-410). However, despite widespread use, antibodies are not yet optimized for clinical use and many have suboptimal anticancer potency. Thus, there is a significant need to enhance the capacity of antibodies to destroy targeted cancer cells. Methods for enhancing the anti-tumor-potency of antibodies via enhancement of their ability to mediate cytotoxic effector functions such as ADCC and CDC are particularly promising. The importance of FcγR-mediated effector functions for the anti-cancer activity of antibodies has been demonstrated in mice (Clynes et al., 1998, Proc Natl Acad Sci 95:652-656; Clynes et al., 2000, Nat Med 6:443-446), and the affinity of the interaction between Fc and certain FcγRs correlates with targeted cytotoxicity in cell-based assays (Shields et al., 2001, J Biol Chem 276:6591-6604; Presta et al., 2002, Biochem Soc Trans 30:487-490; Shields et al., 2002, J Biol Chem 277:26733-26740). Together these data suggest that manipulating the binding ability of the Fc region of an IgG1 antibody to certain FcγRs may enhance effector functions resulting in more effective destruction of cancer cells in patients. Furthermore, because FcγRs can mediate antigen uptake and processing by antigen presenting cells, enhanced Fc/FcγR affinity may also improve the capacity of antibody therapeutics to elicit an adaptive immune response.

Because ADCC activity is initiated by the binding of FcγRIII (referred to as “CD16” hereinafter) to the Fc region of IgGs, numerous studies have been carried out on the Fc region. It has been reported that the engineering of human IgGs for lack of fucose would result in an about 1 to 2 logs increase in both IgG binding to Human CD 16 and ADCC activity. See Niva et al., 2004, Clinic Cancer Research 10:6248-6255. The structural analysis of an afucosylated Fc region of human IgG suggested that the molecular basis for ADCC enhancement only involved subtle conformational changes. See Mutasumiya et al., 2007, J. Mol. Biol. 368:767-779. Further, by using computational design algorithms and high-throughput screening, various Fc variants exhibiting improved binding to CD16 have been identified. See Lazar et al., 2006, Proc. Natl. Acad. Sci. 103:4005-4010. One Fc triple mutant, designated Fc/3M, with three substitutions S239D/A330L/I332E, exhibited about 2 logs increase in human IgG1 binding to both F/V 158 allotypes of human CD16 and in ADCC activity. See Lazar et al., 2006, Proc. Natl. Acad. Sci. 103:4005-4010; Dall'Acqua et al., 2006, J Biol. Chem. 281:23514-23524.

The three-dimensional structure coordinates of a crystalline Fc region with enhanced CD 16 binding affinity, such as Fc/3M, would enable one to elucidate the molecular mechanism of the enhanced interaction between Fc/3M and human CD16. This atomic resolution information could also be used to design and/or select Fc variants with altered (e.g., enhanced) CD16 binding affinity and ADCC activity. The present invention provides the atomic structure coordinate of such Fc variants, particularly Fc/3M.

### 4. SUMMARY OF THE INVENTION

In one aspect, the invention provides crystalline forms of a human IgG Fc variant, wherein the human Fc variant comprises one or more high effector function amino acid residue and has an increased binding affinity for an FcγR as compared to a wild type human Fc region not comprising the one or more high effector function amino acid residue. In certain embodiments, the human IgG Fc variant comprises at least one high effector function amino acid residue selected from the group consisting of 239D, 330L or 332E, as numbered by the EU index as set forth in Kabat. In certain embodiments, the human IgG Fc variant comprises each of the high effector function amino acid residue mutations 239D, 330L and 332E, as numbered by the EU index as set forth in Kabat. In particular embodiments, the Fc variant comprises the amino acid sequence of SEQ ID NO:1. In some embodiments, the Fc variant consists of, or alternatively consists essentially of, the amino acid sequence of SEQ ID NO:1.

The crystals of the invention include native crystals, in which the crystallized human IgG Fc variant is substantially pure; heavy-atom atom derivative crystals, in which the crystallized human IgG Fc variant is in association with one or more heavy-metal atoms; and co-crystals, in which the crystallized human IgG Fc variant is in association with one or more binding compounds, including but not limited to, Fc receptors, cofactors, ligands, substrates, substrate analogs, inhibitors, effectors, etc. to form a crystalline complex. Preferably, such binding compounds bind a catalytic or active site, such as the cleft formed by the C_{H}2 and C_{H}3 domains of the human IgG Fc variant. The co-crystals may be native poly-crystals, in which the complex is substantially pure, or they may be heavy-atom derivative co-crystals, in which the complex is in association with one or more heavy-metal atoms.

In certain embodiments, the crystals of the invention are generally characterized by an orthorhombic space group C222₁ with unit cell of a=49.87+/−0.2 Å, b=147.49+/−0.2 Å, c=74.32 +/−0.2 Å, and are preferably of diffraction quality. A typical diffraction pattern is illustrated in FIG. 8. In more preferred embodiments, the crystals of the invention are of sufficient quality to permit the determination of the three-dimensional X-ray diffraction structure of the crystalline polypeptide(s) to high resolution, preferably to a resolution of greater than about 3 Å, typically in the range of about 2 Å to about 3 Å. The three-dimensional structural information may be used in a variety of methods to design and screen for compounds that bind a human IgG Fc region, as described in more detail below

The invention also provides methods of making the crystals of the invention. Generally, crystals of the invention are grown by dissolving substantially pure human IgG Fc variant in an aqueous buffer that includes a precipitant at a concentration just below that necessary to precipitate the polypeptide. Water is then removed by controlled evaporation to produce precipitating conditions, which are maintained until crystal growth ceases.

Co-crystals of the invention are prepared by soaking a native crystal prepared according to the above method in a liquor comprising the binding compound of the desired complexes. Alternatively, the co-crystals may be prepared by co-crystallizing the complexes in the presence of the compound according to the method discussed above or by forming a complex comprising the polypeptide and the binding compound and crystallizing the complex.

Heavy-atom derivative crystals of the invention may be prepared by soaking native crystals or co-crystals prepared according to the above method in a liquor comprising a salt of a heavy atom or an organometallic compound. Alternatively, heavy-atom derivative crystals may be prepared by crystallizing a polypeptide comprising selenomethionine and/or selenocysteine residues according to the methods described previously for preparing native crystals.

In another aspect, the invention provides machine and/or computer-readable media embedded with the three-dimensional structural information obtained from the crystals of the invention, or portions or subsets thereof Such three-dimensional structural information will typically include the atomic structure coordinates of the crystalline human IgG Fc variant, either alone or in a complex with a binding compound, or the atomic structure coordinates of a portion thereof such as, for example, the atomic structure coordinates of residues comprising an antigen binding site, but may include other structural information, such as vector representations of the atomic structures coordinates, etc. The types of machine- or computer-readable media into which the structural information is embedded typically include magnetic tape, floppy discs, hard disc storage media, optical discs, CD-ROM, electrical storage media such as RAM or ROM, and hybrids of any of these storage media. Such media further include paper on which is recorded the structural information that can be read by a scanning device and converted into a three-dimensional structure with an OCR and also include stereo diagrams of three-dimensional structures from which coordinates can be derived. The machine readable media of the invention may further comprise additional information that is useful for representing the three-dimensional structure, including, but not limited to, thermal parameters, chain identifiers, and connectivity information.

The invention is illustrated by way of working examples demonstrating the crystallization and characterization of crystals, the collection of diffraction data, and the determination and analysis of the three-dimensional structure of human IgG Fc variant.

The atomic structure coordinates and machine-readable media of the invention have a variety of uses. For example, the coordinates are useful for solving the three-dimensional X-ray diffraction and/or solution structures of other proteins, including, both alone or in complex with a binding compound. Structural information may also be used in a variety of molecular modeling and computer-based screening applications to, for example, intelligently screen or design human IgG Fc variants or antibody comprising Fc variant, or fragments thereof, that have altered biological activity, particularly altered binding affinity to a FcγR and/or altered ADCC activity, to identify compounds that bind to a human IgG Fc region, or fragments thereof, for example, C_{H}2 or C_{H}3 domain of Fc region. Such compounds may be used to lead compounds in pharmaceutical efforts to identify compounds that mimic the human IgG Fc variant with enhanced FcγR binding affinity and/or ADCC activity.

In still another aspect the invention provides a recombinant polypeptide comprising a human IgG Fc variant, wherein the human Fc variant comprises one or more amino acid residue substitutions and/or deletions and has an reduced binding affinity for an FcγR as compared to a comparable polypeptide comprising a wild type human Fc region not comprising the one or more amino acid residue substitutions and/or deletions. In certain embodiments, the human IgG Fc variant comprises the deletion of at least one amino acid residue selected from the group consisting of 294, 295, 296, 298 and 299 as numbered by the EU index as set forth in Kabat. In certain embodiments, the human IgG Fc variant comprises the substitution of at least one amino acid residue selected from the group consisting of 300S and 301T as numbered by the EU index as set forth in Kabat. In particular embodiments, the recombinant polypeptide comprises the amino acid sequence of any one of SEQ ID NOS:8-10

### 4.1 ABBREVIATIONS

The amino acid notations used herein for the twenty genetically encoded L-amino acids are conventional and are as follows:

| | One-Letter | Three-Letter |
|---|---|---|
| Amino Acid | Symbol | Symbol |
| Alanine | A | Ala |
| Arginine | R | Arg |
| Asparagine | N | Asn |
| Aspartic acid | D | Asp |
| Cysteine | C | Cys |
| Glutamine | Q | Gln |
| Glutamic acid | E | Glu |
| Glycine | G | Gly |
| Histidine | H | His |
| Isoleucine | I | Ile |
| Leucine | L | Leu |
| Lysine | K | Lys |
| Methionine | M | Met |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |

As used herein, unless specifically delineated otherwise, the three-letter amino acid abbreviations designate amino acids in the L-configuration. Amino acids in the D-configuration are preceded with a “D-.” For example, Arg designates L-arginine and D-Arg designates D-arginine. Likewise, the capital one-letter abbreviations refer to amino acids in the L-configuration. Lower-case one-letter abbreviations designate amino acids in the D-configuration. For example, “R” designates L-arginine and “r” designates D-arginine.

Unless noted otherwise, when polypeptide sequences are presented as a series of one-letter and/or three-letter abbreviations, the sequences are presented in the N→C direction, in accordance with common practice.

### 4.2 DEFINITIONS

As used herein, the following terms shall have the following meanings:

“Genetically Encoded Amino Acid” refers to L-isomers of the twenty amino acids that are defined by genetic codons. The genetically encoded amino acids are the L-isomers of glycine, alanine, valine, leucine, isoleucine, serine, methionine, threonine, phenylalanine, tyrosine, tryptophan, cysteine, proline, histidine, aspartic acid, asparagine, glutamic acid, glutamine, arginine and lysine.

“Genetically Non-Encoded Amino Acid” refers to amino acids that are not defined by genetic codons. Genetically non-encoded amino acids include derivatives or analogs of the genetically-encoded amino acids that are capable of being enzymatically incorporated into nascent polypeptides using conventional expression systems, such as selenomethionine (SeMet) and selenocysteine (SeCys); isomers of the genetically-encoded amino acids that are not capable of being enzymatically incorporated into nascent polypeptides using conventional expression systems, such as D-isomers of the genetically-encoded amino acids; L- and D-isomers of naturally occurring a-amino acids that are not defined by genetic codons, such as α-aminoisobutyric acid (Aib); L- and D-isomers of synthetic α-amino acids that are not defined by genetic codons; and other amino acids such as β-amino acids, γ-amino acids, etc. In addition to the D-isomers of the genetically-encoded amino acids, common genetically non-encoded amino acids include, but are not limited to norleucine (Nle), penicillamine (Pen), N-methylvaline (MeVal), homocysteine (hCys), homoserine (hSer), 2,3-diaminobutyric acid (Dab) and ornithine (Orn). Additional exemplary genetically non-encoded amino acids are found, for example, in Practical Handbook of Biochemistry and Molecular Biology, 1989, Fasman, Ed., CRC Press, Inc., Boca Raton, Fla., pp. 3-76 and the various references cited therein.

“Hydrophilic Amino Acid” refers to an amino acid having a side chain exhibiting a hydrophobicity of less than zero according to the normalized consensus hydrophobicity scale of Eisenberg et al., 1984, J. Mol. Biol. 179:125-142. Genetically encoded hydrophilic amino acids include Thr (T), Ser (S), His (H), Glu (E), Asn (N), Gln (Q), Asp (D), Lys (K) and Arg (R). Genetically non-encoded hydrophilic amino acids include the D-isomers of the above-listed genetically-encoded amino acids, ornithine (Orn), 2,3-diaminobutyric acid (Dab) and homoserine (hSer).

“Acidic Amino Acid” refers to a hydrophilic amino acid having a side chain pK value of less than 7 under physiological conditions. Acidic amino acids typically have negatively charged side chains at physiological pH due to loss of a hydrogen ion. Genetically encoded acidic amino acids include Glu (E) and Asp (D). Genetically non-encoded acidic amino acids include D-Glu (e) and D-Asp (d).

“Basic Amino Acid” refers to a hydrophilic amino acid having a side chain pK value of greater than 7 under physiological conditions. Basic amino acids typically have positively charged side chains at physiological pH due to association with hydronium ion. Genetically encoded basic amino acids include His (H), Arg (R) and Lys (K). Genetically non-encoded basic amino acids include the D-isomers of the above-listed genetically-encoded amino acids, ornithine (Orn) and 2,3-diaminobutyric acid (Dab).

“Polar Amino Acid” refers to a hydrophilic amino acid having a side chain that is uncharged at physiological pH, but which comprises at least one covalent bond in which the pair of electrons shared in common by two atoms is held more closely by one of the atoms. Genetically encoded polar amino acids include Asn (N), Gln (Q), Ser (S), and Thr (T). Genetically non-encoded polar amino acids include the D-isomers of the above-listed genetically-encoded amino acids and homoserine (hSer).

“Hydrophobic Amino Acid” refers to an amino acid having a side chain exhibiting a hydrophobicity of greater than zero according to the normalized consensus hydrophobicity scale of Eisenberg et al., 1984, J. Mol. Biol. 179:125-142. Genetically encoded hydrophobic amino acids include Pro (P), Ile (I), Phe (F), Val (V), Leu (L), Trp (W), Met (M), Ala (A), Gly (G) and Tyr (Y). Genetically non-encoded hydrophobic amino acids include the D-isomers of the above-listed genetically-encoded amino acids, norleucine (Nle) and N-methyl valine (MeVal).

“Aromatic Amino Acid” refers to a hydrophobic amino acid having a side chain comprising at least one aromatic or heteroaromatic ring. The aromatic or heteroaromatic ring may contain one or more substituents such as —OH, —SH, —CN, —F, —Cl, —Br, —I, —NO₂, —NO, —NH₂, —NHR, —NRR, —C(O)R, —C(O)OH, —C(O)OR, —C(O)NH₂, —C(O)NHR, —C(O)NRR and the like where each R is independently (C₁-C₆) alkyl, (C₁-C₆) alkenyl, or (C₁-C₆) alkynyl. Genetically encoded aromatic amino acids include Phe (F), Tyr (Y), Trp (W) and His (H). Genetically non-encoded aromatic amino acids include the D-isomers of the above-listed genetically-encoded amino acids.

“Apolar Amino Acid” refers to a hydrophobic amino acid having a side chain that is uncharged at physiological pH and which has bonds in which the pair of electrons shared in common by two atoms is generally held equally by each of the two atoms (i.e., the side chain is not polar). Genetically encoded apolar amino acids include Leu (L), Val (V), Ile (I), Met (M), Gly (G) and Ala (A). Genetically non-encoded apolar amino acids include the D-isomers of the above-listed genetically-encoded amino acids, norleucine (Nle) and N-methyl valine (MeVal).

“Aliphatic Amino Acid” refers to a hydrophobic amino acid having an aliphatic hydrocarbon side chain. Genetically encoded aliphatic amino acids include Ala (A), Val (V), Leu (L) and Ile (I). Genetically non-encoded aliphatic amino acids include the D-isomers of the above-listed genetically-encoded amino acids, norleucine (Nle) and N-methyl valine (MeVal).

“Helix-Breaking Amino Acid” refers to those amino acids that have a propensity to disrupt the structure of a-helices when contained at internal positions within the helix. Amino acid residues exhibiting helix-breaking properties are well-known in the art (see, e.g., Chou & Fasman, 1978, Ann. Rev. Biochem. 47:251-276) and include Pro (P), D-Pro (p), Gly (G) and potentially all D-amino acids (when contained in an L-polypeptide; conversely, L-amino acids disrupt helical structure when contained in a D-polypeptide).

“Cysteine-like Amino Acid” refers to an amino acid having a side chain capable of participating in a disulfide linkage. Thus, cysteine-like amino acids generally have a side chain containing at least one thiol (—SH) group. Cysteine-like amino acids are unusual in that they can form disulfide bridges with other cysteine-like amino acids. The ability of Cys (C) residues and other cysteine-like amino acids to exist in a polypeptide in either the reduced free -SH or oxidized disulfide-bridged form affects whether they contribute net hydrophobic or hydrophilic character to a polypeptide. Thus, while Cys (C) exhibits a hydrophobicity of 0.29 according to the consensus scale of Eisenberg (Eisenberg, 1984, supra), it is to be understood that for purposes of the present invention Cys (C) is categorized as a polar hydrophilic amino acid, notwithstanding the general classifications defined above. Other cysteine-like amino acids are similarly categorized as polar hydrophilic amino acids. Typical cysteine-like residues include, for example, penicillamine (Pen), homocysteine (hCys), etc.

As will be appreciated by those of skill in the art, the above-defined classes or categories are not mutually exclusive. Thus, amino acids having side chains exhibiting two or more physico-chemical properties can be included in multiple categories. For example, amino acid side chains having aromatic groups that are further substituted with polar substituents, such as Tyr (Y), may exhibit both aromatic hydrophobic properties and polar or hydrophilic properties, and could therefore be included in both the aromatic and polar categories. Typically, amino acids will be categorized in the class or classes that most closely define their net physico-chemical properties. The appropriate categorization of any amino acid will be apparent to those of skill in the art.

The classifications of the genetically encoded and common non-encoded amino acids according to the categories defined above are summarized in Table 1, below. It is to be understood that Table 1 is for illustrative purposes only and does not purport to be an exhaustive list of the amino acid residues belonging to each class. Other amino acid residues not specifically mentioned herein can be readily categorized based on their observed physical and chemical properties in light of the definitions provided herein.

TABLE 1
CLASSIFICATIONS OF COMMONLY ENCOUNTERED
AMINO ACIDS

| | Genetically | Genetically |
|---|---|---|
| Classification | Encoded | Non-Encoded |
| Hydrophobic | | |
| Aromatic | F, Y, W, H | f, y, w, h |
| Apolar | L, V, I, M, G, A, P | l, v, i, m, a, p, Nle, MeVal |
| Aliphatic | A, V, L, I | a, v, l, I, Nle, MeVal |
| Hydrophilic | | |
| Acidic | D, E | d, e |
| Basic | H, K, R | h, k, r, Orn, Dab |
| Polar | C, Q, N, S, T | c, q, n, s, t, hSer |
| Helix-Breaking | P, G | P |

An “antibody” or “antibodies” refers to monoclonal antibodies, multispecific antibodies, human antibodies, humanized antibodies, synthetic antibodies, chimeric antibodies, camelized antibodies, single-chain Fvs (scFv), single chain antibodies, Fab fragments, F(ab′) fragments, disulfide-linked Fvs (sdFv), intrabodies, and anti-idiotypic (anti-Id) antibodies (including, e.g., anti-Id and anti-anti-Id antibodies), bispecific, and epitope-binding fragments of any of the above. In particular, antibodies include immunoglobulin molecules and immunologically active fragments of immunoglobulin molecules, i.e., molecules that contain an antigen binding site. Immunoglobulin molecules can be of any type (e.g., IgG, IgE, IgM, IgD, IgA and IgY), class (e.g., IgG₁, IgG₂, IgG₃, IgG₄, IgA₁ and IgA₂) or subclass.

“Fc,” “Fc region,” or “Fc polypeptide,” as used herein interchangeably, includes the polypeptides comprising the constant region of an antibody excluding the first constant region immunoglobulin domain. Thus Fc refers to the last two constant region immunoglobulin domains of IgA, IgD, and IgG, and the last three constant region immunoglobulin domains of IgE and IgM, and the flexible hinge N-terminal to these domains. For IgA and IgM Fc may include the J chain. For IgG, Fc comprises immunoglobulin domains Cγ2 and Cγ3 (Cγ2 and Cγ3) and the hinge between Cγ1 (Cγ1) and Cγ2 (Cγ2). Although the boundaries of the Fc region may vary, the human IgG heavy chain Fc region is usually defined to comprise residues T223, or C226 or P230 to its carboxyl-terminus, wherein the numbering is according to the EU index as in Kabat et al. (1991, NIH Publication 91-3242, National Technical Information Service, Springfield, Va.).

The “EU index as set forth in Kabat” refers to the residue numbering of the human IgG1 EU antibody as described in Kabat et al. supra. Fc may refer to this region in isolation, or this region in the context of an antibody, antibody fragment, or Fc fusion protein. Note: Polymorphisms have been observed at a number of Fc positions, including but not limited to Kabat 270, 272, 312, 315, 356, and 358, and thus slight differences between the presented sequence and sequences in the prior art may exist.

“Human IgG Fc variant” or simply “Fc variant” refers to a human IgG Fc region comprises one or more amino acid substitution, deletion, insertion or modification (e.g., carbohydrate chemical modification) introduced at any position within the Fc region. In certain embodiments a human IgG Fc variant comprises a high effector function amino acid residue and has an increased binding affinity for an FcγR as compared to the wild type Fc region not comprising the one or more high effector function amino acid residue. Fc binding interactions are essential for a variety of effector functions and downstream signaling events including, but not limited to, antibody dependent cell-mediated cytotoxicity (ADCC) and complement dependent cytotoxicity (CDC). Accordingly, in certain embodiments, human IgG Fc variants exhibit altered binding affinity for at least one or more Fc ligands (e.g., FcγRs) relative to an antibody having the same amino acid sequence but not comprising the one or more amino acid substitution, deletion, insertion or modification (referred to herein as a “comparable molecule”) such as, for example, an unmodified Fc region containing naturally occurring amino acid residues at the corresponding position in the Fc region.

“Wild type human IgG Fc region” refers to a human IgG Fc region that comprises the amino acid sequence of SEQ ID NO: 2 or a fragment thereof (from residue T223 to residue K447 of human IgG heavy chain, wherein the numbering is according to the EU index as in Kabat).

“High effector amino acid residue” refers to the substitution of an amino acid residue of a human IgG Fc region that confers enhanced binding to one or more Fc ligands (e.g., FcγRs) relative to an antibody having the same amino acid sequence but not comprising the high effector amino acids residues. Such high effector amino acid residue is described in detail in U.S. Pat. App. Pub. No. 2006/0039904, the contents of which is hereby incorporated by reference in its entirety. In certain embodiments, the human IgG Fc variant comprises a human IgG Fc region comprising at least one high effector function amino acid residue selected from the group consisting of: 234E, 235R, 235A, 235W, 235P, 235V, 235Y, 236E, 239D, 265L, 269S, 269G, 2981, 298T, 298F, 327N, 327G, 327W, 328S, 328V, 329H, 329Q, 330K, 330V, 330G, 330Y, 330T, 330L, 3301, 330R, 330C, 332E, 332H, 332S, 332W, 332F, 332D, and 332Y, wherein the numbering system is that of the EU index as set forth in Kabat et al. (1991, NIH Publication 91-3242, National Technical Information Service, Springfield, Va.).

In some embodiments, the human IgG Fc variant comprises a human IgG Fc region comprising at least one high effector function amino acid residue selected from the group consisting of: 239D, 330K, 330V, 330G, 330Y, 330T, 330L, 3301, 330R, 330C, 332E, 332H, 332S, 332W, 332F, 332D, and 332Y wherein the numbering system is that of the EU index as set forth in Kabat.

In some embodiments, the human IgG Fc variant comprises a human IgG Fc region comprising at least one high effector function amino acid residue selected from the group consisting of: 239D, 330L and 332E, wherein the numbering system is that of the EU index as set forth in Kabat. In some embodiments, the human IgG Fc variant comprises human IgG Fc region comprising the high effector function amino acid residues 239D, 330L and 332E. Such human IgG Fc variant is designated as the Fc/3M variant. In particular embodiments, the human IgG Fc variant comprises the amino acid sequence of SEQ ID NO:1.

In addition to the high effector function amino acid residues described above, the human IgG Fc variant may comprise one or more additional substitution of at least one amino acid residue of the wild-type sequence(s) with a different amino acid residue and/or by the addition and/or deletion of one or more amino acid residues to or from the wild-type sequence(s). Such human IgG Fc variant is referred to as a Fc variant mutant. The additions and/or deletions can be from an internal region of the wild-type sequence and/or at either or both of the N- or C-termini. In certain embodiments, 1, 2, 3, 4 or 5 amino acid substitutions, deletions or additions are present.

“Conservative Mutant” refers to a mutant in which at least one amino acid residue from the wild-type sequence(s) is substituted with a different amino acid residue that has similar physical and chemical properties, i.e., an amino acid residue that is a member of the same class or category, as defined above. For example, a conservative mutant may be a polypeptide or combination of polypeptides that differs in amino acid sequence from the wild-type sequence(s) by the substitution of a specific aromatic Phe (F) residue with an aromatic Tyr (Y) or Trp (W) residue.

“Non-Conservative Mutant” refers to a mutant in which at least one amino acid residue from the wild-type sequence(s) is substituted with a different amino acid residue that has dissimilar physical and/or chemical properties, i.e., an amino acid residue that is a member of a different class or category, as defined above. For example, a non-conservative mutant may be a polypeptide or combination of polypeptides that differs in amino acid sequence from the wild-type sequence by the substitution of an acidic Glu (E) residue with a basic Arg (R), Lys (K) or Orn residue.

“Deletion Mutant” refers to a mutant having an amino acid sequence or sequences that differs from the wild-type sequence(s) by the deletion of one or more amino acid residues from the wild-type sequence(s). The residues may be deleted from internal regions of the wild-type sequence(s) and/or from one or both termini.

“Truncated Mutant” refers to a deletion mutant in which the deleted residues are from the N- and/or C-terminus of the wild-type sequence(s).

“Extended Mutant” refers to a mutant in which additional residues are added to the N- and/or C-terminus of the wild-type sequence(s).

“Methionine mutant” refers to (1) a mutant in which at least one methionine residue of the wild-type sequence(s) is replaced with another residue, preferably with an aliphatic residue, most preferably with a Leu (L) or Ile (I) residue; or (2) a mutant in which a non-methionine residue, preferably an aliphatic residue, most preferably a Leu (L) or Ile (I) residue, of the wild-type sequence(s) is replaced with a methionine residue.

“Selenomethionine mutant” refers to (1) a mutant which includes at least one selenomethionine (SeMet) residue, typically by substitution of a Met residue of the wild-type sequence(s)with a SeMet residue, or by addition of one or more SeMet residues at one or both termini, or (2) a methionine mutant in which at least one Met residue is substituted with a SeMet residue. Preferred SeMet mutants are those in which each Met residue is substituted with a SeMet residue.

“Cysteine mutant” refers to (1) a mutant in which at least one cysteine residue of the wild-type sequence(s) is replaced with another residue, preferably with a Ser (S) residue; or (2) a mutant in which a non-cysteine residue, preferably a Ser (S) residue, of the wild-type sequence(s) is replaced with a cysteine residue.

“Selenocysteine mutant” refers to (1) a mutant which includes at least one selenocysteine (SeCys) residue, typically by substitution of a Cys residue of the wild-type sequence(s) with a SeCys residue, or by addition of one or more SeCys residues at one or both termini, or (2) a cysteine mutant in which at least one Cys residue is substituted with a SeCys residue. Preferred SeCys mutants are those in which each Cys residue is substituted with a SeCys residue.

“Homologue” refers to a polypeptide having at least 80% amino acid sequence identity or having a BLAST score of 1×10⁻⁶ over at least 100 amino acids (Altschul et al., 1997, Nucleic Acids Res. 25:3389-402) with human IgG Fc variant or any functional domain, e.g., C_{H}2 or C_{H}3, of Fc region.

“3F2” refers to a humanized IgG1 antibody specific for human EphA2. 3F2 comprises an immunoglobulin complex of a 3F2 heavy chain comprising the amino acid sequence of SEQ ID NO: 3 and a 3F2 light chain comprising the amino acid sequence of SEQ ID NO: 4. The 3F2 antibody may comprise a a wild type human IgG Fc region or a human IgG Fc variant region.

“Antibody-dependent cell-mediated cytotoxicity” or “ADCC” refers to a form of cytotoxicity in which secreted Ig bound onto Fc receptors (FcRs) present on certain cytotoxic cells (e.g., Natural Killer (NK) cells, neutrophils, and macrophages) enables these cytotoxic effector cells to bind specifically to an antigen-bearing target cell and subsequently kill the target cell with cytotoxins. Specific high-affinity IgG antibodies directed to the surface of target cells “arm” the cytotoxic cells and are absolutely required for such killing. Lysis of the target cell is extracellular, requires direct cell-to-cell contact, and does not involve complement.

The ability of any particular antibody to mediate lysis of the target cell by ADCC can be assayed. To assess ADCC activity an antibody of interest is added to target cells in combination with immune effector cells, which may be activated by the antigen antibody complexes resulting in cytolysis of the target cell. Cytolysis is generally detected by the release of label (e.g. radioactive substrates, fluorescent dyes or natural intracellular proteins) from the lysed cells. Useful effector cells for such assays include peripheral blood mononuclear cells (PBMC) and Natural Killer (NK) cells. Specific examples of in vitro ADCC assays are described in Wisecarver et al., 1985, 79:277; Bruggemann et al., 1987, J. Exp Med 166:1351; Wilkinson et al., 2001, J Immunol Methods 258:183; Patel et al., 1995 J Immunol Methods 184:29 (each of which is incorporated by reference) and herein (see example 3). Alternatively, or additionally, ADCC activity of the antibody of interest may be assessed in vivo, e.g., in a animal model such as that disclosed in Clynes et al., 1998, PNAS USA 95:652, the contents of which is incorporated by reference in its entirety.

“Association” refers to a condition of proximity between a chemical entity or compound, or portions or fragments thereof, and a polypeptide, or portions or fragments thereof. The association may be non-covalent, i.e., where the juxtaposition is energetically favored by, e.g., hydrogen-bonding, van der Waals, electrostatic or hydrophobic interactions, or it may be covalent.

“Complex” refers to a complex between a human IgG Fc variant and a binding compound, for example, a FcγR.

“Crystal” refers to a composition comprising a polypeptide complex in crystalline form. The term “crystal” includes native crystals, heavy-atom derivative crystals and poly-crystals, as defined herein.

“Crystallized human IgG Fc variant” refers to a human IgG Fc variant which is in the crystalline form.

“Native Crystal” refers to a crystal wherein the polypeptide complex is substantially pure. As used herein, native crystals do not include crystals of polypeptide complexes comprising amino acids that are modified with heavy atoms, such as crystals of selenomethionine mutants, selenocysteine mutants, etc.

“Heavy-atom Derivative Crystal” refers to a crystal wherein the polypeptide complex is in association with one or more heavy-metal atoms. As used herein, heavy-atom derivative crystals include native crystals into which a heavy metal atom is soaked, as well as crystals of selenomethionine mutants and selenocysteine mutants.

“Co-Crystal” refers to a composition comprising a complex, as defined above, in crystalline form. Co-crystals include native co-crystals and heavy-atom derivative co-crystals.

“Diffraction Quality Crystal” refers to a crystal that is well-ordered and of a sufficient size, i.e., at least 10 μm, preferably at least 50 μm, and most preferably at least 100 μm in its smallest dimension such that it produces measurable diffraction to at least 3 Å resolution, preferably to at least 2 Å resolution, and most preferably to at least 1.5 Å resolution or lower. Diffraction quality crystals include native crystals, heavy-atom derivative crystals, and poly-crystals.

“Unit Cell” refers to the smallest and simplest volume element (i.e., parallelpiped-shaped block) of a crystal that is completely representative of the unit or pattern of the crystal, such that the entire crystal can be generated by translation of the unit cell. The dimensions of the unit cell are defined by six numbers: dimensions a, b and c and angles α, β and γ (Blundel et al., 1976, Protein Crystallography, Academic Press). A crystal is an efficiently packed array of many unit cells.

“Triclinic Unit Cell” refers to a unit cell in which a≠b≠c and α≠β≠γ.

“Monoclinic Unit Cell” refers to a unit cell in which a≠b≠c; α=γ=90°; and β≠90°, defined to be ≧90°.

“Orthorhombic Unit Cell” refers to a unit cell in which a≠b≠c; and α=β=γ=90°.

“Tetragonal Unit Cell” refers to a unit cell in which a=b=c; and α=β=γ=90°.

“Trigonal/Rhombohedral Unit Cell” refers to a unit cell in which a=b=c; and α=β=γ90°.

“Trigonal/Hexagonal Unit Cell” refers to a unit cell in which a=b=c; α=β=γ90°; and γ=120°.

“Cubic Unit Cell” refers to a unit cell in which a=b=c; and α=β=γ=90°.

“Crystal Lattice” refers to the array of points defined by the vertices of packed unit cells.

“Space Group” refers to the set of symmetry operations of a unit cell. In a space group designation (e.g., C2) the capital letter indicates the lattice type and the other symbols represent symmetry operations that can be carried out on the unit cell without changing its appearance.

“Asymmetric Unit” refers to the largest aggregate of molecules in the unit cell that possesses no symmetry elements that are part of the space group symmetry, but that can be juxtaposed on other identical entities by symmetry operations.

“Crystallographically-Related Dimer” refers to a dimer of two molecules wherein the symmetry axes or planes that relate the two molecules comprising the dimer coincide with the symmetry axes or planes of the crystal lattice.

“Non-Crystallographically-Related Dimer” refers to a dimer of two molecules wherein the symmetry axes or planes that relate the two molecules comprising the dimer do not coincide with the symmetry axes or planes of the crystal lattice.

“Isomorphous Replacement” refers to the method of using heavy-atom derivative crystals to obtain the phase information necessary to elucidate the three-dimensional structure of a crystallized polypeptide (Blundel et al., 1976, Protein Crystallography, Academic Press).

“Multi-Wavelength Anomalous Dispersion or MAD” refers to a crystallographic technique in which X-ray diffraction data are collected at several different wavelengths from a single heavy-atom derivative crystal, wherein the heavy atom has absorption edges near the energy of incoming X-ray radiation. The resonance between X-rays and electron orbitals leads to differences in X-ray scattering from absorption of the X-rays (known as anomalous scattering) and permits the locations of the heavy atoms to be identified, which in turn provides phase information for a crystal of a polypeptide. A detailed discussion of MAD analysis can be found in Hendrickson, 1985, Trans. Am. Crystallogr. Assoc., 21:11; Hendrickson et al., 1990, EMBO J. 9:1665; and Hendrickson, 1991, Science 4:91.

“Single Wavelength Anomalous Dispersion or SAD” refers to a crystallographic technique in which X-ray diffraction data are collected at a single wavelength from a single native or heavy-atom derivative crystal, and phase information is extracted using anomalous scattering information from atoms such as sulfur or chlorine in the native crystal or from the heavy atoms in the heavy-atom derivative crystal. The wavelength of X-rays used to collect data for this phasing technique need not be close to the absorption edge of the anomalous scatterer. A detailed discussion of SAD analysis can be found in Brodersen et al., 2000, Acta Cryst., D56:431-441.

“Single Isomorphous Replacement With Anomalous Scattering or SIRAS” refers to a crystallographic technique that combines isomorphous replacement and anomalous scattering techniques to provide phase information for a crystal of a polypeptide. X-ray diffraction data are collected at a single wavelength, usually from a single heavy-atom derivative crystal. Phase information obtained only from the location of the heavy atoms in a single heavy-atom derivative crystal leads to an ambiguity in the phase angle, which is resolved using anomalous scattering from the heavy atoms. Phase information is therefore extracted from both the location of the heavy atoms and from anomalous scattering of the heavy atoms. A detailed discussion of SIRAS analysis can be found in North, 1965, Acta Cryst. 18:212-216; Matthews, 1966, Acta Cryst. 20:82-86.

“Molecular Replacement” refers to the method of calculating initial phases for a new crystal of a polypeptide whose structure coordinates are unknown by orienting and positioning a polypeptide whose structure coordinates are known within the unit cell of the new crystal so as to best account for the observed diffraction pattern of the new crystal. Phases are then calculated from the oriented and positioned polypeptide and combined with observed amplitudes to provide an approximate Fourier synthesis of the structure of the polypeptides comprising the new crystal. (Jones et al., 1991, Acta Crystallogr. 47:753-70; Brunger et al., 1998, Acta Crystallogr. D. Biol. Crystallogr. 54:905-21)

“Having substantially the same three-dimensional structure” refers to a polypeptide that is characterized by a set of atomic structure coordinates that have a root mean square deviation (r.m.s.d.) of less than or equal to about 2 Å when superimposed onto the atomic structure coordinates of Table 5 when at least about 50% to 100% of the Cα atoms of the coordinates are included in the superposition.

“Cα:” As used herein, “Cα” refers to the alpha carbon of an amino acid residue.

“Purified,” when used in relation to an antibody, refers to a composition of antibodies that each have substantially similar specificities; e.g., the antibodies in the composition each bind essentially the same epitope. One method to obtain a purified antibody is to affinity purify the antibody from a polyclonal antibody preparation using a molecule that comprises the epitope of interest but not undesirable epitope(s). For example, a molecule comprising a neutralizing epitope but not an enhancing epitope can be used to obtain a purified antibody that binds the neutralizing epitope that is substantially free (e.g., antibodies of other specificity constitute less than about 0.1% of the total preparation) of antibodies that specifically bind the enhancing epitope.

### 5. BRIEF DESCRIPTION OF THE FIGURES

FIG. 1A provides a stereographic view of the asymmetric unit contents of the Fc/3M crystal. The S239D/A330L/1332E substitutions comprising 3M are indicated in red.

FIG. 1B provides a three-dimensional view of the entire Fc/3M molecule. The conventional ‘horseshoe’-shaped homodimeric Fc region was achieved by invoking a crystallographic symmetry operator. Positions corresponding to 3M are indicated by arrows.

FIG. 1C provides a stereographic view of the carbohydrate residues attached to N297, after modeling according to their electron density. GlcNAc: N-acetyl glucosamine; Fuc: fucose; Gal: galactose; Man: mannose. This and subsequent illustrations were prepared using PyMOL (DeLano, 2002, The PyMOL Molecular Graphics System, DeLano Scientific, Palo Alto, Calif., USA. http://www.pymol.org).

FIG. 2 provides a local environment around H310 and H435 in Fc/3M. One Zn²⁺ ion is chelated by both spatially-close histidine residues. The arrow indicate the conformation of the Fc polypeptide in the absence of histidine-chelating ions, as seen in the human Fc structure corresponding to PDB ID number 2DTQ (Matsumiya et al., 2007. Mol. Biol. 368, 767-779). WAT stands for water; ZN stands for zinc ion.

FIG. 3 provides stereographic representation of various human Fc regions superimposed through their respective C_{H}3 domain. All other publicly available human Fc structures not shown here exhibited intermediate structural flexibility.

FIG. 4 provides overlay of the DSC thermograms for 3F2, 3F2/3M, 3F2/Fab, Fc/3M and unmutated human Fc. The corresponding Tm values are reported in Table 4. For comparison purposes, all thermograms with the exception of 3F2 were moved along the ordinate axis.

FIGS. 5A and 5B provide a stereographic model of Fc/3M residues potentially involved in the interaction with human CD16, assuming a similar interface when compared with unmutated human Fc. The model was constructed by superimposing the Cct atoms of Fc/3M and 1E4K (Sondermann et al. 2000, Nature 406, 267-273) C_{H}2 domains (residues 236 through 342) using “lsqkab”. See Kabsch, W. 1976, Acta Cryst. A32, 922-923 For each chain, the rms displacement was estimated at 1.94 Å with a maximum displacement of 6.0 Å for Cct/286 in chain A and of 6.4 Å for Cα/286 in chain B.

FIG. 5A provides that one chain of Fc/3M (at the top) utilizes the entire set of the S239D/A330L/1332E triple mutation to contact human CD16 (at the bottom).

FIG. 5B provides that the other chain of Fc/3M (at the top) establishes additional contacts with human CD16 (at the bottom) through the S239D substitution. In both (A) and (B) panels, the carbohydrate residues are indicated by arrows.

FIG. 6 provides the amino acid sequences of wild type human IgG Fc region (T223 to K447) (SEQ ID NO: 2). Amino acid residues 239D, 330L and 332E are bolded underlined.

FIG. 7 provides the amino acid sequences of Fc/3M with S239D, A330L and I332E amino acid substitution (SEQ ID NO: 1) used in Examples.

FIG. 8 provides a diffraction pattern of the Fc/3M as described in the Examples.

FIG. 9 provides electron density maps for the region of Fc/3M comprising the three amino acid substitution of S239D, A330L and I332E. The corresponding residues are shown as sticks. The map is contoured at 1.0 σ.

FIG. 10 provides the amino acid sequences of Fc/Mut1 (Panel A), FcMut2 (Panel B) and FcMut3 (Panel C). Amino acid deletions are shown as dashes; substitutions are bolded and underlined.

FIG. 11 provides the binding affinity of wild type human IgG Fc and several mutations to CD16 as determined by surface plasmon resonance detection using a BlAcore 3000 instrument. The binding of the wild type human IgG Fc at increasing concentrations of CD16 (1 nm to 8 μM, each in duplicate) are shown in panel A while the results for Mut1, Mut2 and Mut3 are shown in panels B, C and D respectively.

FIG. 12 provides the binding affinity of wild type human IgG Fc and several mutations to FcγRI as determined by surface plasmon resonance detection using a BIAcore 3000 instrument. The binding of the wild type human IgG Fc, Mut1 and Mut2 at a single concentration of FcγRI (8 μM) are shown in panel A while the results for the wild type human IgG Fc and Mut3 are shown in panel B.

FIG. 13 provides the binding affinity of wild type human IgG Fc and several mutations to FcγRIIA as determined by surface plasmon resonance detection using a BIAcore 3000 instrument. The binding of the wild type human IgG Fc, Mut1 and Mut2 at a single concentration of FcγRIIA (8 μM) are shown in panel A while the results for the wild type human IgG Fc and Mut3 are shown in panel B.

FIG. 14 provides the binding affinity of wild type human IgG Fc and several mutations to FcγRIIB as determined by surface plasmon resonance detection using a BIAcore 3000 instrument. The binding of the wild type human IgG Fc, Mut1 and Mut2 at a single concentration of FcγRIIB (8 μM) are shown in panel A while the results for the wild type human IgG Fc and Mut3 are shown in panel B.

### 5.1 BRIEF DESCRIPTION OF THE TABLES

Table 1 provides classification of commonly encountered amino acids;

Table 2 summarizes the X-ray crystallography data sets of Fc/3M crystals that were used to determine the structures of the crystalline Fc/3M of the invention.

Table 3 summarizes the X-ray crystallography refinement parameters of the structures of crystalline Fc-3M of the invention.

Table 4 provides the thermal melting temperature Tm of unmutated human Fc, Fc/3M and 3F2 variant.

Table 5 provides the atomic structure coordinates of native Fc/3M crystals of the invention as determined by X-ray crystallography.

Table 6 provides structural properties of various human IgG and IgG/Fc molecules.

Table 7 provides dissociation constants for the binding of unmutated human Fc and Fc/3M to human CD16 (V158).

### 6. DETAILED DESCRIPTION OF THE INVENTION

### 6.1 CRYSTALLINE FC VARIANT

The present invention provides crystalline forms of a human IgG Fc variant, wherein the human IgG Fc variant comprises one or more high effector function amino acid residue and has an increased binding affinity for an FcγR as compared to a wild type human IgG Fc region not comprising the one or more high effector function amino acid residue. In certain embodiments, the human IgG Fc variant comprises at least one high effector function amino acid residue selected from the group consisting of 239D, 330L or 332E, as numbered by the EU index as set forth in Kabat. In certain embodiments, the human IgG Fc variant comprises each of the high effector function amino acid residue mutations 239D, 330L and 332E, as numbered by the EU index as set forth in Kabat. In particular embodiments, the Fc variant comprises the amino acid sequence of SEQ ID NO:1.

The crystals of the invention may be obtained include native crystals and heavy-atom crystals. Native crystals generally comprise substantially pure polypeptides corresponding to the human IgG Fc variant in crystalline form. In certain embodiments, the crystals of the invention are native crystals. In certain embodiments, the crystals of the invention are heavy-atom crystals.

It is to be understood that the crystalline of human IgG Fc variant may comprise one or mutation other than the high effector function amino acid residues. Indeed, the crystals may comprise mutants of human IgG Fc variant. Mutants of human IgG Fc variant are obtained by replacing at least one amino acid residue in the sequence of human IgG Fc variant with a different amino acid residue, or by adding or deleting one or more amino acid residues within the wild-type sequence and/or at the N- and/or C-terminus of the wild-type Fc region. Preferably, such mutants will crystallize under crystallization conditions that are substantially similar to those used to crystallize the corresponding human IgG Fc variant.

The types of mutants contemplated by this invention include conservative mutants, non-conservative mutants, deletion mutants, truncated mutants, extended mutants, methionine mutants, selenomethionine mutants, cysteine mutants and selenocysteine mutants. Preferably, a mutant displays biological activity that is substantially similar to that of the corresponding human IgG Fc variant. Methionine, selenomethionine, cysteine, and selenocysteine mutants are particularly useful for producing heavy-atom derivative crystals, as described in detail, below.

It will be recognized by one of skill in the art that the types of mutants contemplated herein are not mutually exclusive; that is, for example, a polypeptide having a conservative mutation in one amino acid may in addition have a truncation of residues at the N-terminus, and several Leu or Ile→Met mutations.

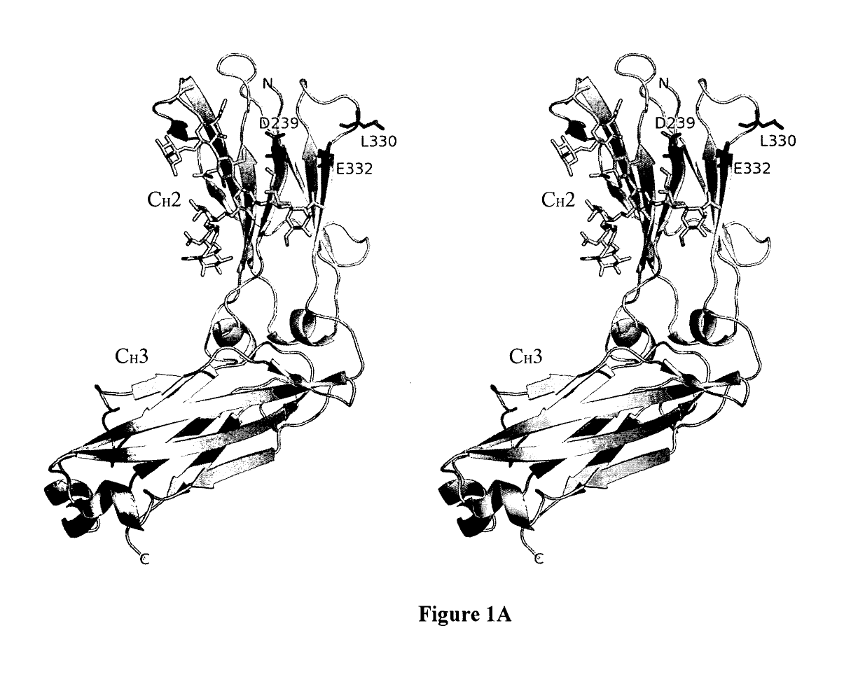

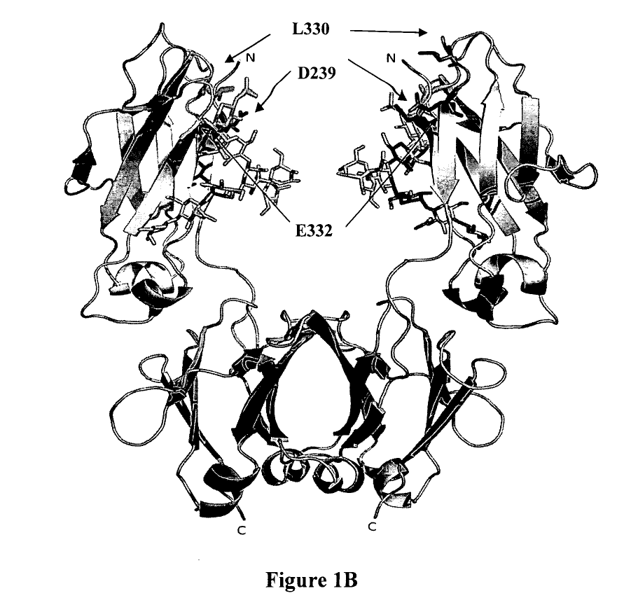

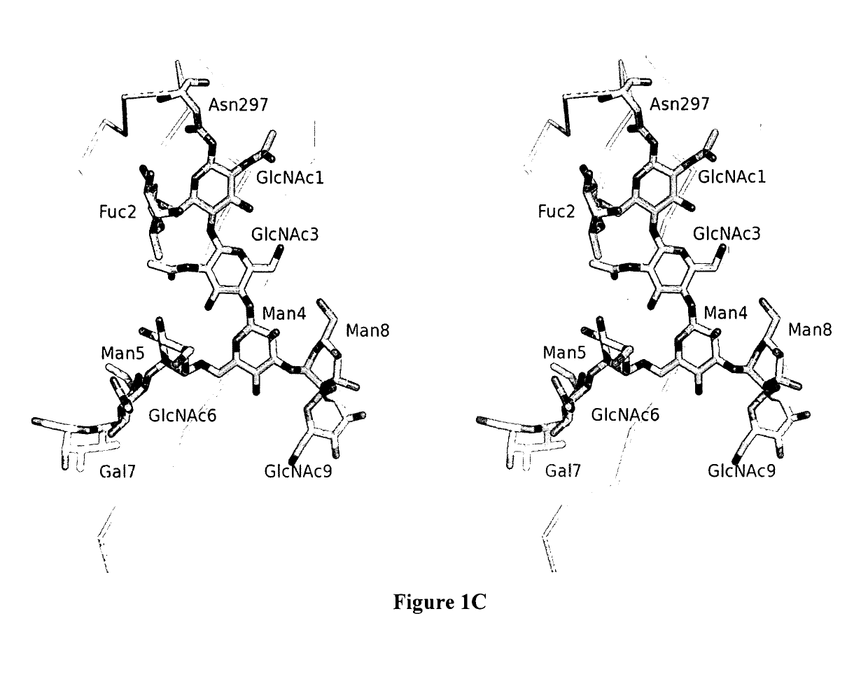

Sequence alignments of polypeptides in a protein family or of homologous polypeptide domains can be used to identify potential amino acid residues in the polypeptide sequence that are candidates for mutation. Identifying mutations that do not significantly interfere with the three-dimensional structure of the human IgG Fc variant and/or that do not deleteriously affect, and that may even enhance, the activity of the human IgG Fc variant will depend, in part, on the region where the mutation occurs. In framework regions, or regions containing significant secondary structure, such as those regions shown in FIG. 1, conservative amino acid substitutions are preferred.

Conservative amino acid substitutions are well-known in the art, and include substitutions made on the basis of a similarity in polarity, charge, solubility, hydrophobicity and/or the hydrophilicity of the amino acid residues involved. Typical conservative substitutions are those in which the amino acid is substituted with a different amino acid that is a member of the same class or category, as those classes are defined herein. Thus, typical conservative substitutions include aromatic to aromatic, apolar to apolar, aliphatic to aliphatic, acidic to acidic, basic to basic, polar to polar, etc. Other conservative amino acid substitutions are well known in the art. It will be recognized by those of skill in the art that generally, a total of about 20% or fewer, typically about 10% or fewer, most usually about 5% or fewer, of the amino acids in the wild-type polypeptide sequence can be conservatively substituted with other amino acids without deleteriously affecting the biological activity and/or three-dimensional structure of the molecule, provided that such substitutions do not involve residues that are critical for activity, as discussed above.

In some embodiments, it may be desirable to make mutations in the active site of a protein, e.g., to reduce or completely eliminate protein activity. Mutations that will reduce or completely eliminate the activity of a particular protein will be apparent to those of skill in the art.

The amino acid residue Cys (C) is unusual in that it can form disulfide bridges with other Cys (C) residues or other sulfhydryl-containing amino acids (“cysteine-like amino acids”). The ability of Cys (C) residues and other cysteine-like amino acids to exist in a polypeptide in either the reduced free —SH or oxidized disulfide-bridged form affects whether Cys (C) residues contribute net hydrophobic or hydrophilic character to a polypeptide. While Cys (C) exhibits a hydrophobicity of 0.29 according to the consensus scale of Eisenberg (Eisenberg, 1984, supra), it is to be understood that for purposes of the present invention Cys (C) is categorized as a polar hydrophilic amino acid, notwithstanding the general classifications defined above. Preferably, Cys residues that are known to participate in disulfide bridges, such as those linking the heavy chain to the light chain of an antibody, or a portion thereof, are not substituted or are conservatively substituted with other cysteine-like amino acids so that the residue can participate in a disulfide bridge. Typical cysteine-like residues include, for example, Pen, hCys, etc. Substitutions for Cys residues that interfere with crystallization are discussed infra.

While in most instances the amino acids of human IgG Fc variant will be substituted with genetically-encoded amino acids, in certain circumstances mutants may include genetically non-encoded amino acids. For example, non-encoded derivatives of certain encoded amino acids, such as SeMet and/or SeCys, may be incorporated into the polypeptide chain using biological expression systems (such SeMet and SeCys mutants are described in more detail, infra).

Alternatively, in instances where the mutant will be prepared in whole or in part by chemical synthesis, virtually any non-encoded amino acids may be used, ranging from D-isomers of the genetically encoded amino acids to non-encoded naturally-occurring natural and synthetic amino acids.

Conservative amino acid substitutions for many of the commonly known non-genetically encoded amino acids are well known in the art. Conservative substitutions for other non-encoded amino acids can be determined based on their physical properties as compared to the properties of the genetically encoded amino acids.

In some instances, it may be particularly advantageous or convenient to substitute, delete from and/or add amino acid residues to human IgG Fc variant in order to provide convenient cloning sites in cDNA encoding the polypeptide, to aid in purification of the polypeptide, etc. Such substitutions, deletions and/or additions that do not substantially alter the three dimensional structure of the wile type human IgG Fc region will be apparent to those having skills in the art. These substitutions, deletions and/or additions include, but are not limited to, His tags, BirA tags, intein-containing self-cleaving tags, maltose binding protein fusions, glutathione S-transferase protein fusions, antibody fusions, green fluorescent protein fusions, signal peptide fusions, biotin accepting peptide fusions, and the like. In certain embodiments, the human IgG Fc variant comprises a His tag. In other embodiments, the human IgG Fc variant comprises a BirA tag. In a preferred embodiment, the human IgG Fc variant comprises a His tag and a BirA tag.

Mutations may also be introduced into a polypeptide sequence where there are residues, e.g., cysteine residues, that interfere with crystallization. Such cysteine residues can be substituted with an appropriate amino acid that does not readily form covalent bonds with other amino acid residues under crystallization conditions; e.g., by substituting the cysteine with Ala, Ser or Gly. Any cysteine located in a non-helical or non-β-stranded segment, based on secondary structure assignments, are good candidates for replacement.

The heavy-atom derivative crystals from which the atomic structure coordinates of the invention are obtained generally comprise a crystalline human IgG Fc variant. There are two types of heavy-atom derivatives of polypeptides: heavy-atom derivatives resulting from exposure of the protein to a heavy metal in solution, wherein crystals are grown in medium comprising the heavy metal, or in crystalline form, wherein the heavy metal diffuses into the crystal, and heavy-atom derivatives wherein the polypeptide comprises heavy-atom containing amino acids, e.g., selenomethionine and/or selenocysteine mutants.

In practice, heavy-atom derivatives of the first type can be formed by soaking a native crystal in a solution comprising heavy metal atom salts, or organometallic compounds, e.g., lead chloride, gold thiomalate, ethylmercurithiosalicylic acid-sodium salt (thimerosal), uranyl acetate, platinum tetrachloride, osmium tetraoxide, zinc sulfate, and cobalt hexamine, which can diffuse through the crystal and bind to the crystalline polypeptide complex.

Heavy-atom derivatives of this type can also be formed by adding to a crystallization solution comprising the polypeptide complex to be crystallized an amount of a heavy metal atom salt, which may associate with the protein complex and be incorporated into the crystal. The location(s) of the bound heavy metal atom(s) can be determined by X-ray diffraction analysis of the crystal. This information, in turn, is used to generate the phase information needed to construct the three-dimensional structure of the protein.

Heavy-atom derivative crystals may also be prepared from human IgG Fc variant. Such selenocysteine or selenomethionine mutants may be made from human IgG Fc variant or its mutant by expression of human IgG Fc variant in auxotrophic E. coli strains. Hendrickson et al., 1990, EMBO J. 9:1665-1672. In this method, the human IgG Fc variant or its mutant may be expressed in a host organism on a growth medium depleted of either natural cysteine or methionine (or both) but enriched in selenocysteine or selenomethionine (or both). Alternatively, selenocysteine or selenomethionine mutants may be made using nonauxotrophic E. coli strains, e.g., by inhibiting methionine biosynthesis in these strains with high concentrations of Ile, Lys, Phe, Leu, Val or Thr and then providing selenomethionine in the medium (Doublié, 1997, Methods in Enzymology 276:523-530). Furthermore, selenocysteine can be selectively incorporated into polypeptides by exploiting the prokaryotic and eukaryotic mechanisms for selenocysteine incorporation into certain classes of proteins in vivo, as described in U.S. Pat. No. 5,700,660 to Leonard et al. (filed Jun. 7, 1995). One of skill in the art will recognize that selenocysteine is preferably not incorporated in place of cysteine residues that form disulfide bridges, as these may be important for maintaining the three-dimensional structure of the protein and are preferably not to be eliminated. One of skill in the art will further recognize that, in order to obtain accurate phase information, approximately one selenium atom should be incorporated for every 140 amino acid residues of the polypeptide chain. The number of selenium atoms incorporated into the polypeptide chain can be conveniently controlled by designing a Met or Cys mutant having an appropriate number of Met and/or Cys residues, as described more fully below.

In some instances, a polypeptide to be crystallized may not contain cysteine or methionine residues. Therefore, if selenomethionine and/or selenocysteine mutants are to be used to obtain heavy-atom derivative crystals, methionine and/or cysteine residues must be introduced into the polypeptide chain. Likewise, Cys residues may be introduced into the polypeptide chain if the use of a cysteine-binding heavy metal, such as mercury, is contemplated for production of a heavy-atom derivative crystal.

Such mutations are preferably introduced into the polypeptide sequence at sites that will not disturb the overall protein fold. For example, a residue that is conserved among many members of the protein family or that is thought to be involved in maintaining its activity or structural integrity, as determined by, e.g., sequence alignments, should not be mutated to a Met or Cys. In addition, conservative mutations, such as Ser to Cys, or Leu or Ile to Met, are preferably introduced. One additional consideration is that, in order for a heavy-atom derivative crystal to provide phase information for structure determination, the location of the heavy atom(s) in the crystal unit cell should be determinable and provide phase information. Therefore, a mutation is preferably not introduced into a portion of the protein that is likely to be mobile, e.g., at, or within about 1-5 residues of, the N- and C-termini.

Conversely, if there are too many methionine and/or cysteine residues in a polypeptide sequence, over-incorporation of the selenium-containing side chains can lead to the inability of the polypeptide to fold and/or crystallize, and may potentially lead to complications in solving the crystal structure. In this case, methionine and/or cysteine mutants are prepared by substituting one or more of these Met and/or Cys residues with another residue. The considerations for these substitutions are the same as those discussed above for mutations that introduce methionine and/or cysteine residues into the polypeptide. Specifically, the Met and/or Cys residues are preferably conservatively substituted with Leu/Ile and Ser, respectively.

As DNA encoding cysteine and methionine mutants can be used in the methods described above for obtaining SeCys and SeMet heavy-atom derivative crystals, the preferred Cys or Met mutant will have one Cys or Met residue for every 140 amino acids.

### 6.2 PRODUCTION OF POLYPEPTIDES

The human IgG Fc variants or mutants thereof may be chemically synthesized in whole or part using techniques that are well-known in the art (see, e.g., Creighton, 1983, Proteins: Structures and Molecular Principles, W.H. Freeman & Co., NY.). Alternatively, methods that are well known to those skilled in the art can be used to construct expression vectors containing the human IgG Fc variant polypeptide coding sequence and appropriate transcriptional/translational control signals. These methods include in vitro recombinant DNA techniques, synthetic techniques and in vivo recombination/genetic recombination. See, for example, the techniques described in the current editions of Sambrook et al., 2001, Molecular Cloning: A Laboratory Manual, 3d Ed., Cold Spring Harbor Laboratory, NY and Ausubel et al., 2004, Current Protocols in Molecular Biology, Greene Publishing Associates and Wiley Interscience, NY. The human IgG Fc variant may also be produced by digesting an IgG with papain.

A variety of host-expression vector systems may be utilized to express the human IgG Fc variant coding sequences. These include but are not limited to microorganisms such as bacteria transformed with recombinant bacteriophage DNA, plasmid DNA or cosmid DNA expression vectors containing the human IgG Fc region coding sequences; yeast transformed with recombinant yeast expression vectors containing the Fc coding sequences; insect cell systems infected with recombinant virus expression vectors (e.g., baculovirus) containing the Fc coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing the Fc coding sequences; or animal cell systems. The expression elements of these systems vary in their strength and specificities.

Specifically designed vectors allow the shuttling of DNA between hosts such as bacteria-yeast or bacteria-animal cells. An appropriately constructed expression vector may contain: an origin of replication for autonomous replication in host cells, selectable markers, a limited number of useful restriction enzyme sites, a potential for high copy number, and active promoters. A promoter is defined as a DNA sequence that directs RNA polymerase to bind to DNA and initiate RNA synthesis. A strong promoter is one that causes mRNAs to be initiated at high frequency.

Depending on the host/vector system utilized, any of a number of suitable transcription and translation elements, including constitutive and inducible promoters, may be used in the expression vector. For example, when cloning in bacterial systems, inducible promoters such as the T7 promoter, pL of bacteriophage λ, plac, ptrp, ptac (ptrp-lac hybrid promoter) and the like may be used; when cloning in insect cell systems, promoters such as the baculovirus polyhedrin promoter may be used; when cloning in plant cell systems, promoters derived from the genome of plant cells (e.g., heat shock promoters; the promoter for the small subunit of RUBISCO; the promoter for the chlorophyll a/b binding protein) or from plant viruses (e.g., the 35S RNA promoter of CaMV; the coat protein promoter of TMV) may be used; when cloning in mammalian cell systems, promoters derived from the genome of mammalian cells (e.g., metallothionein promoter) or from mammalian viruses (e.g., the adenovirus late promoter; the vaccinia virus 7.5K promoter) may be used; when generating cell lines that contain multiple copies of the tyrosine kinase domain DNA, SV40-, BPV- and EBV-based vectors may be used with an appropriate selectable marker.

The expression vector may be introduced into host cells via any one of a number of techniques including but not limited to transformation, transfection, infection, protoplast fusion, and electroporation. The expression vector-containing cells are clonally propagated and individually analyzed to determine whether they produce human IgG Fc variant. Identification of human IgG Fc variant-expressing host cell clones may be done by several means, including but not limited to immunological reactivity with anti-human IgG Fc variant or anti-immunoglobulin antibodies, and the presence of host cell-associated Fc biological activity.

Expression of human IgG Fc variant may also be performed using in vitro produced synthetic mRNA. Synthetic mRNA can be efficiently translated in various cell-free systems, including but not limited to wheat germ extracts and reticulocyte extracts, as well as efficiently translated in cell based systems, including but not limited to microinjection into frog oocytes. Further, nucleic acids expressing human IgG Fc variant can be constructed and expressed by gene synthesis using oligonucleotides. See Hoover & Lubkowski, 2002, Nucleic Acids Res 30:e43.

To determine the human IgG Fc variant DNA sequences that yields optimal levels of Fc biological activity, modified Fc variant molecules are constructed. Host cells are transformed with the cDNA molecules and the levels of Fc RNA and/or protein are measured.

Levels of Fc protein in host cells are quantitated by a variety of methods such as immunoaffinity and/or ligand affinity techniques, Fc specific beads or Fc specific antibodies are used to isolate ³⁵S-methionine labeled or unlabeled Fc. Labeled or unlabeled Fc is analyzed by SDS-PAGE. Unlabeled Fc is detected by Western blotting, ELISA or RIA employing Fc-specific antibodies.

Following expression of human IgG Fc variant in a recombinant host cell, Fc may be recovered to provide human IgG Fc variant in active form. Several human IgG Fc variant purification procedures are available and suitable for use. Recombinant Fc may be purified from cell lysates or from conditioned culture media, by various combinations of, or individual application of, fractionation, or chromatography steps that are known in the art.

In addition, recombinant human IgG Fc variant can be separated from other cellular proteins by use of an immuno-affinity column made with monoclonal or polyclonal antibodies specific for full length nascent Fc or polypeptide fragments thereof.

Alternatively, human IgG Fc variant may be recovered from a host cell in an unfolded, inactive form, e.g., from inclusion bodies of bacteria. Proteins recovered in this form may be solublized using a denaturant, e.g., guanidinium hydrochloride, and then refolded into an active form using methods known to those skilled in the art, such as dialysis. See, for example, the techniques described in Sambrook et al., 2001, Molecular Cloning: A Laboratory Manual, 3d Ed., Cold Spring Harbor Laboratory, NY and Ausubel et al., 2004, Current Protocols in Molecular Biology, Greene Publishing Associates and Wiley Interscience, NY.

Still further, human IgG Fc variant can be prepared from an antibody according to any known method without limitation. Generally, Fc region are prepared by Papain digestion of an antibody; however, any technique that cleaves an antibody heavy chain at or near the hinge region can be used to prepare the Fc variants. Repetitive protocols for making Fc fragments from antibodies, including monoclonal antibodies, are described in, e.g., Harlow et al., 1988, Antibodies: A Laboratory Manual, Cold Spring Harbor Laboratory Press, 2nd ed. These techniques can be used to prepare Fc variants from an antibody according to any of the methods described herein.

### 6.3 CRYSTALLIZATION OF POLYPEPTIDES AND CHARACTERIZATION OF CRYSTAL

The native, heavy-atom derivative, and/or co-crystals from which the atomic structure coordinates of the invention can be obtained by conventional means as are well-known in the art of protein crystallography, including batch, liquid bridge, dialysis, and vapor diffusion methods (see, e.g., McPherson, 1998, Crystallization of Biological Macromolecules, Cold Spring Harbor Press, New York; McPherson, 1990, Eur. J. Biochem. 189:1-23.; Weber, 1991, Adv. Protein Chem. 41:1-36).

Generally, native crystals are grown by dissolving substantially pure human IgG Fc variant in an aqueous buffer containing a precipitant at a concentration just below that necessary to precipitate the protein. Examples of precipitants include, but are not limited to, polyethylene glycol, ammonium sulfate, 2-methyl-2,4-pentanediol, sodium citrate, sodium chloride, glycerol, isopropanol, lithium sulfate, sodium acetate, sodium formate, potassium sodium tartrate, ethanol, hexanediol, ethylene glycol, dioxane, t-butanol and combinations thereof. Water is removed by controlled evaporation to produce precipitating conditions, which are maintained until crystal growth ceases.

In a preferred embodiment, native crystals are grown by vapor diffusion in sitting drops (McPherson, 1982, Preparation and Analysis of Protein Crystals, John Wiley, New York; McPherson, 1990, Eur. J. Biochem. 189:1-23). In this method, the polypeptide/precipitant solution is allowed to equilibrate in a closed container with a larger aqueous reservoir having a precipitant concentration optimal for producing crystals. Generally, less than about 25 pL of substantially pure polypeptide solution is mixed with an equal volume of reservoir solution, giving a precipitant concentration about half that required for crystallization. The sealed container is allowed to stand, usually for about 2-6 weeks, until crystals grow.

In certain embodiments, the crystals of the present invention are produced by a method comprising the steps of (a) mixing a volume of a solution comprising a human IgG Fc variant with a volume of a reservoir solution comprising a precipitant; and (b) incubating the mixture obtained in step (a) over the reservoir solution in a closed container, under conditions suitable for crystallization until the crystal forms. The mixture comprising the Fc variant and reservoir solution can be incubated at a temperature between 0° C.-100° C., between 5° C.-50° C., 5° C.-40° C., preferably between 20° C.-25° C.

For native crystals from which the atomic structure coordinates of the invention are obtained, it has been found that hanging drops of about 2 μL containing about 1 μL of 0.9 mg/ml human IgG Fc variant in 0.1 M imidazole-malate (pH 8.0), 8% polyethylene glycol (PEG) 3350, 200 mM zinc acetate, 5% glycerol suspended over 300 μl reservoir solution for about 5 days at about 20-25° C. provide diffraction quality crystals

Of course, those having skill in the art will recognize that the above-described crystallization conditions can be varied. Such variations may be used alone or in combination, and include polypeptide solutions containing polypeptide concentrations between 0.01 mg/mL and 100 mg/mL, preferably, between 0.1 mg/ml and 10 mg/ml; imidazole malate concentrations between 0.001 mM and 10 mM, preferably, between 0.01 mM and 1 mM; zinc acetate concentrations between 1 mM and 1000 mM, preferably, between 50 mM and 500 mM; glycerol concentration between 0.1% to 50% (w/v), preferably, between 1% and 10% (w/v); pH ranges between 4.0 and 12.0, preferably, between 6.0 and 10.0; and reservoir solutions containing PEG molecular weights of 2000 to 8000, at concentrations between about 0.1% and 50% (w/v), preferably, between 6.0% and 8.0% (w/v). Other buffer solutions may be used such as HEPES, CAPS, CAPSO, BIS TRIS, MES, MOPS, MOPSO, PIPES, TRIS, and the like, so long as the desired pH range is maintained.

Heavy-atom derivative crystals can be obtained by soaking native crystals in mother liquor containing salts of heavy metal atoms.

Heavy-atom derivative crystals can also be obtained from SeMet and/or SeCys mutants, as described above for native crystals.

Mutant proteins may crystallize under slightly different crystallization conditions than wild-type protein, or under very different crystallization conditions, depending on the nature of the mutation, and its location in the protein. For example, a non-conservative mutation may result in alteration of the hydrophilicity of the mutant, which may in turn make the mutant protein either more soluble or less soluble than the wild-type protein. Typically, if a protein becomes more hydrophilic as a result of a mutation, it will be more soluble than the wild-type protein in an aqueous solution and a higher precipitant concentration will be needed to cause it to crystallize. Conversely, if a protein becomes less hydrophilic as a result of a mutation, it will be less soluble in an aqueous solution and a lower precipitant concentration will be needed to cause it to crystallize. If the mutation happens to be in a region of the protein involved in crystal lattice contacts, crystallization conditions may be affected in more unpredictable ways.

Co-crystals can be obtained by soaking a native crystal in mother liquor containing compound that binds human IgG Fc such as an FcγR, or by co-crystallizing human IgG Fc variant in the presence of one or more binding compounds

### 6.4 CHARACTERIZATION OF CRYSTALS

The dimensions of a unit cell of a crystal are defined by six numbers, the lengths of three unique edges, a, b, and c, and three unique angles, α, β, and γ. The type of unit cell that comprises a crystal is dependent on the values of these variables, as discussed above.

When a crystal is placed in an X-ray beam, the incident X-rays interact with the electron cloud of the molecules that make up the crystal, resulting in X-ray scatter. The combination of X-ray scatter with the lattice of the crystal gives rise to nonuniformity of the scatter; areas of high intensity are called diffracted X-rays. The angle at which diffracted beams emerge from the crystal can be computed by treating diffraction as if it were reflection from sets of equivalent, parallel planes of atoms in a crystal (Bragg's Law). The most obvious sets of planes in a crystal lattice are those that are parallel to the faces of the unit cell. These and other sets of planes can be drawn through the lattice points. Each set of planes is identified by three indices, hkl. The h index gives the number of parts into which the a edge of the unit cell is cut, the k index gives the number of parts into which the b edge of the unit cell is cut, and the 1 index gives the number of parts into which the c edge of the unit cell is cut by the set of hkl planes. Thus, for example, the 235 planes cut the a edge of each unit cell into halves, the b edge of each unit cell into thirds, and the c edge of each unit cell into fifths. Planes that are parallel to the be face of the unit cell are the 100 planes; planes that are parallel to the ac face of the unit cell are the 010 planes; and planes that are parallel to the ab face of the unit cell are the 001 planes.

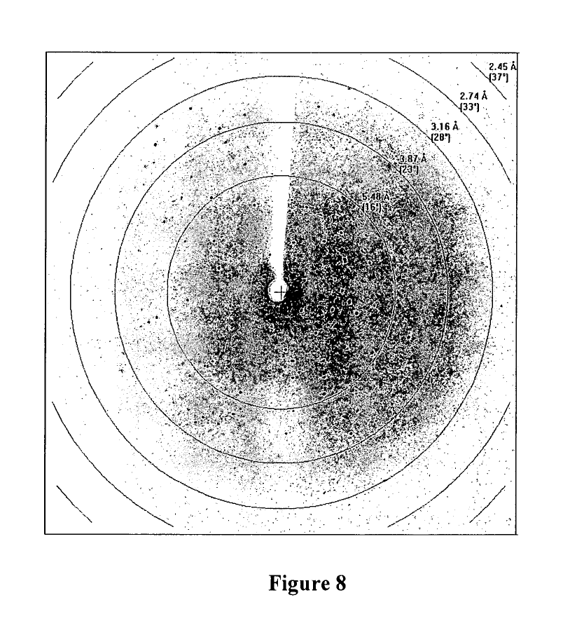

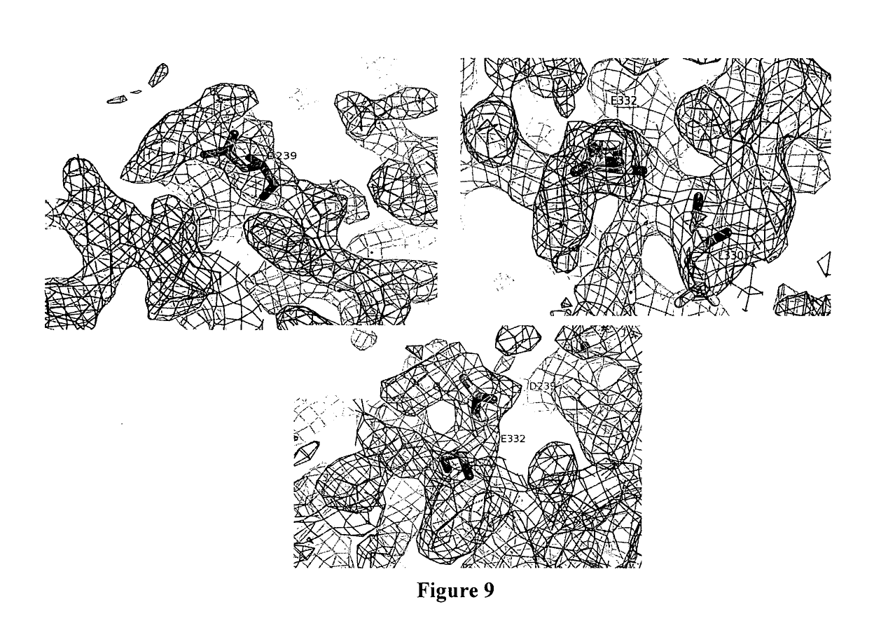

When a detector is placed in the path of the diffracted X-rays, in effect cutting into the sphere of diffraction, a series of spots, or reflections, are recorded to produce a “still” diffraction pattern. Each reflection is the result of X-rays reflecting off one set of parallel planes, and is characterized by an intensity, which is related to the distribution of molecules in the unit cell, and hkl indices, which correspond to the parallel planes from which the beam producing that spot was reflected. If the crystal is rotated about an axis perpendicular to the X-ray beam, a large number of reflections is recorded on the detector, resulting in a diffraction pattern as shown, for example, in FIG. 8.

The unit cell dimensions and space group of a crystal can be determined from its diffraction pattern. First, the spacing of reflections is inversely proportional to the lengths of the edges of the unit cell. Therefore, if a diffraction pattern is recorded when the X-ray beam is perpendicular to a face of the unit cell, two of the unit cell dimensions may be deduced from the spacing of the reflections in the x and y directions of the detector, the crystal-to-detector distance, and the wavelength of the X-rays. Those of skill in the art will appreciate that, in order to obtain all three unit cell dimensions, the crystal can be rotated such that the X-ray beam is perpendicular to another face of the unit cell. Second, the angles of a unit cell can be determined by the angles between lines of spots on the diffraction pattern. Third, the absence of certain reflections and the repetitive nature of the diffraction pattern, which may be evident by visual inspection, indicate the internal symmetry, or space group, of the crystal. Therefore, a crystal may be characterized by its unit cell and space group, as well as by its diffraction pattern.

Once the dimensions of the unit cell are determined, the likely number of polypeptides in the asymmetric unit can be deduced from the size of the polypeptide, the density of the average protein, and the typical solvent content of a protein crystal, which is usually in the range of 30-70% of the unit cell volume (Matthews, 1968, J. Mol. Biol. 33 (2):491 -497).

The human IgG Fc variant crystals of the present invention are generally characterized by a diffraction pattern that is substantially similar to the diffraction pattern as shown in FIG. 8. The crystals are further characterized by unit cell dimensions and space group symmetry information obtained from the diffraction patterns, as described above. The crystals, which may be native crystals, heavy-atom derivative crystals or poly-crystals, have an orthorhombic unit cell (i.e., unit cells wherein α≠b≠c and α=β=γ=90° and space group symmetry C222₁.

One form of crystalline human IgG Fc variant was obtained. In this form (designated “C222₁ form”), the unit cell has dimensions of a=49.87+/−0.2 Å, b=147.49+/−0.2 Å, c=74.32+/−0.2 Å. There is one human IgG Fc variant in the asymmetric unit.

### 6.5 COLLECTION OF DATA AND DETERMINATION OF STRUCTURE SOLUTIONS

The diffraction pattern is related to the three-dimensional shape of the molecule by a Fourier transform. The process of determining the solution is in essence a re-focusing of the diffracted X-rays to produce a three-dimensional image of the molecule in the crystal. Since re-focusing of X-rays cannot be done with a lens at this time, it is done via mathematical operations.

The sphere of diffraction has symmetry that depends on the internal symmetry of the crystal, which means that certain orientations of the crystal will produce the same set of reflections. Thus, a crystal with high symmetry has a more repetitive diffraction pattern, and there are fewer unique reflections that need to be recorded in order to have a complete representation of the diffraction. The goal of data collection, a dataset, is a set of consistently measured, indexed intensities for as many reflections as possible. A complete dataset is collected if at least 80%, preferably at least 90%, most preferably at least 95% of unique reflections are recorded. In one embodiment, a complete dataset is collected using one crystal. In another embodiment, a complete dataset is collected using more than one crystal of the same type.

Sources of X-rays include, but are not limited to, a rotating anode X-ray generator such as a Rigaku RU-200 or a beamline at a synchrotron light source, such as the Advanced Photon Source at Argonne National Laboratory. Suitable detectors for recording diffraction patterns include, but are not limited to, X-ray sensitive film, multiwire area detectors, image plates coated with phosphorus, and CCD cameras. Typically, the detector and the X-ray beam remain stationary, so that, in order to record diffraction from different parts of the crystal's sphere of diffraction, the crystal itself is moved via an automated system of moveable circles called a goniostat.

One of the biggest problems in data collection, particularly from macromolecular crystals having a high solvent content, is the rapid degradation of the crystal in the X-ray beam. In order to slow the degradation, data is often collected from a crystal at liquid nitrogen temperatures. In order for a crystal to survive the initial exposure to liquid nitrogen, the formation of ice within the crystal can be prevented by the use of a cryoprotectant. Suitable cryoprotectants include, but are not limited to, low molecular weight polyethylene glycols, ethylene glycol, sucrose, glycerol, xylitol, and combinations thereof. Crystals may be soaked in a solution comprising the one or more cryoprotectants prior to exposure to liquid nitrogen, or the one or more cryoprotectants may be added to the crystallization solution. Data collection at liquid nitrogen temperatures may allow the collection of an entire dataset from one crystal.

Once a dataset is collected, the information is used to determine the three-dimensional structure of the molecule in the crystal. However, this cannot be done from a single measurement of reflection intensities because certain information, known as phase information, is lost between the three-dimensional shape of the molecule and its Fourier transform, the diffraction pattern. This phase information can be acquired by methods described below in order to perform a Fourier transform on the diffraction pattern to obtain the three-dimensional structure of the molecule in the crystal. It is the determination of phase information that in effect refocuses X-rays to produce the image of the molecule.

One method of obtaining phase information is by isomorphous replacement, in which heavy-atom derivative crystals are used. In this method, the positions of heavy atoms bound to the molecules in the heavy-atom derivative crystal are determined, and this information is then used to obtain the phase information necessary to elucidate the three-dimensional structure of a native crystal. (Blundel et al., 1976, Protein Crystallography, Academic Press.)

Another method of obtaining phase information is by molecular replacement, which is a method of calculating initial phases for a new crystal of a polypeptide whose structure coordinates are unknown by orienting and positioning a polypeptide whose structure coordinates are known within the unit cell of the new crystal so as to best account for the observed diffraction pattern of the new crystal. Phases are then calculated from the oriented and positioned polypeptide and combined with observed amplitudes to provide an approximate Fourier synthesis of the structure of the molecules comprising the new crystal. (Lattman, 1985, Methods in Enzymology 115:55-77; Rossmann, 1972, “The Molecular Replacement Method,” Int. Sci. Rev. Ser. No. 13, Gordon & Breach, New York.)

A third method of phase determination is multi-wavelength anomalous diffraction or MAD. In this method, X-ray diffraction data are collected at several different wavelengths from a single crystal containing at least one heavy atom with absorption edges near the energy of incoming X-ray radiation. The resonance between X-rays and electron orbitals leads to differences in X-ray scattering that permits the locations of the heavy atoms to be identified, which in turn provides phase information for a crystal of a polypeptide. A detailed discussion of MAD analysis can be found in Hendrickson, 1985, Trans. Am. Crystallogr. Assoc. 21:11; Hendrickson et al., 1990, EMBO J. 9:1665; and Hendrickson, 1991, Science 4:91.

A fourth method of determining phase information is single wavelength anomalous dispersion or SAD. In this technique, X-ray diffraction data are collected at a single wavelength from a single native or heavy-atom derivative crystal, and phase information is extracted using anomalous scattering information from atoms such as sulfur or chlorine in the native crystal or from the heavy atoms in the heavy-atom derivative crystal. The wavelength of X-rays used to collect data for this phasing technique need not be close to the absorption edge of the anomalous scatterer. A detailed discussion of SAD analysis can be found in Brodersen et al., 2000, Acta Cryst. D56:431-441.

A fifth method of determining phase information is single isomorphous replacement with anomalous scattering or SIRAS. This technique combines isomorphous replacement and anomalous scattering techniques to provide phase information for a crystal of a polypeptide. X-ray diffraction data are collected at a single wavelength, usually from a single heavy-atom derivative crystal. Phase information obtained only from the location of the heavy atoms in a single heavy-atom derivative crystal leads to an ambiguity in the phase angle, which is resolved using anomalous scattering from the heavy atoms. Phase information is therefore extracted from both the location of the heavy atoms and from anomalous scattering of the heavy atoms. A detailed discussion of SIRAS analysis can be found in North, 1965, Acta Cryst. 18:212-216; Matthews, 1966, Acta Cryst. 20:82-86.

Once phase information is obtained, it is combined with the diffraction data to produce an electron density map, an image of the electron clouds that surround the molecules in the unit cell. The higher the resolution of the data, the more distinguishable are the features of the electron density map, e.g., amino acid side chains and the positions of carbonyl oxygen atoms in the peptide backbones, because atoms that are closer together are resolvable. A model of the macromolecule is then built into the electron density map with the aid of a computer, using as a guide all available information, such as the polypeptide sequence and the established rules of molecular structure and stereochemistry. Interpreting the electron density map is a process of finding the chemically reasonable conformation that fits the map precisely.

After a model is generated, a structure is refined. Refinement is the process of minimizing the

$${R}_{factor} = \frac{{\sum }_{hkl} ‖ {F}_{obs} ( hkl ) \vert - \vert {F}_{calc} ( hkl ) ‖}{{\sum }_{hkl} \vert {F}_{obs} ( hkl ) \vert}$$

which is the difference between observed and calculated intensity values (measured by an R-factor), and which is a function of the position, temperature factor, and occupancy of each non-hydrogen atom in the model. This usually involves alternate cycles of real space refinement, i.e., calculation of electron density maps and model building, and reciprocal space refinement, i.e., computational attempts to improve the agreement between the original intensity data and intensity data generated from each successive model. Refinement ends when the function Φ converges on a minimum wherein the model fits the electron density map and is stereochemically and conformationally reasonable. During refinement, ordered solvent molecules are added to the structure.

### 6.5.1 STRUCTURES OF HUMAN IGG FC VARIANT

The present invention provides, for the first time, the high-resolution three-dimensional structures and atomic structure coordinates of a crystalline human IgG Fc variant, particularly Fc/3M, determined by X-ray crystallography. The specific methods used to obtain the structure coordinates are provided in the examples, infra. The atomic structure coordinates of crystalline Fc/3M, obtained from the C222₁ form of the crystal to 2.5 Å resolution, are listed in Table 5.

The atomic coordinates and experimental structure factors of Fc/3M have been deposited to the Protein Data Bank under accession number 2QL1.

Those having skill in the art will recognize that atomic structure coordinates as determined by X-ray crystallography are not without error. Thus, it is to be understood that any set of structure coordinates obtained for crystals of human IgG Fc variant, whether native crystals, heavy-atom derivative crystals or poly-crystals, that have a root mean square deviation (“r.m.s.d.”) of less than or equal to about 2 Å when superimposed, using backbone atoms (N, Ca, C and O), on the structure coordinates listed in Table 5 are considered to be identical with the structure coordinates listed in the Table when at least about 50% to 100% of the backbone atoms of the constituents of the human IgG Fc variant are included in the superposition.

The overall three-dimensional structure of Fc/3M is very similar to previously reported structures of human Fc regions. See Deisenhofer et al. 1981, Biochemistry 20: 2361-2370; Sondermann et al. 2000, Nature 406, 267-273; Krapp et al. 2003, J. Mol. Biol. 325: 979-989, Matsumiya et al. 2007, J. Mol. Biol. 368, 767-779.

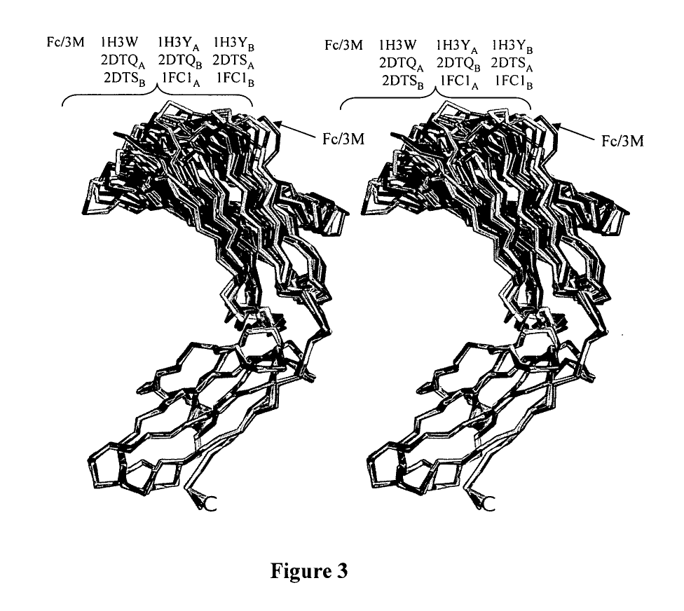

In particular, the structure of the unmutated human Fc described by Krapp et al., 2003, J. Mol. Biol. 325: 979-989, exhibited the most similarity in cell parameters, space group and packing when compared with Fc/3M. All C_{H}2 and C_{H}3 domains showed considerable structural conservation and rigidity when considered separately. A domain-by-domain comparison suggested that C_{H}3 was the most conformationally conserved domain. Indeed, superimposition of C_{H}3 domains from various crystal structures hardly showed RMS deviations in excess of 0.5-0.6 Å for C_{α}. However, C_{H}2 and C_{H}3 domains exhibited substantial relative flexibility. Fc/3M C_{H}3 domains were superimposed with those of other human Fc portions and evaluated differences in the positions of the various C_{H}2 domains, as shown in FIG. 3.

This comparison was carried out using the following human Fc structures: PDB ID numbers 1FC1 and 1FC2 (Deisenhofer et al. 1981, Biochemistry 20: 2361-2370), PDB ID numbers 1H3T/U/V/W/X/Y (Krapp et al. 2003, J. Mol. Biol. 325: 979-989), PDB ID numbers 2DTQ and 2DTS (Matsumiya et al. 2007, J. Mol. Biol. 368, 767-779), PDB ID number 1E4K (Sondermann et al. 2000, Nature 406, 267-273) and PDB ID number 1T83 (Radaev et al. 2001, J. Biol. Chem. 276, 16469-16477).

Similar results were obtained when the Fc/3M structure was compared to the human Fc structure with PDB ID number 3DO3, and the deglycosylated human Fc structure with PDB ID number 3DNK.

Fc/3M exhibited the most “open” conformation of all known Fc structures, as defined by (i) the inter-molecular distance between select portions of the polypeptide chains, and (ii) the angle between C_{H}2 and C_{H}3 domains.

The inter-molecular distance was measured of the four most open structures using the Cα atom of P329, whose close proximity to the Fc N-terminus in each polypeptide chain makes it a useful reference point. These were estimated at 39.1, 33.8, 31.3, 30.3, 23,50 and 27.60 Å, for Fc/3M, human Fc PDB ID number 1H3W (Krapp et al. 2003, J. Mol. Biol. 325: 979-989), human Fc PDB ID number 1T83 (Radaev et al. 2001, J. Biol. Chem. 276, 16469-16477), human Fc PDB ID number 1E4K (Sondermann et al. 2000, Nature 406, 267-273), human Fc PDB ID number 3DO3 and human Fc PDB ID number 3DNK, respectively. See Table 6.

Alternatively, the Cα atom of core β-barrel residue V323 was also used to calculate inter-molecular distances. When V323 was used, Fc/3M also exhibited the most open conformation. Intermolecular distances for the three most open unliganded human Fc structures were estimated at 43.6, 41.3, 36.8, 35.10 and 37.97 Å, for Fc/3M, human Fc PDB ID number 1H3W (Krapp et al. 2003), human Fc PDB ID number 1FC1 (Deisenhofer et al., 1981, Biochemistry 20: 2361-2370), human Fc PDB ID number 3DO3 and human Fc PDB ID number 3DNK, respectively. See Table 6.

In addition, the angle defined by C_{H}2 and C_{H}3 could be assessed for each chain by the angle formed by a Cα atom in the C_{H}3 domain close to the Fc C terminus (for example, L443), a Cα atom in the hinge between C_{H}2 and C_{H}3 domains (for example, Q342) and a Cα atom in the C_{H}2 domain close to the Fc N terminus (for example, P329). When so defined, the respective C_{H}2/C_{H}3 angles for the four most open structures were 124.2, 124.7, 122.9, 119.8, 119.4, 118.43 and 115.23° for Fc/3M, chain B of human Fc PDB ID number 1E4K (Sondermann et al. 2000, Nature 406, 267-273), chain B of human Fc PDB ID number 1H3Y (Krapp et al. 2003, J. Mol. Biol. 325, 979-989), chain A of human Fc PDB ID number 1T83 (Radaev et al. 2001, J. Biol. Chem. 276, 16469-16477), human Fc PDB ID number 1H3W (Krapp et al. 2003, J. Mol. Biol. 325, 979-989), human Fc PDB ID number 3DO3 and human Fc PDB ID number 3DNK, respectively. See Table 6.

The angle defined by C_{H}2 and C_{H}3 could alternatively be assessed by the angle formed by three atoms: a Cα atom in the core β-barrel of the C_{H}3 domain spatially close to the Fc C terminus (for example, F423), a Cα atom in the core β-barrel of the C_{H}3 domain close to the C_{H}2/C_{H}3 junction (for example, E430) and a Cα atom in the core β-barrel of the C_{H}2 domain spatially close to the Fc N terminus (for example, V323). When so defined, Fc/3M exhibited the most open conformation when compared with other unliganded human Fc structures. More specifically, the respective C_{H}2/C_{H}3 angles for the three most open unliganded human Fc structures were estimated at 129.0, 128.7, 125.3, 122.44 and 117.71° for Fc/3M, chain B of human Fc PDB ID number 1H3Y (Krapp et al. 2003) and chain A of human Fc PDB ID number 1H3Y (Krapp et al. 2003), human Fc PDB ID number 3DO3 and human Fc PDB ID number 3DNK, respectively. See Table 6.

### 6.6 STRUCTURE COORDINATES

The atomic structure coordinates can be used in molecular modeling and design, as described more fully below. The present invention encompasses the structure coordinates and other information, e.g., amino acid sequence, connectivity tables, vector-based representations, temperature factors, etc., used to generate the three-dimensional structure of the polypeptide for use in the software programs described below and other software programs.

The invention encompasses machine-readable media embedded with information that corresponds to a three-dimensional structural representation of a crystal comprising a human IgG Fc variant in crystalline form or with portions thereof described herein. In certain embodiments, the crystal is diffraction quality. In certain embodiments, the crystal is a native crystal. In certain embodiments, the crystal is a heavy-atom derivative crystal. In certain embodiments, the information comprises the atomic structure coordinates of a human IgG Fc variant, or a subset thereof. In certain embodiments, the information comprises the atomic structure coordinates of Table 5 or a subset thereof.

As used herein, “machine-readable medium” refers to any medium that can be read and accessed directly by a computer or scanner. Such media include, but are not limited to: magnetic storage media, such as floppy discs, hard disc storage medium and magnetic tape; optical storage media such as optical discs or CD-ROM; electrical storage media such as RAM or ROM; and hybrids of these categories such as magnetic/optical storage media. Such media further include paper on which is recorded a representation of the atomic structure coordinates, e.g., Cartesian coordinates, that can be read by a scanning device and converted into a three-dimensional structure with an OCR.

A variety of data storage structures are available to a skilled artisan for creating a computer readable medium having recorded thereon the atomic structure coordinates of the invention or portions thereof and/or X-ray diffraction data. The choice of the data storage structure will generally be based on the means chosen to access the stored information. In addition, a variety of data processor programs and formats can be used to store the sequence and X-ray data information on a computer readable medium. Such formats include, but are not limited to, Protein Data Bank (“PDB”) format (Research Collaboratory for Structural Bioinformatics; Cambridge Crystallographic Data Centre format; Structure-data (“SD”) file format (MDL Information Systems, Inc.; Dalby et al., 1992, J. Chem. Inf. Comp. Sci. 32:244-255), and line-notation, e.g., as used in SMILES (Weininger, 1988, J. Chem. Inf. Comp. Sci. 28:31-36). Methods of converting between various formats read by different computer software will be readily apparent to those of skill in the art, e.g., BABEL (v. 1.06, Walters & Stahl, ©1992, 1993, 1994). All format representations of the polypeptide coordinates described herein, or portions thereof, are contemplated by the present invention. By providing computer readable medium having stored thereon the atomic coordinates of the invention, one of skill in the art can routinely access the atomic coordinates of the invention, or portions thereof, and related information for use in modeling and design programs, described in detail below.

While Cartesian coordinates are important and convenient representations of the three-dimensional structure of a polypeptide, those of skill in the art will readily ecognize that other representations of the structure are also useful. Therefore, the three-dimensional structure of a polypeptide, as discussed herein, includes not only the Cartesian coordinate representation, but also all alternative representations of the three-dimensional distribution of atoms. For example, atomic coordinates may be represented as a Z-matrix, wherein a first atom of the protein is chosen, a second atom is placed at a defined distance from the first atom, a third atom is placed at a defined distance from the second atom so that it makes a defined angle with the first atom. Each subsequent atom is placed at a defined distance from a previously placed atom with a specified angle with respect to the third atom, and at a specified torsion angle with respect to a fourth atom. Atomic coordinates may also be represented as a Patterson function, wherein all interatomic vectors are drawn and are then placed with their tails at the origin. This representation is particularly useful for locating heavy atoms in a unit cell. In addition, atomic coordinates may be represented as a series of vectors having magnitude and direction and drawn from a chosen origin to each atom in the polypeptide structure. Furthermore, the positions of atoms in a three-dimensional structure may be represented as fractions of the unit cell (fractional coordinates), or in spherical polar coordinates.

Additional information, such as thermal parameters, which measure the motion of each atom in the structure, chain identifiers, which identify the particular chain of a multi-chain protein in which an atom is located, and connectivity information, which indicates to which atoms a particular atom is bonded, is also useful for representing a three-dimensional molecular structure.

### 6.7 USES OF THE ATOMIC STRUCTURE COORDINATES

Structure information, typically in the form of the atomic structure coordinates, can be used in a variety of computational or computer-based methods to, for example, design, screen for and/or identify compounds that bind the crystallized polypeptide or a portion or fragment thereof, to intelligently design mutants that have altered biological properties, to intelligently design and/or modify antibodies that have desirable binding characteristics, and the like. The three-dimensional structural representation of the human IgG Fc variant can be visually inspected or compared with a three-dimensional structural representation of a wild type human IgG Fc region.

In one embodiment, the crystals and structure coordinates obtained therefrom are useful for identifying and/or designing compounds that bind human IgG Fc region as an approach towards developing new therapeutic agents. For example, a high resolution X-ray structure will often show the locations of ordered solvent molecules around the protein, and in particular at or near putative binding sites on the protein. This information can then be used to design molecules that bind these sites, the compounds synthesized and tested for binding in biological assays. See Travis, 1993, Science 262:1374.

In another embodiment, the structure is probed with a plurality of molecules to determine their ability to bind to human IgG Fc region at various sites. Such compounds can be used as targets or leads in medicinal chemistry efforts to identify, for example, inhibitors of potential therapeutic importance.

In yet another embodiment, the structure can be used to computationally screen small molecule data bases for chemical entities or compounds that can bind in whole, or in part, to human IgG Fc region, particularly, bind in the cleft formed between the Fc C_{H}2 and C_{H}3 domain of Fc region. In this screening, the quality of fit of such entities or compounds to the binding site may be judged either by shape complementarity or by estimated interaction energy. See Meng et al., 1992, J. Comp. Chem. 13:505-524.

The design of compounds that bind to or inhibit human IgG Fc region, according to this invention generally involves consideration of two factors. First, the compound should be capable of physically and structurally associating with human IgG Fc region. This association can be covalent or non-covalent. For example, covalent interactions may be important for designing irreversible inhibitors of a protein. Non-covalent molecular interactions important in the association of human IgG Fc region with its ligand include hydrogen bonding, ionic interactions and van der Waals and hydrophobic interactions. Second, the compound should be able to assume a conformation that allows it to associate with human IgG Fc region. Although certain portions of the compound will not directly participate in this association with IgG Fc region, those portions may still influence the overall conformation of the molecule. This, in turn, may impact potency. Such conformational requirements include the overall three-dimensional structure and orientation of the chemical group or compound in relation to all or a portion of the binding site, or the spacing between functional groups of a compound comprising several chemical groups that directly interact with human IgG Fc region.

The potential inhibitory or binding effect of a chemical compound on human IgG Fc region may be analyzed prior to its actual synthesis and testing by the use of computer modeling techniques. If the theoretical structure of the given compound suggests insufficient interaction and association between it and human IgG Fc region, synthesis and testing of the compound is unnecessary. However, if computer modeling indicates a strong interaction, the molecule may then be synthesized and tested for its ability to bind to human IgG Fc region and inhibit its binding activity. In this manner, synthesis of ineffective compounds may be avoided.

An inhibitory or other binding compound of human IgG Fc region may be computationally evaluated and designed by means of a series of steps in which chemical groups or fragments are screened and selected for their ability to associate with the cleft formed between the Fc C_{H}2 and C_{H}3 domain of Fc region or other areas of human IgG Fc region. One skilled in the art may use one of several methods to screen chemical groups or fragments for their ability to associate with human IgG Fc region. This process may begin by visual inspection of, for example, the binding site on the computer screen based on the cleft formed between the Fc C_{H}2 and C_{H}3 domain of Fc variant coordinates. Selected fragments or chemical groups may then be positioned in a variety of orientations, or docked, within the cleft formed between the Fc C_{H}2 and C_{H}3 domain of Fc region. Docking may be accomplished using software such as QUANTA and SYBYL, followed by energy minimization and molecular dynamics with standard molecular mechanics force fields, such as CHARMM and AMBER.

These principles may also be used to design and evaluate compounds that can mimic human IgG Fc variant with the high effector function amino acid residues, or to design and evaluate a modification of a human IgG Fc region that would result in an increased binding affinity for a FcγR or an increased ADCC activity compared to the comparable human IgG Fc region not comprising the modification. These principles may also be used to design and evaluate a modification of a human IgG Fc region that would result in decreased binding affinity for a FcγR or a decreased ADCC activity compared to the comparable human IgG Fc region not comprising the modification. Such modifications include and are not limited to amino acid substitution with a natural or a non-natural amino acid residue, or a carbohydrate chemical modification. In certain embodiments, modifications are designed or screened, which would result in larger inter-molecular distance between from the Cα atoms of P329 than that in a wild type human IgG region, preferably, greater than 33, 34, 35, 36, 37, 38, 39, 40, 41 or 42 Å. In certain embodiments, modifications are designed or screened, which would result in larger inter-molecular distance between from the Cα atoms of V323 than that in a wild type human IgG region, preferably, greater than 37, 38, 39, 40, 41, 42, 43, 44, 45 or 46 Å.

In certain embodiments, modifications are designed or screened, which would result in a larger angle between the C_{H}2 domain and C_{H}3 domain of the human IgG Fc than that in a wild type human IgG region. The angle between the C_{H}2 domain and C_{H}3 domain can be defined as the angle formed by a Cα atom in the C_{H}3 domain close to the Fc C terminus such as, L443, a Cα atom in the hinge between C_{H}2 and C_{H}3 domains, such as Q342, and a Cα atom in the C_{H}2 domain close to the Fc N terminus, such as P329. When so defined, in some embodiments, modifications are designed or screened, which would result in larger angle formed by L443, Q342 and P329 of the human IgG Fc than that in a wild type human IgG region, preferably, greater than 122, 123, 124, 125, 126 or 127°.

Alternatively, the angel between the C_{H}2 domain and C_{H}3 domain can be defined as the angle formed by a Cα atom in the core β-barrel of the C_{H}3 domain spatially close to the Fc C terminus, such as F423, a Cα atom in the core β-barrel of the C_{H}3 domain close to the C_{H}2/C_{H}3 junction, such as E430 and a Cα atom in the core β-barrel of the C_{H}2 domain spatially close to the Fc N terminus, such as for example, V323. When so defined, in some embodiments, modifications are designed or screened, which would result in larger angle formed by F423, E430 and V323 of the human IgG Fc than that in a wild type human IgG region, preferably, greater than 127, 128, 129, 130, 131 or 132°.

Specialized computer programs may also assist in the process of selecting fragments or chemical groups. These include:

1. GRID (Goodford, 1985, J. Med. Chem. 28:849-857). GRID is available from Oxford University, Oxford, UK;

2. MCSS (Miranker & Karplus, 1991, Proteins: Structure, Function and Genetics 11:29-34). MCSS is available from Molecular Simulations, Burlington, Mass.;

3. AUTODOCK (Goodsell & Olsen, 1990, Proteins: Structure, Function, and Genetics 8:195-202). AUTODOCK is available from Scripps Research Institute, La Jolla, Calif.; and

4. DOCK (Kuntz et al., 1982, J. Mol. Biol. 161:269-288). DOCK is available from University of California, San Francisco, Calif.

Once suitable chemical groups or fragments have been selected, they can be assembled into a single compound or inhibitor. Assembly may proceed by visual inspection of the relationship of the fragments to each other in the three-dimensional image displayed on a computer screen in relation to the structure coordinates of human IgG Fc variant. This would be followed by manual model building using software such as QUANTA or SYBYL.

Useful programs to aid one of skill in the art in connecting the individual chemical groups or fragments include:

1. CAVEAT (Bartlett et al., 1989, “CAVEAT: A Program to Facilitate the Structure-Derived Design of Biologically Active Molecules,” In Molecular Recognition in Chemical and Biological Problems', Special Pub., Royal Chem. Soc. 78:182-196). CAVEAT is available from the University of California, Berkeley, Calif.;

2. 3D Database systems such as MACCS-3D (MDL Information Systems, San Leandro, Calif.). This area is reviewed in Martin, 1992, J. Med. Chem. 35:2145-2154); and

3. HOOK (available from Molecular Simulations, Burlington, Mass.).

Instead of proceeding to build a human IgG Fc binding compound in a step-wise fashion one fragment or chemical group at a time, as described above, Fc region binding compounds may be designed as a whole or “de novo” using either an empty Fc region binding site or optionally including some portion(s) of a known inhibitor(s). These methods include:

1. LUDI (Bohm, 1992, J. Comp. Aid. Molec. Design 6:61-78). LUDI is available from Molecular Simulations, Inc., San Diego, Calif.;

2. LEGEND (Nishibata & Itai, 1991, Tetrahedron 47:8985). LEGEND is available from Molecular Simulations, Burlington, Mass.; and

3. LeapFrog (available from Tripos, Inc., St. Louis, Mo.).

Other molecular modeling techniques may also be employed in accordance with this invention. See, e.g., Cohen et al., 1990, J. Med. Chem. 33:883-894. See also Navia & Murcko, 1992, Cur. Op. Struct. Biol. 2:202-210.

Once a compound or a modification has been designed or selected by the above methods, the efficiency with which that compound may bind to Fc region or a ligand of a Fc region may be tested and optimized by computational evaluation. For example, a compound that has been designed or selected to function as a Fc region binding compound should also preferably occupy a volume not overlapping the volume occupied by the binding site residues when the native receptor is bound. An effective Fc region compound preferably demonstrates a relatively small difference in energy between its bound and free states (i.e., it should have a small deformation energy of binding). Thus, the most efficient Fc region binding compounds should preferably be designed with a deformation energy of binding of not greater than about 10 kcal/mol, preferably, not greater than 7 kcal/mol. Fc region binding compounds may interact with the protein in more than one conformation that is similar in overall binding energy. In those cases, the deformation energy of binding is taken to be the difference between the energy of the free compound and the average energy of the conformations observed when the inhibitor binds to the enzyme.

A compound selected or designed for binding to human IgG Fc region may be further computationally optimized so that in its bound state it would preferably lack repulsive electrostatic interaction with the target protein. Such non-complementary electrostatic interactions include repulsive charge-charge, dipole-dipole and charge-dipole interactions. Specifically, the sum of all electrostatic interactions between the inhibitor and the protein when the inhibitor is bound to it preferably make a neutral or favorable contribution to the enthalpy of binding.

Specific computer software is available in the art to evaluate compound deformation energy and electrostatic interaction. Examples of programs designed for such uses include: Gaussian 92, revision C (Frisch, Gaussian, Inc., Pittsburgh, Pa. ©1992); AMBER, version 4.0 (Kollman, University of California at San Francisco, ©1994); QUANTA/CHARMM (Molecular Simulations, Inc., Burlington, Mass., ©1994); and Insight II/Discover (Biosym Technologies Inc., San Diego, Calif., ©1994). These programs may be implemented, for instance, using a computer workstation, as are well-known in the art. Other hardware systems and software packages will be known to those skilled in the art.

Once a compound has been optimally selected or designed, as described above, substitutions may then be made in some of its atoms or chemical groups in order to improve or modify its binding properties. Generally, initial substitutions are conservative, i.e., the replacement group will have approximately the same size, shape, hydrophobicity and charge as the original group. One of skill in the art will understand that substitutions known in the art to alter conformation should be avoided. Such altered chemical compounds may then be analyzed for efficiency of binding to Fc region by the same computer methods described in detail above.

Specific computer software is available in the art to evaluate compound deformation energy and electrostatic interaction. Examples of programs designed for such uses include: Gaussian 92, revision C (Frisch, Gaussian, Inc., Pittsburgh, Pa. ©1992); AMBER, version 4.0 (Kollman, University of California at San Francisco, ©1994); QUANTA/CHARMM (Molecular Simulations, Inc., Burlington, Mass., ©1994); and Insight II/Discover (Biosym Technologies Inc., San Diego, Calif, ©1994). These programs may be implemented, for instance, using a computer workstation, as are well-known in the art. Other hardware systems and software packages will be known to those skilled in the art. Once a Fc region-binding compound has been optimally selected or designed, as described above, substitutions may then be made in some of its atoms or chemical groups in order to improve or modify its binding properties. Generally, initial substitutions are conservative, i.e., the replacement group will have approximately the same size, shape, hydrophobicity and charge as the original group. One of skill in the art will understand that substitutions known in the art to alter conformation should be avoided. Such altered chemical compounds may then be analyzed for efficiency of binding to human IgG Fc region by the same computer methods described in detail above.

The structure coordinates of human IgG Fc variant, or portions thereof, are particularly useful to solve the structure of those other crystal forms of human IgG Fc region or fragments. They may also be used to solve the structure of human IgG Fc variant mutants, IgG Fc-complexes, fragments thereof, or of the crystalline form of any other protein that shares significant amino acid sequence homology with a structural domain of IgG Fc region.

One method that may be employed for this purpose is molecular replacement. In this method, the unknown crystal structure, whether it is another crystal form of human IgG Fc variant, or its mutant or complex, or the crystal of some other protein with significant amino acid sequence homology to any functional domain of human IgG Fc region, may be determined using phase information from the human IgG Fc variant structure coordinates. The phase information may also be used to determine the crystal structure of human IgG Fc variant mutants or complexes thereof, and other proteins with significant homology to human IgG Fc variant or a fragment thereof. This method will provide an accurate three-dimensional structure for the unknown protein in the new crystal more quickly and efficiently than attempting to determine such information ab initio. In addition, in accordance with this invention, human IgG Fc variant may be crystallized in complex with known Fc binding compound, such as FcγR such as human CD 16. The crystal structures of a series of such complexes may then be solved by molecular replacement and compared with that of human IgG Fc variant. Potential sites for modification within the various binding sites of the protein may thus be identified. This information provides an additional tool for determining the most efficient binding interactions, for example, increased hydrophobic interactions, between human IgG Fc region and a chemical group or compound.

If an unknown crystal form has the same space group as and similar cell dimensions to the known human IgG Fc variant crystal form, then the phases derived from the known crystal form can be directly applied to the unknown crystal form, and in turn, an electron density map for the unknown crystal form can be calculated. Difference electron density maps can then be used to examine the differences between the unknown crystal form and the-known crystal form. A difference electron density map is a subtraction of one electron density map, e.g., that derived from the known crystal form, from another electron density map, e.g., that derived from the unknown crystal form. Therefore, all similar features of the two electron density maps are eliminated in the subtraction and only the differences between the two structures remain. For example, if the unknown crystal form is of a human IgG Fc variant complex, then a difference electron density map between this map and the map derived from the native, uncomplexed crystal will ideally show only the electron density of the ligand. Similarly, if amino acid side chains have different conformations in the two crystal forms, then those differences will be highlighted by peaks (positive electron density) and valleys (negative electron density) in the difference electron density map, making the differences between the two crystal forms easy to detect. However, if the space groups and/or cell dimensions of the two crystal forms are different, then this approach will not work and molecular replacement must be used in order to derive phases for the unknown crystal form.

All of the complexes referred to above may be studied using well-known X-ray diffraction techniques and may be refined versus 5 Å to 1.5 Å, or greater resolution X-ray data to an R value of about 0.20 or less using computer software, such as X-PLOR (Yale University, (c) 1992, distributed by Molecular Simulations, Inc.). See, e.g., Blundel et al., 1976, Protein Crystallography, Academic Press.; Methods in Enzymology, vol. 114 & 115, Wyckoff et al., eds., Academic Press, 1985. This information may thus be used to optimize known classes of human IgG Fc binding compounds, and more importantly, to design and synthesize novel classes of IgG Fc binding compounds.

The structure coordinates of human IgG Fc variant will also facilitate the identification of related proteins or enzymes analogous to human IgG Fc in function, structure or both, thereby further leading to novel therapeutic modes for treating or preventing human IgG Fc mediated diseases.

Subsets of the atomic structure coordinates can be used in any of the above methods. Particularly useful subsets of the coordinates include, but are not limited to, coordinates of single domains, coordinates of residues lining an antigen binding site, coordinates of residues of a CDR, coordinates of residues that participate in important protein-protein contacts at an interface, and Ca coordinates. For example, the coordinates of a fragment of an antibody that contains the antigen binding site may be used to design inhibitors that bind to that site, even though the antibody is fully described by a larger set of atomic coordinates. Therefore, a set of atomic coordinates that define the entire polypeptide chain, although useful for many applications, do not necessarily need to be used for the methods described herein.

Exemplary molecular screening or designing methods by using the three-dimensional structural representation of a human IgG Fc variant comprising one or more high effector function amino acid residues and has an increased binding affinity for a FcγR compared to a wild type human IgG Fc region not comprising the high effector function amino acid residues, or portion thereof, particularly that of the human IgG Fc variant comprise may comprise at least one high effector function amino acid residue selected from the group consisting of 239D, 330L and 332E, as numbered by the EU index as set forth in Kabat, and preferably that of the human IgG Fc variant comprises the amino acid sequence of SEQ ID NO:1, are described below.

In one aspect, the present invention provides methods of identifying or designing compounds that binds a human IgG or a human IgG Fc region, comprising using a three-dimensional structural representation of a human IgG Fc variant.

In certain embodiments, the present invention provides a method of identifying a compound that binds a human IgG or a human IgG Fc region, comprising using a three-dimensional structural representation of a human IgG Fc variant comprising one or more high effector function amino acid residues and has an increased binding affinity for a FcγR compared to a wild type human IgG Fc region not comprising the high effector function amino acid residues, or portion thereof, to computationally screen a candidate compound for an ability to bind the human IgG or the human IgG Fc region. The computational screen may comprise the steps of synthesizing the candidate compound; and screening the candidate compound for an ability to bind a human IgG or a human IgG Fc. In such methods, the three-dimensional structural representation of the human IgG Fc variant may be visually inspected to identify a candidate compound. The method may further comprise comparing a three-dimensional structural representation of a wild type human IgG Fc region with that of the human IgG Fc variant.

In certain embodiments, the present invention provides a method of designing a compound that binds a human IgG or a human IgG Fc region, comprising using a three-dimensional structural representation of a human IgG Fc variant comprising one or more high effector function amino acid residues and has an increased binding affinity for a FcγR compared to a wild type human IgG Fc region not comprising the high effector function amino acid residues, or portion thereof, to computationally design a synthesizable candidate compound for an ability to bind the human IgG or the human IgG Fc region. The computational design may comprise the steps of synthesizing the candidate compound; and screening the candidate compound for an ability to bind a human IgG or a human IgG Fc. In such methods, the three-dimensional structural representation of the human IgG Fc variant may be visually inspected to identify a candidate compound. The method may further comprise comparing a three-dimensional structural representation of a wild type human IgG Fc region with that of the human IgG Fc variant.

In another aspects, the present invention provides methods of identifying or designing a modification of a human IgG Fc region that would result in an altered binding affinity for a FcγR or an altered ADCC activity compared to the comparable human IgG Fc region not comprising the modification, by using a three-dimensional structural representation of a human IgG Fc variant.

In another aspects, the present invention provides methods of identifying or designing a modification of a human IgG Fc region that would result in a more open structure compared to the comparable human IgG Fc region not comprising the modification, by using a three-dimensional structural representation of a human IgG Fc variant. In certain embodiments, the modification may result in an altered, e.g., increased, binding affinity for a FcγR or an altered, e.g., increased ADCC activity compared to the comparable human IgG Fc region not comprising the modification. The openness of the structure may be determined by any technique known in the art, such as by the inter-molecular distance between selected residues of the polypeptide chinas or by the angel between C_{H}2 and C_{H}3 domains.

In another aspects, the present invention provides methods of identifying or designing a modification of a human IgG Fc region that would result in a more close structure compared to the comparable human IgG Fc region not comprising the modification, by using a three-dimensional structural representation of a human IgG Fc variant. In certain embodiments, the modification may result in an altered, e.g., reduced, binding affinity for a FcγR or an altered, e.g., reduced ADCC activity compared to the comparable human IgG Fc region not comprising the modification.

Such modification includes but is not limited to an amino acid insertion, an amino acid deletion, an amino acid substitution by a natural or an unnatural amino acid residue, and a carbohydrate chemical modification

In certain embodiments, the present invention provides a method of identifying a modification of a human IgG Fc region that would result in an altered binding affinity for a FcγR or an altered ADCC activity compared to the comparable human IgG Fc region not comprising the modification, comprising using a three-dimensional structural representation of a human IgG Fc variant comprising one or more high effector function amino acid residues, wherein said human IgG Fc variant has an increased binding affinity for a FcγR compared to a wild type human IgG Fc region not comprising the high effector function amino acid residues, or portion thereof, to computationally screen a modification that result in an altered binding affinity for a FcγR or an altered ADCC activity. In such methods, the three-dimensional structural representation of the human IgG Fc variant may be visually inspected to identify a candidate compound. The method may further comprise comparing a three-dimensional structural representation of a wild type human IgG Fc region with that of the human IgG Fc variant.

In certain embodiments, the present invention provides a method of identifying a modification of a human IgG Fc region that would result in a more close structure compared to the comparable human IgG Fc region not comprising the modification, comprising using a three-dimensional structural representation of a human IgG Fc variant comprising one or more high effector function amino acid residues, wherein said human IgG Fc variant has an increased binding affinity for a FcγR compared to a wild type human IgG Fc region not comprising the high effector function amino acid residues, or portion thereof, to computationally screen a modification that result in a more close structure. In certain embodiments, the modification may result in an altered, e.g., reduced, binding affinity for a FcγR or an altered, e.g., reduced ADCC activity compared to the comparable human IgG Fc region not comprising the modification. In some embodiments, the modification may result in an inter-molecular distance between from the Cα atoms of P329 less that that in a wild type human IgG region or less than 28, 29, 30, 31, 32, 33, 34, 35, 36, 37, 38, 39, 40, 41 or 42 Å. In some embodiments, the modification may result in an inter-molecular distance between from the Cα atoms of V323 less that that in a wild type human IgG region or less than 33, 34, 35, 36, 37, 38, 39, 40, 41, 42, 43, 44, 45 or 46 Å. In some embodiments, the modification may result in the angle between the C_{H}2 domain and C_{H}3 domain of the human IgG Fc is less that that in a wild type human IgG region or less than 132°. In some embodiments, the modification may result in the angle formed by L443, Q342 and V323 of the human IgG Fc less than that in a wild type human IgG region or less than 119, 120, 121, 122, 123, 124, 125, 126 or 127°. In some embodiments, the modification may result in the angle formed by F423, E430 and V323 of the human IgG Fc less than that in a wild type human IgG region or less than 122, 123, 124, 125, 126, 127, 128, 129, 130, 131 or 132°.

In certain embodiments, the present invention provides a method of identifying a modification of a human IgG Fc region that would result in an increased binding affinity for a FcγR or an increased ADCC activity compared to the comparable human IgG Fc region not comprising the modification, comprising using a three-dimensional structural representation of a human IgG Fc variant comprising one or more high effector function amino acid residues, wherein said human IgG Fc variant has an increased binding affinity for a FcγR compared to a wild type human IgG Fc region not comprising the high effector function amino acid residues, or portion thereof, to computationally screen a modification that result in an increased binding affinity for a FcγR or an increased ADCC activity. In some embodiments, the modification may result in an inter-molecular distance between from the Cα atoms of P329 greater than that in a wild type human IgG region or greater than 33, 34, 35, 36, 37, 38, 39, 40, 41 or 42 Å. In some embodiments, the modification may result in an inter-molecular distance between from the Cα atoms of V323 greater than that in a wild type human IgG region or greater than 37, 38, 39, 40, 41, 42, 43, 44, 45 or 46 Å. In some embodiments, the modification may result in the angle between the C_{H}2 domain and C_{H}3 domain of the human IgG Fc is greater than that in a wild type human IgG region or greater than 132°. In some embodiments, the modification may result in the angle formed by L443, Q342 and V323 of the human IgG Fc greater than that in a wild type human IgG region or greater than 119, 120, 121, 122, 123, 124, 125, 126 or 127°. In some embodiments, the modification may result in the angle formed by F423, E430 and V323 of the human IgG Fc greater than that in a wild type human IgG region or greater than 124, 125, 126, 127, 128, 129, 130, 131 or 132°.

In certain embodiments, the present invention provides a method of designing a modification of a human IgG Fc region that would result in an altered binding affinity for a FcγR or an altered ADCC activity compared to the comparable human IgG Fc region not comprising the modification, comprising using a three-dimensional structural representation of a human IgG Fc variant comprising one or more high effector function amino acid residues, wherein said human IgG Fc variant has an increased binding affinity for a FcγR compared to a wild type human IgG Fc region not comprising the high effector function amino acid residues, or portion thereof, to computationally design a modification that result in an altered binding affinity for a FcγR or an increased ADCC activity. In such methods, the three-dimensional structural representation of the human IgG Fc variant may be visually inspected to identify a candidate compound. The method may further comprise comparing a three-dimensional structural representation of a wild type human IgG Fc region with that of the human IgG Fc variant.

In certain embodiments, the present invention provides a method of designing a modification of a human IgG Fc region that would result in a more close structure compared to the comparable human IgG Fc region not comprising the modification, comprising using a three-dimensional structural representation of a human IgG Fc variant comprising one or more high effector function amino acid residues, wherein said human IgG Fc variant has an increased binding affinity for a FcγR compared to a wild type human IgG Fc region not comprising the high effector function amino acid residues, or portion thereof, to computationally design a modification that result in a more close structure. In certain embodiments, the modification may result in an altered, e.g., reduced, binding affinity for a FcγR or an altered, e.g., reduced ADCC activity compared to the comparable human IgG Fc region not comprising the modification. In some embodiments, the modification may result in an inter-molecular distance between from the Cα atoms of P329 less that that in a wild type human IgG region or less than 28, 29, 30, 31, 32, 33, 34, 35, 36, 37, 38, 39, 40, 41 or 42 Å. In some embodiments, the modification may result in an inter-molecular distance between from the Cα atoms of V323 less that that in a wild type human IgG region or less than 33, 34, 35, 36, 37, 38, 39, 40, 41, 42, 43, 44, 45 or 46 Å. In some embodiments, the modification may result in the angle between the C_{H}2 domain and C_{H}3 domain of the human IgG Fc is less that that in a wild type human IgG region or less than 122°. In some embodiments, the modification may result in the angle formed by L443, Q342 and V323 of the human IgG Fc less than that in a wild type human IgG region or less than 122, 123, 124, 125, 126 or 127°. In some embodiments, the modification may result in the angle formed by F423, E430 and V323 of the human IgG Fc less than that in a wild type human IgG region or less than 127, 128, 129, 130, 131 or 132°.

In certain embodiments, the present invention provides a method of designing a modification of a human IgG Fc region that would result in an increased binding affinity for a FcγR or an increased ADCC activity compared to the comparable human IgG Fc region not comprising the modification, comprising using a three-dimensional structural representation of a human IgG Fc variant comprising one or more high effector function amino acid residues, wherein said human IgG Fc variant has an increased binding affinity for a FcγR compared to a wild type human IgG Fc region not comprising the high effector function amino acid residues, or portion thereof, to computationally design a modification that result in an increased binding affinity for a FcγR or an increased ADCC activity. In some embodiments, the modification may result in an inter-molecular distance between from the Cα atoms of P329 greater than that in a wild type human IgG region or greater than 33, 34, 35, 36, 37, 38, 39, 40, 41 or 42 Å. In some embodiments, the modification may result in an inter-molecular distance between from the Cα atoms of V323 greater than that in a wild type human IgG region or greater than 37, 38, 39, 40, 41, 42, 43, 44, 45 or 46 Å. In some embodiments, the modification may result in the angle between the C_{H}2 domain and C_{H}3 domain of the human IgG Fc is greater than that in a wild type human IgG region or greater than 122°. In some embodiments, the modification may result in the angle formed by L443, Q342 and V323 of the human IgG Fc greater than that in a wild type human IgG region or greater than 122, 123, 124, 125, 126 or 127°. In some embodiments, the modification may result in the angle formed by F423, E430 and V323 of the human IgG Fc greater than that in a wild type human IgG region or greater than 127, 128, 129, 130, 131 or 132°.

### 6.8 HUMAN IGG FC VARIANTS

Using the structure coordinates of human IgG Fc variant and the methods disclosed herein the inventors have identified additional human IgG Fc variants with decreased binding affinity for a number of FcγRs. Accordingly, the present invention provides human IgG Fc variants having decreased binding affinity to at least one FcγR.

In certain embodiments, the present invention provides a recombinant polypeptide comprising a human IgG Fc region that comprises one or more amino acid residue deletions compared to a wild type human IgG Fc region. In some embodiments, the deletion is selected from the group consisting of amino acid residues 294, 295, 296, 298 and 299 as numbered by the EU index as set forth in Kabat. In certain embodiments, the present invention provides a recombinant polypeptide comprising a human IgG Fc region that comprises at least one amino acid residue deletions compared to a wild type human IgG Fc region, wherein the Fc region comprises a deletion of amino acid residues 295 and 296; or a deletion of amino acid residues 294, 295 and 296; or a deletion of amino acid residues 294, 295, 296, 298 and 299 as numbered by the EU index as set forth in Kabat. In specific embodiments, the recombinant polypeptide comprises SEQ ID NO:8, 9, or 10.

In certain embodiments, the present invention provides a recombinant polypeptide comprising a human IgG Fc region that comprises one or more amino acid residue substitutions compared to a wild type human IgG Fc region. In some embodiments, the substitution is selected from the group consisting of 300S and 301T as numbered by the EU index as set forth in Kabat. In specific embodiments, the recombinant polypeptide comprises the substitution of amino acid residues 300S and 301T.

In certain embodiments, the present invention provides a recombinant polypeptide comprising a human IgG Fc region that comprises one or more amino acid residue deletion and one or more amino acid residue substitutions compared to a wild type human IgG Fc region. In some embodiments, the Fc region comprises one or more amino acid residue deletions selected from the group consisting of 294, 295, 296, 298 and 299 and further comprises one or more amino acid residue substitutions selected from the group consisting of 300S and 301T as numbered by the EU index as set forth in Kabat. In specific embodiments, the Fc region comprises the substitution of amino acid residues 300S and 301T and further comprises the deletion of amino acid residues 295 and 296, or the deletion of amino acid residues 294, 295 and 296, or the deletion of 294, 295, 296, 298 and 299. In particular embodiments, the recombinant polypeptide comprises SEQ ID NO: 8, 9 or 10. In a particular embodiments, the the recombinant polypeptide consists of SEQ ID NO: 8, 9 or 10.

In other embodiments, the recombinant polypeptide has decreased binding affinity to at least one FcγR selected from the group consisting of FcγRIIIA (CD16), FcγRIIA, FcγRIIB and FcγRI. In a specific embodiment, a human IgG Fc variant having decreased binding affinity to at least one FcγR has decrease binding affinity to FcγRIIIA (CD16), FcγRIIA, FcγRIIB and FcγRI.

In addition to the amino acid residue deletions and/or substitutions described above, the human IgG Fc region may comprise one or more additional amino acid residue substitutions of the wild-type sequence(s) with a different amino acid residue and/or by the addition and/or deletion of one or more amino acid residues to or from the wild-type sequence(s). The additions and/or deletions can be from an internal region of the wild-type sequence and/or at either or both of the N- or C-termini. In certain embodiments, the human IgG Fc variant having decreased binding affinity to at least one FcγR further comprises 1, 2, 3, 4 or 5 amino acid substitutions, deletions or additions.

The following examples are provided to illustrate aspects of the invention, and are not intended to limit the scope of the invention in any way.

### 7. EXAMPLES

The subsections below describe the production of a human IgG Fc variant Fc/3M, and the preparation and characterization of diffraction quality Fc/3M crystals.

### 7.1 PRODUCTION AND PURIFICATION OF 3F2/3M

### 7.1.1 GENERATION, EXPRESSION AND PURIFICATION OF 3F2/3M

The heavy and light chains of 3F2 (IgG1, κ), an affinity optimized version of the previously described 2G6/12C8 anti-human EphA2 monoclonal antibody, (Dall'Acqua et al., 2005, Methods 36;43-60), were cloned into a mammalian expression vector encoding a human cytomegalovirus major immediate early (hCMVie) enhancer, promoter and 5′-untranslated region (Boshart et al. 1985, Cell 41, 521-530). In this system, a human γ1 chain is secreted along with a human κ chain (Johnson et al. 1997, J. Infect. Dis. 176, 1215-1224.). The 3M combination of mutations (S239D/A330L/I332E) was introduced into the heavy chain of 3F2. Generation of these mutations was carried out by site-directed mutagenesis using a Quick Change XL Mutagenesis Kit according to the manufacturer's instructions (Stratagene, La Jolla, Calif.). This generated 3F2/3M. NS0 (murine myeloma) cells were then stably transfected with the corresponding antibody constructs, and the secreted immunoglobulins were purified using protein A and standard protocols.

The 3F2/Fab fragment used in DSC (differential scanning clorimetry) experiments was directly expressed from the 3F2/3M expression construct described in the previous section into which a TAA stop codon was introduced prior to heavy chain residue K222. The corresponding heavy and light chain constructs were then transiently transfected into HEK 293 cells using Lipofectamine (Invitrogen, Inc.) and standard protocols. 3F2/Fab was typically harvested at 72, 144 and 216 hours post-transfection and purified from the conditioned media directly on protein L columns according to the manufacturer's instructions (Pierce, Rockford, Ill.). Purified 3F2/Fab (typically >95% homogeneity, as judged by SDS-PAGE) was then dialyzed against PBS.

The unmutated human Fc fragment used in DSC experiments was obtained from the enzymatic cleavage of two human IgG1 molecules, 3F2 (see above) and MEDI-524. See Boshart et al., 1985, Cell 41:521-530. Digestions were carried out using immobilized ficin according to the manufacturer's instructions (Pierce). Purification was performed on HiTrap protein A columns according to the manufacturer's instructions (APBiotech, Inc). Purified human Fc (typically >95% homogeneity, as judged by SDS-PAGE) was then dialyzed against PBS.

### 7.1.2 GENERATION OF RECOMBINANT FC/3M

Recombinant human Fc/3M (amino acids 223-447) was PCR-amplified from the 3F2/3M expression construct described in the previous section and cloned as an XbaI/EcoRI fragment into the same vector. This was carried out using standard protocols and the oligonucleotides:

(SEQ ID NO: 5)
5′TATATATATCTAGACATATATATGGGTGACAATGACATCCACTTTGCCT
TTCTCTCC3′,
(SEQ ID NO: 6)
5′TCCACTTTGCCTTTCTCTCCACAGGTGTCCACTCCACTCACACATGCCC
ACCGTGCCC3′,
and
(SEQ ID NO: 7)
5′GATCAATGAATTCGCGGCCGCTCATTTACCCGGAGACAGG3′.

The Fc/3M construct was then transiently transfected into Human Embryonic Kidney (HEK) 293 cells using Lipofectamine (Invitrogen, Inc., Carlsbad, Calif.) and standard protocols. Fc/3M was typically harvested at 72, 144 and 216 hours post-transfection and purified from the conditioned media directly on 1 ml HiTrap protein A columns according to the manufacturer's instructions (APBiotech, Inc., Piscataway, N.J.). Purified Fc/3M (typically >95% homogeneity, as judged by reducing and non-reducing SDS-PAGE) was then dialyzed against phosphate buffered saline (PBS) and submitted to crystallization trials.

Purified Fc/3M was concentrated to about 13 mg/ml using a Centricon concentrator (30 KDa cutoff). Crystallization conditions were identified using Index, Crystal Screen I, Crystal Screen II (Hampton Research, Aliso Viejo, Calif.), Wizard 1 and Wizard 2 (Emerald BioSystems, Inc., Bainbridge Island, Wash.) screens. Each screen yielded several potentially usable crystallization conditions. Upon optimization, diffraction-quality crystals of about 150 μm were obtained from 0.1 M Imidazole-Malate pH 8.0, 8% polyethylene glycol (PEG) 3350, 200 mM zinc acetate, 5% glycerol at a protein concentration of 0.9 mg/ml. Prior to data collection, the crystal was soaked in the mother liquor supplemented with 10, 15, 20 and 25% glycerol, consecutively.

### 7.2 ANALYSIS AND CHARACTERIZATION OF FC/3M CRYSTALS

This example describes the methods used to generate and collect diffraction data from Fc/3M crystals and determine the structure of the Fc/3M from such data.

### 7.2.1 DIFFRACTION DATA COLLECTION

Diffraction data were collected at the Center for Advanced Research in Biotechnology (CARB, University of Maryland Biotechnology Institute, Rockville, Md.) using a Rigaku Micro Max 007 rotating anode generator with an RAXIS IV++area detector (Rigaku/MSC, The Woodlands, Tex.). The crystal was cooled to 105 K with an X-stream 2000 Cryogenic cooler (Rigaku/MSC). The initial diffraction pattern only showed a 3.8 Å fuzzy anisotropic diffraction. For annealing purposes, the crystal was taken from the goniometer head and placed into a fresh drop of mother liquor containing 25% glycerol. This procedure substantially improved its diffraction properties. During data collection, 160 consecutive images with an oscillation range of 0.5° and an exposure time of 600 seconds were measured. Data collected from a single crystal yielded a nearly complete set at resolution of 2.5 Å. It was observed that the number of reflections on every image remained unpredictable during processing. Thus, the crystal probably contained satellites which contributed to the diffraction pattern and compromised the data quality. This fact probably explains the relatively high R_{sym} value and high R-factors in refinement and in Sfcheck (Vaguine et al. 1999, Acta Cryst. D55, 191-205.). Data were processed with HKL 2000 (Otwinowski and Minor, 1997, Mode. Methods in Enzymology 276A, 307-326.). Data reduction, molecular replacement, refinement, and electron density calculation were carried out using the CCP4 (Collaborative Computational Project) program suite. The three amino acid substitutions which comprised 3M were first modeled as alanine residues and then incorporated as such (D239, L330, E332) when allowed by the corresponding electron densities.

### 7.2.2 STRUCTURE DETERMINATION

The crystal structure of a human IgG1 Fc fragment containing the S239D/A330L/I332E triple substitution (Fc/3M) was determined by molecular replacement and refined at a 2.5 Å resolution. More precisely, various human Fc regions deposited with the Protein Data Bank (PDB; Berman et al. 2000, Nucl. Acids Res. 28, 235-242) were evaluated as potential models for molecular replacement. All but one required that the C_{H}2 and C_{H}3 domains be considered separately to produce a solution. Only PDB ID number 1H3W (Krapp et al. 2003, J. Mol. Biol. 325, 979-989.) yielded a solution when both C_{H}2 and C_{H}3 domains were considered simultaneously. While all provided similar results, the human Fc structure corresponding to PDB ID number 2DTQ (Matsumiya et al. 2007, J. Mol. Biol. 368, 767-779) was used as the model in the present study because of its high resolution and unliganded state. Furthermore, the use of C_{H}2 and C_{H}3 domains separately provided less bias from the replacement structure in terms of the domain relative orientation. After several rounds of refinement using “Refmac 5” (Murshudov et al. 1997, Acta Cryst. D53, 240-255) and manual re-building using the “O” software (Jones et al. 1991, Acta Cryst. A47, 110-119), the model was analyzed using the TLS Motion Determination (TLSMD) program running on its web Server (Painter et al. 2006, Acta Cryst. D62, 439-450). Further refinement was then carried out with Refmac 5 in TLSMD mode using two distinct groups of residues (238-347 and 348-444). Both of these groups, as expected, corresponded to the C_{H}2 and C_{H}3 domains of Fc/3M. Amino acids corresponding to positions 223-237 and 445-447 were excluded from the final model due to the absence of corresponding electron density. Most atoms of the side chains at mutated positions 239, 330 and 332 were well-defined. See FIG. 9. Four peaks of electron density (˜8σ in Fo−Fc difference density maps) were modeled as Zn²⁺ ions based on the tetrahedral shape of their electron density map. Attempts to visualize peaks on anomalous difference density maps for Zn²⁺ ions failed, probably because of marginal data quality (R_{sym}=0.159).

Thus, in summary, the resulting model contained amino acids corresponding to positions 236 to 444, one branched carbohydrate chain, four Zn²⁺ ions as well as twenty four water molecules. Data collection and refinement statistics for the data set and the model are shown in Table 2 and Table 3, respectively. The asymmetric unit contents of the Fc/3M crystal and the overall three-dimensional structure of the entire Fc/3M molecule are shown in FIGS. 1A and 1B, respectively.

The atomic coordinates and experimental structure factors of Fc/3M have been deposited to the Protein Data Bank under accession number 2QL1.

### 7.2.3 CARBOHYDRATES ANALYSIS

The N-linked glycan chains attached to N297 were modeled at a later stage of refinement in accordance with their electron density and are shown in FIG. 1C. Overall, nine carbohydrate residues were located in each chain. The three-residue sequence Man5-GlcNAc6-Gal7 could be successfully modeled in one branch of the bi-antennary chain, while the other branch was missing its terminal Gal residue. There was no evidence in the form of electron density for this particular terminal Gal residue in that branch. The GlcNAc6 and Gal7 residues of the longer carbohydrate antenna exhibited a number of hydrogen bonds formed with protein residues, unlike the terminal GlcNAc9 residue of the shorter chain which was found to be in an unbound state. None of the mannose residues of each antenna (Man5 and Man8) were involved in any interaction with the polypeptide chain. Carbohydrate chains present in the two Fc/3M symmetry-related polypeptides did not form inter-molecular hydrogen bonds with each other at a set threshold of 3.5 Å. The lack of such interactions was observed in only three previously described human Fc structures, alone (PDB ID number 1H3W described in Krapp et al. 2003, J. Mol. Biol. 325, 979-989) or in complex with human CD16 (PDB ID numbers 1E4K and 1T83 described in Sondermann et al. 2000, Nature 406, 267-273 and Radaev et al. 2001, J. Biol. Chem. 276, 16469-16477 respectively).

### 7.2.4 ANALYSIS OF METAL BINDING

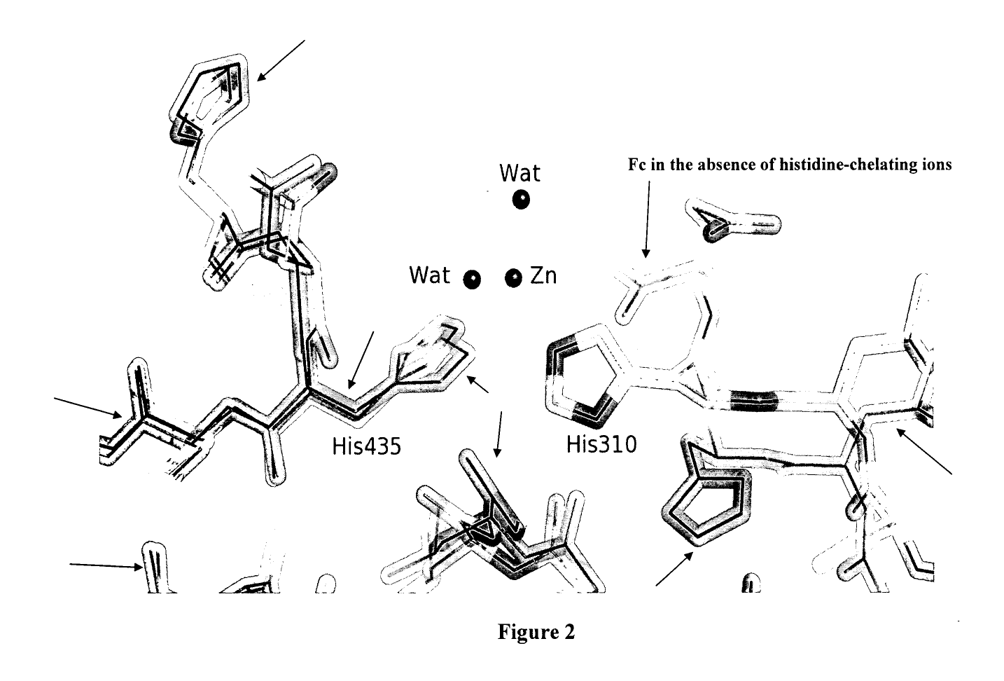

Four peaks of electron density (˜8σ in Fo−Fc difference density maps) were modeled as Zn²⁺ ions based on the tetrahedral shape of their electron density map. Attempts to visualize peaks on anomalous difference density maps for Zn²⁺ ions failed, probably because of marginal data quality (R_{sym}=0.159). The presence of two Zn²⁺ ions near solvent exposed positions E318 and E345 may be the result of very high zinc acetate concentrations in the crystallization buffer, since glutamate side chains alone do not typically bind transition state metal ions. Two other Zn²⁺ ions near semi-buried positions H310 and H435 on one hand, and solvent exposed position H433 on the other hand, may explain the ability of human IgGs to be directly purified using immobilized metal affinity chromatography (IMAC; Porath and Olin, 1983, Biochemistry 22, 1621-1630). This observation is in good agreement with previous work suggesting that the stretch of amino acids spanning positions 429-447 in human IgG1s could account for this purification property (Hale and Beidler, 1994, Anal. Biochem. 222, 29-33). The present study provides a more detailed molecular mechanism. More particularly, structural analysis of Fc/3M showed that the side chains of H310 and 1-1435 approach each other through a rotation around their Cα-Cβ bond (Chi 1 rotamers). In the presence of Zn²⁺ ions, the two imidazole rings coordinate the ion on the surface of the protein which then fulfills its tetrahedral coordination sphere by binding to two water molecules as shown in FIG. 2. The Zn²⁺ ion bound to H433 also fulfills its tetrahedral coordination sphere by binding to three water molecules. This is reminiscent of the human Fc structure described by Deisenhofer et al., 1981, Biochemistry 20, 2361-2370, in which a cadmium and zinc ions were found to be chelated by H310/H435 and H433, respectively.

### 7.2.5 STRUCTURAL ANALYSIS

The overall three-dimensional structure of Fc/3M is very similar to previously reported structures of human Fc regions (Deisenhofer et al. 1981, Biochemistry 20, 2361-2370; Sondermann et al. 2000, Nature 406, 267-273; Krapp et al. 2003, J. Mol. Biol. 325, 979-989; Matsumiya et al. 2007, J. Mol. Biol. 368, 767-779). In particular, the structure of the unmutated human Fc described by Krapp et al., 2003, J. Mol. Biol. 325, 979-989, with PDB ID number 1H3W, exhibited the most similarity in cell parameters, space group and packing when compared with Fc/3M. However, the respective crystallization conditions were different. Despite differences in terms of asymmetric unit contents, resolution and intrinsic crystal properties amongst other human Fc structures (including Fc/3M), all C_{H}2 and C_{H}3 domains showed considerable structural conservation and rigidity when considered separately. A domain-by-domain comparison suggested that C_{H}3 was the most conformationally conserved domain. Indeed, superimposition of C_{H}2 and C_{H}3 domains from various crystal structures hardly showed RMS deviations in excess of 0.5-0.6 Å for C_{α}.

However, C_{H}2 and C_{H}3 domains exhibited substantial relative flexibility. Thus, to better quantify this type of structural variation at the CD16 binding interface, Fc/3M C_{H}3 domains were superimposed with those of other unliganded human Fc portions and evaluated differences in the positions of the various C_{H}2 domains, as shown in FIG. 3. As shown in FIG. 3, the overall conformation of Fc/3M appeared more “open” when compared with other unliganded human Fc molecules. This comparison was carried out using the following human Fc structures: PDB ID numbers 1FC1 and 1FC2 (Deisenhofer et al. 1981), PDB ID numbers 1H3T/U/V/W/X/Y (Krapp et al. 2003, J. Mol. Biol. 325, 979-989), PDB ID numbers 2DTQ and 2DTS (Matsumiya et al. 2007, J. Mol. Biol. 368, 767-779), PDB ID number 1E4K (Sondermann et al. 2000, Nature 406, 267-273) and PDB ID number 1T83 (Radaev et al. 2001).

The extent of openness was assessed for all previously described human Fc structures as defined by (i) the inter-molecular distance between select portions of the polypeptide chains, and (ii) the angel between C_{H}2 and C_{H}3 domains, as summarized in Table 6.

The inter-molecular distances were measured using the Cα atom of P329, whose close proximity to the N-terminus in Fc polypeptide chains makes it a useful reference point as was previously shown. See Krapp et al. 2003, J. Mol. Biol. 325, 979-989. When so defined, Fc/3M exhibited the most open conformation of all known unliganded Fc structures. Intermolecular distances for the three most open unliganded human Fc structures were estimated at 39.1. 33.8 and 29.6 Å for Fc/3M, human Fc PDB ID number 1H3W (Krapp et al. 2003, J. Mol. Biol. 325, 979-989) and human Fc PDB ID number 1H3Y (Krapp et al. 2003, J. Mol. Biol. 325, 979-989), respectively. See Table 6.

Alternatively, the core β-barrel residue V323 was also used to calculate inter-molecular distances. In this situation, Fc/3M also exhibited the most open conformation. Intermolecular distances for the three most open unliganded human Fc structures were estimated at 43.6, 41.3 and 36.8 Å for Fc/3M, human Fc PDB ID number 1H3W (Krapp et al. 2003) and human Fc PDB ID number 1FC1 (Deisenhofer et al., 1981, Biochemistry 20: 2361-2370), respectively. See Table 6.

In addition, the angle defined by C_{H}2 and C_{H}3 could be assessed for each chain by the angle formed by a Cα atom in the C_{H}3 domain close to the Fc C terminus (for example, L443), a Cα atom in the hinge between C_{H}2 and C_{H}3 domains (for example, Q342) and a Cα atom in the C_{H}2 domain close to the Fc N terminus (for example, P329). When so defined, the respective C_{H}2/C_{H}3 angles for the four most open structures were 124.2, 124.7, 122.9, 119.8 and 119.4° for Fc/3M, chain B of human Fc PDB ID number 1E4K (Sondermann et al. 2000, Nature 406, 267-273), chain B of human Fc PDB ID number 1H3Y (Krapp et al. 2003, J. Mol. Biol. 325, 979-989), chain A of human Fc PDB ID number 1T83 (Radaev et al. 2001, J. Biol. Chem. 276, 16469-16477) and human Fc PDB ID number 1H3W (Krapp et al. 2003, J. Mol. Biol. 325, 979-989) respectively. See Table 6.

The angle defined by C_{H}2 and C_{H}3 could alternatively be assessed by the angle formed by three atoms: a Cα atom in the core β-barrel of the C_{H}3 domain spatially close to the Fc C terminus (for example, F423), a Cα atom in the core β-barrel of the C_{H}3 domain close to the C_{H}2/C_{H}3 junction (for example, E430) and a Cα atom in the core β-barrel of the C_{H}2 domain spatially close to the Fc N terminus (for example, V323). Here again, Fc/3M exhibited the most open conformation when compared with other unliganded human Fc structures. More specifically, the respective C_{H}2/C_{H}3 angles for the three most open unliganded human Fc structures were estimated at 129.0, 128.7 and 125.3° for Fc/3M, chain B of human Fc PDB ID number 1H3Y (Krapp et al. 2003) and chain A of human Fc PDB ID number 1H3Y (Krapp et al. 2003), respectively. See Table 6.

In summary, Fc/3M exhibited the most open conformation when compared with unliganded human Fc structures. This large opening between Fc/3M C_{H}2 domains cannot be easily explained through direct effects of the 3M mutation, since the corresponding amino acids do not form any intermolecular interaction.

It is possible that the values for Fc/3M inter-molecular distances and angles are within their range of intrinsic variability in human Fc. Large variations exist when intermolecular distances or C_{H}2/C_{H}3 angles are compared amongst similar proteins. For instance, as shown in Table 6, intermolecular distances (as measured by P329/P329) vary by as much as 7 Å between unliganded Fc molecules (PDB ID numbers 1FC1 and 1H3W). Similarly, intermolecular distances (as measured by V323/V323) vary by as much as 8 Å between unliganded Fc molecules (such as PDB ID numbers 2DTQ and 1H3W). Likewise, C_{H}2/CH3 angles vary by as much as 7.2° between CD16-bound Fc molecules (chain B of PDB ID numbers 1E4K and 1T83), when L443, Q342 and P329 were used in measurement. Similarly, C_{H}2/C_{H}3 angles can vary by as much as 10.4° between CD16-bound Fc molecules (such as the respective A chains of PDB ID numbers 1E4K and 1T89), when F423, E430 and V323 were used in measurement.

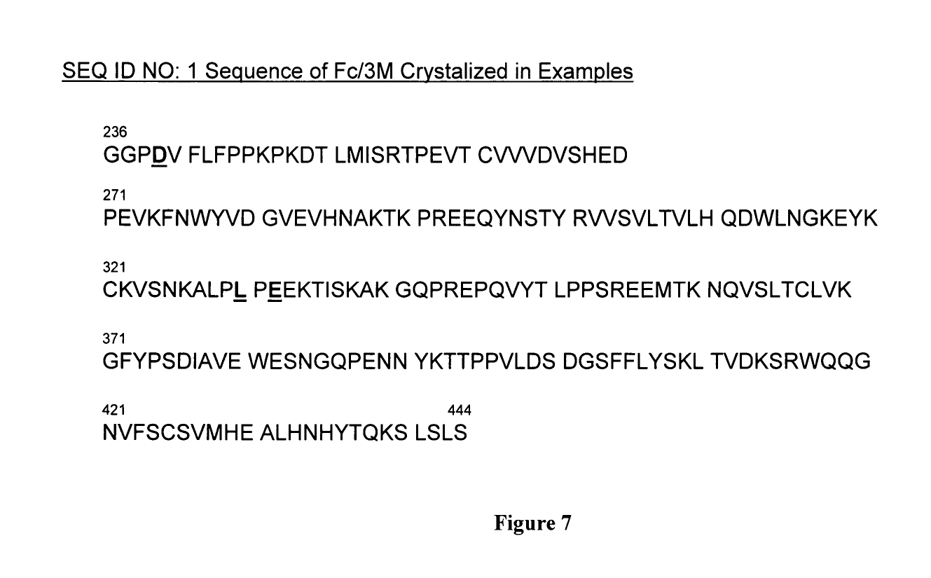

Table 5, following below, provides the atomic structure coordinates of Fc/3M. In the Table, coordinates for Fc/3Mare provided. The amino acid residue numbers coincide with those used in FIGS. 7.

The following abbreviations are used in Table 5:

“Atom Type” refers to the element whose coordinates are provided. The first letter in the column defines the element.

“A.A.” refers to amino acid.

“X, Y and Z” provide the Cartesian coordinates of the element.

“B” is a thermal factor that measures movement of the atom around its atomic center.

“OCC” refers to occupancy, and represents the percentage of time the atom type occupies the particular coordinate. OCC values range from 0 to 1, with 1 being 100%.

### 7.2.6 FC/3M STRUCTURE/PROPERTIES RELATIONSHIP

Differential Scanning Calorimetry

Differential scanning calorimetry (DSC) measurements were measured with a VP-DSC instrument (MicroCal, LLC, Northampton, Mass.) using a typical scan rate of 1.0° C./min and a temperature range of 25-110° C. A filter period of 8 s was used along with a 15 min pre-scan thermostating. 3F2, 3F2/3M, 3F2/Fab, Fc/3M and unmutated human Fc samples were prepared by dialysis into 10 mM histidine-HCl, pH 6.0 and used at a concentration of 0.1 mg/ml as determined by their absorbance at 280 nm. Multiple baselines were run in the same buffer in both the sample and reference cell to establish thermal equilibrium. After the baseline was subtracted from the sample thermogram, the data were concentration-normalized and the melting temperatures determined using the “Origin 7” software (OriginLab Corporation, Northampton, Mass.).

Thermostability

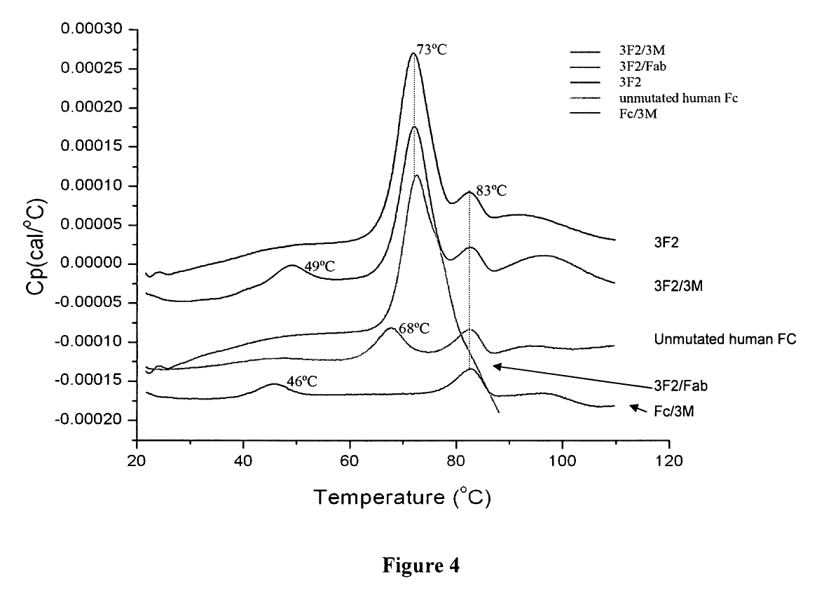

The effect of 3M on protein stability was assessed by differential scanning calorimetry (DSC) experiments which compared the thermostability of both a humanized anti-human EphA2 IgG1/κ (namely 3F2) and unmutated human Fc fragment (γ1) with that of their 3M-mutated counterparts (3F2/3M and Fc/3M, respectively). Deconvolution of 3F2, 3F2/3M, unmutated human Fc and Fc/3M thermograms revealed two, three, two and two, respectively, major transitions. Typical thermograms are shown in FIG. 4 and the corresponding melting temperatures (Tm) are reported in Table 4. Data suggested that 3F2/3M exhibited a significantly decreased thermal stability when compared with 3F2 due to the existence of a low temperature Tm peak (49° C.) in its thermogram. Because of the reported thermodynamic and unfolding independence of the Fab and Fc portions within an IgG (Tischenko et al. 1982, Eur. J. Biochem. 126, 517-521; Vermeer et al. 2000, Biophys. J. 79, 2150-2154) and in light of 3F2 and 3F2/3M identical Fab regions, we attributed this additional transition to the premature unfolding of 3F2/3M mutated Fc. This was confirmed by the analysis of the DSC thermograms of individual Fab and Fc regions. In this situation, we could attribute the 73° C. transition seen for the full-length 3F2 IgG to its Fab portion. Analysis of the unmutated human Fc revealed two discrete transitions at 83 and 68° C. potentially attributable to its C_{H}3 and C_{H}2 regions, respectively. Indeed, the thermogram corresponding to Fc/3M, whose C_{H}3 region is identical to the unmutated human Fc, also exhibited a transition at 83° C. Fc/3M second transition at 46° C. likely corresponded to its mutated C_{H}2 portion and was similar to the lowest transition observed in the full-length 3F2/3M IgG (namely 49° C.). Thus, the 3M-mediated decrease in protein thermostability was estimated at between 19 and 22° C. when in the context of a full-length IgG and isolated Fc fragment, respectively.

The analysis of our Fc/3M structure did not provide a straightforward explanation as to the nature of the molecular mechanisms responsible for this markedly decreased thermostability. Indeed, no net loss on intra- or inter-molecular interaction could be observed when compared with unmutated human Fc fragments. It is possible that this result be due to the increased distance between C_{H}2 domains (see section above), resulting in an increased lability of the entire Fc. Alternatively, dynamic conformational changes occurring within the Fc regions and not visualized using X-ray crystallography techniques could also be invoked.

### 7.2.7 INTERACTION WITH HUMAN CD16

Generation of Human CD 16

Human CD 16 (VI58 allotype) used in BIAcore measurements was generated from the human CD 16 construct (F158 allotype) previously described.” The cloned CD16/F158 was mutated at position 158 (F to V) using a QuickChange XL mutagenesis kit according to the manufacturer's instructions (Stratagene). The expression and purification of human CD16/V158 were then carried out essentially as described in Dall'Acqua et al., 2006, J. Biol. Chem. 281:23514-23524.

BIAcore Measurements

The interaction of soluble CD 16 (VI58 allotype) with immobilized unmutated human Fc and Fc/3M was monitored by surface plasmon resonance detection using a BIAcore 3000 instrument (Biacore International AB, Uppsala, Sweden). Unmutated human Fc and Fc/3M were first coupled to the dextran matrix of a CM5 sensor chip (Biacore International AB) using an Amine Coupling Kit at a surface density of between 2523 and 2543 RU according to the manufacturer's instructions. Human CD 16 was buffer-exchanged against PBS buffer and used in equilibrium binding experiments at concentrations ranging from 1 nM to 1.6 uM at a flow rate of 5 uL/min. Dilutions and binding experiments were carried out at 25° C. in 50 mM HBS buffer containing 0.01 M HEPES, pH 7.4, 0.15 M NaCl3 mM EDTA and 0.005% P-20. Steady-state binding data were collected for 50 min. Fc surfaces were regenerated with a 1 min injection of 5 mM HCl Human CD 16 was allowed to flow over an uncoated cell and the sensorgrams from these blank runs subtracted from those obtained with Fc-coupled chips. Dissociation constants (Kns) were determined by fitting the corresponding binding isotherms and are recorded in Table 7.

Interaction with CD 16

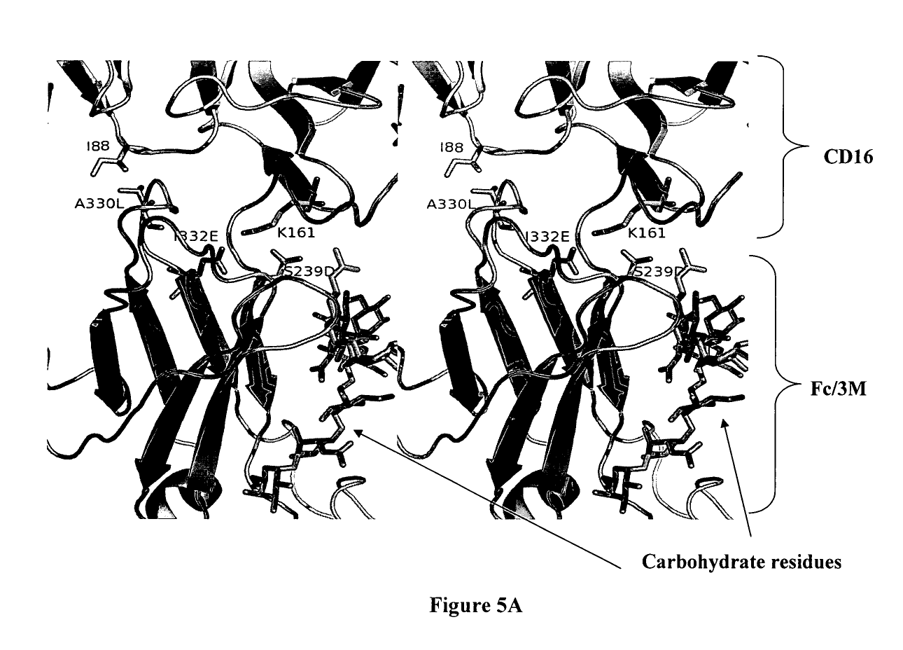

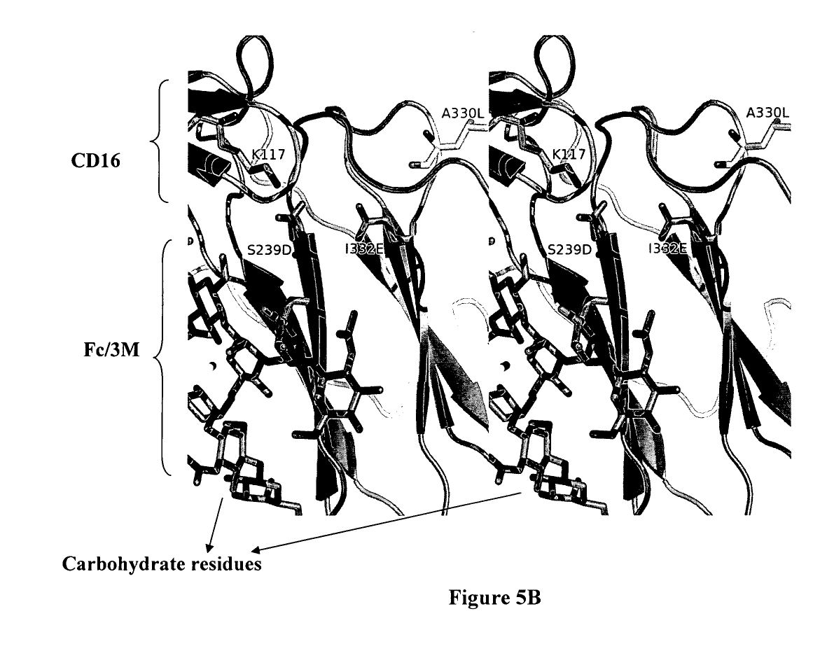

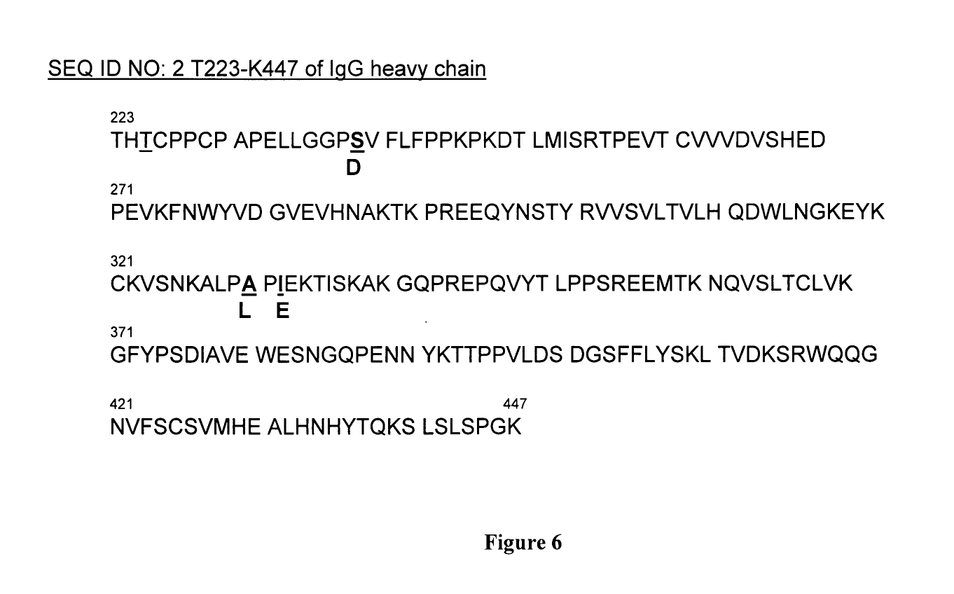

The three-dimensional structure of the Fc/3M-human CD 16 complex would likely provide a robust molecular explanation for the increased binding affinity between 3M-modified human IgG1s and human CD16. By using the publicly available structure of a human Fc-human CD16 complex (Radaev et al. 2001, J. Biol. Chem. 276, 16469-16477) and assuming a similar interaction interface for Fc/3M, some important clues may be obtained. For this purpose, a model of the complex between Fc/3M and CD16 was constructed. Due to the asymmetric nature of the interaction between human CD16 and homodimeric human Fc (Radaev et al. 2001, J. Biol. Chem. 276, 16469-16477), the three mutations introduced are likely to be playing different roles depending on the polypeptide chain of the Fc region they are located in. In one chain (FIG. 5A), mutations S239D and I332E may establish two additional hydrogen bonds and/or additional electrostatic interaction with the side chain of human CD16 K158 (K161 in standard NCBI numbering), whereas A330L may contribute to additional hydrophobic interactions with human CD16 I85 (187 in standard NCBI numbering).

In the other chain (FIG. 5B), the S239D substitution may create an additional hydrogen bond and/or additional electrostatic interaction with the side chain of human CD16 K117 (K120 in standard NCBI numbering), whereas mutations A330L and I332E may not play any significant role since they are located away from the contact interface. None of these new contacts would be either substituting or breaking pre-existing contacts within the Fc region. Thus, the enhanced interaction with human CD16 mediated by 3M could probably be explained by the formation of additional hydrogen bonds, hydrophobic contacts and/or additional electrostatic interaction, as opposed to large conformational changes in the Fc region.

Conceivably, the open state of Fc/3M C_{H}2 and C_{H}3 domains could also contribute to the increased association constant with human CD 16 by holding the Fc region in a conformation more favorable for binding CD16. It was noted that human Fc fragments in complex with human CD 16 comprised one chain exhibiting a similar openness of their C_{H}2 domains. When L443, Q342 and P329 were used in measurement, the angles between C_{H}2 and C_{H}3 domains are 124.7° and 122.5° vs. 124.2° for Fc/3M; 1E4K (Sondermannn et al. 2000, Nature 406, 267-273); 1T83 (Radaev et al. 2001, J. Biol. Chem. 276, 16469-16477)). See Table 6. However, the unliganded human Fc corresponding to PDB ID number 1H3Y (Krapp et al. 2003, J. Mol. Biol. 325, 979-989) comprised one chain (chain B) with a similarly large C_{H}2/C_{H}3 angle (namely 122.9°). See Table 6.

Similarly, when F423, E430 and V323 were used in measurement, the angles between C_{H}2 and C_{H}3 domains are 127.9° and 128.4°1E4K and 1T83. See Table 6. The unliganded human Fc corresponding to PDB ID number 1H3Y comprised one chain (chain B) with a similarly large C_{H}2/C_{H}3 angle 128.7°. See Table 6.

Thus, as previously mentioned, Fc/3M conformational parameters could conceivably represent just one snapshot within their normal intrinsic variability range in human Fc. Furthermore, the unliganded human Fc corresponding to PDB ID number 1H3W (Krapp et al. 2003, J. Mol. Biol. 325, 979-989) exhibited both a relatively open conformation as defined by P329/P329 and V323N323 interchain distances (33.8 Å and 41.3 Å respectively, Table 6) as well as the same space group as Fc/3M (C222₁). Thus, the openness seen in Fc/3M could also be related to the crystal's intrinsic properties as opposed to the 3M mutations. In this situation, no significant 3M-mediated structural changes could be invoked.

It is possible that specific structural characteristics present in IgG but not in isolated Fc fragments may have gone unnoticed in the present study. Likewise, certain of the structural features seen in Fc/3M may not occur within a full-length human IgG1. However, it is believed that Fc/3M constituted a relevant model since the increase in its binding affinity to human CD16N158 when compared with an unmutated human Fc fragment (˜30-fold; Table 7) was comparable to what was observed using human IgG1s (Lazar et al., 2006, Proc. Natl. Acad. Sci. 103:4005-4010; Dall'Acqua et al., 2006, J. Biol. Chem. 281:23514-23524).

### 7.3 MODULATION OF ADCC ACTIVITY

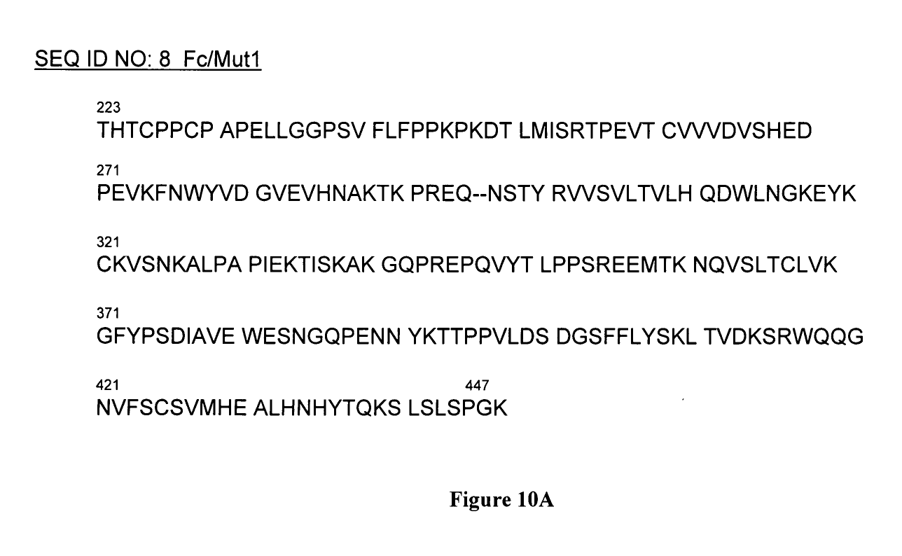

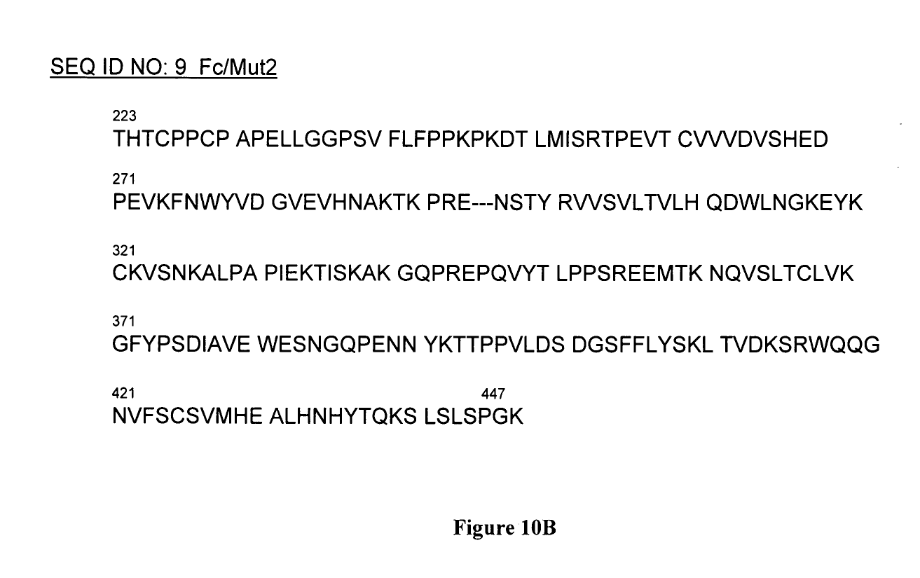

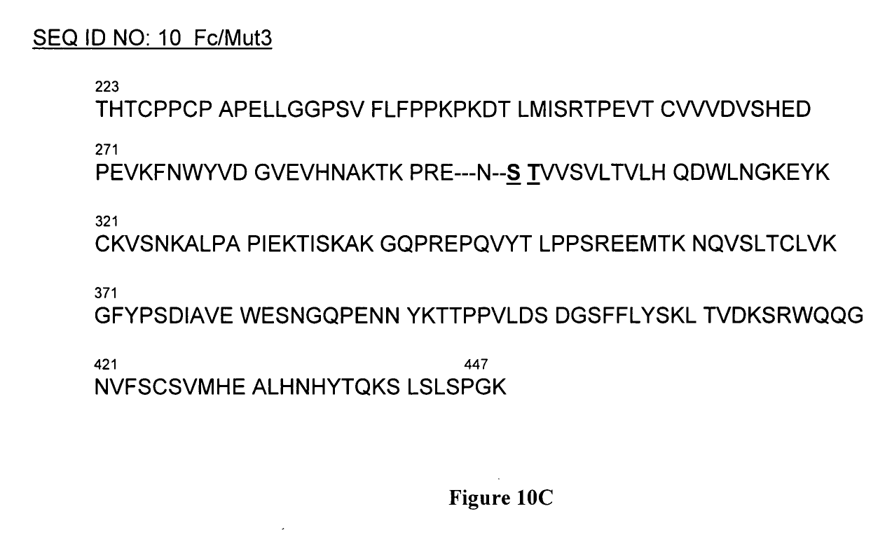

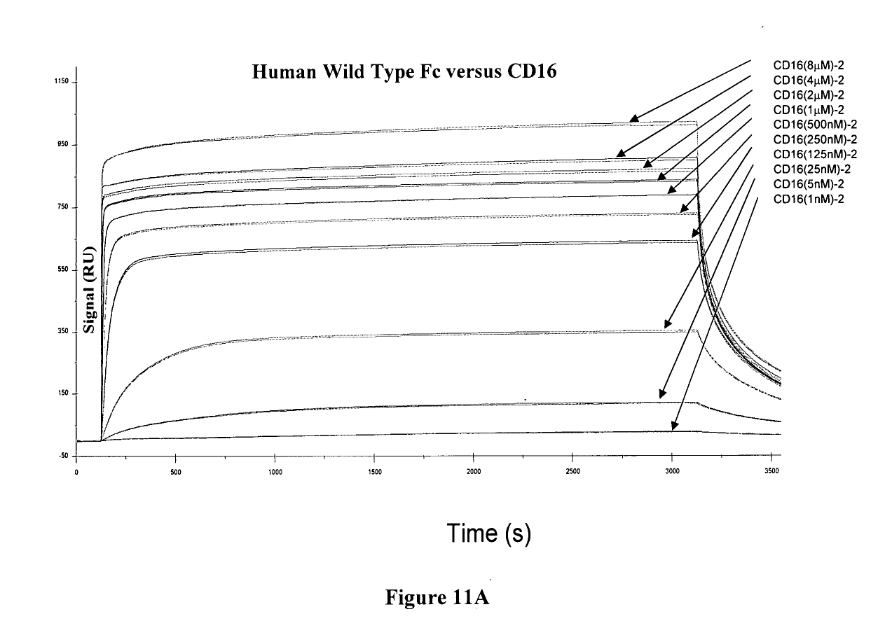

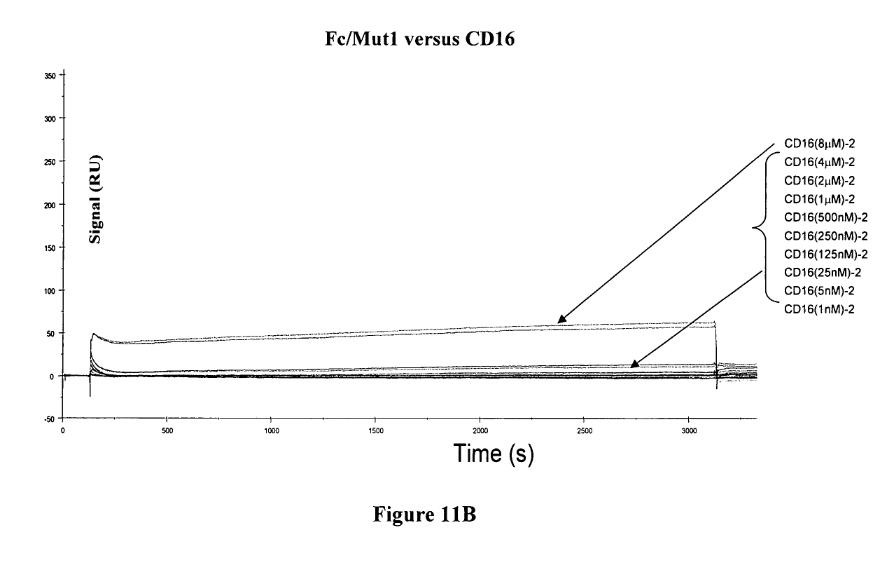

Based on the the structural features seen in Fc/3M three human IgG Fc variants were designed:
Fc/Mut1: Comprising SEQ ID NO: 8 as depicted in FIG. 10A, contains deletion of residues at positions 295 and 296 (according to EU numbering).
Fc/Mut2: Comprising SEQ ID NO: 9 as depicted in FIG. 10B, contains deletion of residues at positions 294, 295 and 296 (according to EU numbering).
Fc/Mut3: Comprising SEQ ID NO: 10 as depicted in FIG. 10C, contains deletion of residues at positions 294, 295, 296, 298 and 299 as well as substitutions at positions 300 and 301 by Serine and Threonine, respectively (all according to EU numbering).

These human IgG Fc variants all have the potential to lead to conformational changes at the human Fc/human CD 16 binding interface and/or to modulate the corresponding interaction. Characterization of the binding of these three human IgG Fc variants demonstrates that each exhibits a significantly reduced binding to each FcγR tested This in turn would impact the ADCC activity of said human IgG variants.

### 7.3.1 GENERATION, EXPRESSION AND PURIFICATION OF HUMAN FC CONSTRUCTS

Recombinant human IgG Fc (γ1 isotype) was cloned into a mammalian expression vector encoding a human cytomegalovirus major immediate early (hCMVie) enhancer, promoter and 5′-untranslated region. Fc/Mut1, Fc/Mut2 and Fc/Mut3 were generated using the polymerase chain reaction (PCR) by overlap extension and standard protocols. These were then cloned into the same mammalian expression construct as the unmutated human Fc.

All Fc constructs were transiently transfected into Human Embryonic Kidney (HEK) 293 cells using Lipofectamine (Invitrogen, Inc., Carlsbad, Calif.) and standard protocols. Proteins were typically harvested at 72, 144 and 216 hours post-transfection and purified from the conditioned media directly on HiTrap protein A columns according to the manufacturer's instructions (APBiotech, Inc., Piscataway, N.J.). Purified human Fc, Fc/Mut1, Fc/Mut2 and Fc/Mut3 (typically >95% homogeneity, as judged by SDS-PAGE) were then submitted to various binding measurements using BIAcore (see below).

### 7.3.2 BINDING MEASUREMENTS

The interaction of soluble human CD16 (F158 allotype) with immobilized human IgG Fc, Fc/Mut1, Fc/Mut2 and Fc/Mut3 was monitored by surface plasmon resonance detection using a BIAcore 3000 instrument (Biacore International AB, Uppsala, Sweden). Human IgG Fc molecules and their variants were first coupled to the dextran matrix of a CM5 sensor chip (Biacore International AB) using an Amine Coupling Kit at a surface density of between 2645 and 3011 RU according to the manufacturer's instructions. Human CD 16 was used in equilibrium binding experiments at concentrations ranging from 1 nM to 8 μM at a flow rate of 5 4/min. Dilutions and binding experiments were carried out at 25° C. in 50 mM HBS buffer containing 0.01 M HEPES, pH 7.4, 0.15 M NaCl, 3 mM EDTA and 0.005% P-20. Steady-state binding data were collected for approximately 50 min. Human IgG Fc surfaces were regenerated with a 1 min injection of 5 mM HCl. Human CD16 was also allowed to flow over an uncoated cell, and the sensorgrams from these blank runs subtracted from those obtained with human Fc-coupled chips.

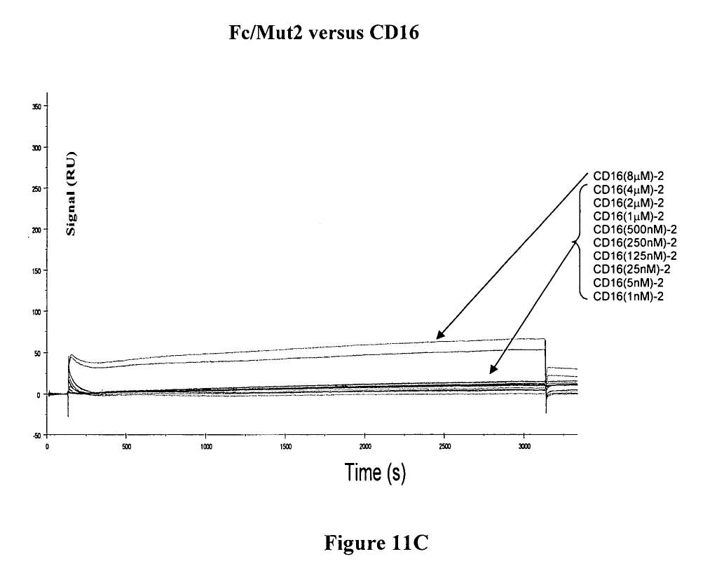

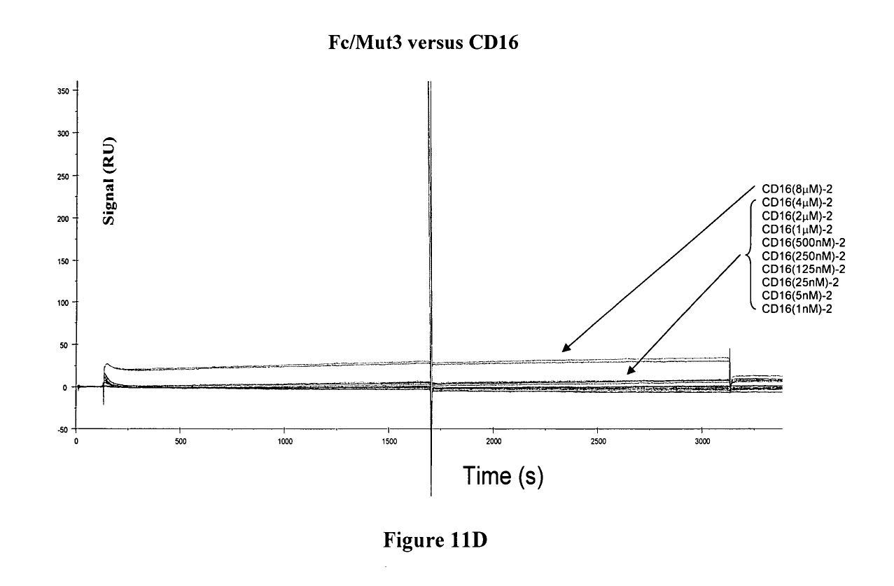

The dissociation constant (K_{D}) for the unmutated human IgG Fc/human CD16 (F158 allotype) interaction was determined by fitting the corresponding binding isotherms (FIG. 11A) and was estimated at 41±2 nM (the error was estimated as the standard deviations of 2 independent experiments). In contrast, human CD16 binding to Fc/Mut1, Fc/Mut2 and Fc/Mut3 could only be detected at a the highest human CD16 concentration tested (namely 8 μM; Compare FIG. 11A to 11B-D). This demonstrated that the binding of human CD16 to Fc/Mut1, Fc/Mut2 and Fc/Mut3 was essentially knocked-out.

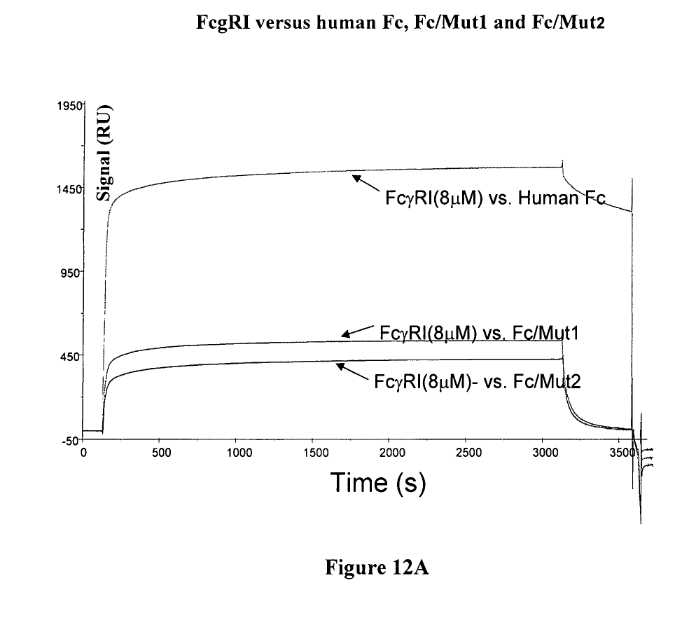

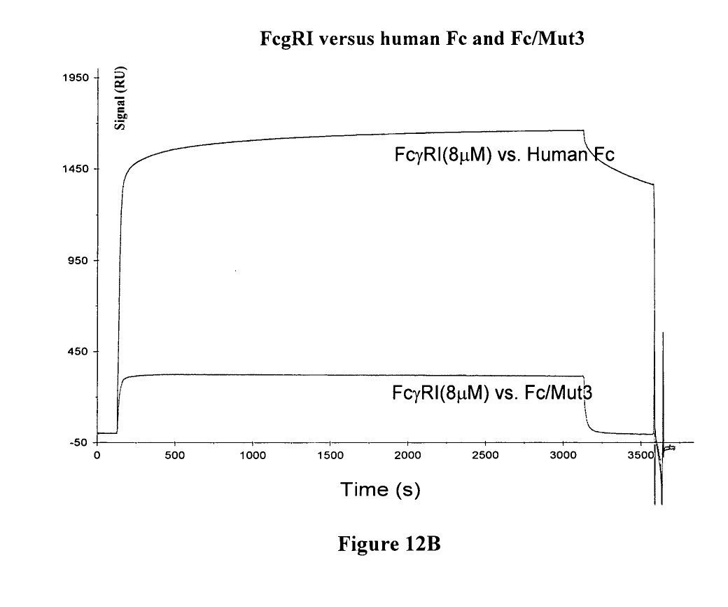

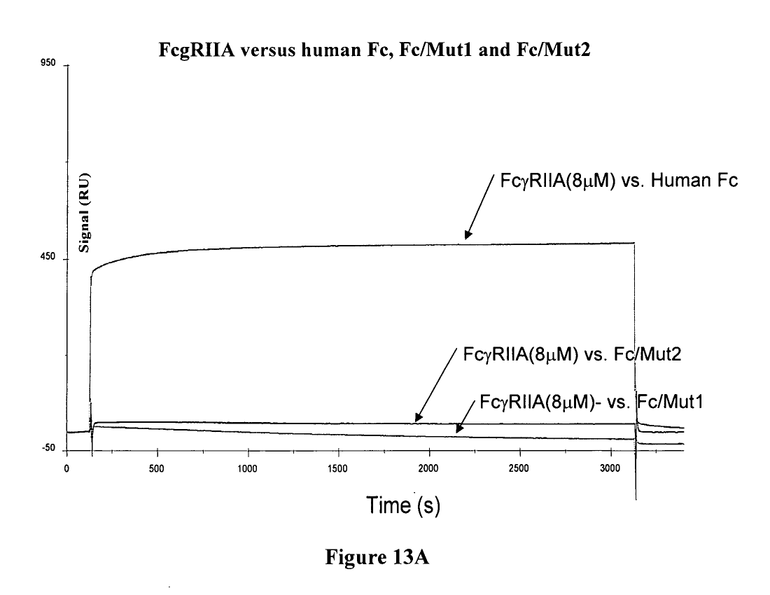

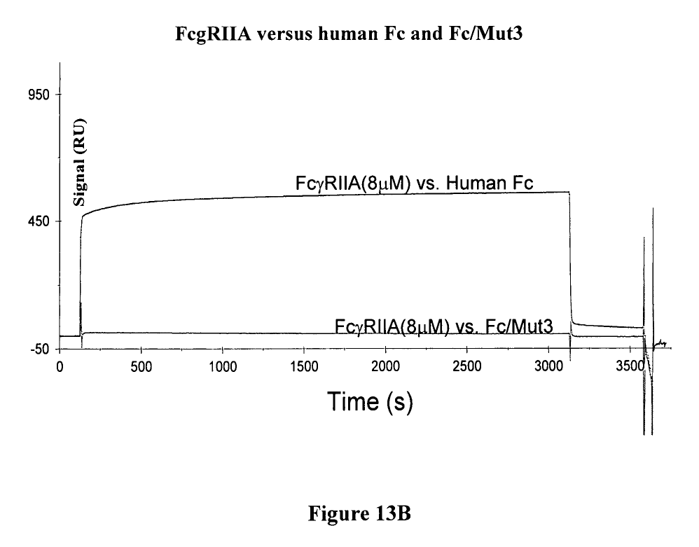

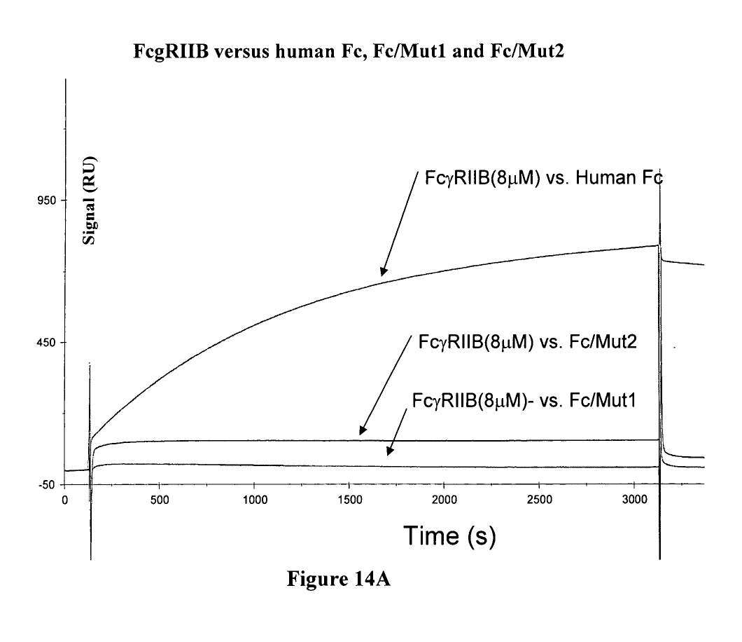

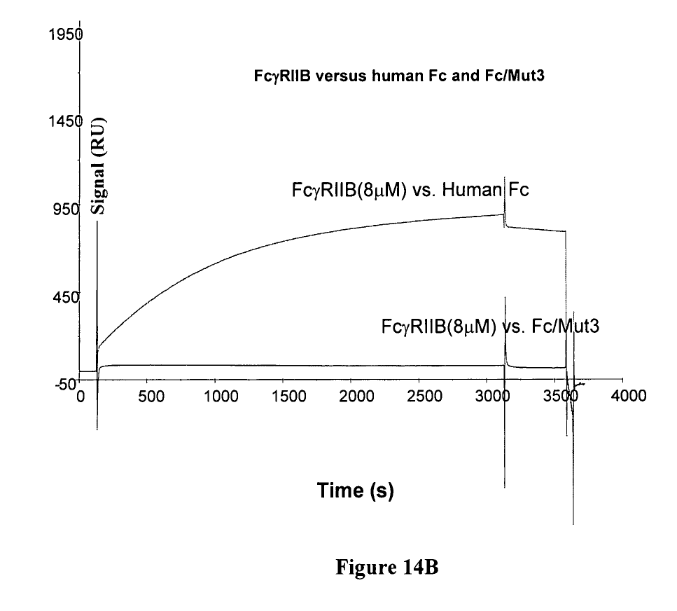

Binding of human FcγRIIA and FcγRIIB to human IgG Fc, Fc/Mut1, Fc/Mut2 and Fc/Mut3 revealed that all Fc variants also exhibited an essentially knocked-out binding to these receptors (FIGS. 13 and 14). Finally, Fc/Mut1, Fc/Mut2 and Fc/Mut3 exhibited a significantly decreased binding to human FcγRI (FIG. 12).

The present invention is not to be limited in scope by the exemplified embodiments, which are intended as illustrations of single aspects of the invention. Indeed, various modifications of the invention in addition to those described herein will become apparent to those having skill in the art from the foregoing description and accompanying drawings. Such modifications are intended to fall with in the scope of the appended claims.

All documents referenced in this application, whether patents, published or unpublished patent applications, either U.S. or foreign, literature references, nucleotide or amino acid sequences identified by Accession No. or otherwise, are hereby incorporated by reference in their entireties for any and all purposes.

TABLE 2
Summary of Data Collection
Data Collection for Fc/3M

| | |
|---|---|
| Wavelength, \acute{Å} | 1.54 |
| Resolution, \acute{Å} | 2.53 (2.61 − 2.53) ^{a} |
| Space group | C222₁ |
| Cell parameters, \acute{Å} | 49.87, 147.49, 74.32 |
| Total reflections | 25,943 |
| Unique reflections | 9,484 |
| R_{sym} | 0.159 (0.614) ^{a} |
| Completeness, % | 92.6 (100.0) ^{a} |
| I/σ(I) | 7.5 (1.9) ^{a} |

^{a} Values in parenthesis correspond to the highest resolution shell
R_{sym} = 100 × ΣₕΣᵢ | Iᵢ(h) − <I(h)> |/ΣₕΣᵢIᵢ(h)

TABLE 3
Refinement Statistics

| | |
|---|---|
| Resolution range, \acute{Å} | 47.0 − 2.5 |
| R factor (Free-R factor) | 0.223 (0.290) |
| RMSD bonds, \acute{Å} | 0.015 |
| RMSD angles, ° | 1.72 |
| Residues in most favored region of | 91.2 |
| {φ, ψ} space, % | |
| Residues in additionally allowed | 8.8 |
| region of (φ, ψ} space, % | |
| Number of protein atoms | 6220 |
| Number of non-protein atoms | 487 |
| B factor (Model/Wilson), \acute{Å}² | 46.2/44 |

R-value = Σ ₕ|| F_{obs} (h)| − | F_{calc} (h) ||/Σ ₕ| F_{obs} (h)| for all reflections.

TABLE 4
Thermal melting temperatures (Tm) of unmutated human
Fc, Fc/3M and 3F2 variants.

| Molecule | Tm (° C.) |
|---|---|
| 3F2 | 83/73 ^{b} |
| 3F2/3M | 83/73/49 ^{b} |
| 3F2/Fab | 73 ^{b} |
| Fc/3M | 83/46 ^{b} |
| Unmutated human Fc | 83/68 ^{b} |

^{a} Tm values were determined as described in Materials and Methods.
^{b} One to three major transitions were observed in these samples. Values reflect each of the individual thermogram peaks.

TABLE 5
Atom Coordinate Structures of Fc/3M

| Atom | A.A. | Type | | | | X | Y | Z | Occ | B | |
|---|---|---|---|---|---|---|---|---|---|---|---|
| ATOM | 1 | N | GLY | A | 236 | −4.926 | 40.715 | −10.771 | 1.00 | 50.18 | N |
| ATOM | 2 | CA | GLY | A | 236 | −6.393 | 40.517 | −10.995 | 1.00 | 50.49 | C |
| ATOM | 3 | C | GLY | A | 236 | −6.752 | 40.163 | −12.440 | 1.00 | 50.55 | C |
| ATOM | 4 | O | GLY | A | 236 | −5.979 | 40.469 | −13.367 | 1.00 | 50.61 | O |
| ATOM | 5 | N | GLY | A | 237 | −7.919 | 39.536 | −12.660 | 1.00 | 50.51 | N |
| ATOM | 6 | CA | GLY | A | 237 | −8.936 | 39.236 | −11.614 | 1.00 | 50.20 | C |
| ATOM | 7 | C | GLY | A | 237 | −9.368 | 37.773 | −11.404 | 1.00 | 49.62 | C |
| ATOM | 8 | O | GLY | A | 237 | −8.720 | 36.840 | −11.951 | 1.00 | 49.66 | O |
| ATOM | 9 | N | PRO | A | 238 | −10.479 | 37.565 | −10.634 | 1.00 | 48.84 | N |
| ATOM | 10 | CA | PRO | A | 238 | −10.845 | 36.256 | −10.055 | 1.00 | 48.21 | C |
| ATOM | 11 | CB | PRO | A | 238 | −12.206 | 36.516 | −9.402 | 1.00 | 47.81 | C |
| ATOM | 12 | CG | PRO | A | 238 | −12.216 | 37.956 | −9.129 | 1.00 | 48.10 | C |
| ATOM | 13 | CD | PRO | A | 238 | −11.473 | 38.594 | −10.274 | 1.00 | 48.80 | C |
| ATOM | 14 | C | PRO | A | 238 | −10.924 | 35.117 | −11.065 | 1.00 | 47.74 | C |
| ATOM | 15 | O | PRO | A | 238 | −11.401 | 35.315 | −12.184 | 1.00 | 48.11 | O |
| ATOM | 16 | N | ASP | A | 239 | −10.446 | 33.951 | −10.669 | 1.00 | 46.85 | N |
| ATOM | 17 | CA | ASP | A | 239 | −10.459 | 32.759 | −11.477 | 1.00 | 46.06 | C |
| ATOM | 18 | CB | ASP | A | 239 | −9.053 | 32.414 | −11.969 | 1.00 | 46.47 | C |
| ATOM | 19 | CG | ASP | A | 239 | −8.279 | 33.648 | −12.454 | 1.00 | 48.77 | C |
| ATOM | 20 | OD1 | ASP | A | 239 | −8.673 | 34.239 | −13.489 | 1.00 | 51.90 | O |
| ATOM | 21 | OD2 | ASP | A | 239 | −7.285 | 34.030 | −11.797 | 1.00 | 49.73 | O |
| ATOM | 22 | C | ASP | A | 239 | −11.059 | 31.648 | −10.606 | 1.00 | 45.50 | C |
| ATOM | 23 | O | ASP | A | 239 | −10.660 | 31.479 | −9.461 | 1.00 | 45.43 | O |
| ATOM | 24 | N | VAL | A | 240 | −12.035 | 30.924 | −11.186 | 1.00 | 44.28 | N |
| ATOM | 25 | CA | VAL | A | 240 | −12.714 | 29.814 | −10.554 | 1.00 | 42.81 | C |
| ATOM | 26 | CB | VAL | A | 240 | −14.260 | 29.958 | −10.706 | 1.00 | 43.26 | C |
| ATOM | 27 | CG1 | VAL | A | 240 | −14.986 | 28.969 | −9.795 | 1.00 | 41.27 | C |
| ATOM | 28 | CG2 | VAL | A | 240 | −14.702 | 31.374 | −10.386 | 1.00 | 43.38 | C |
| ATOM | 29 | C | VAL | A | 240 | −12.351 | 28.464 | −11.130 | 1.00 | 42.42 | C |
| ATOM | 30 | O | VAL | A | 240 | −12.388 | 28.267 | −12.345 | 1.00 | 41.72 | O |
| ATOM | 31 | N | PHE | A | 241 | −12.039 | 27.538 | −10.230 | 1.00 | 41.79 | N |
| ATOM | 32 | CA | PHE | A | 241 | −11.770 | 26.112 | −10.539 | 1.00 | 40.81 | C |
| ATOM | 33 | CB | PHE | A | 241 | −10.312 | 25.715 | −10.288 | 1.00 | 40.37 | C |
| ATOM | 34 | CG | PHE | A | 241 | −9.374 | 26.505 | −11.127 | 1.00 | 40.12 | C |
| ATOM | 35 | CD1 | PHE | A | 241 | −8.651 | 27.535 | −10.555 | 1.00 | 41.56 | C |
| ATOM | 36 | CE1 | PHE | A | 241 | −7.785 | 28.302 | −11.342 | 1.00 | 43.74 | C |
| ATOM | 37 | CZ | PHE | A | 241 | −7.659 | 28.032 | −12.711 | 1.00 | 42.07 | C |
| ATOM | 38 | CE2 | PHE | A | 241 | −8.398 | 27.000 | −13.275 | 1.00 | 41.41 | C |
| ATOM | 39 | CD2 | PHE | A | 241 | −9.248 | 26.260 | −12.487 | 1.00 | 41.10 | C |
| ATOM | 40 | C | PHE | A | 241 | −12.669 | 25.232 | −9.683 | 1.00 | 40.19 | C |
| ATOM | 41 | O | PHE | A | 241 | −12.755 | 25.403 | −8.476 | 1.00 | 40.19 | O |
| ATOM | 42 | N | LEU | A | 242 | −13.323 | 24.297 | −10.373 | 1.00 | 38.61 | N |
| ATOM | 43 | CA | LEU | A | 242 | −14.272 | 23.395 | −9.778 | 1.00 | 36.99 | C |
| ATOM | 44 | CB | LEU | A | 242 | −15.638 | 23.616 | −10.391 | 1.00 | 35.26 | C |
| ATOM | 45 | CG | LEU | A | 242 | −16.754 | 22.790 | −9.796 | 1.00 | 33.69 | C |
| ATOM | 46 | CD1 | LEU | A | 242 | −17.126 | 23.193 | −8.404 | 1.00 | 28.51 | C |
| ATOM | 47 | CD2 | LEU | A | 242 | −17.923 | 22.935 | −10.704 | 1.00 | 33.04 | C |
| ATOM | 48 | C | LEU | A | 242 | −13.728 | 21.988 | −9.995 | 1.00 | 37.10 | C |
| ATOM | 49 | O | LEU | A | 242 | −13.482 | 21.599 | −11.143 | 1.00 | 36.96 | O |
| ATOM | 50 | N | PHE | A | 243 | −13.501 | 21.275 | −8.880 | 1.00 | 37.35 | N |
| ATOM | 51 | CA | PHE | A | 243 | −12.837 | 19.958 | −8.844 | 1.00 | 38.12 | C |
| ATOM | 52 | CB | PHE | A | 243 | −11.654 | 19.929 | −7.870 | 1.00 | 37.70 | C |
| ATOM | 53 | CG | PHE | A | 243 | −10.599 | 20.946 | −8.166 | 1.00 | 38.21 | C |
| ATOM | 54 | CD1 | PHE | A | 243 | −9.612 | 20.685 | −9.131 | 1.00 | 36.93 | C |
| ATOM | 55 | CE1 | PHE | A | 243 | −8.615 | 21.643 | −9.415 | 1.00 | 37.94 | C |
| ATOM | 56 | CZ | PHE | A | 243 | −8.609 | 22.896 | −8.720 | 1.00 | 37.04 | C |
| ATOM | 57 | CE2 | PHE | A | 243 | −9.588 | 23.162 | −7.756 | 1.00 | 34.40 | C |
| ATOM | 58 | CD2 | PHE | A | 243 | −10.588 | 22.185 | −7.483 | 1.00 | 36.89 | C |
| ATOM | 59 | C | PHE | A | 243 | −13.799 | 18.815 | −8.484 | 1.00 | 38.77 | C |
| ATOM | 60 | O | PHE | A | 243 | −14.708 | 18.989 | −7.656 | 1.00 | 38.55 | O |
| ATOM | 61 | N | PRO | A | 244 | −13.625 | 17.658 | −9.143 | 1.00 | 38.83 | N |
| ATOM | 62 | CA | PRO | A | 244 | −14.488 | 16.538 | −8.851 | 1.00 | 39.51 | C |
| ATOM | 63 | CB | PRO | A | 244 | −14.176 | 15.547 | −9.978 | 1.00 | 39.59 | C |
| ATOM | 64 | CG | PRO | A | 244 | −12.824 | 15.925 | −10.484 | 1.00 | 38.55 | C |
| ATOM | 65 | CD | PRO | A | 244 | −12.707 | 17.392 | −10.267 | 1.00 | 38.83 | C |
| ATOM | 66 | C | PRO | A | 244 | −14.126 | 15.905 | −7.542 | 1.00 | 40.11 | C |
| ATOM | 67 | O | PRO | A | 244 | −13.075 | 16.197 | −7.013 | 1.00 | 41.03 | O |
| ATOM | 68 | N | PRO | A | 245 | −14.975 | 15.001 | −7.045 | 1.00 | 40.44 | N |
| ATOM | 69 | CA | PRO | A | 245 | −14.584 | 14.110 | −5.943 | 1.00 | 40.66 | C |
| ATOM | 70 | CB | PRO | A | 245 | −15.870 | 13.342 | −5.615 | 1.00 | 40.47 | C |
| ATOM | 71 | CG | PRO | A | 245 | −16.796 | 13.578 | −6.831 | 1.00 | 41.46 | C |
| ATOM | 72 | CD | PRO | A | 245 | −16.367 | 14.815 | −7.504 | 1.00 | 40.30 | C |
| ATOM | 73 | C | PRO | A | 245 | −13.468 | 13.160 | −6.384 | 1.00 | 41.43 | C |
| ATOM | 74 | O | PRO | A | 245 | −13.069 | 13.189 | −7.550 | 1.00 | 42.05 | O |
| ATOM | 75 | N | LYS | A | 246 | −12.948 | 12.342 | −5.472 | 1.00 | 41.52 | N |
| ATOM | 76 | CA | LYS | A | 246 | −12.034 | 11.265 | −5.864 | 1.00 | 41.85 | C |
| ATOM | 77 | CB | LYS | A | 246 | −11.165 | 10.874 | −4.640 | 1.00 | 42.73 | C |
| ATOM | 78 | CG | LYS | A | 246 | −10.041 | 11.901 | −4.289 | 1.00 | 43.80 | C |
| ATOM | 79 | CD | LYS | A | 246 | −9.176 | 12.247 | −5.595 | 1.00 | 47.17 | C |
| ATOM | 80 | CE | LYS | A | 246 | −8.356 | 13.514 | −5.451 | 1.00 | 45.20 | C |
| ATOM | 81 | NZ | LYS | A | 246 | −8.927 | 14.342 | −4.326 | 1.00 | 47.15 | N |
| ATOM | 82 | C | LYS | A | 246 | −12.820 | 10.043 | −6.451 | 1.00 | 41.72 | C |
| ATOM | 83 | O | LYS | A | 246 | −13.900 | 9.705 | −5.935 | 1.00 | 41.43 | O |
| ATOM | 84 | N | PRO | A | 247 | −12.320 | 9.400 | −7.553 | 1.00 | 41.43 | N |
| ATOM | 85 | CA | PRO | A | 247 | −13.088 | 8.297 | −8.144 | 1.00 | 40.71 | C |
| ATOM | 86 | CB | PRO | A | 247 | −12.076 | 7.593 | −9.050 | 1.00 | 40.06 | C |
| ATOM | 87 | CG | PRO | A | 247 | −11.154 | 8.645 | −9.462 | 1.00 | 40.48 | C |
| ATOM | 88 | CD | PRO | A | 247 | −11.101 | 9.679 | −8.347 | 1.00 | 41.20 | C |
| ATOM | 89 | C | PRO | A | 247 | −13.614 | 7.311 | −7.144 | 1.00 | 40.92 | C |
| ATOM | 90 | O | PRO | A | 247 | −14.703 | 6.813 | −7.317 | 1.00 | 42.48 | O |
| ATOM | 91 | N | LYS | A | 248 | −12.859 | 6.997 | −6.108 | 1.00 | 40.86 | N |
| ATOM | 92 | CA | LYS | A | 248 | −13.248 | 5.866 | −5.302 | 1.00 | 40.17 | C |
| ATOM | 93 | CB | LYS | A | 248 | −12.030 | 5.132 | −4.748 | 1.00 | 40.45 | C |
| ATOM | 94 | CG | LYS | A | 248 | −11.304 | 5.807 | −3.653 | 1.00 | 42.34 | C |
| ATOM | 95 | CD | LYS | A | 248 | −10.176 | 4.919 | −3.174 | 1.00 | 42.64 | C |
| ATOM | 96 | CE | LYS | A | 248 | −10.639 | 3.601 | −2.665 | 1.00 | 41.46 | C |
| ATOM | 97 | NZ | LYS | A | 248 | −9.511 | 3.084 | −1.804 | 1.00 | 43.28 | N |
| ATOM | 98 | C | LYS | A | 248 | −14.248 | 6.253 | −4.241 | 1.00 | 39.63 | C |
| ATOM | 99 | O | LYS | A | 248 | −15.049 | 5.433 | −3.822 | 1.00 | 41.30 | O |
| ATOM | 100 | N | ASP | A | 249 | −14.286 | 7.536 | −3.911 | 1.00 | 38.27 | N |
| ATOM | 101 | CA | ASP | A | 249 | −15.325 | 8.062 | −3.037 | 1.00 | 37.07 | C |
| ATOM | 102 | CB | ASP | A | 249 | −14.969 | 9.487 | −2.620 | 1.00 | 37.33 | C |
| ATOM | 103 | CG | ASP | A | 249 | −13.878 | 9.517 | −1.566 | 1.00 | 38.27 | C |
| ATOM | 104 | OD1 | ASP | A | 249 | −13.607 | 8.462 | −0.971 | 1.00 | 37.61 | O |
| ATOM | 105 | OD2 | ASP | A | 249 | −13.292 | 10.609 | −1.344 | 1.00 | 40.53 | O |
| ATOM | 106 | C | ASP | A | 249 | −16.690 | 8.006 | −3.747 | 1.00 | 36.05 | C |
| ATOM | 107 | O | ASP | A | 249 | −17.748 | 8.058 | −3.100 | 1.00 | 35.31 | O |
| ATOM | 108 | N | THR | A | 250 | −16.641 | 7.895 | −5.077 | 1.00 | 35.18 | N |
| ATOM | 109 | CA | THR | A | 250 | −17.863 | 7.844 | −5.926 | 1.00 | 34.97 | C |
| ATOM | 110 | CB | THR | A | 250 | −17.649 | 8.514 | −7.283 | 1.00 | 35.40 | C |
| ATOM | 111 | OG1 | THR | A | 250 | −16.790 | 7.681 | −8.057 | 1.00 | 33.25 | O |
| ATOM | 112 | CG2 | THR | A | 250 | −17.028 | 9.887 | −7.101 | 1.00 | 33.61 | C |
| ATOM | 113 | C | THR | A | 250 | −18.276 | 6.381 | −6.122 | 1.00 | 35.52 | C |
| ATOM | 114 | O | THR | A | 250 | −19.408 | 6.090 | −6.468 | 1.00 | 34.88 | O |
| ATOM | 115 | N | LEU | A | 251 | −17.301 | 5.487 | −5.907 | 1.00 | 36.99 | N |
| ATOM | 116 | CA | LEU | A | 251 | −17.529 | 4.095 | −6.193 | 1.00 | 38.71 | C |
| ATOM | 117 | CB | LEU | A | 251 | −16.343 | 3.579 | −7.010 | 1.00 | 37.13 | C |
| ATOM | 118 | CG | LEU | A | 251 | −16.090 | 4.229 | −8.388 | 1.00 | 35.95 | C |
| ATOM | 119 | CD1 | LEU | A | 251 | −14.778 | 3.757 | −8.996 | 1.00 | 35.85 | C |
| ATOM | 120 | CD2 | LEU | A | 251 | −17.244 | 3.938 | −9.342 | 1.00 | 31.98 | C |
| ATOM | 121 | C | LEU | A | 251 | −17.885 | 3.232 | −4.965 | 1.00 | 40.91 | C |
| ATOM | 122 | O | LEU | A | 251 | −18.141 | 2.042 | −5.113 | 1.00 | 42.29 | O |
| ATOM | 123 | N | MET | A | 252 | −17.886 | 3.838 | −3.787 | 1.00 | 42.41 | N |
| ATOM | 124 | CA | MET | A | 252 | −18.211 | 3.136 | −2.555 | 1.00 | 44.24 | C |
| ATOM | 125 | CB | MET | A | 252 | −16.995 | 3.045 | −1.663 | 1.00 | 44.32 | C |
| ATOM | 126 | CG | MET | A | 252 | −15.881 | 2.201 | −2.174 | 1.00 | 45.61 | C |
| ATOM | 127 | SD | MET | A | 252 | −14.526 | 2.604 | −1.045 | 1.00 | 50.61 | S |
| ATOM | 128 | CE | MET | A | 252 | −13.340 | 1.252 | −1.231 | 1.00 | 50.62 | C |
| ATOM | 129 | C | MET | A | 252 | −19.264 | 3.920 | −1.805 | 1.00 | 42.62 | C |
| ATOM | 130 | O | MET | A | 252 | −19.036 | 5.068 | −1.414 | 1.00 | 42.95 | O |
| ATOM | 131 | N | ILE | A | 253 | −20.417 | 3.293 | −1.606 | 1.00 | 41.62 | N |
| ATOM | 132 | CA | ILE | A | 253 | −21.526 | 3.921 | −0.913 | 1.00 | 39.64 | C |
| ATOM | 133 | CB | ILE | A | 253 | −22.785 | 3.011 | −0.873 | 1.00 | 39.82 | C |
| ATOM | 134 | CG1 | ILE | A | 253 | −24.090 | 3.833 | −0.943 | 1.00 | 39.47 | C |
| ATOM | 135 | CD1 | ILE | A | 253 | −25.373 | 2.974 | −1.326 | 1.00 | 38.55 | C |
| ATOM | 136 | CG2 | ILE | A | 253 | −22.745 | 2.038 | 0.285 | 1.00 | 37.86 | C |
| ATOM | 137 | C | ILE | A | 253 | −21.066 | 4.376 | 0.455 | 1.00 | 39.15 | C |
| ATOM | 138 | O | ILE | A | 253 | −21.528 | 5.390 | 0.925 | 1.00 | 38.76 | O |
| ATOM | 139 | N | SER | A | 254 | −20.101 | 3.681 | 1.056 | 1.00 | 39.05 | N |
| ATOM | 140 | CA | SER | A | 254 | −19.643 | 4.034 | 2.425 | 1.00 | 39.18 | C |
| ATOM | 141 | CB | SER | A | 254 | −18.839 | 2.880 | 3.080 | 1.00 | 38.86 | C |
| ATOM | 142 | OG | SER | A | 254 | −17.701 | 2.460 | 2.320 | 1.00 | 39.99 | O |
| ATOM | 143 | C | SER | A | 254 | −18.923 | 5.394 | 2.539 | 1.00 | 38.95 | C |
| ATOM | 144 | O | SER | A | 254 | −18.639 | 5.858 | 3.636 | 1.00 | 39.32 | O |
| ATOM | 145 | N | ARG | A | 255 | −18.684 | 6.060 | 1.413 | 1.00 | 39.14 | N |
| ATOM | 146 | CA | ARG | A | 255 | −17.873 | 7.288 | 1.400 | 1.00 | 39.73 | C |
| ATOM | 147 | CB | ARG | A | 255 | −16.644 | 7.097 | 0.523 | 1.00 | 38.93 | C |
| ATOM | 148 | CG | ARG | A | 255 | −15.783 | 6.016 | 1.083 | 1.00 | 40.09 | C |
| ATOM | 149 | CD | ARG | A | 255 | −14.694 | 5.670 | 0.167 | 1.00 | 44.27 | C |
| ATOM | 150 | NE | ARG | A | 255 | −13.548 | 6.541 | 0.356 | 1.00 | 47.39 | N |
| ATOM | 151 | CZ | ARG | A | 255 | −12.285 | 6.144 | 0.504 | 1.00 | 46.75 | C |
| ATOM | 152 | NH1 | ARG | A | 255 | −11.948 | 4.856 | 0.486 | 1.00 | 43.90 | N |
| ATOM | 153 | NH2 | ARG | A | 255 | −11.353 | 7.072 | 0.664 | 1.00 | 47.10 | N |
| ATOM | 154 | C | ARG | A | 255 | −18.639 | 8.540 | 1.006 | 1.00 | 40.14 | C |
| ATOM | 155 | O | ARG | A | 255 | −19.665 | 8.440 | 0.324 | 1.00 | 41.26 | O |
| ATOM | 156 | N | THR | A | 256 | −18.137 | 9.704 | 1.435 | 1.00 | 40.10 | N |
| ATOM | 157 | CA | THR | A | 256 | −18.762 | 11.022 | 1.200 | 1.00 | 40.03 | C |
| ATOM | 158 | CB | THR | A | 256 | −18.816 | 11.867 | 2.509 | 1.00 | 40.64 | C |
| ATOM | 159 | OG1 | THR | A | 256 | −17.632 | 11.625 | 3.303 | 1.00 | 41.43 | O |
| ATOM | 160 | CG2 | THR | A | 256 | −20.074 | 11.551 | 3.323 | 1.00 | 40.69 | C |
| ATOM | 161 | C | THR | A | 256 | −18.037 | 11.897 | 0.181 | 1.00 | 39.16 | C |
| ATOM | 162 | O | THR | A | 256 | −17.231 | 12.766 | 0.590 | 1.00 | 39.72 | O |
| ATOM | 163 | N | PRO | A | 257 | −18.374 | 11.737 | −1.115 | 1.00 | 38.13 | N |
| ATOM | 164 | CA | PRO | A | 257 | −17.790 | 12.496 | −2.234 | 1.00 | 38.09 | C |
| ATOM | 165 | CB | PRO | A | 257 | −18.464 | 11.888 | −3.497 | 1.00 | 38.18 | C |
| ATOM | 166 | CG | PRO | A | 257 | −19.771 | 11.294 | −3.002 | 1.00 | 37.78 | C |
| ATOM | 167 | CD | PRO | A | 257 | −19.452 | 10.831 | −1.568 | 1.00 | 38.24 | C |
| ATOM | 168 | C | PRO | A | 257 | −18.127 | 13.979 | −2.167 | 1.00 | 38.09 | C |
| ATOM | 169 | O | PRO | A | 257 | −19.253 | 14.340 | −1.837 | 1.00 | 36.67 | O |
| ATOM | 170 | N | GLU | A | 258 | −17.150 | 14.822 | −2.493 | 1.00 | 39.11 | N |
| ATOM | 171 | CA | GLU | A | 258 | −17.337 | 16.266 | −2.465 | 1.00 | 40.25 | C |
| ATOM | 172 | CB | GLU | A | 258 | −16.502 | 16.913 | −1.358 | 1.00 | 39.98 | C |
| ATOM | 173 | CG | GLU | A | 258 | −16.497 | 16.192 | 0.003 | 1.00 | 43.25 | C |
| ATOM | 174 | CD | GLU | A | 258 | −15.183 | 16.349 | 0.770 | 1.00 | 46.47 | C |
| ATOM | 175 | OE1 | GLU | A | 258 | −15.192 | 16.268 | 2.015 | 1.00 | 45.50 | O |
| ATOM | 176 | OE2 | GLU | A | 258 | −14.122 | 16.516 | 0.127 | 1.00 | 49.95 | O |
| ATOM | 177 | C | GLU | A | 258 | −16.840 | 16.812 | −3.778 | 1.00 | 40.80 | C |
| ATOM | 178 | O | GLU | A | 258 | −15.788 | 16.368 | −4.256 | 1.00 | 40.81 | O |
| ATOM | 179 | N | VAL | A | 259 | −17.591 | 17.778 | −4.326 | 1.00 | 41.16 | N |
| ATOM | 180 | CA | VAL | A | 259 | −17.135 | 18.710 | −5.366 | 1.00 | 41.52 | C |
| ATOM | 181 | CB | VAL | A | 259 | −18.305 | 19.118 | −6.326 | 1.00 | 41.96 | C |
| ATOM | 182 | CG1 | VAL | A | 259 | −17.913 | 20.253 | −7.236 | 1.00 | 42.65 | C |
| ATOM | 183 | CG2 | VAL | A | 259 | −18.744 | 17.962 | −7.191 | 1.00 | 40.58 | C |
| ATOM | 184 | C | VAL | A | 259 | −16.554 | 19.960 | −4.684 | 1.00 | 42.23 | C |
| ATOM | 185 | O | VAL | A | 259 | −17.176 | 20.536 | −3.794 | 1.00 | 42.68 | O |
| ATOM | 186 | N | THR | A | 260 | −15.350 | 20.377 | −5.074 | 1.00 | 43.14 | N |
| ATOM | 187 | CA | THR | A | 260 | −14.701 | 21.554 | −4.443 | 1.00 | 42.59 | C |
| ATOM | 188 | CB | THR | A | 260 | −13.277 | 21.228 | −3.921 | 1.00 | 42.95 | C |
| ATOM | 189 | OG1 | THR | A | 260 | −13.272 | 19.952 | −3.280 | 1.00 | 45.16 | O |
| ATOM | 190 | CG2 | THR | A | 260 | −12.793 | 22.280 | −2.928 | 1.00 | 43.14 | C |
| ATOM | 191 | C | THR | A | 260 | −14.581 | 22.746 | −5.400 | 1.00 | 42.13 | C |
| ATOM | 192 | O | THR | A | 260 | −13.985 | 22.637 | −6.511 | 1.00 | 41.37 | O |
| ATOM | 193 | N | CYS | A | 261 | −15.120 | 23.885 | −4.946 | 1.00 | 41.07 | N |
| ATOM | 194 | CA | CYS | A | 261 | −15.091 | 25.125 | −5.731 | 1.00 | 40.77 | C |
| ATOM | 195 | CB | CYS | A | 261 | −16.477 | 25.791 | −5.780 | 1.00 | 40.47 | C |
| ATOM | 196 | SG | CYS | A | 261 | −16.581 | 27.015 | −7.132 | 1.00 | 41.40 | S |
| ATOM | 197 | C | CYS | A | 261 | −14.050 | 26.123 | −5.215 | 1.00 | 39.95 | C |
| ATOM | 198 | O | CYS | A | 261 | −14.165 | 26.630 | −4.099 | 1.00 | 39.91 | O |
| ATOM | 199 | N | VAL | A | 262 | −13.064 | 26.431 | −6.043 | 1.00 | 39.19 | N |
| ATOM | 200 | CA | VAL | A | 262 | −11.982 | 27.271 | −5.599 | 1.00 | 38.84 | C |
| ATOM | 201 | CB | VAL | A | 262 | −10.613 | 26.519 | −5.643 | 1.00 | 39.03 | C |
| ATOM | 202 | CG1 | VAL | A | 262 | −9.457 | 27.423 | −5.185 | 1.00 | 36.30 | C |
| ATOM | 203 | CG2 | VAL | A | 262 | −10.668 | 25.196 | −4.832 | 1.00 | 35.65 | C |
| ATOM | 204 | C | VAL | A | 262 | −11.931 | 28.563 | −6.403 | 1.00 | 39.47 | C |
| ATOM | 205 | O | VAL | A | 262 | −11.916 | 28.525 | −7.640 | 1.00 | 40.69 | O |
| ATOM | 206 | N | VAL | A | 263 | −11.914 | 29.698 | −5.687 | 1.00 | 38.93 | N |
| ATOM | 207 | CA | VAL | A | 263 | −11.788 | 31.036 | −6.290 | 1.00 | 37.51 | C |
| ATOM | 208 | CB | VAL | A | 263 | −12.884 | 32.028 | −5.829 | 1.00 | 37.56 | C |
| ATOM | 209 | CG1 | VAL | A | 263 | −12.985 | 33.206 | −6.782 | 1.00 | 36.51 | C |
| ATOM | 210 | CG2 | VAL | A | 263 | −14.221 | 31.310 | −5.708 | 1.00 | 36.60 | C |
| ATOM | 211 | C | VAL | A | 263 | −10.444 | 31.626 | −5.937 | 1.00 | 36.83 | C |
| ATOM | 212 | O | VAL | A | 263 | −10.053 | 31.671 | −4.771 | 1.00 | 36.03 | O |
| ATOM | 213 | N | VAL | A | 264 | −9.735 | 32.076 | −6.964 | 1.00 | 36.73 | N |
| ATOM | 214 | CA | VAL | A | 264 | −8.397 | 32.689 | −6.744 | 1.00 | 36.34 | C |
| ATOM | 215 | CB | VAL | A | 264 | −7.227 | 31.772 | −7.226 | 1.00 | 36.31 | C |
| ATOM | 216 | CG1 | VAL | A | 264 | −7.003 | 30.667 | −6.204 | 1.00 | 33.36 | C |
| ATOM | 217 | CG2 | VAL | A | 264 | −7.536 | 31.178 | −8.585 | 1.00 | 36.18 | C |
| ATOM | 218 | C | VAL | A | 264 | −8.382 | 34.092 | −7.384 | 1.00 | 36.83 | C |
| ATOM | 219 | O | VAL | A | 264 | −9.330 | 34.482 | −8.064 | 1.00 | 36.66 | O |
| ATOM | 220 | N | ASP | A | 265 | −7.299 | 34.844 | −7.149 | 1.00 | 37.37 | N |
| ATOM | 221 | CA | ASP | A | 265 | −7.105 | 36.203 | −7.675 | 1.00 | 37.54 | C |
| ATOM | 222 | CB | ASP | A | 265 | −6.999 | 36.142 | −9.211 | 1.00 | 37.21 | C |
| ATOM | 223 | CG | ASP | A | 265 | −5.620 | 35.702 | −9.696 | 1.00 | 37.20 | C |
| ATOM | 224 | OD1 | ASP | A | 265 | −5.446 | 35.596 | −10.930 | 1.00 | 36.82 | O |
| ATOM | 225 | OD2 | ASP | A | 265 | −4.728 | 35.459 | −8.849 | 1.00 | 36.73 | O |
| ATOM | 226 | C | ASP | A | 265 | −8.130 | 37.193 | −7.192 | 1.00 | 38.10 | C |
| ATOM | 227 | O | ASP | A | 265 | −8.285 | 38.277 | −7.774 | 1.00 | 38.55 | O |
| ATOM | 228 | N | VAL | A | 266 | −8.844 | 36.823 | −6.128 | 1.00 | 38.52 | N |
| ATOM | 229 | CA | VAL | A | 266 | −9.725 | 37.757 | −5.402 | 1.00 | 39.30 | C |
| ATOM | 230 | CB | VAL | A | 266 | −10.501 | 37.029 | −4.287 | 1.00 | 39.34 | C |
| ATOM | 231 | CG1 | VAL | A | 266 | −11.224 | 38.005 | −3.362 | 1.00 | 38.00 | C |
| ATOM | 232 | CG2 | VAL | A | 266 | −11.483 | 36.030 | −4.903 | 1.00 | 39.73 | C |
| ATOM | 233 | C | VAL | A | 266 | −8.885 | 38.916 | −4.831 | 1.00 | 39.96 | C |
| ATOM | 234 | O | VAL | A | 266 | −7.858 | 38.689 | −4.173 | 1.00 | 40.54 | O |
| ATOM | 235 | N | SER | A | 267 | −9.299 | 40.149 | −5.101 | 1.00 | 40.18 | N |
| ATOM | 236 | CA | SER | A | 267 | −8.409 | 41.280 | −4.883 | 1.00 | 40.85 | C |
| ATOM | 237 | CB | SER | A | 267 | −8.874 | 42.546 | −5.647 | 1.00 | 41.08 | C |
| ATOM | 238 | OG | SER | A | 267 | −10.060 | 43.121 | −5.115 | 1.00 | 40.41 | O |
| ATOM | 239 | C | SER | A | 267 | −8.066 | 41.585 | −3.409 | 1.00 | 41.28 | C |
| ATOM | 240 | O | SER | A | 267 | −8.953 | 41.660 | −2.544 | 1.00 | 41.34 | O |
| ATOM | 241 | N | HIS | A | 268 | −6.753 | 41.717 | −3.164 | 1.00 | 41.42 | N |
| ATOM | 242 | CA | HIS | A | 268 | −6.132 | 42.331 | −1.981 | 1.00 | 40.82 | C |
| ATOM | 243 | CB | HIS | A | 268 | −4.799 | 42.947 | −2.488 | 1.00 | 40.24 | C |
| ATOM | 244 | CG | HIS | A | 268 | −3.898 | 43.587 | −1.453 | 1.00 | 37.88 | C |
| ATOM | 245 | ND1 | HIS | A | 268 | −3.835 | 43.196 | −0.131 | 1.00 | 35.86 | N |
| ATOM | 246 | CE1 | HIS | A | 268 | −2.923 | 43.914 | 0.498 | 1.00 | 29.66 | C |
| ATOM | 247 | NE2 | HIS | A | 268 | −2.369 | 44.730 | −0.372 | 1.00 | 29.11 | N |
| ATOM | 248 | CD2 | HIS | A | 268 | −2.951 | 44.546 | −1.597 | 1.00 | 31.66 | C |
| ATOM | 249 | C | HIS | A | 268 | −7.140 | 43.355 | −1.426 | 1.00 | 41.66 | C |
| ATOM | 250 | O | HIS | A | 268 | −7.215 | 43.557 | −0.207 | 1.00 | 42.45 | O |
| ATOM | 251 | N | GLU | A | 269 | −7.965 | 43.927 | −2.325 | 1.00 | 41.81 | N |
| ATOM | 252 | CA | GLU | A | 269 | −8.861 | 45.075 | −2.035 | 1.00 | 41.77 | C |
| ATOM | 253 | CB | GLU | A | 269 | −8.767 | 46.146 | −3.153 | 1.00 | 41.86 | C |
| ATOM | 254 | CG | GLU | A | 269 | −7.648 | 47.204 | −2.997 | 1.00 | 42.18 | C |
| ATOM | 255 | CD | GLU | A | 269 | −6.390 | 46.954 | −3.853 | 1.00 | 42.85 | C |
| ATOM | 256 | OE1 | GLU | A | 269 | −6.232 | 45.856 | −4.459 | 1.00 | 42.51 | O |
| ATOM | 257 | OE2 | GLU | A | 269 | −5.553 | 47.889 | −3.910 | 1.00 | 42.12 | O |
| ATOM | 258 | C | GLU | A | 269 | −10.338 | 44.735 | −1.772 | 1.00 | 41.50 | C |
| ATOM | 259 | O | GLU | A | 269 | −10.856 | 45.080 | −0.721 | 1.00 | 41.40 | O |
| ATOM | 260 | N | ASP | A | 270 | −10.985 | 44.060 | −2.732 | 1.00 | 41.75 | N |
| ATOM | 261 | CA | ASP | A | 270 | −12.455 | 43.805 | −2.778 | 1.00 | 41.50 | C |
| ATOM | 262 | CB | ASP | A | 270 | −12.964 | 44.002 | −4.212 | 1.00 | 41.54 | C |
| ATOM | 263 | CG | ASP | A | 270 | −12.774 | 45.404 | −4.713 | 1.00 | 42.16 | C |
| ATOM | 264 | OD1 | ASP | A | 270 | −11.824 | 46.102 | −4.279 | 1.00 | 40.56 | O |
| ATOM | 265 | OD2 | ASP | A | 270 | −13.593 | 45.799 | −5.561 | 1.00 | 43.78 | O |
| ATOM | 266 | C | ASP | A | 270 | −12.915 | 42.401 | −2.356 | 1.00 | 41.11 | C |
| ATOM | 267 | O | ASP | A | 270 | −13.392 | 41.642 | −3.195 | 1.00 | 40.76 | O |
| ATOM | 268 | N | PRO | A | 271 | −12.884 | 42.093 | −1.051 | 1.00 | 41.00 | N |
| ATOM | 269 | CA | PRO | A | 271 | −12.763 | 40.699 | −0.595 | 1.00 | 40.91 | C |
| ATOM | 270 | CB | PRO | A | 271 | −12.078 | 40.848 | 0.778 | 1.00 | 41.28 | C |
| ATOM | 271 | CG | PRO | A | 271 | −12.237 | 42.321 | 1.178 | 1.00 | 41.06 | C |
| ATOM | 272 | CD | PRO | A | 271 | −13.013 | 43.019 | 0.086 | 1.00 | 40.81 | C |
| ATOM | 273 | C | PRO | A | 271 | −14.046 | 39.832 | −0.479 | 1.00 | 40.81 | C |
| ATOM | 274 | O | PRO | A | 271 | −13.941 | 38.608 | −0.245 | 1.00 | 40.83 | O |
| ATOM | 275 | N | GLU | A | 272 | −15.231 | 40.433 | −0.630 | 1.00 | 40.29 | N |
| ATOM | 276 | CA | GLU | A | 272 | −16.482 | 39.647 | −0.590 | 1.00 | 39.74 | C |
| ATOM | 277 | CB | GLU | A | 272 | −17.724 | 40.539 | −0.443 | 1.00 | 39.82 | C |
| ATOM | 278 | CG | GLU | A | 272 | −17.754 | 41.413 | 0.841 | 1.00 | 40.47 | C |
| ATOM | 279 | CD | GLU | A | 272 | −19.082 | 42.146 | 1.025 | 1.00 | 40.16 | C |
| ATOM | 280 | OE1 | GLU | A | 272 | −19.505 | 42.317 | 2.184 | 1.00 | 41.46 | O |
| ATOM | 281 | OE2 | GLU | A | 272 | −19.709 | 42.554 | 0.021 | 1.00 | 40.80 | O |
| ATOM | 282 | C | GLU | A | 272 | −16.623 | 38.725 | −1.809 | 1.00 | 39.08 | C |
| ATOM | 283 | O | GLU | A | 272 | −16.417 | 39.132 | −2.959 | 1.00 | 39.12 | O |
| ATOM | 284 | N | VAL | A | 273 | −16.944 | 37.465 | −1.538 | 1.00 | 38.04 | N |
| ATOM | 285 | CA | VAL | A | 273 | −17.248 | 36.519 | −2.580 | 1.00 | 36.81 | C |
| ATOM | 286 | CB | VAL | A | 273 | −16.139 | 35.473 | −2.756 | 1.00 | 36.79 | C |
| ATOM | 287 | CG1 | VAL | A | 273 | −16.277 | 34.789 | −4.113 | 1.00 | 35.64 | C |
| ATOM | 288 | CG2 | VAL | A | 273 | −14.771 | 36.124 | −2.634 | 1.00 | 36.77 | C |
| ATOM | 289 | C | VAL | A | 273 | −18.528 | 35.847 | −2.185 | 1.00 | 36.49 | C |
| ATOM | 290 | O | VAL | A | 273 | −18.721 | 35.508 | −1.040 | 1.00 | 35.75 | O |
| ATOM | 291 | N | LYS | A | 274 | −19.420 | 35.655 | −3.132 | 1.00 | 36.75 | N |
| ATOM | 292 | CA | LYS | A | 274 | −20.606 | 34.914 | −2.818 | 1.00 | 37.47 | C |
| ATOM | 293 | CB | LYS | A | 274 | −21.834 | 35.724 | −3.187 | 1.00 | 37.15 | C |
| ATOM | 294 | CG | LYS | A | 274 | −23.145 | 35.214 | −2.594 | 1.00 | 37.26 | C |
| ATOM | 295 | CD | LYS | A | 274 | −24.302 | 36.013 | −3.171 | 1.00 | 36.65 | C |
| ATOM | 296 | CE | LYS | A | 274 | −24.220 | 36.067 | −4.697 | 1.00 | 36.43 | C |
| ATOM | 297 | NZ | LYS | A | 274 | −25.114 | 37.076 | −5.329 | 1.00 | 35.82 | N |
| ATOM | 298 | C | LYS | A | 274 | −20.552 | 33.634 | −3.620 | 1.00 | 38.01 | C |
| ATOM | 299 | O | LYS | A | 274 | −20.042 | 33.632 | −4.735 | 1.00 | 38.80 | O |
| ATOM | 300 | N | PHE | A | 275 | −21.064 | 32.544 | −3.051 | 1.00 | 38.32 | N |
| ATOM | 301 | CA | PHE | A | 275 | −21.296 | 31.312 | −3.824 | 1.00 | 37.66 | C |
| ATOM | 302 | CB | PHE | A | 275 | −20.547 | 30.114 | −3.244 | 1.00 | 37.84 | C |
| ATOM | 303 | CG | PHE | A | 275 | −19.052 | 30.300 | −3.176 | 1.00 | 38.72 | C |
| ATOM | 304 | CD1 | PHE | A | 275 | −18.477 | 31.119 | −2.192 | 1.00 | 37.71 | C |
| ATOM | 305 | CE1 | PHE | A | 275 | −17.112 | 31.276 | −2.110 | 1.00 | 35.95 | C |
| ATOM | 306 | CZ | PHE | A | 275 | −16.302 | 30.629 | −3.028 | 1.00 | 36.68 | C |
| ATOM | 307 | CE2 | PHE | A | 275 | −16.855 | 29.807 | −4.011 | 1.00 | 36.34 | C |
| ATOM | 308 | CD2 | PHE | A | 275 | −18.220 | 29.650 | −4.087 | 1.00 | 37.36 | C |
| ATOM | 309 | C | PHE | A | 275 | −22.744 | 30.953 | −3.837 | 1.00 | 37.21 | C |
| ATOM | 310 | O | PHE | A | 275 | −23.439 | 31.005 | −2.822 | 1.00 | 36.66 | O |
| ATOM | 311 | N | ASN | A | 276 | −23.178 | 30.562 | −5.015 | 1.00 | 37.44 | N |
| ATOM | 312 | CA | ASN | A | 276 | −24.474 | 29.953 | −5.216 | 1.00 | 37.82 | C |
| ATOM | 313 | CB | ASN | A | 276 | −25.331 | 30.896 | −6.045 | 1.00 | 38.50 | C |
| ATOM | 314 | CG | ASN | A | 276 | −25.261 | 32.303 | −5.543 | 1.00 | 38.63 | C |
| ATOM | 315 | OD1 | ASN | A | 276 | −24.364 | 33.080 | −5.902 | 1.00 | 40.02 | O |
| ATOM | 316 | ND2 | ASN | A | 276 | −26.187 | 32.637 | −4.671 | 1.00 | 40.71 | N |
| ATOM | 317 | C | ASN | A | 276 | −24.217 | 28.650 | −5.953 | 1.00 | 37.42 | C |
| ATOM | 318 | O | ASN | A | 276 | −23.301 | 28.598 | −6.763 | 1.00 | 37.26 | O |
| ATOM | 319 | N | TRP | A | 277 | −25.006 | 27.611 | −5.654 | 1.00 | 37.28 | N |
| ATOM | 320 | CA | TRP | A | 277 | −24.748 | 26.227 | −6.103 | 1.00 | 36.02 | C |
| ATOM | 321 | CB | TRP | A | 277 | −24.329 | 25.349 | −4.927 | 1.00 | 35.49 | C |
| ATOM | 322 | CG | TRP | A | 277 | −22.921 | 25.385 | −4.425 | 1.00 | 35.01 | C |
| ATOM | 323 | CD1 | TRP | A | 277 | −22.460 | 26.074 | −3.341 | 1.00 | 34.71 | C |
| ATOM | 324 | NE1 | TRP | A | 277 | −21.126 | 25.809 | −3.136 | 1.00 | 33.78 | N |
| ATOM | 325 | CE2 | TRP | A | 277 | −20.707 | 24.916 | −4.084 | 1.00 | 33.58 | C |
| ATOM | 326 | CD2 | TRP | A | 277 | −21.811 | 24.619 | −4.907 | 1.00 | 34.08 | C |
| ATOM | 327 | CE3 | TRP | A | 277 | −21.645 | 23.705 | −5.947 | 1.00 | 34.49 | C |
| ATOM | 328 | CZ3 | TRP | A | 277 | −20.390 | 23.128 | −6.139 | 1.00 | 34.74 | C |
| ATOM | 329 | CH2 | TRP | A | 277 | −19.319 | 23.456 | −5.319 | 1.00 | 35.43 | C |
| ATOM | 330 | CZ2 | TRP | A | 277 | −19.458 | 24.348 | −4.281 | 1.00 | 34.22 | C |
| ATOM | 331 | C | TRP | A | 277 | −26.033 | 25.603 | −6.625 | 1.00 | 36.14 | C |
| ATOM | 332 | O | TRP | A | 277 | −27.083 | 25.748 | −6.022 | 1.00 | 36.08 | O |
| ATOM | 333 | N | TYR | A | 278 | −25.937 | 24.878 | −7.727 | 1.00 | 36.64 | N |
| ATOM | 334 | CA | TYR | A | 278 | −27.073 | 24.197 | −8.314 | 1.00 | 37.71 | C |
| ATOM | 335 | CB | TYR | A | 278 | −27.574 | 24.943 | −9.531 | 1.00 | 36.96 | C |
| ATOM | 336 | CG | TYR | A | 278 | −27.603 | 26.414 | −9.316 | 1.00 | 36.44 | C |
| ATOM | 337 | CD1 | TYR | A | 278 | −26.423 | 27.174 | −9.406 | 1.00 | 36.18 | C |
| ATOM | 338 | CE1 | TYR | A | 278 | −26.433 | 28.539 | −9.191 | 1.00 | 35.38 | C |
| ATOM | 339 | CZ | TYR | A | 278 | −27.639 | 29.159 | −8.886 | 1.00 | 35.47 | C |
| ATOM | 340 | OH | TYR | A | 278 | −27.674 | 30.519 | −8.704 | 1.00 | 34.04 | O |
| ATOM | 341 | CE2 | TYR | A | 278 | −28.823 | 28.418 | −8.796 | 1.00 | 35.90 | C |
| ATOM | 342 | CD2 | TYR | A | 278 | −28.796 | 27.061 | −9.008 | 1.00 | 35.71 | C |
| ATOM | 343 | C | TYR | A | 278 | −26.785 | 22.753 | −8.727 | 1.00 | 39.49 | C |
| ATOM | 344 | O | TYR | A | 278 | −25.680 | 22.388 | −9.200 | 1.00 | 39.78 | O |
| ATOM | 345 | N | VAL | A | 279 | −27.815 | 21.926 | −8.534 | 1.00 | 40.99 | N |
| ATOM | 346 | CA | VAL | A | 279 | −27.824 | 20.557 | −9.035 | 1.00 | 41.50 | C |
| ATOM | 347 | CB | VAL | A | 279 | −28.080 | 19.538 | −7.902 | 1.00 | 41.40 | C |
| ATOM | 348 | CG1 | VAL | A | 279 | −28.021 | 18.118 | −8.436 | 1.00 | 40.52 | C |
| ATOM | 349 | CG2 | VAL | A | 279 | −27.074 | 19.744 | −6.748 | 1.00 | 41.86 | C |
| ATOM | 350 | C | VAL | A | 279 | −28.891 | 20.516 | −10.145 | 1.00 | 41.90 | C |
| ATOM | 351 | O | VAL | A | 279 | −30.105 | 20.614 | −9.872 | 1.00 | 42.30 | O |
| ATOM | 352 | N | ASP | A | 280 | −28.408 | 20.452 | −11.383 | 1.00 | 41.75 | N |
| ATOM | 353 | CA | ASP | A | 280 | −29.242 | 20.431 | −12.582 | 1.00 | 42.74 | C |
| ATOM | 354 | CB | ASP | A | 280 | −30.069 | 19.126 | −12.662 | 1.00 | 43.05 | C |
| ATOM | 355 | CG | ASP | A | 280 | −29.209 | 17.885 | −12.994 | 1.00 | 44.95 | C |
| ATOM | 356 | OD1 | ASP | A | 280 | −28.052 | 18.041 | −13.450 | 1.00 | 46.60 | O |
| ATOM | 357 | OD2 | ASP | A | 280 | −29.691 | 16.745 | −12.798 | 1.00 | 46.68 | O |
| ATOM | 358 | C | ASP | A | 280 | −30.117 | 21.695 | −12.786 | 1.00 | 42.69 | C |
| ATOM | 359 | O | ASP | A | 280 | −30.997 | 21.706 | −13.654 | 1.00 | 43.04 | O |
| ATOM | 360 | N | GLY | A | 281 | −29.850 | 22.757 | −12.021 | 1.00 | 42.25 | N |
| ATOM | 361 | CA | GLY | A | 281 | −30.627 | 23.972 | −12.110 | 1.00 | 41.79 | C |
| ATOM | 362 | C | GLY | A | 281 | −31.270 | 24.396 | −10.801 | 1.00 | 42.18 | C |
| ATOM | 363 | O | GLY | A | 281 | −31.342 | 25.588 | −10.509 | 1.00 | 42.13 | O |
| ATOM | 364 | N | VAL | A | 282 | −31.746 | 23.439 | −10.003 | 1.00 | 42.71 | N |
| ATOM | 365 | CA | VAL | A | 282 | −32.369 | 23.755 | −8.680 | 1.00 | 42.83 | C |
| ATOM | 366 | CB | VAL | A | 282 | −33.079 | 22.499 | −8.044 | 1.00 | 42.95 | C |
| ATOM | 367 | CG1 | VAL | A | 282 | −34.458 | 22.877 | −7.490 | 1.00 | 44.01 | C |
| ATOM | 368 | CG2 | VAL | A | 282 | −33.227 | 21.357 | −9.058 | 1.00 | 41.95 | C |
| ATOM | 369 | C | VAL | A | 282 | −31.297 | 24.323 | −7.725 | 1.00 | 42.22 | C |
| ATOM | 370 | O | VAL | A | 282 | −30.191 | 23.798 | −7.673 | 1.00 | 43.36 | O |
| ATOM | 371 | N | GLU | A | 283 | −31.542 | 25.399 | −7.003 | 1.00 | 41.26 | N |
| ATOM | 372 | CA | GLU | A | 283 | −30.435 | 25.852 | −6.173 | 1.00 | 40.91 | C |
| ATOM | 373 | CB | GLU | A | 283 | −30.506 | 27.332 | −5.814 | 1.00 | 40.73 | C |
| ATOM | 374 | CG | GLU | A | 283 | −29.159 | 27.923 | −5.438 | 1.00 | 40.16 | C |
| ATOM | 375 | CD | GLU | A | 283 | −29.271 | 29.214 | −4.630 | 1.00 | 40.38 | C |
| ATOM | 376 | OE1 | GLU | A | 283 | −30.310 | 29.418 | −3.949 | 1.00 | 41.64 | O |
| ATOM | 377 | OE2 | GLU | A | 283 | −28.313 | 30.026 | −4.679 | 1.00 | 37.50 | O |
| ATOM | 378 | C | GLU | A | 283 | −30.350 | 24.986 | −4.933 | 1.00 | 40.99 | C |
| ATOM | 379 | O | GLU | A | 283 | −31.373 | 24.514 | −4.427 | 1.00 | 41.13 | O |
| ATOM | 380 | N | VAL | A | 284 | −29.092 | 24.769 | −4.482 | 1.00 | 40.82 | N |
| ATOM | 381 | CA | VAL | A | 284 | −28.902 | 23.967 | −3.247 | 1.00 | 40.67 | C |
| ATOM | 382 | CB | VAL | A | 284 | −28.296 | 22.589 | −3.480 | 1.00 | 40.70 | C |
| ATOM | 383 | CG1 | VAL | A | 284 | −29.090 | 21.826 | −4.531 | 1.00 | 40.38 | C |
| ATOM | 384 | CG2 | VAL | A | 284 | −26.835 | 22.719 | −3.895 | 1.00 | 38.42 | C |
| ATOM | 385 | C | VAL | A | 284 | −28.188 | 24.808 | −2.178 | 1.00 | 41.33 | C |
| ATOM | 386 | O | VAL | A | 284 | −27.344 | 25.657 | −2.479 | 1.00 | 40.91 | O |
| ATOM | 387 | N | HIS | A | 285 | −28.574 | 24.544 | −0.940 | 1.00 | 41.57 | N |
| ATOM | 388 | CA | HIS | A | 285 | −28.147 | 25.367 | 0.156 | 1.00 | 41.32 | C |
| ATOM | 389 | CB | HIS | A | 285 | −29.427 | 25.895 | 0.743 | 1.00 | 41.36 | C |
| ATOM | 390 | CG | HIS | A | 285 | −30.408 | 26.602 | −0.258 | 1.00 | 41.46 | C |
| ATOM | 391 | ND1 | HIS | A | 285 | −31.629 | 26.066 | −0.693 | 1.00 | 41.59 | N |
| ATOM | 392 | CE1 | HIS | A | 285 | −32.214 | 26.924 | −1.516 | 1.00 | 41.85 | C |
| ATOM | 393 | NE2 | HIS | A | 285 | −31.430 | 27.982 | −1.644 | 1.00 | 41.22 | N |
| ATOM | 394 | CD2 | HIS | A | 285 | −30.300 | 27.791 | −0.880 | 1.00 | 42.23 | C |
| ATOM | 395 | C | HIS | A | 285 | −27.264 | 24.699 | 1.252 | 1.00 | 41.47 | C |
| ATOM | 396 | O | HIS | A | 285 | −27.119 | 25.225 | 2.346 | 1.00 | 40.74 | O |
| ATOM | 397 | N | ASN | A | 286 | −26.719 | 23.502 | 0.918 | 1.00 | 41.33 | N |
| ATOM | 398 | CA | ASN | A | 286 | −25.995 | 22.654 | 1.875 | 1.00 | 41.07 | C |
| ATOM | 399 | CB | ASN | A | 286 | −26.387 | 21.187 | 1.634 | 1.00 | 41.08 | C |
| ATOM | 400 | CG | ASN | A | 286 | −26.430 | 20.834 | 0.163 | 1.00 | 42.87 | C |
| ATOM | 401 | OD1 | ASN | A | 286 | −26.983 | 21.589 | −0.642 | 1.00 | 45.64 | O |
| ATOM | 402 | ND2 | ASN | A | 286 | −25.844 | 19.692 | −0.179 | 1.00 | 43.48 | N |
| ATOM | 403 | C | ASN | A | 286 | −24.479 | 22.756 | 1.887 | 1.00 | 40.60 | C |
| ATOM | 404 | O | ASN | A | 286 | −23.838 | 22.152 | 2.746 | 1.00 | 40.55 | O |
| ATOM | 405 | N | ALA | A | 287 | −23.906 | 23.513 | 0.951 | 1.00 | 40.49 | N |
| ATOM | 406 | CA | ALA | A | 287 | −22.434 | 23.587 | 0.808 | 1.00 | 40.81 | C |
| ATOM | 407 | CB | ALA | A | 287 | −22.046 | 24.334 | −0.466 | 1.00 | 40.45 | C |
| ATOM | 408 | C | ALA | A | 287 | −21.735 | 24.213 | 2.027 | 1.00 | 40.89 | C |
| ATOM | 409 | O | ALA | A | 287 | −22.299 | 25.071 | 2.691 | 1.00 | 40.81 | O |
| ATOM | 410 | N | LYS | A | 288 | −20.508 | 23.777 | 2.316 | 1.00 | 41.39 | N |
| ATOM | 411 | CA | LYS | A | 288 | −19.731 | 24.291 | 3.452 | 1.00 | 41.30 | C |
| ATOM | 412 | CB | LYS | A | 288 | −19.085 | 23.137 | 4.257 | 1.00 | 41.56 | C |
| ATOM | 413 | CG | LYS | A | 288 | −19.949 | 22.418 | 5.340 | 1.00 | 41.61 | C |
| ATOM | 414 | CD | LYS | A | 288 | −21.379 | 21.983 | 4.909 | 1.00 | 41.28 | C |
| ATOM | 415 | CE | LYS | A | 288 | −22.229 | 21.587 | 6.117 | 1.00 | 40.86 | C |
| ATOM | 416 | NZ | LYS | A | 288 | −22.003 | 22.469 | 7.344 | 1.00 | 40.15 | N |
| ATOM | 417 | C | LYS | A | 288 | −18.669 | 25.239 | 2.872 | 1.00 | 41.49 | C |
| ATOM | 418 | O | LYS | A | 288 | −17.786 | 24.817 | 2.069 | 1.00 | 41.31 | O |
| ATOM | 419 | N | THR | A | 289 | −18.801 | 26.520 | 3.231 | 1.00 | 41.06 | N |
| ATOM | 420 | CA | THR | A | 289 | −17.883 | 27.564 | 2.777 | 1.00 | 40.83 | C |
| ATOM | 421 | CB | THR | A | 289 | −18.606 | 28.852 | 2.299 | 1.00 | 40.70 | C |
| ATOM | 422 | OG1 | THR | A | 289 | −19.457 | 28.528 | 1.184 | 1.00 | 38.99 | O |
| ATOM | 423 | CG2 | THR | A | 289 | −17.577 | 29.958 | 1.886 | 1.00 | 38.80 | C |
| ATOM | 424 | C | THR | A | 289 | −16.962 | 27.893 | 3.915 | 1.00 | 41.41 | C |
| ATOM | 425 | O | THR | A | 289 | −17.425 | 28.068 | 5.044 | 1.00 | 40.91 | O |
| ATOM | 426 | N | LYS | A | 290 | −15.663 | 27.940 | 3.611 | 1.00 | 42.06 | N |
| ATOM | 427 | CA | LYS | A | 290 | −14.631 | 28.251 | 4.593 | 1.00 | 42.95 | C |
| ATOM | 428 | CB | LYS | A | 290 | −13.327 | 27.507 | 4.288 | 1.00 | 43.27 | C |
| ATOM | 429 | CG | LYS | A | 290 | −13.425 | 26.104 | 3.719 | 1.00 | 43.11 | C |
| ATOM | 430 | CD | LYS | A | 290 | −11.994 | 25.623 | 3.403 | 1.00 | 44.55 | C |
| ATOM | 431 | CE | LYS | A | 290 | −11.874 | 24.145 | 2.895 | 1.00 | 47.44 | C |
| ATOM | 432 | NZ | LYS | A | 290 | −10.522 | 23.573 | 3.321 | 1.00 | 48.13 | N |
| ATOM | 433 | C | LYS | A | 290 | −14.351 | 29.757 | 4.548 | 1.00 | 42.82 | C |
| ATOM | 434 | O | LYS | A | 290 | −14.423 | 30.363 | 3.466 | 1.00 | 43.02 | O |
| ATOM | 435 | N | PRO | A | 291 | −14.104 | 30.378 | 5.722 | 1.00 | 42.94 | N |
| ATOM | 436 | CA | PRO | A | 291 | −13.470 | 31.720 | 5.780 | 1.00 | 42.79 | C |
| ATOM | 437 | CB | PRO | A | 291 | −13.212 | 31.938 | 7.287 | 1.00 | 42.55 | C |
| ATOM | 438 | CG | PRO | A | 291 | −14.286 | 31.123 | 7.968 | 1.00 | 43.15 | C |
| ATOM | 439 | CD | PRO | A | 291 | −14.527 | 29.912 | 7.064 | 1.00 | 43.15 | C |
| ATOM | 440 | C | PRO | A | 291 | −12.171 | 31.845 | 4.953 | 1.00 | 42.02 | C |
| ATOM | 441 | O | PRO | A | 291 | −11.285 | 30.983 | 5.042 | 1.00 | 41.63 | O |
| ATOM | 442 | N | ARG | A | 292 | −12.103 | 32.929 | 4.174 | 1.00 | 41.29 | N |
| ATOM | 443 | CA | ARG | A | 292 | −11.045 | 33.217 | 3.198 | 1.00 | 40.71 | C |
| ATOM | 444 | CB | ARG | A | 292 | −11.311 | 34.578 | 2.570 | 1.00 | 40.92 | C |
| ATOM | 445 | CG | ARG | A | 292 | −11.386 | 35.729 | 3.568 | 1.00 | 41.29 | C |
| ATOM | 446 | CD | ARG | A | 292 | −12.622 | 36.583 | 3.307 | 1.00 | 44.00 | C |
| ATOM | 447 | NE | ARG | A | 292 | −12.439 | 37.961 | 3.762 | 1.00 | 44.97 | N |
| ATOM | 448 | CZ | ARG | A | 292 | −13.272 | 38.976 | 3.514 | 1.00 | 45.07 | C |
| ATOM | 449 | NH1 | ARG | A | 292 | −14.385 | 38.795 | 2.802 | 1.00 | 44.97 | N |
| ATOM | 450 | NH2 | ARG | A | 292 | −12.979 | 40.190 | 3.979 | 1.00 | 45.22 | N |
| ATOM | 451 | C | ARG | A | 292 | −9.630 | 33.201 | 3.757 | 1.00 | 40.43 | C |
| ATOM | 452 | O | ARG | A | 292 | −9.398 | 33.657 | 4.864 | 1.00 | 39.60 | O |
| ATOM | 453 | N | GLU | A | 293 | −8.688 | 32.688 | 2.973 | 1.00 | 40.19 | N |
| ATOM | 454 | CA | GLU | A | 293 | −7.308 | 32.601 | 3.405 | 1.00 | 40.41 | C |
| ATOM | 455 | CB | GLU | A | 293 | −6.838 | 31.145 | 3.394 | 1.00 | 40.73 | C |
| ATOM | 456 | CG | GLU | A | 293 | −7.310 | 30.276 | 4.592 | 1.00 | 42.13 | C |
| ATOM | 457 | CD | GLU | A | 293 | −6.444 | 28.982 | 4.817 | 1.00 | 42.08 | C |
| ATOM | 458 | OE1 | GLU | A | 293 | −6.054 | 28.698 | 5.981 | 1.00 | 43.35 | O |
| ATOM | 459 | OE2 | GLU | A | 293 | −6.157 | 28.248 | 3.842 | 1.00 | 43.07 | O |
| ATOM | 460 | C | GLU | A | 293 | −6.410 | 33.473 | 2.529 | 1.00 | 39.84 | C |
| ATOM | 461 | O | GLU | A | 293 | −6.393 | 33.333 | 1.311 | 1.00 | 39.32 | O |
| ATOM | 462 | N | GLU | A | 294 | −5.665 | 34.382 | 3.156 | 1.00 | 39.72 | N |
| ATOM | 463 | CA | GLU | A | 294 | −4.795 | 35.288 | 2.409 | 1.00 | 39.68 | C |
| ATOM | 464 | CB | GLU | A | 294 | −4.479 | 36.530 | 3.234 | 1.00 | 39.38 | C |
| ATOM | 465 | CG | GLU | A | 294 | −3.464 | 37.451 | 2.594 | 1.00 | 39.19 | C |
| ATOM | 466 | CD | GLU | A | 294 | −3.129 | 38.665 | 3.453 | 1.00 | 40.06 | C |
| ATOM | 467 | OE1 | GLU | A | 294 | −2.275 | 39.460 | 3.008 | 1.00 | 39.83 | O |
| ATOM | 468 | OE2 | GLU | A | 294 | −3.710 | 38.838 | 4.560 | 1.00 | 40.21 | O |
| ATOM | 469 | C | GLU | A | 294 | −3.512 | 34.583 | 1.976 | 1.00 | 39.79 | C |
| ATOM | 470 | O | GLU | A | 294 | −2.797 | 34.033 | 2.809 | 1.00 | 40.02 | O |
| ATOM | 471 | N | GLN | A | 295 | −3.222 | 34.598 | 0.678 | 1.00 | 39.83 | N |
| ATOM | 472 | CA | GLN | A | 295 | −2.036 | 33.915 | 0.147 | 1.00 | 39.99 | C |
| ATOM | 473 | CB | GLN | A | 295 | −2.262 | 33.509 | −1.310 | 1.00 | 39.81 | C |
| ATOM | 474 | CG | GLN | A | 295 | −3.559 | 32.727 | −1.544 | 1.00 | 37.88 | C |
| ATOM | 475 | CD | GLN | A | 295 | −3.666 | 31.458 | −0.691 | 1.00 | 35.96 | C |
| ATOM | 476 | OE1 | GLN | A | 295 | −2.972 | 30.462 | −0.933 | 1.00 | 36.53 | O |
| ATOM | 477 | NE2 | GLN | A | 295 | −4.546 | 31.491 | 0.308 | 1.00 | 34.78 | N |
| ATOM | 478 | C | GLN | A | 295 | −0.801 | 34.790 | 0.274 | 1.00 | 40.62 | C |
| ATOM | 479 | O | GLN | A | 295 | −0.930 | 36.003 | 0.402 | 1.00 | 41.15 | O |
| ATOM | 480 | N | TYR | A | 296 | 0.390 | 34.180 | 0.247 | 1.00 | 41.18 | N |
| ATOM | 481 | CA | TYR | A | 296 | 1.667 | 34.909 | 0.382 | 1.00 | 41.14 | C |
| ATOM | 482 | CB | TYR | A | 296 | 2.814 | 33.939 | 0.700 | 1.00 | 40.95 | C |
| ATOM | 483 | CG | TYR | A | 296 | 3.094 | 33.718 | 2.179 | 1.00 | 40.61 | C |
| ATOM | 484 | CD1 | TYR | A | 296 | 2.112 | 33.215 | 3.046 | 1.00 | 40.39 | C |
| ATOM | 485 | CE1 | TYR | A | 296 | 2.382 | 33.001 | 4.409 | 1.00 | 40.40 | C |
| ATOM | 486 | CZ | TYR | A | 296 | 3.653 | 33.285 | 4.906 | 1.00 | 40.69 | C |
| ATOM | 487 | OH | TYR | A | 296 | 3.953 | 33.091 | 6.236 | 1.00 | 40.78 | O |
| ATOM | 488 | CE2 | TYR | A | 296 | 4.638 | 33.782 | 4.065 | 1.00 | 40.61 | C |
| ATOM | 489 | CD2 | TYR | A | 296 | 4.353 | 33.990 | 2.707 | 1.00 | 40.18 | C |
| ATOM | 490 | C | TYR | A | 296 | 2.004 | 35.752 | −0.851 | 1.00 | 41.59 | C |
| ATOM | 491 | O | TYR | A | 296 | 3.088 | 36.312 | −0.941 | 1.00 | 41.87 | O |
| ATOM | 492 | N | ASN | A | 297 | 1.069 | 35.829 | −1.797 | 1.00 | 42.43 | N |
| ATOM | 493 | CA | ASN | A | 297 | 1.173 | 36.723 | −2.957 | 1.00 | 43.29 | C |
| ATOM | 494 | CB | ASN | A | 297 | 1.115 | 35.954 | −4.294 | 1.00 | 43.87 | C |
| ATOM | 495 | CG | ASN | A | 297 | −0.274 | 35.321 | −4.596 | 1.00 | 47.77 | C |
| ATOM | 496 | OD1 | ASN | A | 297 | −1.209 | 35.371 | −3.785 | 1.00 | 45.63 | O |
| ATOM | 497 | ND2 | ASN | A | 297 | −0.385 | 34.720 | −5.799 | 1.00 | 55.36 | N |
| ATOM | 498 | C | ASN | A | 297 | 0.170 | 37.886 | −2.922 | 1.00 | 42.86 | C |
| ATOM | 499 | O | ASN | A | 297 | −0.245 | 38.393 | −3.966 | 1.00 | 42.80 | O |
| ATOM | 500 | N | SER | A | 298 | −0.203 | 38.299 | −1.712 | 1.00 | 42.60 | N |
| ATOM | 501 | CA | SER | A | 298 | −1.087 | 39.442 | −1.491 | 1.00 | 42.30 | C |
| ATOM | 502 | CB | SER | A | 298 | −0.330 | 40.745 | −1.789 | 1.00 | 42.15 | C |
| ATOM | 503 | OG | SER | A | 298 | 0.898 | 40.771 | −1.075 | 1.00 | 41.40 | O |
| ATOM | 504 | C | SER | A | 298 | −2.440 | 39.330 | −2.246 | 1.00 | 42.27 | C |
| ATOM | 505 | O | SER | A | 298 | −2.905 | 40.286 | −2.877 | 1.00 | 42.41 | O |
| ATOM | 506 | N | THR | A | 299 | −3.066 | 38.157 | −2.133 | 1.00 | 41.90 | N |
| ATOM | 507 | CA | THR | A | 299 | −4.292 | 37.800 | −2.866 | 1.00 | 42.32 | C |
| ATOM | 508 | CB | THR | A | 299 | −3.943 | 37.126 | −4.226 | 1.00 | 42.37 | C |
| ATOM | 509 | OG1 | THR | A | 299 | −3.274 | 38.060 | −5.077 | 1.00 | 44.16 | O |
| ATOM | 510 | CG2 | THR | A | 299 | −5.163 | 36.648 | −4.938 | 1.00 | 43.10 | C |
| ATOM | 511 | C | THR | A | 299 | −5.128 | 36.798 | −2.062 | 1.00 | 42.11 | C |
| ATOM | 512 | O | THR | A | 299 | −4.564 | 35.942 | −1.356 | 1.00 | 42.29 | O |
| ATOM | 513 | N | TYR | A | 300 | −6.457 | 36.863 | −2.192 | 1.00 | 41.62 | N |
| ATOM | 514 | CA | TYR | A | 300 | −7.332 | 35.892 | −1.507 | 1.00 | 41.19 | C |
| ATOM | 515 | CB | TYR | A | 300 | −8.626 | 36.543 | −1.004 | 1.00 | 41.79 | C |
| ATOM | 516 | CG | TYR | A | 300 | −8.471 | 37.505 | 0.181 | 1.00 | 42.61 | C |
| ATOM | 517 | CD1 | TYR | A | 300 | −8.452 | 37.027 | 1.500 | 1.00 | 42.41 | C |
| ATOM | 518 | CE1 | TYR | A | 300 | −8.334 | 37.893 | 2.570 | 1.00 | 42.06 | C |
| ATOM | 519 | CZ | TYR | A | 300 | −8.241 | 39.249 | 2.328 | 1.00 | 42.14 | C |
| ATOM | 520 | OH | TYR | A | 300 | −8.120 | 40.106 | 3.381 | 1.00 | 42.92 | O |
| ATOM | 521 | CE2 | TYR | A | 300 | −8.263 | 39.756 | 1.042 | 1.00 | 42.10 | C |
| ATOM | 522 | CD2 | TYR | A | 300 | −8.381 | 38.888 | −0.023 | 1.00 | 41.40 | C |
| ATOM | 523 | C | TYR | A | 300 | −7.691 | 34.637 | −2.302 | 1.00 | 40.83 | C |
| ATOM | 524 | O | TYR | A | 300 | −7.553 | 34.569 | −3.533 | 1.00 | 41.03 | O |
| ATOM | 525 | N | ARG | A | 301 | −8.193 | 33.659 | −1.552 | 1.00 | 40.02 | N |
| ATOM | 526 | CA | ARG | A | 301 | −8.510 | 32.328 | −2.018 | 1.00 | 38.68 | C |
| ATOM | 527 | CB | ARG | A | 301 | −7.286 | 31.417 | −1.831 | 1.00 | 38.61 | C |
| ATOM | 528 | CG | ARG | A | 301 | −7.496 | 29.968 | −2.244 | 1.00 | 38.11 | C |
| ATOM | 529 | CD | ARG | A | 301 | −6.259 | 29.137 | −1.965 | 1.00 | 37.72 | C |
| ATOM | 530 | NE | ARG | A | 301 | −6.387 | 27.799 | −2.517 | 1.00 | 36.16 | N |
| ATOM | 531 | CZ | ARG | A | 301 | −7.082 | 26.822 | −1.943 | 1.00 | 36.82 | C |
| ATOM | 532 | NH1 | ARG | A | 301 | −7.707 | 27.059 | −0.801 | 1.00 | 37.12 | N |
| ATOM | 533 | NH2 | ARG | A | 301 | −7.167 | 25.613 | −2.508 | 1.00 | 32.90 | N |
| ATOM | 534 | C | ARG | A | 301 | −9.638 | 31.833 | −1.133 | 1.00 | 38.26 | C |
| ATOM | 535 | O | ARG | A | 301 | −9.473 | 31.715 | 0.077 | 1.00 | 38.32 | O |
| ATOM | 536 | N | VAL | A | 302 | −10.784 | 31.584 | −1.727 | 1.00 | 37.49 | N |
| ATOM | 537 | CA | VAL | A | 302 | −11.934 | 31.076 | −0.950 | 1.00 | 36.96 | C |
| ATOM | 538 | CB | VAL | A | 302 | −13.077 | 32.109 | −0.783 | 1.00 | 37.08 | C |
| ATOM | 539 | CG1 | VAL | A | 302 | −13.796 | 31.874 | 0.538 | 1.00 | 36.24 | C |
| ATOM | 540 | CG2 | VAL | A | 302 | −12.542 | 33.541 | −0.869 | 1.00 | 36.69 | C |
| ATOM | 541 | C | VAL | A | 302 | −12.468 | 29.816 | −1.598 | 1.00 | 36.58 | C |
| ATOM | 542 | O | VAL | A | 302 | −12.609 | 29.709 | −2.802 | 1.00 | 35.83 | O |
| ATOM | 543 | N | VAL | A | 303 | −12.779 | 28.896 | −0.717 | 1.00 | 36.37 | N |
| ATOM | 544 | CA | VAL | A | 303 | −13.256 | 27.556 | −1.030 | 1.00 | 36.20 | C |
| ATOM | 545 | CB | VAL | A | 303 | −12.280 | 26.519 | −0.372 | 1.00 | 35.90 | C |
| ATOM | 546 | CG1 | VAL | A | 303 | −12.712 | 25.085 | −0.674 | 1.00 | 36.28 | C |
| ATOM | 547 | CG2 | VAL | A | 303 | −10.852 | 26.768 | −0.843 | 1.00 | 35.96 | C |
| ATOM | 548 | C | VAL | A | 303 | −14.705 | 27.282 | −0.621 | 1.00 | 36.08 | C |
| ATOM | 549 | O | VAL | A | 303 | −15.175 | 27.759 | 0.416 | 1.00 | 36.06 | O |
| ATOM | 550 | N | SER | A | 304 | −15.416 | 26.492 | −1.429 | 1.00 | 35.74 | N |
| ATOM | 551 | CA | SER | A | 304 | −16.787 | 26.051 | −1.150 | 1.00 | 36.30 | C |
| ATOM | 552 | CB | SER | A | 304 | −17.816 | 26.809 | −1.993 | 1.00 | 36.64 | C |
| ATOM | 553 | OG | SER | A | 304 | −19.065 | 26.882 | −1.317 | 1.00 | 36.50 | O |
| ATOM | 554 | C | SER | A | 304 | −16.878 | 24.556 | −1.474 | 1.00 | 36.58 | C |
| ATOM | 555 | O | SER | A | 304 | −16.787 | 24.171 | −2.626 | 1.00 | 37.80 | O |
| ATOM | 556 | N | VAL | A | 305 | −17.054 | 23.727 | −0.449 | 1.00 | 36.65 | N |
| ATOM | 557 | CA | VAL | A | 305 | −17.238 | 22.294 | −0.612 | 1.00 | 35.33 | C |
| ATOM | 558 | CB | VAL | A | 305 | −16.514 | 21.498 | 0.532 | 1.00 | 35.30 | C |
| ATOM | 559 | CG1 | VAL | A | 305 | −16.945 | 20.000 | 0.541 | 1.00 | 31.70 | C |
| ATOM | 560 | CG2 | VAL | A | 305 | −15.020 | 21.664 | 0.423 | 1.00 | 32.17 | C |
| ATOM | 561 | C | VAL | A | 305 | −18.724 | 21.951 | −0.610 | 1.00 | 35.72 | C |
| ATOM | 562 | O | VAL | A | 305 | −19.433 | 22.202 | 0.387 | 1.00 | 35.47 | O |
| ATOM | 563 | N | LEU | A | 306 | −19.205 | 21.414 | −1.732 | 1.00 | 35.72 | N |
| ATOM | 564 | CA | LEU | A | 306 | −20.501 | 20.714 | −1.729 | 1.00 | 35.71 | C |
| ATOM | 565 | CB | LEU | A | 306 | −21.408 | 21.123 | −2.898 | 1.00 | 35.63 | C |
| ATOM | 566 | CG | LEU | A | 306 | −22.846 | 20.541 | −2.951 | 1.00 | 35.50 | C |
| ATOM | 567 | CD1 | LEU | A | 306 | −23.816 | 21.242 | −1.938 | 1.00 | 33.45 | C |
| ATOM | 568 | CD2 | LEU | A | 306 | −23.410 | 20.598 | −4.394 | 1.00 | 34.54 | C |
| ATOM | 569 | C | LEU | A | 306 | −20.332 | 19.189 | −1.695 | 1.00 | 36.57 | C |
| ATOM | 570 | O | LEU | A | 306 | −19.528 | 18.610 | −2.470 | 1.00 | 37.23 | O |
| ATOM | 571 | N | THR | A | 307 | −21.087 | 18.560 | −0.783 | 1.00 | 36.81 | N |
| ATOM | 572 | CA | THR | A | 307 | −21.236 | 17.094 | −0.703 | 1.00 | 36.88 | C |
| ATOM | 573 | CB | THR | A | 307 | −21.613 | 16.638 | 0.755 | 1.00 | 37.15 | C |
| ATOM | 574 | OG1 | THR | A | 307 | −20.530 | 16.890 | 1.668 | 1.00 | 36.35 | O |
| ATOM | 575 | CG2 | THR | A | 307 | −21.996 | 15.147 | 0.798 | 1.00 | 36.59 | C |
| ATOM | 576 | C | THR | A | 307 | −22.340 | 16.628 | −1.676 | 1.00 | 37.34 | C |
| ATOM | 577 | O | THR | A | 307 | −23.509 | 17.092 | −1.578 | 1.00 | 38.08 | O |
| ATOM | 578 | N | VAL | A | 308 | −21.982 | 15.720 | −2.599 | 1.00 | 36.99 | N |
| ATOM | 579 | CA | VAL | A | 308 | −22.938 | 15.096 | −3.550 | 1.00 | 36.25 | C |
| ATOM | 580 | CB | VAL | A | 308 | −22.359 | 15.052 | −4.998 | 1.00 | 36.36 | C |
| ATOM | 581 | CG1 | VAL | A | 308 | −22.090 | 16.443 | −5.436 | 1.00 | 36.44 | C |
| ATOM | 582 | CG2 | VAL | A | 308 | −21.050 | 14.236 | −5.075 | 1.00 | 34.81 | C |
| ATOM | 583 | C | VAL | A | 308 | −23.346 | 13.689 | −3.095 | 1.00 | 36.02 | C |
| ATOM | 584 | O | VAL | A | 308 | −22.578 | 13.034 | −2.358 | 1.00 | 36.71 | O |
| ATOM | 585 | N | LEU | A | 309 | −24.524 | 13.213 | −3.529 | 1.00 | 34.86 | N |
| ATOM | 586 | CA | LEU | A | 309 | −24.953 | 11.862 | −3.162 | 1.00 | 33.52 | C |
| ATOM | 587 | CB | LEU | A | 309 | −26.451 | 11.762 | −3.059 | 1.00 | 32.62 | C |
| ATOM | 588 | CG | LEU | A | 309 | −27.095 | 12.823 | −2.180 | 1.00 | 33.64 | C |
| ATOM | 589 | CD1 | LEU | A | 309 | −28.570 | 12.633 | −2.275 | 1.00 | 35.12 | C |
| ATOM | 590 | CD2 | LEU | A | 309 | −26.612 | 12.811 | −0.706 | 1.00 | 30.67 | C |
| ATOM | 591 | C | LEU | A | 309 | −24.422 | 10.833 | −4.144 | 1.00 | 34.07 | C |
| ATOM | 592 | O | LEU | A | 309 | −24.340 | 11.089 | −5.350 | 1.00 | 34.29 | O |
| ATOM | 593 | N | HIS | A | 310 | −24.030 | 9.679 | −3.596 | 1.00 | 33.93 | N |
| ATOM | 594 | CA | HIS | A | 310 | −23.547 | 8.572 | −4.352 | 1.00 | 33.21 | C |
| ATOM | 595 | CB | HIS | A | 310 | −23.493 | 7.282 | −3.488 | 1.00 | 32.47 | C |
| ATOM | 596 | CG | HIS | A | 310 | −23.226 | 6.025 | −4.294 | 1.00 | 32.40 | C |
| ATOM | 597 | ND1 | HIS | A | 310 | −21.949 | 5.579 | −4.586 | 1.00 | 28.92 | N |
| ATOM | 598 | CE1 | HIS | A | 310 | −22.027 | 4.491 | −5.338 | 1.00 | 30.18 | C |
| ATOM | 599 | NE2 | HIS | A | 310 | −23.301 | 4.223 | −5.566 | 1.00 | 29.45 | N |
| ATOM | 600 | CD2 | HIS | A | 310 | −24.076 | 5.152 | −4.905 | 1.00 | 29.86 | C |
| ATOM | 601 | C | HIS | A | 310 | −24.479 | 8.429 | −5.581 | 1.00 | 34.17 | C |
| ATOM | 602 | O | HIS | A | 310 | −24.019 | 8.408 | −6.753 | 1.00 | 32.85 | O |
| ATOM | 603 | N | GLN | A | 311 | −25.787 | 8.360 | −5.317 | 1.00 | 35.47 | N |
| ATOM | 604 | CA | GLN | A | 311 | −26.721 | 8.121 | −6.430 | 1.00 | 37.22 | C |
| ATOM | 605 | CB | GLN | A | 311 | −28.170 | 7.796 | −5.983 | 1.00 | 36.44 | C |
| ATOM | 606 | CG | GLN | A | 311 | −28.478 | 8.087 | −4.497 | 1.00 | 38.65 | C |
| ATOM | 607 | CD | GLN | A | 311 | −29.985 | 8.297 | −4.227 | 1.00 | 39.86 | C |
| ATOM | 608 | OE1 | GLN | A | 311 | −30.363 | 9.043 | −3.313 | 1.00 | 42.35 | O |
| ATOM | 609 | NE2 | GLN | A | 311 | −30.853 | 7.654 | −5.045 | 1.00 | 43.40 | N |
| ATOM | 610 | C | GLN | A | 311 | −26.640 | 9.289 | −7.417 | 1.00 | 36.53 | C |
| ATOM | 611 | O | GLN | A | 311 | −26.654 | 9.071 | −8.602 | 1.00 | 36.16 | O |
| ATOM | 612 | N | ASP | A | 312 | −26.513 | 10.523 | −6.933 | 1.00 | 36.53 | N |
| ATOM | 613 | CA | ASP | A | 312 | −26.636 | 11.641 | −7.846 | 1.00 | 36.96 | C |
| ATOM | 614 | CB | ASP | A | 312 | −26.670 | 12.935 | −7.087 | 1.00 | 37.12 | C |
| ATOM | 615 | CG | ASP | A | 312 | −28.018 | 13.266 | −6.538 | 1.00 | 38.37 | C |
| ATOM | 616 | OD1 | ASP | A | 312 | −29.033 | 12.580 | −6.845 | 1.00 | 38.13 | O |
| ATOM | 617 | OD2 | ASP | A | 312 | −28.040 | 14.280 | −5.801 | 1.00 | 41.63 | O |
| ATOM | 618 | C | ASP | A | 312 | −25.471 | 11.685 | −8.834 | 1.00 | 37.55 | C |
| ATOM | 619 | O | ASP | A | 312 | −25.695 | 11.851 | −10.031 | 1.00 | 39.15 | O |
| ATOM | 620 | N | TRP | A | 313 | −24.248 | 11.515 | −8.329 | 1.00 | 36.41 | N |
| ATOM | 621 | CA | TRP | A | 313 | −23.038 | 11.651 | −9.094 | 1.00 | 35.37 | C |
| ATOM | 622 | CB | TRP | A | 313 | −21.788 | 11.464 | −8.203 | 1.00 | 34.92 | C |
| ATOM | 623 | CG | TRP | A | 313 | −20.501 | 11.745 | −8.975 | 1.00 | 34.68 | C |
| ATOM | 624 | CD1 | TRP | A | 313 | −19.573 | 10.810 | −9.444 | 1.00 | 33.90 | C |
| ATOM | 625 | NE1 | TRP | A | 313 | −18.514 | 11.468 | −10.077 | 1.00 | 34.24 | N |
| ATOM | 626 | CE2 | TRP | A | 313 | −18.751 | 12.823 | −10.060 | 1.00 | 36.02 | C |
| ATOM | 627 | CD2 | TRP | A | 313 | −19.989 | 13.041 | −9.353 | 1.00 | 33.65 | C |
| ATOM | 628 | CE3 | TRP | A | 313 | −20.450 | 14.363 | −9.173 | 1.00 | 30.92 | C |
| ATOM | 629 | CZ3 | TRP | A | 313 | −19.703 | 15.424 | −9.739 | 1.00 | 33.35 | C |
| ATOM | 630 | CH2 | TRP | A | 313 | −18.477 | 15.179 | −10.442 | 1.00 | 33.15 | C |
| ATOM | 631 | CZ2 | TRP | A | 313 | −17.981 | 13.897 | −10.603 | 1.00 | 36.01 | C |
| ATOM | 632 | C | TRP | A | 313 | −22.964 | 10.631 | −10.186 | 1.00 | 35.30 | C |
| ATOM | 633 | O | TRP | A | 313 | −22.585 | 10.979 | −11.323 | 1.00 | 36.36 | O |
| ATOM | 634 | N | LEU | A | 314 | −23.253 | 9.380 | −9.810 | 1.00 | 33.66 | N |
| ATOM | 635 | CA | LEU | A | 314 | −23.279 | 8.255 | −10.695 | 1.00 | 32.67 | C |
| ATOM | 636 | CB | LEU | A | 314 | −23.474 | 6.961 | −9.915 | 1.00 | 32.60 | C |
| ATOM | 637 | CG | LEU | A | 314 | −22.200 | 6.373 | −9.257 | 1.00 | 34.43 | C |
| ATOM | 638 | CD1 | LEU | A | 314 | −22.453 | 5.003 | −8.655 | 1.00 | 34.54 | C |
| ATOM | 639 | CD2 | LEU | A | 314 | −21.053 | 6.247 | −10.200 | 1.00 | 34.99 | C |
| ATOM | 640 | C | LEU | A | 314 | −24.371 | 8.418 | −11.770 | 1.00 | 32.64 | C |
| ATOM | 641 | O | LEU | A | 314 | −24.248 | 7.900 | −12.900 | 1.00 | 32.65 | O |
| ATOM | 642 | N | ASN | A | 315 | −25.424 | 9.153 | −11.420 | 1.00 | 31.97 | N |
| ATOM | 643 | CA | ASN | A | 315 | −26.581 | 9.330 | −12.275 | 1.00 | 31.66 | C |
| ATOM | 644 | CB | ASN | A | 315 | −27.809 | 9.372 | −11.385 | 1.00 | 30.77 | C |
| ATOM | 645 | CG | ASN | A | 315 | −28.397 | 8.016 | −11.180 | 1.00 | 30.83 | C |
| ATOM | 646 | OD1 | ASN | A | 315 | −28.077 | 7.084 | −11.915 | 1.00 | 34.78 | O |
| ATOM | 647 | ND2 | ASN | A | 315 | −29.264 | 7.878 | −10.196 | 1.00 | 29.82 | N |
| ATOM | 648 | C | ASN | A | 315 | −26.497 | 10.541 | −13.256 | 1.00 | 32.40 | C |
| ATOM | 649 | O | ASN | A | 315 | −27.481 | 10.920 | −13.918 | 1.00 | 32.17 | O |
| ATOM | 650 | N | GLY | A | 316 | −25.314 | 11.151 | −13.312 | 1.00 | 32.65 | N |
| ATOM | 651 | CA | GLY | A | 316 | −25.016 | 12.211 | −14.243 | 1.00 | 33.58 | C |
| ATOM | 652 | C | GLY | A | 316 | −25.477 | 13.593 | −13.832 | 1.00 | 34.97 | C |
| ATOM | 653 | O | GLY | A | 316 | −25.548 | 14.502 | −14.703 | 1.00 | 35.12 | O |
| ATOM | 654 | N | LYS | A | 317 | −25.805 | 13.795 | −12.544 | 1.00 | 35.29 | N |
| ATOM | 655 | CA | LYS | A | 317 | −26.339 | 15.115 | −12.177 | 1.00 | 36.95 | C |
| ATOM | 656 | CB | LYS | A | 317 | −27.014 | 15.163 | −10.804 | 1.00 | 36.09 | C |
| ATOM | 657 | CG | LYS | A | 317 | −28.182 | 14.265 | −10.728 | 1.00 | 36.60 | C |
| ATOM | 658 | CD | LYS | A | 317 | −29.163 | 14.686 | −9.658 | 1.00 | 38.58 | C |
| ATOM | 659 | CE | LYS | A | 317 | −30.385 | 13.767 | −9.653 | 1.00 | 37.79 | C |
| ATOM | 660 | NZ | LYS | A | 317 | −31.456 | 14.355 | −8.787 | 1.00 | 42.02 | N |
| ATOM | 661 | C | LYS | A | 317 | −25.239 | 16.176 | −12.400 | 1.00 | 37.88 | C |
| ATOM | 662 | O | LYS | A | 317 | −24.058 | 15.820 | −12.391 | 1.00 | 38.73 | O |
| ATOM | 663 | N | GLU | A | 318 | −25.635 | 17.431 | −12.647 | 1.00 | 38.31 | N |
| ATOM | 664 | CA | GLU | A | 318 | −24.689 | 18.502 | −12.976 | 1.00 | 39.11 | C |
| ATOM | 665 | CB | GLU | A | 318 | −25.062 | 19.203 | −14.285 | 1.00 | 39.62 | C |
| ATOM | 666 | CG | GLU | A | 318 | −24.684 | 18.473 | −15.525 | 1.00 | 40.55 | C |
| ATOM | 667 | CD | GLU | A | 318 | −24.895 | 19.314 | −16.734 | 1.00 | 42.44 | C |
| ATOM | 668 | OE1 | GLU | A | 318 | −25.983 | 19.936 | −16.886 | 1.00 | 42.51 | O |
| ATOM | 669 | OE2 | GLU | A | 318 | −23.953 | 19.350 | −17.553 | 1.00 | 46.08 | O |
| ATOM | 670 | C | GLU | A | 318 | −24.594 | 19.567 | −11.867 | 1.00 | 39.40 | C |
| ATOM | 671 | O | GLU | A | 318 | −25.609 | 19.973 | −11.264 | 1.00 | 39.87 | O |
| ATOM | 672 | N | TYR | A | 319 | −23.368 | 20.031 | −11.632 | 1.00 | 38.82 | N |
| ATOM | 673 | CA | TYR | A | 319 | −23.048 | 20.784 | −10.438 | 1.00 | 38.11 | C |
| ATOM | 674 | CB | TYR | A | 319 | −22.123 | 19.955 | −9.520 | 1.00 | 38.42 | C |
| ATOM | 675 | CG | TYR | A | 319 | −22.781 | 18.663 | −8.951 | 1.00 | 37.86 | C |
| ATOM | 676 | CD1 | TYR | A | 319 | −22.688 | 17.411 | −9.631 | 1.00 | 37.92 | C |
| ATOM | 677 | CE1 | TYR | A | 319 | −23.299 | 16.256 | −9.104 | 1.00 | 37.14 | C |
| ATOM | 678 | CZ | TYR | A | 319 | −23.996 | 16.362 | −7.873 | 1.00 | 37.23 | C |
| ATOM | 679 | OH | TYR | A | 319 | −24.614 | 15.279 | −7.269 | 1.00 | 39.31 | O |
| ATOM | 680 | CE2 | TYR | A | 319 | −24.071 | 17.578 | −7.204 | 1.00 | 34.06 | C |
| ATOM | 681 | CD2 | TYR | A | 319 | −23.459 | 18.694 | −7.726 | 1.00 | 35.56 | C |
| ATOM | 682 | C | TYR | A | 319 | −22.447 | 22.119 | −10.836 | 1.00 | 37.85 | C |
| ATOM | 683 | O | TYR | A | 319 | −21.274 | 22.220 | −11.260 | 1.00 | 36.59 | O |
| ATOM | 684 | N | LYS | A | 320 | −23.297 | 23.134 | −10.739 | 1.00 | 37.38 | N |
| ATOM | 685 | CA | LYS | A | 320 | −22.886 | 24.480 | −10.989 | 1.00 | 38.49 | C |
| ATOM | 686 | CB | LYS | A | 320 | −24.029 | 25.285 | −11.596 | 1.00 | 38.82 | C |
| ATOM | 687 | CG | LYS | A | 320 | −23.605 | 26.657 | −12.131 | 1.00 | 39.52 | C |
| ATOM | 688 | CD | LYS | A | 320 | −24.500 | 27.116 | −13.262 | 1.00 | 41.14 | C |
| ATOM | 689 | CE | LYS | A | 320 | −25.755 | 27.794 | −12.756 | 1.00 | 43.94 | C |
| ATOM | 690 | NZ | LYS | A | 320 | −26.841 | 27.687 | −13.792 | 1.00 | 46.17 | N |
| ATOM | 691 | C | LYS | A | 320 | −22.404 | 25.142 | −9.702 | 1.00 | 39.16 | C |
| ATOM | 692 | O | LYS | A | 320 | −23.088 | 25.127 | −8.687 | 1.00 | 39.50 | O |
| ATOM | 693 | N | CYS | A | 321 | −21.204 | 25.698 | −9.756 | 1.00 | 39.96 | N |
| ATOM | 694 | CA | CYS | A | 321 | −20.743 | 26.660 | −8.774 | 1.00 | 40.72 | C |
| ATOM | 695 | CB | CYS | A | 321 | −19.377 | 26.248 | −8.207 | 1.00 | 41.09 | C |
| ATOM | 696 | SG | CYS | A | 321 | −18.566 | 27.499 | −7.225 | 1.00 | 40.10 | S |
| ATOM | 697 | C | CYS | A | 321 | −20.688 | 28.022 | −9.494 | 1.00 | 41.83 | C |
| ATOM | 698 | O | CYS | A | 321 | −20.006 | 28.166 | −10.539 | 1.00 | 41.94 | O |
| ATOM | 699 | N | LYS | A | 322 | −21.445 | 28.988 | −8.951 | 1.00 | 42.07 | N |
| ATOM | 700 | CA | LYS | A | 322 | −21.560 | 30.356 | −9.475 | 1.00 | 42.34 | C |
| ATOM | 701 | CB | LYS | A | 322 | −23.052 | 30.743 | −9.603 | 1.00 | 42.17 | C |
| ATOM | 702 | CG | LYS | A | 322 | −23.330 | 32.074 | −10.335 | 1.00 | 42.75 | C |
| ATOM | 703 | CD | LYS | A | 322 | −24.742 | 32.629 | −10.058 | 1.00 | 42.18 | C |
| ATOM | 704 | CE | LYS | A | 322 | −25.548 | 32.928 | −11.344 | 1.00 | 40.93 | C |
| ATOM | 705 | NZ | LYS | A | 322 | −26.525 | 34.051 | −11.131 | 1.00 | 40.44 | N |
| ATOM | 706 | C | LYS | A | 322 | −20.844 | 31.288 | −8.495 | 1.00 | 42.24 | C |
| ATOM | 707 | O | LYS | A | 322 | −21.154 | 31.292 | −7.302 | 1.00 | 42.55 | O |
| ATOM | 708 | N | VAL | A | 323 | −19.885 | 32.061 | −8.978 | 1.00 | 42.22 | N |
| ATOM | 709 | CA | VAL | A | 323 | −19.099 | 32.902 | −8.083 | 1.00 | 42.58 | C |
| ATOM | 710 | CB | VAL | A | 323 | −17.589 | 32.618 | −8.177 | 1.00 | 42.31 | C |
| ATOM | 711 | CG1 | VAL | A | 323 | −16.847 | 33.528 | −7.241 | 1.00 | 42.80 | C |
| ATOM | 712 | CG2 | VAL | A | 323 | −17.298 | 31.207 | −7.808 | 1.00 | 41.21 | C |
| ATOM | 713 | C | VAL | A | 323 | −19.372 | 34.376 | −8.353 | 1.00 | 43.54 | C |
| ATOM | 714 | O | VAL | A | 323 | −19.106 | 34.892 | −9.442 | 1.00 | 43.92 | O |
| ATOM | 715 | N | SER | A | 324 | −19.908 | 35.058 | −7.352 | 1.00 | 44.15 | N |
| ATOM | 716 | CA | SER | A | 324 | −20.208 | 36.454 | −7.501 | 1.00 | 44.72 | C |
| ATOM | 717 | CB | SER | A | 324 | −21.627 | 36.762 | −7.027 | 1.00 | 44.77 | C |
| ATOM | 718 | OG | SER | A | 324 | −22.567 | 36.463 | −8.041 | 1.00 | 44.81 | O |
| ATOM | 719 | C | SER | A | 324 | −19.205 | 37.264 | −6.723 | 1.00 | 45.30 | C |
| ATOM | 720 | O | SER | A | 324 | −19.119 | 37.149 | −5.501 | 1.00 | 45.16 | O |
| ATOM | 721 | N | ASN | A | 325 | −18.434 | 38.059 | −7.457 | 1.00 | 46.01 | N |
| ATOM | 722 | CA | ASN | A | 325 | −17.622 | 39.097 | −6.863 | 1.00 | 46.99 | C |
| ATOM | 723 | CB | ASN | A | 325 | −16.143 | 38.876 | −7.202 | 1.00 | 46.75 | C |
| ATOM | 724 | CG | ASN | A | 325 | −15.201 | 39.664 | −6.285 | 1.00 | 46.12 | C |
| ATOM | 725 | OD1 | ASN | A | 325 | −15.611 | 40.152 | −5.232 | 1.00 | 46.32 | O |
| ATOM | 726 | ND2 | ASN | A | 325 | −13.938 | 39.792 | −6.691 | 1.00 | 44.33 | N |
| ATOM | 727 | C | ASN | A | 325 | −18.107 | 40.469 | −7.353 | 1.00 | 47.94 | C |
| ATOM | 728 | O | ASN | A | 325 | −18.804 | 40.544 | −8.374 | 1.00 | 48.36 | O |
| ATOM | 729 | N | LYS | A | 326 | −17.770 | 41.538 | −6.627 | 1.00 | 48.55 | N |
| ATOM | 730 | CA | LYS | A | 326 | −18.064 | 42.891 | −7.100 | 1.00 | 49.51 | C |
| ATOM | 731 | CB | LYS | A | 326 | −18.146 | 43.887 | −5.936 | 1.00 | 49.50 | C |
| ATOM | 732 | CG | LYS | A | 326 | −19.271 | 43.551 | −4.956 | 1.00 | 49.40 | C |
| ATOM | 733 | CD | LYS | A | 326 | −19.449 | 44.614 | −3.904 | 1.00 | 49.78 | C |
| ATOM | 734 | CE | LYS | A | 326 | −20.692 | 44.353 | −3.061 | 1.00 | 49.99 | C |
| ATOM | 735 | NZ | LYS | A | 326 | −21.191 | 45.600 | −2.410 | 1.00 | 50.06 | N |
| ATOM | 736 | C | LYS | A | 326 | −17.013 | 43.295 | −8.122 | 1.00 | 50.05 | C |
| ATOM | 737 | O | LYS | A | 326 | −17.192 | 44.236 | −8.878 | 1.00 | 49.82 | O |
| ATOM | 738 | N | ALA | A | 327 | −15.957 | 42.506 | −8.172 | 1.00 | 51.28 | N |
| ATOM | 739 | CA | ALA | A | 327 | −14.871 | 42.662 | −9.138 | 1.00 | 52.63 | C |
| ATOM | 740 | CB | ALA | A | 327 | −13.558 | 42.199 | −8.537 | 1.00 | 52.54 | C |
| ATOM | 741 | C | ALA | A | 327 | −15.182 | 41.886 | −10.409 | 1.00 | 53.81 | C |
| ATOM | 742 | O | ALA | A | 327 | −14.436 | 41.965 | −11.391 | 1.00 | 54.15 | O |
| ATOM | 743 | N | LEU | A | 328 | −16.318 | 41.170 | −10.414 | 1.00 | 55.21 | N |
| ATOM | 744 | CA | LEU | A | 328 | −16.726 | 40.359 | −11.605 | 1.00 | 56.11 | C |
| ATOM | 745 | CB | LEU | A | 328 | −16.968 | 38.903 | −11.204 | 1.00 | 55.91 | C |
| ATOM | 746 | CG | LEU | A | 328 | −15.776 | 38.161 | −10.597 | 1.00 | 55.02 | C |
| ATOM | 747 | CD1 | LEU | A | 328 | −16.156 | 36.731 | −10.246 | 1.00 | 54.91 | C |
| ATOM | 748 | CD2 | LEU | A | 328 | −14.589 | 38.182 | −11.548 | 1.00 | 53.07 | C |
| ATOM | 749 | C | LEU | A | 328 | −17.974 | 40.984 | −12.276 | 1.00 | 57.04 | C |
| ATOM | 750 | O | LEU | A | 328 | −19.007 | 41.165 | −11.634 | 1.00 | 57.20 | O |
| ATOM | 751 | N | PRO | A | 329 | −17.939 | 41.325 | −13.560 | 1.00 | 57.70 | N |
| ATOM | 752 | CA | PRO | A | 329 | −19.123 | 41.939 | −14.209 | 1.00 | 57.93 | C |
| ATOM | 753 | CB | PRO | A | 329 | −18.536 | 42.474 | −15.527 | 1.00 | 58.01 | C |
| ATOM | 754 | CG | PRO | A | 329 | −17.156 | 42.862 | −15.158 | 1.00 | 58.09 | C |
| ATOM | 755 | CD | PRO | A | 329 | −16.716 | 41.740 | −14.261 | 1.00 | 57.92 | C |
| ATOM | 756 | C | PRO | A | 329 | −20.330 | 40.991 | −14.370 | 1.00 | 57.91 | C |
| ATOM | 757 | O | PRO | A | 329 | −21.476 | 41.388 | −14.179 | 1.00 | 58.06 | O |
| ATOM | 758 | N | LEU | A | 330 | −20.004 | 39.790 | −14.681 | 1.00 | 57.68 | N |
| ATOM | 759 | CA | LEU | A | 330 | −20.977 | 38.757 | −14.710 | 1.00 | 57.20 | C |
| ATOM | 760 | CB | LEU | A | 330 | −20.996 | 38.078 | −16.079 | 1.00 | 57.42 | C |
| ATOM | 761 | CG | LEU | A | 330 | −21.320 | 38.973 | −17.275 | 1.00 | 58.75 | C |
| ATOM | 762 | CD1 | LEU | A | 330 | −21.305 | 38.171 | −18.567 | 1.00 | 59.46 | C |
| ATOM | 763 | CD2 | LEU | A | 330 | −22.666 | 39.656 | −17.086 | 1.00 | 59.40 | C |
| ATOM | 764 | C | LEU | A | 330 | −20.663 | 37.861 | −13.541 | 1.00 | 56.66 | C |
| ATOM | 765 | O | LEU | A | 330 | −19.532 | 37.914 | −13.054 | 1.00 | 56.65 | O |
| ATOM | 766 | N | PRO | A | 331 | −21.579 | 37.016 | −13.037 | 1.00 | 56.19 | N |
| ATOM | 767 | CA | PRO | A | 331 | −21.150 | 36.046 | −12.012 | 1.00 | 55.74 | C |
| ATOM | 768 | CB | PRO | A | 331 | −22.427 | 35.408 | −11.479 | 1.00 | 55.86 | C |
| ATOM | 769 | CG | PRO | A | 331 | −23.482 | 36.408 | −11.790 | 1.00 | 56.01 | C |
| ATOM | 770 | CD | PRO | A | 331 | −22.803 | 37.578 | −12.434 | 1.00 | 55.98 | C |
| ATOM | 771 | C | PRO | A | 331 | −20.269 | 34.971 | −12.713 | 1.00 | 55.35 | C |
| ATOM | 772 | O | PRO | A | 331 | −20.781 | 34.310 | −13.623 | 1.00 | 55.41 | O |
| ATOM | 773 | N | GLU | A | 332 | −18.990 | 34.741 | −12.344 | 1.00 | 55.03 | N |
| ATOM | 774 | CA | GLU | A | 332 | −18.317 | 33.666 | −13.085 | 1.00 | 54.24 | C |
| ATOM | 775 | CB | GLU | A | 332 | −16.782 | 33.734 | −12.983 | 1.00 | 54.49 | C |
| ATOM | 776 | CG | GLU | A | 332 | −16.027 | 32.784 | −13.922 | 1.00 | 55.57 | C |
| ATOM | 777 | CD | GLU | A | 332 | −15.669 | 33.420 | −15.271 | 1.00 | 57.34 | C |
| ATOM | 778 | OE1 | GLU | A | 332 | −15.500 | 34.646 | −15.339 | 1.00 | 55.02 | O |
| ATOM | 779 | OE2 | GLU | A | 332 | −15.561 | 32.679 | −16.275 | 1.00 | 56.86 | O |
| ATOM | 780 | C | GLU | A | 332 | −18.885 | 32.300 | −12.599 | 1.00 | 53.71 | C |
| ATOM | 781 | O | GLU | A | 332 | −18.921 | 32.006 | −11.400 | 1.00 | 53.77 | O |
| ATOM | 782 | N | GLU | A | 333 | −19.332 | 31.483 | −13.567 | 1.00 | 52.83 | N |
| ATOM | 783 | CA | GLU | A | 333 | −19.993 | 30.173 | −13.325 | 1.00 | 51.22 | C |
| ATOM | 784 | CB | GLU | A | 333 | −21.361 | 30.117 | −14.035 | 1.00 | 50.99 | C |
| ATOM | 785 | CG | GLU | A | 333 | −22.235 | 31.375 | −13.778 | 1.00 | 51.38 | C |
| ATOM | 786 | CD | GLU | A | 333 | −23.654 | 31.340 | −14.375 | 1.00 | 51.65 | C |
| ATOM | 787 | OE1 | GLU | A | 333 | −24.311 | 32.419 | −14.386 | 1.00 | 52.39 | O |
| ATOM | 788 | OE2 | GLU | A | 333 | −24.121 | 30.256 | −14.809 | 1.00 | 51.04 | O |
| ATOM | 789 | C | GLU | A | 333 | −19.083 | 29.035 | −13.800 | 1.00 | 50.08 | C |
| ATOM | 790 | O | GLU | A | 333 | −18.185 | 29.277 | −14.596 | 1.00 | 50.34 | O |
| ATOM | 791 | N | LYS | A | 334 | −19.294 | 27.821 | −13.273 | 1.00 | 48.66 | N |
| ATOM | 792 | CA | LYS | A | 334 | −18.569 | 26.592 | −13.653 | 1.00 | 46.57 | C |
| ATOM | 793 | CB | LYS | A | 334 | −17.262 | 26.469 | −12.889 | 1.00 | 46.23 | C |
| ATOM | 794 | CG | LYS | A | 334 | −16.167 | 27.397 | −13.323 | 1.00 | 45.07 | C |
| ATOM | 795 | CD | LYS | A | 334 | −15.494 | 26.895 | −14.547 | 1.00 | 42.21 | C |
| ATOM | 796 | CE | LYS | A | 334 | −14.361 | 27.794 | −14.854 | 1.00 | 43.33 | C |
| ATOM | 797 | NZ | LYS | A | 334 | −14.893 | 28.891 | −15.667 | 1.00 | 43.32 | N |
| ATOM | 798 | C | LYS | A | 334 | −19.396 | 25.332 | −13.354 | 1.00 | 46.00 | C |
| ATOM | 799 | O | LYS | A | 334 | −19.927 | 25.193 | −12.258 | 1.00 | 45.49 | O |
| ATOM | 800 | N | THR | A | 335 | −19.492 | 24.411 | −14.321 | 1.00 | 45.23 | N |
| ATOM | 801 | CA | THR | A | 335 | −20.248 | 23.176 | −14.119 | 1.00 | 43.92 | C |
| ATOM | 802 | CB | THR | A | 335 | −21.468 | 23.058 | −15.093 | 1.00 | 44.09 | C |
| ATOM | 803 | OG1 | THR | A | 335 | −22.372 | 24.155 | −14.881 | 1.00 | 44.97 | O |
| ATOM | 804 | CG2 | THR | A | 335 | −22.240 | 21.804 | −14.804 | 1.00 | 43.81 | C |
| ATOM | 805 | C | THR | A | 335 | −19.354 | 21.940 | −14.181 | 1.00 | 43.27 | C |
| ATOM | 806 | O | THR | A | 335 | −18.333 | 21.889 | −14.873 | 1.00 | 42.28 | O |
| ATOM | 807 | N | ILE | A | 336 | −19.750 | 20.932 | −13.429 | 1.00 | 43.13 | N |
| ATOM | 808 | CA | ILE | A | 336 | −19.038 | 19.674 | −13.400 | 1.00 | 42.86 | C |
| ATOM | 809 | CB | ILE | A | 336 | −17.868 | 19.692 | −12.368 | 1.00 | 43.29 | C |
| ATOM | 810 | CG1 | ILE | A | 336 | −16.752 | 18.709 | −12.752 | 1.00 | 44.01 | C |
| ATOM | 811 | CD1 | ILE | A | 336 | −15.318 | 19.183 | −12.390 | 1.00 | 45.95 | C |
| ATOM | 812 | CG2 | ILE | A | 336 | −18.353 | 19.467 | −10.924 | 1.00 | 43.39 | C |
| ATOM | 813 | C | ILE | A | 336 | −20.028 | 18.526 | −13.154 | 1.00 | 42.93 | C |
| ATOM | 814 | O | ILE | A | 336 | −20.996 | 18.639 | −12.378 | 1.00 | 43.77 | O |
| ATOM | 815 | N | SER | A | 337 | −19.722 | 17.398 | −13.804 | 1.00 | 42.66 | N |
| ATOM | 816 | CA | SER | A | 337 | −20.432 | 16.134 | −13.645 | 1.00 | 40.85 | C |
| ATOM | 817 | CB | SER | A | 337 | −21.638 | 16.034 | −14.580 | 1.00 | 39.73 | C |
| ATOM | 818 | OG | SER | A | 337 | −21.214 | 15.822 | −15.914 | 1.00 | 38.46 | O |
| ATOM | 819 | C | SER | A | 337 | −19.406 | 14.995 | −13.834 | 1.00 | 40.25 | C |
| ATOM | 820 | O | SER | A | 337 | −18.257 | 15.238 | −14.165 | 1.00 | 39.05 | O |
| ATOM | 821 | N | LYS | A | 338 | −19.883 | 13.811 | −13.600 | 1.00 | 40.00 | N |
| ATOM | 822 | CA | LYS | A | 338 | −19.134 | 12.617 | −13.832 | 1.00 | 38.95 | C |
| ATOM | 823 | CB | LYS | A | 338 | −19.951 | 11.423 | −13.362 | 1.00 | 39.06 | C |
| ATOM | 824 | CG | LYS | A | 338 | −19.219 | 10.108 | −13.233 | 1.00 | 37.92 | C |
| ATOM | 825 | CD | LYS | A | 338 | −20.174 | 8.968 | −12.965 | 1.00 | 36.65 | C |
| ATOM | 826 | CE | LYS | A | 338 | −21.207 | 8.818 | −14.080 | 1.00 | 36.99 | C |
| ATOM | 827 | NZ | LYS | A | 338 | −20.602 | 8.256 | −15.319 | 1.00 | 35.15 | N |
| ATOM | 828 | C | LYS | A | 338 | −18.829 | 12.523 | −15.314 | 1.00 | 38.25 | C |
| ATOM | 829 | O | LYS | A | 338 | −19.521 | 13.124 | −16.122 | 1.00 | 39.71 | O |
| ATOM | 830 | N | ALA | A | 339 | −17.788 | 11.792 | −15.668 | 1.00 | 36.91 | N |
| ATOM | 831 | CA | ALA | A | 339 | −17.460 | 11.601 | −17.086 | 1.00 | 35.44 | C |
| ATOM | 832 | CB | ALA | A | 339 | −16.215 | 10.738 | −17.244 | 1.00 | 33.61 | C |
| ATOM | 833 | C | ALA | A | 339 | −18.689 | 10.963 | −17.810 | 1.00 | 34.79 | C |
| ATOM | 834 | O | ALA | A | 339 | −19.201 | 9.936 | −17.400 | 1.00 | 34.21 | O |
| ATOM | 835 | N | LYS | A | 340 | −19.133 | 11.614 | −18.887 | 1.00 | 34.09 | N |
| ATOM | 836 | CA | LYS | A | 340 | −20.292 | 11.154 | −19.659 | 1.00 | 33.97 | C |
| ATOM | 837 | CB | LYS | A | 340 | −20.958 | 12.296 | −20.395 | 1.00 | 33.75 | C |
| ATOM | 838 | CG | LYS | A | 340 | −21.312 | 13.497 | −19.552 | 1.00 | 34.53 | C |
| ATOM | 839 | CD | LYS | A | 340 | −21.551 | 14.719 | −20.429 | 1.00 | 34.06 | C |
| ATOM | 840 | CE | LYS | A | 340 | −22.455 | 15.710 | −19.732 | 1.00 | 36.99 | C |
| ATOM | 841 | NZ | LYS | A | 340 | −22.697 | 16.913 | −20.576 | 1.00 | 41.74 | N |
| ATOM | 842 | C | LYS | A | 340 | −19.907 | 10.109 | −20.699 | 1.00 | 32.88 | C |
| ATOM | 843 | O | LYS | A | 340 | −18.735 | 9.980 | −21.051 | 1.00 | 33.79 | O |
| ATOM | 844 | N | GLY | A | 341 | −20.884 | 9.360 | −21.194 | 1.00 | 32.44 | N |
| ATOM | 845 | CA | GLY | A | 341 | −20.672 | 8.328 | −22.253 | 1.00 | 31.49 | C |
| ATOM | 846 | C | GLY | A | 341 | −21.268 | 6.986 | −21.847 | 1.00 | 31.52 | C |
| ATOM | 847 | O | GLY | A | 341 | −21.461 | 6.729 | −20.670 | 1.00 | 31.14 | O |
| ATOM | 848 | N | GLN | A | 342 | −21.621 | 6.117 | −22.771 | 1.00 | 31.42 | N |
| ATOM | 849 | CA | GLN | A | 342 | −22.230 | 4.895 | −22.278 | 1.00 | 32.13 | C |
| ATOM | 850 | CB | GLN | A | 342 | −22.758 | 4.055 | −23.427 | 1.00 | 31.89 | C |
| ATOM | 851 | CG | GLN | A | 342 | −24.010 | 4.602 | −24.085 | 1.00 | 33.03 | C |
| ATOM | 852 | CD | GLN | A | 342 | −25.232 | 4.510 | −23.180 | 1.00 | 32.85 | C |
| ATOM | 853 | OE1 | GLN | A | 342 | −25.403 | 3.540 | −22.435 | 1.00 | 33.49 | O |
| ATOM | 854 | NE2 | GLN | A | 342 | −26.073 | 5.537 | −23.221 | 1.00 | 31.41 | N |
| ATOM | 855 | C | GLN | A | 342 | −21.222 | 4.091 | −21.451 | 1.00 | 32.86 | C |
| ATOM | 856 | O | GLN | A | 342 | −20.166 | 3.745 | −21.971 | 1.00 | 32.38 | O |
| ATOM | 857 | N | PRO | A | 343 | −21.562 | 3.758 | −20.172 | 1.00 | 33.86 | N |
| ATOM | 858 | CA | PRO | A | 343 | −20.766 | 2.833 | −19.324 | 1.00 | 33.69 | C |
| ATOM | 859 | CB | PRO | A | 343 | −21.649 | 2.602 | −18.112 | 1.00 | 34.04 | C |
| ATOM | 860 | CG | PRO | A | 343 | −23.046 | 2.986 | −18.603 | 1.00 | 35.06 | C |
| ATOM | 861 | CD | PRO | A | 343 | −22.779 | 4.180 | −19.465 | 1.00 | 34.14 | C |
| ATOM | 862 | C | PRO | A | 343 | −20.524 | 1.490 | −19.992 | 1.00 | 33.72 | C |
| ATOM | 863 | O | PRO | A | 343 | −21.407 | 0.961 | −20.680 | 1.00 | 33.73 | O |
| ATOM | 864 | N | ARG | A | 344 | −19.307 | 0.977 | −19.829 | 1.00 | 33.55 | N |
| ATOM | 865 | CA | ARG | A | 344 | −18.951 | −0.339 | −20.289 | 1.00 | 33.74 | C |
| ATOM | 866 | CB | ARG | A | 344 | −18.092 | −0.187 | −21.521 | 1.00 | 33.33 | C |
| ATOM | 867 | CG | ARG | A | 344 | −18.926 | 0.522 | −22.651 | 1.00 | 36.88 | C |
| ATOM | 868 | CD | ARG | A | 344 | −18.172 | 0.767 | −23.936 | 1.00 | 37.00 | C |
| ATOM | 869 | NE | ARG | A | 344 | −17.512 | −0.468 | −24.292 | 1.00 | 40.28 | N |
| ATOM | 870 | CZ | ARG | A | 344 | −16.837 | −0.673 | −25.423 | 1.00 | 44.65 | C |
| ATOM | 871 | NH1 | ARG | A | 344 | −16.705 | 0.313 | −26.336 | 1.00 | 44.41 | N |
| ATOM | 872 | NH2 | ARG | A | 344 | −16.283 | −1.876 | −25.633 | 1.00 | 42.20 | N |
| ATOM | 873 | C | ARG | A | 344 | −18.302 | −1.054 | −19.104 | 1.00 | 33.55 | C |
| ATOM | 874 | O | ARG | A | 344 | −17.523 | −0.438 | −18.402 | 1.00 | 33.87 | O |
| ATOM | 875 | N | GLU | A | 345 | −18.735 | −2.288 | −18.821 | 1.00 | 33.58 | N |
| ATOM | 876 | CA | GLU | A | 345 | −18.267 | −3.117 | −17.678 | 1.00 | 34.17 | C |
| ATOM | 877 | CB | GLU | A | 345 | −19.345 | −4.210 | −17.350 | 1.00 | 34.00 | C |
| ATOM | 878 | CG | GLU | A | 345 | −19.074 | −5.258 | −16.215 | 1.00 | 34.59 | C |
| ATOM | 879 | CD | GLU | A | 345 | −20.304 | −6.160 | −15.899 | 1.00 | 37.09 | C |
| ATOM | 880 | OE1 | GLU | A | 345 | −20.182 | −7.254 | −15.281 | 1.00 | 38.99 | O |
| ATOM | 881 | OE2 | GLU | A | 345 | −21.438 | −5.763 | −16.251 | 1.00 | 42.02 | O |
| ATOM | 882 | C | GLU | A | 345 | −16.890 | −3.731 | −17.988 | 1.00 | 33.27 | C |
| ATOM | 883 | O | GLU | A | 345 | −16.736 | −4.380 | −19.014 | 1.00 | 34.48 | O |
| ATOM | 884 | N | PRO | A | 346 | −15.869 | −3.505 | −17.142 | 1.00 | 32.67 | N |
| ATOM | 885 | CA | PRO | A | 346 | −14.564 | −4.128 | −17.463 | 1.00 | 32.62 | C |
| ATOM | 886 | CB | PRO | A | 346 | −13.634 | −3.616 | −16.353 | 1.00 | 32.96 | C |
| ATOM | 887 | CG | PRO | A | 346 | −14.546 | −3.148 | −15.260 | 1.00 | 32.07 | C |
| ATOM | 888 | CD | PRO | A | 346 | −15.808 | −2.678 | −15.930 | 1.00 | 31.69 | C |
| ATOM | 889 | C | PRO | A | 346 | −14.547 | −5.643 | −17.425 | 1.00 | 33.03 | C |
| ATOM | 890 | O | PRO | A | 346 | −15.238 | −6.223 | −16.594 | 1.00 | 33.16 | O |
| ATOM | 891 | N | GLN | A | 347 | −13.758 | −6.278 | −18.300 | 1.00 | 33.59 | N |
| ATOM | 892 | CA | GLN | A | 347 | −13.337 | −7.675 | −18.108 | 1.00 | 34.09 | C |
| ATOM | 893 | CB | GLN | A | 347 | −13.101 | −8.434 | −19.417 | 1.00 | 34.58 | C |
| ATOM | 894 | CG | GLN | A | 347 | −14.329 | −8.788 | −20.170 | 1.00 | 37.72 | C |
| ATOM | 895 | CD | GLN | A | 347 | −14.796 | −7.605 | −20.986 | 1.00 | 43.32 | C |
| ATOM | 896 | OE1 | GLN | A | 347 | −14.338 | −7.395 | −22.120 | 1.00 | 44.01 | O |
| ATOM | 897 | NE2 | GLN | A | 347 | −15.698 | −6.796 | −20.402 | 1.00 | 44.97 | N |
| ATOM | 898 | C | GLN | A | 347 | −12.044 | −7.700 | −17.329 | 1.00 | 33.64 | C |
| ATOM | 899 | O | GLN | A | 347 | −11.089 | −6.989 | −17.660 | 1.00 | 32.97 | O |
| ATOM | 900 | N | VAL | A | 348 | −12.017 | −8.543 | −16.298 | 1.00 | 34.29 | N |
| ATOM | 901 | CA | VAL | A | 348 | −10.864 | −8.620 | −15.396 | 1.00 | 34.21 | C |
| ATOM | 902 | CB | VAL | A | 348 | −11.229 | −8.341 | −13.941 | 1.00 | 33.80 | C |
| ATOM | 903 | CG1 | VAL | A | 348 | −9.960 | −8.363 | −13.099 | 1.00 | 35.66 | C |
| ATOM | 904 | CG2 | VAL | A | 348 | −11.941 | −6.976 | −13.828 | 1.00 | 32.62 | C |
| ATOM | 905 | C | VAL | A | 348 | −10.165 | −9.940 | −15.520 | 1.00 | 34.04 | C |
| ATOM | 906 | O | VAL | A | 348 | −10.766 | −10.992 | −15.281 | 1.00 | 33.79 | O |
| ATOM | 907 | N | TYR | A | 349 | −8.898 | −9.885 | −15.926 | 1.00 | 34.08 | N |
| ATOM | 908 | CA | TYR | A | 349 | −8.126 | −11.114 | −16.080 | 1.00 | 34.66 | C |
| ATOM | 909 | CB | TYR | A | 349 | −7.799 | −11.394 | −17.545 | 1.00 | 33.73 | C |
| ATOM | 910 | CG | TYR | A | 349 | −8.997 | −11.362 | −18.468 | 1.00 | 33.30 | C |
| ATOM | 911 | CD1 | TYR | A | 349 | −10.052 | −12.283 | −18.332 | 1.00 | 33.37 | C |
| ATOM | 912 | CE1 | TYR | A | 349 | −11.167 | −12.253 | −19.205 | 1.00 | 33.42 | C |
| ATOM | 913 | CZ | TYR | A | 349 | −11.232 | −11.275 | −20.221 | 1.00 | 34.09 | C |
| ATOM | 914 | OH | TYR | A | 349 | −12.311 | −11.225 | −21.094 | 1.00 | 33.59 | O |
| ATOM | 915 | CE2 | TYR | A | 349 | −10.198 | −10.351 | −20.361 | 1.00 | 32.79 | C |
| ATOM | 916 | CD2 | TYR | A | 349 | −9.081 | −10.410 | −19.488 | 1.00 | 33.49 | C |
| ATOM | 917 | C | TYR | A | 349 | −6.872 | −11.049 | −15.247 | 1.00 | 35.21 | C |
| ATOM | 918 | O | TYR | A | 349 | −6.219 | −10.013 | −15.194 | 1.00 | 35.25 | O |
| ATOM | 919 | N | THR | A | 350 | −6.559 | −12.154 | −14.582 | 1.00 | 36.19 | N |
| ATOM | 920 | CA | THR | A | 350 | −5.290 | −12.304 | −13.864 | 1.00 | 37.31 | C |
| ATOM | 921 | CB | THR | A | 350 | −5.508 | −12.943 | −12.485 | 1.00 | 37.46 | C |
| ATOM | 922 | OG1 | THR | A | 350 | −6.230 | −14.176 | −12.626 | 1.00 | 37.47 | O |
| ATOM | 923 | CG2 | THR | A | 350 | −6.312 | −12.011 | −11.557 | 1.00 | 37.38 | C |
| ATOM | 924 | C | THR | A | 350 | −4.349 | −13.174 | −14.706 | 1.00 | 38.06 | C |
| ATOM | 925 | O | THR | A | 350 | −4.804 | −14.115 | −15.347 | 1.00 | 38.48 | O |
| ATOM | 926 | N | LEU | A | 351 | −3.057 | −12.858 | −14.744 | 1.00 | 38.62 | N |
| ATOM | 927 | CA | LEU | A | 351 | −2.115 | −13.640 | −15.554 | 1.00 | 38.93 | C |
| ATOM | 928 | CB | LEU | A | 351 | −1.729 | −12.891 | −16.838 | 1.00 | 38.75 | C |
| ATOM | 929 | CG | LEU | A | 351 | −2.840 | −12.182 | −17.642 | 1.00 | 38.82 | C |
| ATOM | 930 | CD1 | LEU | A | 351 | −2.243 | −11.292 | −18.681 | 1.00 | 39.11 | C |
| ATOM | 931 | CD2 | LEU | A | 351 | −3.796 | −13.156 | −18.318 | 1.00 | 40.70 | C |
| ATOM | 932 | C | LEU | A | 351 | −0.864 | −13.978 | −14.750 | 1.00 | 39.58 | C |
| ATOM | 933 | O | LEU | A | 351 | −0.263 | −13.094 | −14.122 | 1.00 | 39.33 | O |
| ATOM | 934 | N | PRO | A | 352 | −0.481 | −15.269 | −14.754 | 1.00 | 39.77 | N |
| ATOM | 935 | CA | PRO | A | 352 | 0.739 | −15.789 | −14.147 | 1.00 | 39.92 | C |
| ATOM | 936 | CB | PRO | A | 352 | 0.719 | −17.285 | −14.521 | 1.00 | 39.02 | C |
| ATOM | 937 | CG | PRO | A | 352 | −0.204 | −17.401 | −15.623 | 1.00 | 38.64 | C |
| ATOM | 938 | CD | PRO | A | 352 | −1.260 | −16.356 | −15.366 | 1.00 | 40.00 | C |
| ATOM | 939 | C | PRO | A | 352 | 2.022 | −15.149 | −14.690 | 1.00 | 40.58 | C |
| ATOM | 940 | O | PRO | A | 352 | 1.979 | −14.475 | −15.748 | 1.00 | 41.02 | O |
| ATOM | 941 | N | PRO | A | 353 | 3.152 | −15.352 | −13.953 | 1.00 | 40.66 | N |
| ATOM | 942 | CA | PRO | A | 353 | 4.497 | −15.005 | −14.408 | 1.00 | 40.78 | C |
| ATOM | 943 | CB | PRO | A | 353 | 5.389 | −15.315 | −13.172 | 1.00 | 40.91 | C |
| ATOM | 944 | CG | PRO | A | 353 | 4.486 | −15.480 | −12.014 | 1.00 | 40.28 | C |
| ATOM | 945 | CD | PRO | A | 353 | 3.174 | −15.944 | −12.590 | 1.00 | 40.78 | C |
| ATOM | 946 | C | PRO | A | 353 | 4.924 | −15.883 | −15.599 | 1.00 | 40.56 | C |
| ATOM | 947 | O | PRO | A | 353 | 4.655 | −17.072 | −15.599 | 1.00 | 40.37 | O |
| ATOM | 948 | N | SER | A | 354 | 5.586 | −15.299 | −16.594 | 1.00 | 40.69 | N |
| ATOM | 949 | CA | SER | A | 354 | 6.144 | −16.068 | −17.706 | 1.00 | 40.58 | C |
| ATOM | 950 | CB | SER | A | 354 | 6.995 | −15.138 | −18.561 | 1.00 | 40.27 | C |
| ATOM | 951 | OG | SER | A | 354 | 7.640 | −15.854 | −19.600 | 1.00 | 41.10 | O |
| ATOM | 952 | C | SER | A | 354 | 6.989 | −17.246 | −17.212 | 1.00 | 40.74 | C |
| ATOM | 953 | O | SER | A | 354 | 7.488 | −17.211 | −16.088 | 1.00 | 41.16 | O |
| ATOM | 954 | N | ARG | A | 355 | 7.143 | −18.286 | −18.043 | 1.00 | 41.02 | N |
| ATOM | 955 | CA | ARG | A | 355 | 8.125 | −19.374 | −17.816 | 1.00 | 41.30 | C |
| ATOM | 956 | CB | ARG | A | 355 | 8.318 | −20.208 | −19.099 | 1.00 | 41.33 | C |
| ATOM | 957 | CG | ARG | A | 355 | 7.222 | −21.285 | −19.387 | 1.00 | 44.10 | C |
| ATOM | 958 | CD | ARG | A | 355 | 7.073 | −21.700 | −20.915 | 1.00 | 44.13 | C |
| ATOM | 959 | NE | ARG | A | 355 | 5.974 | −20.977 | −21.620 | 1.00 | 48.21 | N |
| ATOM | 960 | CZ | ARG | A | 355 | 4.927 | −21.537 | −22.253 | 1.00 | 48.37 | C |
| ATOM | 961 | NH1 | ARG | A | 355 | 4.771 | −22.861 | −22.321 | 1.00 | 46.93 | N |
| ATOM | 962 | NH2 | ARG | A | 355 | 4.021 | −20.751 | −22.834 | 1.00 | 49.11 | N |
| ATOM | 963 | C | ARG | A | 355 | 9.447 | −18.725 | −17.450 | 1.00 | 40.40 | C |
| ATOM | 964 | O | ARG | A | 355 | 10.133 | −19.124 | −16.507 | 1.00 | 39.74 | O |
| ATOM | 965 | N | GLU | A | 356 | 9.751 | −17.674 | −18.206 | 1.00 | 40.04 | N |
| ATOM | 966 | CA | GLU | A | 356 | 10.969 | −16.903 | −18.090 | 1.00 | 39.83 | C |
| ATOM | 967 | CB | GLU | A | 356 | 11.000 | −15.887 | −19.210 | 1.00 | 40.22 | C |
| ATOM | 968 | CG | GLU | A | 356 | 11.611 | −16.363 | −20.504 | 1.00 | 42.08 | C |
| ATOM | 969 | CD | GLU | A | 356 | 12.195 | −15.184 | −21.293 | 1.00 | 46.20 | C |
| ATOM | 970 | OE1 | GLU | A | 356 | 11.498 | −14.120 | −21.453 | 1.00 | 44.53 | O |
| ATOM | 971 | OE2 | GLU | A | 356 | 13.371 | −15.326 | −21.731 | 1.00 | 47.60 | O |
| ATOM | 972 | C | GLU | A | 356 | 11.208 | −16.157 | −16.776 | 1.00 | 39.01 | C |
| ATOM | 973 | O | GLU | A | 356 | 12.353 | −16.051 | −16.342 | 1.00 | 39.05 | O |
| ATOM | 974 | N | GLU | A | 357 | 10.164 | −15.609 | −16.159 | 1.00 | 38.10 | N |
| ATOM | 975 | CA | GLU | A | 357 | 10.384 | −14.804 | −14.959 | 1.00 | 37.12 | C |
| ATOM | 976 | CB | GLU | A | 357 | 9.171 | −13.941 | −14.628 | 1.00 | 37.37 | C |
| ATOM | 977 | CG | GLU | A | 357 | 9.537 | −12.698 | −13.788 | 1.00 | 35.53 | C |
| ATOM | 978 | CD | GLU | A | 357 | 8.358 | −11.785 | −13.484 | 1.00 | 35.17 | C |
| ATOM | 979 | OE1 | GLU | A | 357 | 7.274 | −11.919 | −14.095 | 1.00 | 33.86 | O |
| ATOM | 980 | OE2 | GLU | A | 357 | 8.526 | −10.928 | −12.608 | 1.00 | 30.76 | O |
| ATOM | 981 | C | GLU | A | 357 | 10.736 | −15.678 | −13.774 | 1.00 | 37.52 | C |
| ATOM | 982 | O | GLU | A | 357 | 11.302 | −15.222 | −12.777 | 1.00 | 37.12 | O |
| ATOM | 983 | N | MET | A | 358 | 10.420 | −16.958 | −13.920 | 1.00 | 38.20 | N |
| ATOM | 984 | CA | MET | A | 358 | 10.484 | −17.918 | −12.828 | 1.00 | 38.45 | C |
| ATOM | 985 | CB | MET | A | 358 | 9.787 | −19.203 | −13.241 | 1.00 | 38.18 | C |
| ATOM | 986 | CG | MET | A | 358 | 8.839 | −19.736 | −12.178 | 1.00 | 39.60 | C |
| ATOM | 987 | SD | MET | A | 358 | 7.494 | −18.597 | −11.750 | 1.00 | 38.38 | S |
| ATOM | 988 | CE | MET | A | 358 | 6.188 | −19.142 | −12.859 | 1.00 | 40.59 | C |
| ATOM | 989 | C | MET | A | 358 | 11.909 | −18.177 | −12.329 | 1.00 | 38.40 | C |
| ATOM | 990 | O | MET | A | 358 | 12.101 | −18.727 | −11.243 | 1.00 | 38.82 | O |
| ATOM | 991 | N | THR | A | 359 | 12.898 | −17.749 | −13.110 | 1.00 | 38.07 | N |
| ATOM | 992 | CA | THR | A | 359 | 14.301 | −17.821 | −12.721 | 1.00 | 37.84 | C |
| ATOM | 993 | CB | THR | A | 359 | 15.181 | −17.168 | −13.788 | 1.00 | 38.15 | C |
| ATOM | 994 | OG1 | THR | A | 359 | 14.826 | −17.671 | −15.079 | 1.00 | 38.58 | O |
| ATOM | 995 | CG2 | THR | A | 359 | 16.658 | −17.433 | −13.517 | 1.00 | 38.68 | C |
| ATOM | 996 | C | THR | A | 359 | 14.514 | −16.999 | −11.481 | 1.00 | 37.35 | C |
| ATOM | 997 | O | THR | A | 359 | 15.199 | −17.407 | −10.552 | 1.00 | 36.85 | O |
| ATOM | 998 | N | LYS | A | 360 | 13.899 | −15.826 | −11.490 | 1.00 | 37.06 | N |
| ATOM | 999 | CA | LYS | A | 360 | 14.252 | −14.765 | −10.577 | 1.00 | 37.01 | C |
| ATOM | 1000 | CB | LYS | A | 360 | 13.755 | −13.418 | −11.122 | 1.00 | 37.55 | C |
| ATOM | 1001 | CG | LYS | A | 360 | 13.968 | −13.190 | −12.638 | 1.00 | 39.03 | C |
| ATOM | 1002 | CD | LYS | A | 360 | 15.385 | −12.689 | −13.002 | 1.00 | 40.85 | C |
| ATOM | 1003 | CE | LYS | A | 360 | 15.777 | −13.091 | −14.442 | 1.00 | 42.07 | C |
| ATOM | 1004 | NZ | LYS | A | 360 | 14.870 | −12.552 | −15.518 | 1.00 | 42.80 | N |
| ATOM | 1005 | C | LYS | A | 360 | 13.730 | −15.012 | −9.162 | 1.00 | 36.65 | C |
| ATOM | 1006 | O | LYS | A | 360 | 13.000 | −15.974 | −8.896 | 1.00 | 36.30 | O |
| ATOM | 1007 | N | ASN | A | 361 | 14.123 | −14.135 | −8.250 | 1.00 | 36.22 | N |
| ATOM | 1008 | CA | ASN | A | 361 | 13.743 | −14.270 | −6.869 | 1.00 | 35.96 | C |
| ATOM | 1009 | CB | ASN | A | 361 | 14.963 | −13.997 | −5.992 | 1.00 | 36.05 | C |
| ATOM | 1010 | CG | ASN | A | 361 | 16.089 | −15.040 | −6.219 | 1.00 | 35.69 | C |
| ATOM | 1011 | OD1 | ASN | A | 361 | 17.169 | −14.713 | −6.717 | 1.00 | 34.68 | O |
| ATOM | 1012 | ND2 | ASN | A | 361 | 15.809 | −16.300 | −5.880 | 1.00 | 33.63 | N |
| ATOM | 1013 | C | ASN | A | 361 | 12.514 | −13.426 | −6.540 | 1.00 | 36.02 | C |
| ATOM | 1014 | O | ASN | A | 361 | 12.043 | −13.373 | −5.406 | 1.00 | 36.31 | O |
| ATOM | 1015 | N | GLN | A | 362 | 11.976 | −12.795 | −7.576 | 1.00 | 36.15 | N |
| ATOM | 1016 | CA | GLN | A | 362 | 10.693 | −12.082 | −7.517 | 1.00 | 36.13 | C |
| ATOM | 1017 | CB | GLN | A | 362 | 10.893 | −10.609 | −7.073 | 1.00 | 36.40 | C |
| ATOM | 1018 | CG | GLN | A | 362 | 11.013 | −10.434 | −5.522 | 1.00 | 38.17 | C |
| ATOM | 1019 | CD | GLN | A | 362 | 12.141 | −9.474 | −5.057 | 1.00 | 41.73 | C |
| ATOM | 1020 | OE1 | GLN | A | 362 | 12.037 | −8.253 | −5.234 | 1.00 | 43.54 | O |
| ATOM | 1021 | NE2 | GLN | A | 362 | 13.213 | −10.034 | −4.444 | 1.00 | 40.37 | N |
| ATOM | 1022 | C | GLN | A | 362 | 9.930 | −12.220 | −8.865 | 1.00 | 35.33 | C |
| ATOM | 1023 | O | GLN | A | 362 | 10.523 | −12.286 | −9.925 | 1.00 | 35.07 | O |
| ATOM | 1024 | N | VAL | A | 363 | 8.611 | −12.316 | −8.802 | 1.00 | 34.94 | N |
| ATOM | 1025 | CA | VAL | A | 363 | 7.794 | −12.554 | −9.979 | 1.00 | 34.08 | C |
| ATOM | 1026 | CB | VAL | A | 363 | 7.131 | −13.965 | −9.945 | 1.00 | 34.26 | C |
| ATOM | 1027 | CG1 | VAL | A | 363 | 8.165 | −15.048 | −9.683 | 1.00 | 33.76 | C |
| ATOM | 1028 | CG2 | VAL | A | 363 | 6.049 | −14.041 | −8.910 | 1.00 | 33.40 | C |
| ATOM | 1029 | C | VAL | A | 363 | 6.728 | −11.479 | −10.062 | 1.00 | 33.76 | C |
| ATOM | 1030 | O | VAL | A | 363 | 6.456 | −10.807 | −9.057 | 1.00 | 33.66 | O |
| ATOM | 1031 | N | SER | A | 364 | 6.143 | −11.336 | −11.257 | 1.00 | 33.39 | N |
| ATOM | 1032 | CA | SER | A | 364 | 5.033 | −10.405 | −11.534 | 1.00 | 32.92 | C |
| ATOM | 1033 | CB | SER | A | 364 | 5.206 | −9.722 | −12.875 | 1.00 | 31.90 | C |
| ATOM | 1034 | OG | SER | A | 364 | 6.469 | −9.154 | −12.948 | 1.00 | 33.43 | O |
| ATOM | 1035 | C | SER | A | 364 | 3.739 | −11.133 | −11.659 | 1.00 | 32.58 | C |
| ATOM | 1036 | O | SER | A | 364 | 3.619 | −12.038 | −12.465 | 1.00 | 33.12 | O |
| ATOM | 1037 | N | LEU | A | 365 | 2.742 | −10.683 | −10.924 | 1.00 | 32.04 | N |
| ATOM | 1038 | CA | LEU | A | 365 | 1.402 | −11.159 | −11.149 | 1.00 | 31.42 | C |
| ATOM | 1039 | CB | LEU | A | 365 | 0.727 | −11.570 | −9.823 | 1.00 | 30.97 | C |
| ATOM | 1040 | CG | LEU | A | 365 | 1.490 | −12.540 | −8.881 | 1.00 | 30.85 | C |
| ATOM | 1041 | CD1 | LEU | A | 365 | 0.587 | −12.883 | −7.739 | 1.00 | 33.27 | C |
| ATOM | 1042 | CD2 | LEU | A | 365 | 2.026 | −13.845 | −9.506 | 1.00 | 27.60 | C |
| ATOM | 1043 | C | LEU | A | 365 | 0.663 | −10.058 | −11.893 | 1.00 | 31.15 | C |
| ATOM | 1044 | O | LEU | A | 365 | 0.735 | −8.883 | −11.543 | 1.00 | 30.86 | O |
| ATOM | 1045 | N | THR | A | 366 | −0.030 | −10.433 | −12.951 | 1.00 | 31.39 | N |
| ATOM | 1046 | CA | THR | A | 366 | −0.585 | −9.402 | −13.839 | 1.00 | 31.81 | C |
| ATOM | 1047 | CB | THR | A | 366 | 0.070 | −9.460 | −15.264 | 1.00 | 30.89 | C |
| ATOM | 1048 | OG1 | THR | A | 366 | 1.452 | −9.110 | −15.133 | 1.00 | 30.04 | O |
| ATOM | 1049 | CG2 | THR | A | 366 | −0.565 | −8.499 | −16.205 | 1.00 | 30.04 | C |
| ATOM | 1050 | C | THR | A | 366 | −2.122 | −9.386 | −13.817 | 1.00 | 32.09 | C |
| ATOM | 1051 | O | THR | A | 366 | −2.769 | −10.428 | −13.935 | 1.00 | 32.27 | O |
| ATOM | 1052 | N | CYS | A | 367 | −2.670 | −8.204 | −13.563 | 1.00 | 32.96 | N |
| ATOM | 1053 | CA | CYS | A | 367 | −4.095 | −7.947 | −13.713 | 1.00 | 33.14 | C |
| ATOM | 1054 | CB | CYS | A | 367 | −4.628 | −7.247 | −12.470 | 1.00 | 33.22 | C |
| ATOM | 1055 | SG | CYS | A | 367 | −6.408 | −7.393 | −12.254 | 1.00 | 36.97 | S |
| ATOM | 1056 | C | CYS | A | 367 | −4.443 | −7.126 | −14.986 | 1.00 | 32.54 | C |
| ATOM | 1057 | O | CYS | A | 367 | −4.225 | −5.903 | −15.029 | 1.00 | 31.39 | O |
| ATOM | 1058 | N | LEU | A | 368 | −4.969 | −7.806 | −16.006 | 1.00 | 32.12 | N |
| ATOM | 1059 | CA | LEU | A | 368 | −5.570 | −7.107 | −17.136 | 1.00 | 32.76 | C |
| ATOM | 1060 | CB | LEU | A | 368 | −5.535 | −7.929 | −18.421 | 1.00 | 33.26 | C |
| ATOM | 1061 | CG | LEU | A | 368 | −6.222 | −7.253 | −19.639 | 1.00 | 33.45 | C |
| ATOM | 1062 | CD1 | LEU | A | 368 | −5.493 | −5.986 | −20.064 | 1.00 | 30.24 | C |
| ATOM | 1063 | CD2 | LEU | A | 368 | −6.279 | −8.226 | −20.819 | 1.00 | 33.36 | C |
| ATOM | 1064 | C | LEU | A | 368 | −7.001 | −6.693 | −16.880 | 1.00 | 32.31 | C |
| ATOM | 1065 | O | LEU | A | 368 | −7.894 | −7.490 | −16.569 | 1.00 | 33.14 | O |
| ATOM | 1066 | N | VAL | A | 369 | −7.232 | −5.424 | −17.059 | 1.00 | 32.68 | N |
| ATOM | 1067 | CA | VAL | A | 369 | −8.590 | −4.861 | −16.978 | 1.00 | 32.99 | C |
| ATOM | 1068 | CB | VAL | A | 369 | −8.672 | −3.836 | −15.816 | 1.00 | 32.70 | C |
| ATOM | 1069 | CG1 | VAL | A | 369 | −10.070 | −3.308 | −15.698 | 1.00 | 34.34 | C |
| ATOM | 1070 | CG2 | VAL | A | 369 | −8.200 | −4.471 | −14.484 | 1.00 | 32.15 | C |
| ATOM | 1071 | C | VAL | A | 369 | −8.877 | −4.161 | −18.336 | 1.00 | 32.54 | C |
| ATOM | 1072 | O | VAL | A | 369 | −8.142 | −3.253 | −18.751 | 1.00 | 32.40 | O |
| ATOM | 1073 | N | LYS | A | 370 | −9.909 | −4.599 | −19.036 | 1.00 | 32.16 | N |
| ATOM | 1074 | CA | LYS | A | 370 | −10.157 | −4.035 | −20.352 | 1.00 | 33.34 | C |
| ATOM | 1075 | CB | LYS | A | 370 | −9.503 | −4.899 | −21.462 | 1.00 | 32.44 | C |
| ATOM | 1076 | CG | LYS | A | 370 | −10.371 | −6.050 | −21.875 | 1.00 | 32.56 | C |
| ATOM | 1077 | CD | LYS | A | 370 | −9.897 | −6.769 | −23.129 | 1.00 | 33.97 | C |
| ATOM | 1078 | CE | LYS | A | 370 | −10.639 | −6.238 | −24.365 | 1.00 | 34.17 | C |
| ATOM | 1079 | NZ | LYS | A | 370 | −10.654 | −7.234 | −25.497 | 1.00 | 34.30 | N |
| ATOM | 1080 | C | LYS | A | 370 | −11.655 | −3.786 | −20.674 | 1.00 | 33.16 | C |
| ATOM | 1081 | O | LYS | A | 370 | −12.531 | −4.378 | −20.079 | 1.00 | 33.52 | O |
| ATOM | 1082 | N | GLY | A | 371 | −11.906 | −2.925 | −21.656 | 1.00 | 33.59 | N |
| ATOM | 1083 | CA | GLY | A | 371 | −13.233 | −2.709 | −22.230 | 1.00 | 32.79 | C |
| ATOM | 1084 | C | GLY | A | 371 | −14.116 | −1.940 | −21.282 | 1.00 | 33.17 | C |
| ATOM | 1085 | O | GLY | A | 371 | −15.351 | −2.063 | −21.358 | 1.00 | 34.36 | O |
| ATOM | 1086 | N | PHE | A | 372 | −13.498 | −1.157 | −20.383 | 1.00 | 31.50 | N |
| ATOM | 1087 | CA | PHE | A | 372 | −14.268 | −0.310 | −19.499 | 1.00 | 29.43 | C |
| ATOM | 1088 | CB | PHE | A | 372 | −13.719 | −0.332 | −18.067 | 1.00 | 29.21 | C |
| ATOM | 1089 | CG | PHE | A | 372 | −12.337 | 0.263 | −17.909 | 1.00 | 28.02 | C |
| ATOM | 1090 | CD1 | PHE | A | 372 | −12.175 | 1.569 | −17.529 | 1.00 | 27.04 | C |
| ATOM | 1091 | CE1 | PHE | A | 372 | −10.898 | 2.115 | −17.311 | 1.00 | 27.19 | C |
| ATOM | 1092 | CZ | PHE | A | 372 | −9.793 | 1.346 | −17.493 | 1.00 | 29.12 | C |
| ATOM | 1093 | CE2 | PHE | A | 372 | −9.944 | 0.019 | −17.858 | 1.00 | 28.40 | C |
| ATOM | 1094 | CD2 | PHE | A | 372 | −11.211 | −0.517 | −18.034 | 1.00 | 28.22 | C |
| ATOM | 1095 | C | PHE | A | 372 | −14.441 | 1.110 | −19.991 | 1.00 | 28.82 | C |
| ATOM | 1096 | O | PHE | A | 372 | −13.626 | 1.678 | −20.741 | 1.00 | 28.00 | O |
| ATOM | 1097 | N | TYR | A | 373 | −15.521 | 1.687 | −19.529 | 1.00 | 28.42 | N |
| ATOM | 1098 | CA | TYR | A | 373 | −15.850 | 3.037 | −19.871 | 1.00 | 29.40 | C |
| ATOM | 1099 | CB | TYR | A | 373 | −16.471 | 3.178 | −21.287 | 1.00 | 28.31 | C |
| ATOM | 1100 | CG | TYR | A | 373 | −16.439 | 4.649 | −21.660 | 1.00 | 30.52 | C |
| ATOM | 1101 | CD1 | TYR | A | 373 | −15.379 | 5.153 | −22.411 | 1.00 | 28.78 | C |
| ATOM | 1102 | CE1 | TYR | A | 373 | −15.301 | 6.471 | −22.709 | 1.00 | 28.54 | C |
| ATOM | 1103 | CZ | TYR | A | 373 | −16.247 | 7.345 | −22.243 | 1.00 | 28.13 | C |
| ATOM | 1104 | OH | TYR | A | 373 | −16.054 | 8.655 | −22.577 | 1.00 | 30.91 | O |
| ATOM | 1105 | CE2 | TYR | A | 373 | −17.319 | 6.927 | −21.445 | 1.00 | 28.02 | C |
| ATOM | 1106 | CD2 | TYR | A | 373 | −17.416 | 5.577 | −21.150 | 1.00 | 30.05 | C |
| ATOM | 1107 | C | TYR | A | 373 | −16.834 | 3.515 | −18.811 | 1.00 | 29.53 | C |
| ATOM | 1108 | O | TYR | A | 373 | −17.774 | 2.795 | −18.530 | 1.00 | 30.17 | O |
| ATOM | 1109 | N | PRO | A | 374 | −16.623 | 4.713 | −18.209 | 1.00 | 30.09 | N |
| ATOM | 1110 | CA | PRO | A | 374 | −15.544 | 5.698 | −18.375 | 1.00 | 30.13 | C |
| ATOM | 1111 | CB | PRO | A | 374 | −16.079 | 6.918 | −17.593 | 1.00 | 30.55 | C |
| ATOM | 1112 | CG | PRO | A | 374 | −16.957 | 6.372 | −16.561 | 1.00 | 28.15 | C |
| ATOM | 1113 | CD | PRO | A | 374 | −17.618 | 5.183 | −17.213 | 1.00 | 30.17 | C |
| ATOM | 1114 | C | PRO | A | 374 | −14.244 | 5.228 | −17.726 | 1.00 | 30.89 | C |
| ATOM | 1115 | O | PRO | A | 374 | −14.236 | 4.189 | −17.044 | 1.00 | 31.03 | O |
| ATOM | 1116 | N | SER | A | 375 | −13.185 | 6.121 | −17.833 | 1.00 | 30.73 | N |
| ATOM | 1117 | CA | SER | A | 375 | −11.847 | 5.768 | −17.337 | 1.00 | 30.37 | C |
| ATOM | 1118 | CB | SER | A | 375 | −10.797 | 6.597 | −18.059 | 1.00 | 30.43 | C |
| ATOM | 1119 | OG | SER | A | 375 | −10.788 | 7.932 | −17.598 | 1.00 | 28.34 | O |
| ATOM | 1120 | C | SER | A | 375 | −11.659 | 5.792 | −15.806 | 1.00 | 30.57 | C |
| ATOM | 1121 | O | SER | A | 375 | −10.707 | 5.179 | −15.304 | 1.00 | 31.14 | O |
| ATOM | 1122 | N | ASP | A | 376 | −12.517 | 6.485 | −15.072 | 1.00 | 30.38 | N |
| ATOM | 1123 | CA | ASP | A | 376 | −12.383 | 6.484 | −13.624 | 1.00 | 30.58 | C |
| ATOM | 1124 | CB | ASP | A | 376 | −13.573 | 7.216 | −12.967 | 1.00 | 31.98 | C |
| ATOM | 1125 | CG | ASP | A | 376 | −14.121 | 8.383 | −13.781 | 1.00 | 33.40 | C |
| ATOM | 1126 | OD1 | ASP | A | 376 | −15.304 | 8.363 | −14.161 | 1.00 | 34.50 | O |
| ATOM | 1127 | OD2 | ASP | A | 376 | −13.354 | 9.339 | −14.043 | 1.00 | 32.77 | O |
| ATOM | 1128 | C | ASP | A | 376 | −12.320 | 5.044 | −13.101 | 1.00 | 30.13 | C |
| ATOM | 1129 | O | ASP | A | 376 | −13.242 | 4.274 | −13.325 | 1.00 | 29.96 | O |
| ATOM | 1130 | N | ILE | A | 377 | −11.245 | 4.700 | −12.412 | 1.00 | 29.05 | N |
| ATOM | 1131 | CA | ILE | A | 377 | −11.097 | 3.327 | −11.943 | 1.00 | 28.59 | C |
| ATOM | 1132 | CB | ILE | A | 377 | −10.574 | 2.450 | −13.083 | 1.00 | 27.66 | C |
| ATOM | 1133 | CG1 | ILE | A | 377 | −10.659 | 0.961 | −12.729 | 1.00 | 28.31 | C |
| ATOM | 1134 | CD1 | ILE | A | 377 | −11.007 | 0.047 | −13.926 | 1.00 | 28.63 | C |
| ATOM | 1135 | CG2 | ILE | A | 377 | −9.190 | 2.850 | −13.449 | 1.00 | 26.17 | C |
| ATOM | 1136 | C | ILE | A | 377 | −10.169 | 3.239 | −10.728 | 1.00 | 28.72 | C |
| ATOM | 1137 | O | ILE | A | 377 | −9.323 | 4.095 | −10.551 | 1.00 | 27.91 | O |
| ATOM | 1138 | N | ALA | A | 378 | −10.327 | 2.222 | −9.878 | 1.00 | 29.44 | N |
| ATOM | 1139 | CA | ALA | A | 378 | −9.364 | 2.007 | −8.764 | 1.00 | 29.96 | C |
| ATOM | 1140 | CB | ALA | A | 378 | −9.946 | 2.359 | −7.436 | 1.00 | 27.95 | C |
| ATOM | 1141 | C | ALA | A | 378 | −9.033 | 0.558 | −8.808 | 1.00 | 30.84 | C |
| ATOM | 1142 | O | ALA | A | 378 | −9.933 | −0.243 | −9.009 | 1.00 | 32.55 | O |
| ATOM | 1143 | N | VAL | A | 379 | −7.752 | 0.227 | −8.655 | 1.00 | 31.69 | N |
| ATOM | 1144 | CA | VAL | A | 379 | −7.278 | −1.158 | −8.664 | 1.00 | 32.46 | C |
| ATOM | 1145 | CB | VAL | A | 379 | −6.650 | −1.513 | −10.052 | 1.00 | 32.35 | C |
| ATOM | 1146 | CG1 | VAL | A | 379 | −6.131 | −2.924 | −10.070 | 1.00 | 33.51 | C |
| ATOM | 1147 | CG2 | VAL | A | 379 | −7.645 | −1.327 | −11.171 | 1.00 | 30.67 | C |
| ATOM | 1148 | C | VAL | A | 379 | −6.263 | −1.387 | −7.517 | 1.00 | 33.25 | C |
| ATOM | 1149 | O | VAL | A | 379 | −5.337 | −0.605 | −7.344 | 1.00 | 33.12 | O |
| ATOM | 1150 | N | GLU | A | 380 | −6.459 | −2.451 | −6.727 | 1.00 | 34.70 | N |
| ATOM | 1151 | CA | GLU | A | 380 | −5.556 | −2.821 | −5.601 | 1.00 | 35.86 | C |
| ATOM | 1152 | CB | GLU | A | 380 | −6.104 | −2.341 | −4.256 | 1.00 | 36.62 | C |
| ATOM | 1153 | CG | GLU | A | 380 | −6.336 | −0.852 | −4.186 | 1.00 | 41.63 | C |
| ATOM | 1154 | CD | GLU | A | 380 | −7.206 | −0.461 | −3.000 | 1.00 | 47.10 | C |
| ATOM | 1155 | OE1 | GLU | A | 380 | −7.275 | −1.284 | −2.035 | 1.00 | 49.24 | O |
| ATOM | 1156 | OE2 | GLU | A | 380 | −7.817 | 0.656 | −3.054 | 1.00 | 47.07 | O |
| ATOM | 1157 | C | GLU | A | 380 | −5.389 | −4.325 | −5.482 | 1.00 | 35.06 | C |
| ATOM | 1158 | O | GLU | A | 380 | −6.269 | −5.060 | −5.909 | 1.00 | 34.83 | O |
| ATOM | 1159 | N | TRP | A | 381 | −4.289 | −4.777 | −4.871 | 1.00 | 34.47 | N |
| ATOM | 1160 | CA | TRP | A | 381 | −4.109 | −6.200 | −4.600 | 1.00 | 34.36 | C |
| ATOM | 1161 | CB | TRP | A | 381 | −2.807 | −6.727 | −5.180 | 1.00 | 33.63 | C |
| ATOM | 1162 | CG | TRP | A | 381 | −2.632 | −6.730 | −6.660 | 1.00 | 33.57 | C |
| ATOM | 1163 | CD1 | TRP | A | 381 | −2.354 | −5.647 | −7.431 | 1.00 | 32.06 | C |
| ATOM | 1164 | NE1 | TRP | A | 381 | −2.248 | −6.005 | −8.739 | 1.00 | 33.45 | N |
| ATOM | 1165 | CE2 | TRP | A | 381 | −2.494 | −7.350 | −8.853 | 1.00 | 32.37 | C |
| ATOM | 1166 | CD2 | TRP | A | 381 | −2.738 | −7.848 | −7.549 | 1.00 | 32.84 | C |
| ATOM | 1167 | CE3 | TRP | A | 381 | −3.018 | −9.222 | −7.406 | 1.00 | 33.13 | C |
| ATOM | 1168 | CZ3 | TRP | A | 381 | −3.037 | −10.022 | −8.544 | 1.00 | 33.33 | C |
| ATOM | 1169 | CH2 | TRP | A | 381 | −2.775 | −9.504 | −9.811 | 1.00 | 33.75 | C |
| ATOM | 1170 | CZ2 | TRP | A | 381 | −2.505 | −8.162 | −9.989 | 1.00 | 32.34 | C |
| ATOM | 1171 | C | TRP | A | 381 | −4.150 | −6.444 | −3.113 | 1.00 | 35.14 | C |
| ATOM | 1172 | O | TRP | A | 381 | −3.835 | −5.559 | −2.312 | 1.00 | 34.80 | O |
| ATOM | 1173 | N | GLU | A | 382 | −4.510 | −7.648 | −2.758 | 1.00 | 35.79 | N |
| ATOM | 1174 | CA | GLU | A | 382 | −4.543 | −8.057 | −1.374 | 1.00 | 36.64 | C |
| ATOM | 1175 | CB | GLU | A | 382 | −5.838 | −7.646 | −0.684 | 1.00 | 36.72 | C |
| ATOM | 1176 | CG | GLU | A | 382 | −7.132 | −8.172 | −1.280 | 1.00 | 37.76 | C |
| ATOM | 1177 | CD | GLU | A | 382 | −8.319 | −7.540 | −0.575 | 1.00 | 38.12 | C |
| ATOM | 1178 | OE1 | GLU | A | 382 | −8.713 | −6.420 | −0.975 | 1.00 | 36.20 | O |
| ATOM | 1179 | OE2 | GLU | A | 382 | −8.862 | −8.163 | 0.355 | 1.00 | 39.34 | O |
| ATOM | 1180 | C | GLU | A | 382 | −4.329 | −9.530 | −1.295 | 1.00 | 36.79 | C |
| ATOM | 1181 | O | GLU | A | 382 | −4.576 | −10.295 | −2.218 | 1.00 | 36.95 | O |
| ATOM | 1182 | N | SER | A | 383 | −3.856 | −9.891 | −0.108 | 1.00 | 36.62 | N |
| ATOM | 1183 | CA | SER | A | 383 | −3.602 | −11.254 | 0.261 | 1.00 | 36.97 | C |
| ATOM | 1184 | CB | SER | A | 383 | −2.112 | −11.549 | 0.291 | 1.00 | 36.33 | C |
| ATOM | 1185 | OG | SER | A | 383 | −1.842 | −12.902 | −0.023 | 1.00 | 34.86 | O |
| ATOM | 1186 | C | SER | A | 383 | −4.259 | −11.565 | 1.587 | 1.00 | 38.29 | C |
| ATOM | 1187 | O | SER | A | 383 | −4.108 | −10.823 | 2.556 | 1.00 | 38.23 | O |
| ATOM | 1188 | N | ASN | A | 384 | −4.985 | −12.678 | 1.589 | 1.00 | 39.73 | N |
| ATOM | 1189 | CA | ASN | A | 384 | −5.795 | −13.130 | 2.693 | 1.00 | 41.42 | C |
| ATOM | 1190 | CB | ASN | A | 384 | −5.288 | −14.487 | 3.249 | 1.00 | 42.15 | C |
| ATOM | 1191 | CG | ASN | A | 384 | −3.910 | −14.450 | 3.912 | 1.00 | 44.28 | C |
| ATOM | 1192 | OD1 | ASN | A | 384 | −3.297 | −13.383 | 4.028 | 1.00 | 46.24 | O |
| ATOM | 1193 | ND2 | ASN | A | 384 | −3.433 | −15.600 | 4.352 | 1.00 | 46.73 | N |
| ATOM | 1194 | C | ASN | A | 384 | −5.957 | −12.056 | 3.749 | 1.00 | 42.06 | C |
| ATOM | 1195 | O | ASN | A | 384 | −5.200 | −12.047 | 4.711 | 1.00 | 41.86 | O |
| ATOM | 1196 | N | GLY | A | 385 | −6.928 | −11.110 | 3.615 | 1.00 | 42.64 | N |
| ATOM | 1197 | CA | GLY | A | 385 | −7.159 | −10.135 | 4.727 | 1.00 | 43.54 | C |
| ATOM | 1198 | C | GLY | A | 385 | −6.633 | −8.689 | 4.564 | 1.00 | 44.27 | C |
| ATOM | 1199 | O | GLY | A | 385 | −7.405 | −7.756 | 4.300 | 1.00 | 45.13 | O |
| ATOM | 1200 | N | GLN | A | 386 | −5.297 | −8.545 | 4.729 | 1.00 | 44.29 | N |
| ATOM | 1201 | CA | GLN | A | 386 | −4.546 | −7.277 | 4.619 | 1.00 | 43.80 | C |
| ATOM | 1202 | CB | GLN | A | 386 | −3.273 | −7.285 | 5.471 | 1.00 | 43.74 | C |
| ATOM | 1203 | CG | GLN | A | 386 | −3.411 | −7.694 | 6.930 | 1.00 | 42.38 | C |
| ATOM | 1204 | CD | GLN | A | 386 | −2.199 | −8.464 | 7.392 | 1.00 | 42.58 | C |
| ATOM | 1205 | OE1 | GLN | A | 386 | −2.130 | −8.891 | 8.540 | 1.00 | 41.05 | O |
| ATOM | 1206 | NE2 | GLN | A | 386 | −1.228 | −8.642 | 6.487 | 1.00 | 41.81 | N |
| ATOM | 1207 | C | GLN | A | 386 | −4.141 | −7.036 | 3.175 | 1.00 | 43.65 | C |
| ATOM | 1208 | O | GLN | A | 386 | −3.973 | −8.024 | 2.448 | 1.00 | 43.98 | O |
| ATOM | 1209 | N | PRO | A | 387 | −3.978 | −5.777 | 2.719 | 1.00 | 43.38 | N |
| ATOM | 1210 | CA | PRO | A | 387 | −3.609 | −5.485 | 1.331 | 1.00 | 42.95 | C |
| ATOM | 1211 | CB | PRO | A | 387 | −4.066 | −4.038 | 1.120 | 1.00 | 43.17 | C |
| ATOM | 1212 | CG | PRO | A | 387 | −4.795 | −3.631 | 2.358 | 1.00 | 43.69 | C |
| ATOM | 1213 | CD | PRO | A | 387 | −4.320 | −4.555 | 3.441 | 1.00 | 43.33 | C |
| ATOM | 1214 | C | PRO | A | 387 | −2.093 | −5.652 | 1.078 | 1.00 | 42.61 | C |
| ATOM | 1215 | O | PRO | A | 387 | −1.341 | −5.870 | 2.019 | 1.00 | 43.00 | O |
| ATOM | 1216 | N | GLU | A | 388 | −1.665 | −5.544 | −0.215 | 1.00 | 42.48 | N |
| ATOM | 1217 | CA | GLU | A | 388 | −0.266 | −5.717 | −0.624 | 1.00 | 41.65 | C |
| ATOM | 1218 | CB | GLU | A | 388 | −0.166 | −6.693 | −1.829 | 1.00 | 41.32 | C |
| ATOM | 1219 | CG | GLU | A | 388 | −0.610 | −8.130 | −1.524 | 1.00 | 40.38 | C |
| ATOM | 1220 | CD | GLU | A | 388 | 0.530 | −9.091 | −1.161 | 1.00 | 41.26 | C |
| ATOM | 1221 | OE1 | GLU | A | 388 | 1.638 | −8.949 | −1.721 | 1.00 | 43.10 | O |
| ATOM | 1222 | OE2 | GLU | A | 388 | 0.298 | −9.988 | −0.327 | 1.00 | 38.41 | O |
| ATOM | 1223 | C | GLU | A | 388 | 0.453 | −4.378 | −0.944 | 1.00 | 41.69 | C |
| ATOM | 1224 | O | GLU | A | 388 | −0.055 | −3.520 | −1.667 | 1.00 | 41.31 | O |
| ATOM | 1225 | N | ASN | A | 389 | 1.649 | −4.231 | −0.381 | 1.00 | 41.27 | N |
| ATOM | 1226 | CA | ASN | A | 389 | 2.541 | −3.088 | −0.641 | 1.00 | 41.05 | C |
| ATOM | 1227 | CB | ASN | A | 389 | 3.751 | −3.241 | 0.303 | 1.00 | 42.00 | C |
| ATOM | 1228 | CG | ASN | A | 389 | 4.855 | −2.215 | 0.061 | 1.00 | 43.43 | C |
| ATOM | 1229 | OD1 | ASN | A | 389 | 5.002 | −1.638 | −1.036 | 1.00 | 43.90 | O |
| ATOM | 1230 | ND2 | ASN | A | 389 | 5.664 | −2.002 | 1.097 | 1.00 | 45.20 | N |
| ATOM | 1231 | C | ASN | A | 389 | 3.011 | −2.916 | −2.107 | 1.00 | 40.16 | C |
| ATOM | 1232 | O | ASN | A | 389 | 2.637 | −1.950 | −2.832 | 1.00 | 40.62 | O |
| ATOM | 1233 | N | ASN | A | 390 | 3.827 | −3.872 | −2.539 | 1.00 | 38.17 | N |
| ATOM | 1234 | CA | ASN | A | 390 | 4.561 | −3.774 | −3.781 | 1.00 | 35.96 | C |
| ATOM | 1235 | CB | ASN | A | 390 | 5.690 | −4.778 | −3.715 | 1.00 | 35.46 | C |
| ATOM | 1236 | CG | ASN | A | 390 | 6.850 | −4.380 | −4.540 | 1.00 | 35.15 | C |
| ATOM | 1237 | OD1 | ASN | A | 390 | 7.920 | −4.959 | −4.411 | 1.00 | 37.32 | O |
| ATOM | 1238 | ND2 | ASN | A | 390 | 6.672 | −3.383 | −5.392 | 1.00 | 32.94 | N |
| ATOM | 1239 | C | ASN | A | 390 | 3.753 | −3.983 | −5.074 | 1.00 | 35.04 | C |
| ATOM | 1240 | O | ASN | A | 390 | 3.973 | −4.958 | −5.801 | 1.00 | 34.33 | O |
| ATOM | 1241 | N | TYR | A | 391 | 2.830 | −3.067 | −5.371 | 1.00 | 34.28 | N |
| ATOM | 1242 | CA | TYR | A | 391 | 2.141 | −3.076 | −6.685 | 1.00 | 33.08 | C |
| ATOM | 1243 | CB | TYR | A | 391 | 0.733 | −3.739 | −6.612 | 1.00 | 33.72 | C |
| ATOM | 1244 | CG | TYR | A | 391 | −0.335 | −2.842 | −6.018 | 1.00 | 34.76 | C |
| ATOM | 1245 | CD1 | TYR | A | 391 | −0.695 | −2.943 | −4.667 | 1.00 | 36.57 | C |
| ATOM | 1246 | CE1 | TYR | A | 391 | −1.653 | −2.069 | −4.090 | 1.00 | 36.53 | C |
| ATOM | 1247 | CZ | TYR | A | 391 | −2.236 | −1.102 | −4.881 | 1.00 | 35.62 | C |
| ATOM | 1248 | OH | TYR | A | 391 | −3.168 | −0.254 | −4.348 | 1.00 | 36.39 | O |
| ATOM | 1249 | CE2 | TYR | A | 391 | −1.895 | −0.985 | −6.230 | 1.00 | 36.43 | C |
| ATOM | 1250 | CD2 | TYR | A | 391 | −0.951 | −1.851 | −6.791 | 1.00 | 35.25 | C |
| ATOM | 1251 | C | TYR | A | 391 | 2.104 | −1.682 | −7.356 | 1.00 | 32.19 | C |
| ATOM | 1252 | O | TYR | A | 391 | 2.174 | −0.643 | −6.698 | 1.00 | 30.85 | O |
| ATOM | 1253 | N | LYS | A | 392 | 2.033 | −1.665 | −8.685 | 1.00 | 31.92 | N |
| ATOM | 1254 | CA | LYS | A | 392 | 1.819 | −0.421 | −9.417 | 1.00 | 31.14 | C |
| ATOM | 1255 | CB | LYS | A | 392 | 3.095 | 0.094 | −10.054 | 1.00 | 29.85 | C |
| ATOM | 1256 | CG | LYS | A | 392 | 4.115 | 0.646 | −9.090 | 1.00 | 30.89 | C |
| ATOM | 1257 | CD | LYS | A | 392 | 3.620 | 1.824 | −8.226 | 1.00 | 30.42 | C |
| ATOM | 1258 | CE | LYS | A | 392 | 4.799 | 2.453 | −7.518 | 1.00 | 31.40 | C |
| ATOM | 1259 | NZ | LYS | A | 392 | 4.374 | 3.538 | −6.599 | 1.00 | 36.61 | N |
| ATOM | 1260 | C | LYS | A | 392 | 0.817 | −0.699 | −10.495 | 1.00 | 31.66 | C |
| ATOM | 1261 | O | LYS | A | 392 | 0.723 | −1.839 | −11.007 | 1.00 | 32.31 | O |
| ATOM | 1262 | N | THR | A | 393 | 0.093 | 0.331 | −10.843 | 1.00 | 30.87 | N |
| ATOM | 1263 | CA | THR | A | 393 | −0.904 | 0.221 | −11.880 | 1.00 | 30.43 | C |
| ATOM | 1264 | CB | THR | A | 393 | −2.302 | 0.322 | −11.302 | 1.00 | 30.19 | C |
| ATOM | 1265 | OG1 | THR | A | 393 | −2.443 | −0.615 | −10.242 | 1.00 | 31.29 | O |
| ATOM | 1266 | CG2 | THR | A | 393 | −3.347 | 0.039 | −12.382 | 1.00 | 29.98 | C |
| ATOM | 1267 | C | THR | A | 393 | −0.717 | 1.275 | −12.943 | 1.00 | 30.84 | C |
| ATOM | 1268 | O | THR | A | 393 | −0.443 | 2.433 | −12.634 | 1.00 | 30.38 | O |
| ATOM | 1269 | N | THR | A | 394 | −0.859 | 0.887 | −14.221 | 1.00 | 29.94 | N |
| ATOM | 1270 | CA | THR | A | 394 | −0.675 | 1.862 | −15.324 | 1.00 | 30.35 | C |
| ATOM | 1271 | CB | THR | A | 394 | −0.608 | 1.175 | −16.702 | 1.00 | 30.14 | C |
| ATOM | 1272 | OG1 | THR | A | 394 | −1.948 | 0.848 | −17.110 | 1.00 | 29.89 | O |
| ATOM | 1273 | CG2 | THR | A | 394 | 0.240 | −0.092 | −16.624 | 1.00 | 29.34 | C |
| ATOM | 1274 | C | THR | A | 394 | −1.831 | 2.838 | −15.382 | 1.00 | 29.98 | C |
| ATOM | 1275 | O | THR | A | 394 | −2.911 | 2.542 | −14.859 | 1.00 | 30.17 | O |
| ATOM | 1276 | N | PRO | A | 395 | −1.674 | 4.003 | −15.967 | 1.00 | 30.09 | N |
| ATOM | 1277 | CA | PRO | A | 395 | −2.852 | 4.801 | −16.124 | 1.00 | 29.83 | C |
| ATOM | 1278 | CB | PRO | A | 395 | −2.342 | 6.133 | −16.703 | 1.00 | 29.93 | C |
| ATOM | 1279 | CG | PRO | A | 395 | −0.968 | 5.824 | −17.214 | 1.00 | 30.75 | C |
| ATOM | 1280 | CD | PRO | A | 395 | −0.456 | 4.633 | −16.447 | 1.00 | 30.22 | C |
| ATOM | 1281 | C | PRO | A | 395 | −3.763 | 4.066 | −17.116 | 1.00 | 29.81 | C |
| ATOM | 1282 | O | PRO | A | 395 | −3.300 | 3.161 | −17.820 | 1.00 | 29.26 | O |
| ATOM | 1283 | N | PRO | A | 396 | −5.059 | 4.387 | −17.185 | 1.00 | 29.84 | N |
| ATOM | 1284 | CA | PRO | A | 396 | −5.933 | 3.746 | −18.166 | 1.00 | 29.15 | C |
| ATOM | 1285 | CB | PRO | A | 396 | −7.317 | 4.298 | −17.855 | 1.00 | 29.11 | C |
| ATOM | 1286 | CG | PRO | A | 396 | −7.091 | 5.513 | −17.016 | 1.00 | 30.04 | C |
| ATOM | 1287 | CD | PRO | A | 396 | −5.780 | 5.326 | −16.300 | 1.00 | 28.20 | C |
| ATOM | 1288 | C | PRO | A | 396 | −5.455 | 4.150 | −19.581 | 1.00 | 29.04 | C |
| ATOM | 1289 | O | PRO | A | 396 | −5.005 | 5.289 | −19.762 | 1.00 | 29.06 | O |
| ATOM | 1290 | N | VAL | A | 397 | −5.552 | 3.277 | −20.576 | 1.00 | 29.66 | N |
| ATOM | 1291 | CA | VAL | A | 397 | −5.193 | 3.659 | −21.933 | 1.00 | 28.16 | C |
| ATOM | 1292 | CB | VAL | A | 397 | −4.036 | 2.778 | −22.481 | 1.00 | 28.09 | C |
| ATOM | 1293 | CG1 | VAL | A | 397 | −3.647 | 3.186 | −23.886 | 1.00 | 26.19 | C |
| ATOM | 1294 | CG2 | VAL | A | 397 | −2.869 | 2.841 | −21.577 | 1.00 | 26.70 | C |
| ATOM | 1295 | C | VAL | A | 397 | −6.411 | 3.541 | −22.856 | 1.00 | 28.66 | C |
| ATOM | 1296 | O | VAL | A | 397 | −7.264 | 2.648 | −22.717 | 1.00 | 28.59 | O |
| ATOM | 1297 | N | LEU | A | 398 | −6.486 | 4.451 | −23.819 | 1.00 | 28.24 | N |
| ATOM | 1298 | CA | LEU | A | 398 | −7.608 | 4.438 | −24.678 | 1.00 | 27.26 | C |
| ATOM | 1299 | CB | LEU | A | 398 | −7.774 | 5.826 | −25.263 | 1.00 | 27.40 | C |
| ATOM | 1300 | CG | LEU | A | 398 | −8.729 | 6.010 | −26.432 | 1.00 | 26.02 | C |
| ATOM | 1301 | CD1 | LEU | A | 398 | −10.121 | 5.961 | −25.888 | 1.00 | 23.75 | C |
| ATOM | 1302 | CD2 | LEU | A | 398 | −8.439 | 7.345 | −27.001 | 1.00 | 24.26 | C |
| ATOM | 1303 | C | LEU | A | 398 | −7.343 | 3.363 | −25.727 | 1.00 | 27.47 | C |
| ATOM | 1304 | O | LEU | A | 398 | −6.242 | 3.234 | −26.307 | 1.00 | 26.87 | O |
| ATOM | 1305 | N | ASP | A | 399 | −8.361 | 2.552 | −25.939 | 1.00 | 27.53 | N |
| ATOM | 1306 | CA | ASP | A | 399 | −8.247 | 1.417 | −26.844 | 1.00 | 26.95 | C |
| ATOM | 1307 | CB | ASP | A | 399 | −8.835 | 0.172 | −26.173 | 1.00 | 26.58 | C |
| ATOM | 1308 | CG | ASP | A | 399 | −8.068 | −1.095 | −26.528 | 1.00 | 28.20 | C |
| ATOM | 1309 | OD1 | ASP | A | 399 | −7.302 | −1.078 | −27.527 | 1.00 | 31.20 | O |
| ATOM | 1310 | OD2 | ASP | A | 399 | −8.222 | −2.117 | −25.818 | 1.00 | 26.20 | O |
| ATOM | 1311 | C | ASP | A | 399 | −8.832 | 1.676 | −28.264 | 1.00 | 26.30 | C |
| ATOM | 1312 | O | ASP | A | 399 | −9.517 | 2.683 | −28.543 | 1.00 | 25.93 | O |
| ATOM | 1313 | N | SER | A | 400 | −8.560 | 0.763 | −29.170 | 1.00 | 25.83 | N |
| ATOM | 1314 | CA | SER | A | 400 | −9.010 | 0.978 | −30.516 | 1.00 | 26.62 | C |
| ATOM | 1315 | CB | SER | A | 400 | −8.319 | −0.002 | −31.469 | 1.00 | 26.74 | C |
| ATOM | 1316 | OG | SER | A | 400 | −8.961 | −1.269 | −31.454 | 1.00 | 27.74 | O |
| ATOM | 1317 | C | SER | A | 400 | −10.535 | 0.905 | −30.641 | 1.00 | 26.78 | C |
| ATOM | 1318 | O | SER | A | 400 | −11.074 | 1.150 | −31.707 | 1.00 | 27.80 | O |
| ATOM | 1319 | N | ASP | A | 401 | −11.251 | 0.561 | −29.582 | 1.00 | 27.05 | N |
| ATOM | 1320 | CA | ASP | A | 401 | −12.707 | 0.612 | −29.685 | 1.00 | 27.86 | C |
| ATOM | 1321 | CB | ASP | A | 401 | −13.307 | −0.765 | −29.414 | 1.00 | 28.20 | C |
| ATOM | 1322 | CG | ASP | A | 401 | −13.198 | −1.193 | −27.935 | 1.00 | 32.06 | C |
| ATOM | 1323 | OD1 | ASP | A | 401 | −12.579 | −0.518 | −27.036 | 1.00 | 25.84 | O |
| ATOM | 1324 | OD2 | ASP | A | 401 | −13.809 | −2.252 | −27.691 | 1.00 | 36.63 | O |
| ATOM | 1325 | C | ASP | A | 401 | −13.394 | 1.695 | −28.828 | 1.00 | 27.42 | C |
| ATOM | 1326 | O | ASP | A | 401 | −14.570 | 1.572 | −28.523 | 1.00 | 27.50 | O |
| ATOM | 1327 | N | GLY | A | 402 | −12.663 | 2.741 | −28.438 | 1.00 | 26.92 | N |
| ATOM | 1328 | CA | GLY | A | 402 | −13.232 | 3.813 | −27.659 | 1.00 | 25.67 | C |
| ATOM | 1329 | C | GLY | A | 402 | −13.336 | 3.382 | −26.225 | 1.00 | 26.80 | C |
| ATOM | 1330 | O | GLY | A | 402 | −13.847 | 4.150 | −25.376 | 1.00 | 28.30 | O |
| ATOM | 1331 | N | SER | A | 403 | −12.853 | 2.183 | −25.901 | 1.00 | 26.26 | N |
| ATOM | 1332 | CA | SER | A | 403 | −12.871 | 1.805 | −24.506 | 1.00 | 26.96 | C |
| ATOM | 1333 | CB | SER | A | 403 | −13.453 | 0.417 | −24.286 | 1.00 | 26.94 | C |
| ATOM | 1334 | OG | SER | A | 403 | −12.468 | −0.602 | −24.319 | 1.00 | 28.84 | O |
| ATOM | 1335 | C | SER | A | 403 | −11.536 | 1.942 | −23.806 | 1.00 | 27.53 | C |
| ATOM | 1336 | O | SER | A | 403 | −10.553 | 2.340 | −24.406 | 1.00 | 29.07 | O |
| ATOM | 1337 | N | PHE | A | 404 | −11.492 | 1.618 | −22.523 | 1.00 | 27.37 | N |
| ATOM | 1338 | CA | PHE | A | 404 | −10.240 | 1.746 | −21.796 | 1.00 | 28.04 | C |
| ATOM | 1339 | CB | PHE | A | 404 | −10.393 | 2.746 | −20.612 | 1.00 | 26.97 | C |
| ATOM | 1340 | CG | PHE | A | 404 | −10.367 | 4.192 | −21.038 | 1.00 | 27.58 | C |
| ATOM | 1341 | CD1 | PHE | A | 404 | −9.144 | 4.842 | −21.316 | 1.00 | 25.97 | C |
| ATOM | 1342 | CE1 | PHE | A | 404 | −9.118 | 6.184 | −21.705 | 1.00 | 25.27 | C |
| ATOM | 1343 | CZ | PHE | A | 404 | −10.337 | 6.905 | −21.833 | 1.00 | 26.26 | C |
| ATOM | 1344 | CE2 | PHE | A | 404 | −11.556 | 6.267 | −21.576 | 1.00 | 25.29 | C |
| ATOM | 1345 | CD2 | PHE | A | 404 | −11.561 | 4.918 | −21.177 | 1.00 | 27.73 | C |
| ATOM | 1346 | C | PHE | A | 404 | −9.647 | 0.401 | −21.331 | 1.00 | 28.20 | C |
| ATOM | 1347 | O | PHE | A | 404 | −10.381 | −0.539 | −20.903 | 1.00 | 27.85 | O |
| ATOM | 1348 | N | PHE | A | 405 | −8.329 | 0.286 | −21.424 | 1.00 | 28.47 | N |
| ATOM | 1349 | CA | PHE | A | 405 | −7.703 | −0.813 | −20.716 | 1.00 | 29.44 | C |
| ATOM | 1350 | CB | PHE | A | 405 | −7.135 | −1.868 | −21.662 | 1.00 | 28.58 | C |
| ATOM | 1351 | CG | PHE | A | 405 | −5.900 | −1.458 | −22.340 | 1.00 | 26.52 | C |
| ATOM | 1352 | CD1 | PHE | A | 405 | −4.682 | −1.871 | −21.866 | 1.00 | 25.27 | C |
| ATOM | 1353 | CE1 | PHE | A | 405 | −3.515 | −1.501 | −22.506 | 1.00 | 24.98 | C |
| ATOM | 1354 | CZ | PHE | A | 405 | −3.587 | −0.743 | −23.657 | 1.00 | 27.74 | C |
| ATOM | 1355 | CE2 | PHE | A | 405 | −4.802 | −0.339 | −24.156 | 1.00 | 25.45 | C |
| ATOM | 1356 | CD2 | PHE | A | 405 | −5.955 | −0.690 | −23.492 | 1.00 | 27.21 | C |
| ATOM | 1357 | C | PHE | A | 405 | −6.658 | −0.312 | −19.737 | 1.00 | 30.60 | C |
| ATOM | 1358 | O | PHE | A | 405 | −6.362 | 0.898 | −19.709 | 1.00 | 31.37 | O |
| ATOM | 1359 | N | LEU | A | 406 | −6.110 | −1.255 | −18.969 | 1.00 | 31.11 | N |
| ATOM | 1360 | CA | LEU | A | 406 | −5.258 | −0.967 | −17.836 | 1.00 | 32.10 | C |
| ATOM | 1361 | CB | LEU | A | 406 | −6.198 | −0.538 | −16.715 | 1.00 | 33.51 | C |
| ATOM | 1362 | CG | LEU | A | 406 | −5.788 | 0.046 | −15.394 | 1.00 | 34.58 | C |
| ATOM | 1363 | CD1 | LEU | A | 406 | −4.594 | 0.804 | −15.824 | 1.00 | 39.84 | C |
| ATOM | 1364 | CD2 | LEU | A | 406 | −6.834 | 1.057 | −14.923 | 1.00 | 30.75 | C |
| ATOM | 1365 | C | LEU | A | 406 | −4.619 | −2.294 | −17.434 | 1.00 | 31.92 | C |
| ATOM | 1366 | O | LEU | A | 406 | −5.223 | −3.351 | −17.665 | 1.00 | 30.56 | O |
| ATOM | 1367 | N | TYR | A | 407 | −3.413 | −2.244 | −16.855 | 1.00 | 31.27 | N |
| ATOM | 1368 | CA | TYR | A | 407 | −2.788 | −3.432 | −16.239 | 1.00 | 31.05 | C |
| ATOM | 1369 | CB | TYR | A | 407 | −1.584 | −3.965 | −17.008 | 1.00 | 30.08 | C |
| ATOM | 1370 | CG | TYR | A | 407 | −1.795 | −4.714 | −18.314 | 1.00 | 30.34 | C |
| ATOM | 1371 | CD1 | TYR | A | 407 | −1.843 | −4.035 | −19.544 | 1.00 | 30.25 | C |
| ATOM | 1372 | CE1 | TYR | A | 407 | −2.000 | −4.731 | −20.763 | 1.00 | 28.04 | C |
| ATOM | 1373 | CZ | TYR | A | 407 | −2.068 | −6.093 | −20.753 | 1.00 | 28.49 | C |
| ATOM | 1374 | OH | TYR | A | 407 | −2.202 | −6.749 | −21.947 | 1.00 | 31.54 | O |
| ATOM | 1375 | CE2 | TYR | A | 407 | −2.011 | −6.796 | −19.571 | 1.00 | 29.80 | C |
| ATOM | 1376 | CD2 | TYR | A | 407 | −1.860 | −6.107 | −18.350 | 1.00 | 31.30 | C |
| ATOM | 1377 | C | TYR | A | 407 | −2.279 | −2.991 | −14.865 | 1.00 | 31.65 | C |
| ATOM | 1378 | O | TYR | A | 407 | −1.815 | −1.864 | −14.701 | 1.00 | 32.04 | O |
| ATOM | 1379 | N | SER | A | 408 | −2.396 | −3.862 | −13.875 | 1.00 | 32.02 | N |
| ATOM | 1380 | CA | SER | A | 408 | −1.797 | −3.610 | −12.595 | 1.00 | 32.52 | C |
| ATOM | 1381 | CB | SER | A | 408 | −2.872 | −3.521 | −11.500 | 1.00 | 32.86 | C |
| ATOM | 1382 | OG | SER | A | 408 | −2.352 | −3.498 | −10.173 | 1.00 | 31.31 | O |
| ATOM | 1383 | C | SER | A | 408 | −0.844 | −4.762 | −12.380 | 1.00 | 33.16 | C |
| ATOM | 1384 | O | SER | A | 408 | −1.194 | −5.924 | −12.606 | 1.00 | 34.35 | O |
| ATOM | 1385 | N | LYS | A | 409 | 0.380 | −4.441 | −11.976 | 1.00 | 33.65 | N |
| ATOM | 1386 | CA | LYS | A | 409 | 1.397 | −5.458 | −11.703 | 1.00 | 33.11 | C |
| ATOM | 1387 | CB | LYS | A | 409 | 2.676 | −5.115 | −12.468 | 1.00 | 33.56 | C |
| ATOM | 1388 | CG | LYS | A | 409 | 3.924 | −5.986 | −12.150 | 1.00 | 33.14 | C |
| ATOM | 1389 | CD | LYS | A | 409 | 4.920 | −5.922 | −13.310 | 1.00 | 32.51 | C |
| ATOM | 1390 | CE | LYS | A | 409 | 5.732 | −4.617 | −13.300 | 1.00 | 33.47 | C |
| ATOM | 1391 | NZ | LYS | A | 409 | 6.385 | −4.251 | −11.971 | 1.00 | 30.77 | N |
| ATOM | 1392 | C | LYS | A | 409 | 1.719 | −5.548 | −10.226 | 1.00 | 32.70 | C |
| ATOM | 1393 | O | LYS | A | 409 | 2.233 | −4.575 | −9.659 | 1.00 | 33.39 | O |
| ATOM | 1394 | N | LEU | A | 410 | 1.429 | −6.717 | −9.643 | 1.00 | 31.85 | N |
| ATOM | 1395 | CA | LEU | A | 410 | 1.899 | −7.168 | −8.298 | 1.00 | 31.18 | C |
| ATOM | 1396 | CB | LEU | A | 410 | 0.835 | −8.033 | −7.584 | 1.00 | 30.89 | C |
| ATOM | 1397 | CG | LEU | A | 410 | 1.086 | −8.686 | −6.191 | 1.00 | 30.84 | C |
| ATOM | 1398 | CD1 | LEU | A | 410 | 1.299 | −7.689 | −5.058 | 1.00 | 26.46 | C |
| ATOM | 1399 | CD2 | LEU | A | 410 | −0.024 | −9.697 | −5.799 | 1.00 | 30.05 | C |
| ATOM | 1400 | C | LEU | A | 410 | 3.218 | −7.950 | −8.355 | 1.00 | 31.22 | C |
| ATOM | 1401 | O | LEU | A | 410 | 3.354 | −8.923 | −9.097 | 1.00 | 31.27 | O |
| ATOM | 1402 | N | THR | A | 411 | 4.185 | −7.507 | −7.569 | 1.00 | 30.82 | N |
| ATOM | 1403 | CA | THR | A | 411 | 5.433 | −8.204 | −7.458 | 1.00 | 31.11 | C |
| ATOM | 1404 | CB | THR | A | 411 | 6.583 | −7.217 | −7.556 | 1.00 | 31.72 | C |
| ATOM | 1405 | OG1 | THR | A | 411 | 6.322 | −6.309 | −8.639 | 1.00 | 32.52 | O |
| ATOM | 1406 | CG2 | THR | A | 411 | 7.946 | −7.952 | −7.714 | 1.00 | 30.41 | C |
| ATOM | 1407 | C | THR | A | 411 | 5.514 | −8.893 | −6.107 | 1.00 | 31.01 | C |
| ATOM | 1408 | O | THR | A | 411 | 5.375 | −8.250 | −5.063 | 1.00 | 30.29 | O |
| ATOM | 1409 | N | VAL | A | 412 | 5.741 | −10.195 | −6.124 | 1.00 | 30.99 | N |
| ATOM | 1410 | CA | VAL | A | 412 | 5.828 | −10.917 | −4.848 | 1.00 | 32.28 | C |
| ATOM | 1411 | CB | VAL | A | 412 | 4.548 | −11.769 | −4.512 | 1.00 | 31.57 | C |
| ATOM | 1412 | CG1 | VAL | A | 412 | 3.362 | −10.865 | −4.226 | 1.00 | 31.71 | C |
| ATOM | 1413 | CG2 | VAL | A | 412 | 4.221 | −12.771 | −5.608 | 1.00 | 30.61 | C |
| ATOM | 1414 | C | VAL | A | 412 | 7.119 | −11.747 | −4.782 | 1.00 | 33.11 | C |
| ATOM | 1415 | O | VAL | A | 412 | 7.696 | −12.048 | −5.838 | 1.00 | 33.84 | O |
| ATOM | 1416 | N | ASP | A | 413 | 7.591 | −12.082 | −3.573 | 1.00 | 33.51 | N |
| ATOM | 1417 | CA | ASP | A | 413 | 8.767 | −12.966 | −3.421 | 1.00 | 34.20 | C |
| ATOM | 1418 | CB | ASP | A | 413 | 9.126 | −13.126 | −1.916 | 1.00 | 34.14 | C |
| ATOM | 1419 | CG | ASP | A | 413 | 10.204 | −12.104 | −1.408 | 1.00 | 35.07 | C |
| ATOM | 1420 | OD1 | ASP | A | 413 | 10.800 | −12.350 | −0.334 | 1.00 | 36.24 | O |
| ATOM | 1421 | OD2 | ASP | A | 413 | 10.486 | −11.070 | −2.052 | 1.00 | 36.11 | O |
| ATOM | 1422 | C | ASP | A | 413 | 8.408 | −14.322 | −4.104 | 1.00 | 34.27 | C |
| ATOM | 1423 | O | ASP | A | 413 | 7.306 | −14.808 | −3.858 | 1.00 | 34.62 | O |
| ATOM | 1424 | N | LYS | A | 414 | 9.269 | −14.899 | −4.975 | 1.00 | 34.30 | N |
| ATOM | 1425 | CA | LYS | A | 414 | 8.968 | −16.205 | −5.665 | 1.00 | 34.24 | C |
| ATOM | 1426 | CB | LYS | A | 414 | 10.107 | −16.653 | −6.590 | 1.00 | 34.31 | C |
| ATOM | 1427 | CG | LYS | A | 414 | 9.959 | −18.102 | −7.119 | 1.00 | 33.96 | C |
| ATOM | 1428 | CD | LYS | A | 414 | 11.059 | −18.519 | −8.092 | 1.00 | 33.99 | C |
| ATOM | 1429 | CE | LYS | A | 414 | 12.407 | −18.775 | −7.406 | 1.00 | 35.51 | C |
| ATOM | 1430 | NZ | LYS | A | 414 | 13.578 | −18.600 | −8.349 | 1.00 | 35.21 | N |
| ATOM | 1431 | C | LYS | A | 414 | 8.570 | −17.383 | −4.735 | 1.00 | 34.84 | C |
| ATOM | 1432 | O | LYS | A | 414 | 7.884 | −18.323 | −5.167 | 1.00 | 35.02 | O |
| ATOM | 1433 | N | SER | A | 415 | 9.005 | −17.324 | −3.472 | 1.00 | 35.03 | N |
| ATOM | 1434 | CA | SER | A | 415 | 8.609 | −18.288 | −2.430 | 1.00 | 35.24 | C |
| ATOM | 1435 | CB | SER | A | 415 | 9.547 | −18.174 | −1.208 | 1.00 | 35.14 | C |
| ATOM | 1436 | OG | SER | A | 415 | 9.656 | −16.846 | −0.733 | 1.00 | 34.55 | O |
| ATOM | 1437 | C | SER | A | 415 | 7.120 | −18.209 | −1.997 | 1.00 | 35.37 | C |
| ATOM | 1438 | O | SER | A | 415 | 6.496 | −19.223 | −1.654 | 1.00 | 35.14 | O |
| ATOM | 1439 | N | ARG | A | 416 | 6.564 | −17.003 | −2.012 | 1.00 | 36.07 | N |
| ATOM | 1440 | CA | ARG | A | 416 | 5.138 | −16.782 | −1.704 | 1.00 | 36.79 | C |
| ATOM | 1441 | CB | ARG | A | 416 | 4.844 | −15.282 | −1.467 | 1.00 | 36.73 | C |
| ATOM | 1442 | CG | ARG | A | 416 | 5.605 | −14.699 | −0.249 | 1.00 | 36.86 | C |
| ATOM | 1443 | CD | ARG | A | 416 | 4.993 | −13.427 | 0.306 | 1.00 | 37.28 | C |
| ATOM | 1444 | NE | ARG | A | 416 | 3.722 | −13.696 | 0.984 | 1.00 | 40.03 | N |
| ATOM | 1445 | CZ | ARG | A | 416 | 2.539 | −13.163 | 0.657 | 1.00 | 39.07 | C |
| ATOM | 1446 | NH1 | ARG | A | 416 | 2.448 | −12.297 | −0.348 | 1.00 | 38.47 | N |
| ATOM | 1447 | NH2 | ARG | A | 416 | 1.446 | −13.499 | 1.353 | 1.00 | 37.54 | N |
| ATOM | 1448 | C | ARG | A | 416 | 4.212 | −17.418 | −2.769 | 1.00 | 36.93 | C |
| ATOM | 1449 | O | ARG | A | 416 | 3.259 | −18.148 | −2.420 | 1.00 | 36.76 | O |
| ATOM | 1450 | N | TRP | A | 417 | 4.546 | −17.177 | −4.046 | 1.00 | 37.07 | N |
| ATOM | 1451 | CA | TRP | A | 417 | 3.854 | −17.739 | −5.224 | 1.00 | 37.35 | C |
| ATOM | 1452 | CB | TRP | A | 417 | 4.500 | −17.209 | −6.504 | 1.00 | 36.93 | C |
| ATOM | 1453 | CG | TRP | A | 417 | 3.942 | −17.805 | −7.764 | 1.00 | 36.44 | C |
| ATOM | 1454 | CD1 | TRP | A | 417 | 4.588 | −18.635 | −8.641 | 1.00 | 36.51 | C |
| ATOM | 1455 | NE1 | TRP | A | 417 | 3.756 | −18.972 | −9.686 | 1.00 | 35.90 | N |
| ATOM | 1456 | CE2 | TRP | A | 417 | 2.545 | −18.364 | −9.489 | 1.00 | 36.14 | C |
| ATOM | 1457 | CD2 | TRP | A | 417 | 2.628 | −17.622 | −8.285 | 1.00 | 35.20 | C |
| ATOM | 1458 | CE3 | TRP | A | 417 | 1.513 | −16.905 | −7.856 | 1.00 | 33.79 | C |
| ATOM | 1459 | CZ3 | TRP | A | 417 | 0.378 | −16.928 | −8.638 | 1.00 | 35.44 | C |
| ATOM | 1460 | CH2 | TRP | A | 417 | 0.319 | −17.678 | −9.827 | 1.00 | 35.53 | C |
| ATOM | 1461 | CZ2 | TRP | A | 417 | 1.387 | −18.400 | −10.268 | 1.00 | 36.28 | C |
| ATOM | 1462 | C | TRP | A | 417 | 3.836 | −19.263 | −5.295 | 1.00 | 37.84 | C |
| ATOM | 1463 | O | TRP | A | 417 | 2.817 | −19.863 | −5.643 | 1.00 | 38.38 | O |
| ATOM | 1464 | N | GLN | A | 418 | 4.964 | −19.888 | −4.969 | 1.00 | 38.48 | N |
| ATOM | 1465 | CA | GLN | A | 418 | 5.077 | −21.351 | −5.044 | 1.00 | 38.55 | C |
| ATOM | 1466 | CB | GLN | A | 418 | 6.523 | −21.760 | −5.230 | 1.00 | 38.72 | C |
| ATOM | 1467 | CG | GLN | A | 418 | 7.256 | −20.902 | −6.220 | 1.00 | 38.90 | C |
| ATOM | 1468 | CD | GLN | A | 418 | 8.359 | −21.656 | −6.865 | 1.00 | 39.60 | C |
| ATOM | 1469 | OE1 | GLN | A | 418 | 9.518 | −21.554 | −6.464 | 1.00 | 40.18 | O |
| ATOM | 1470 | NE2 | GLN | A | 418 | 8.009 | −22.463 | −7.855 | 1.00 | 40.23 | N |
| ATOM | 1471 | C | GLN | A | 418 | 4.472 | −22.085 | −3.858 | 1.00 | 38.54 | C |
| ATOM | 1472 | O | GLN | A | 418 | 4.022 | −23.228 | −3.994 | 1.00 | 38.21 | O |
| ATOM | 1473 | N | GLN | A | 419 | 4.478 | −21.428 | −2.700 | 1.00 | 38.79 | N |
| ATOM | 1474 | CA | GLN | A | 419 | 3.745 | −21.906 | −1.527 | 1.00 | 39.56 | C |
| ATOM | 1475 | CB | GLN | A | 419 | 3.690 | −20.817 | −0.457 | 1.00 | 39.97 | C |
| ATOM | 1476 | CG | GLN | A | 419 | 4.884 | −20.749 | 0.482 | 1.00 | 40.69 | C |
| ATOM | 1477 | CD | GLN | A | 419 | 4.606 | −19.886 | 1.708 | 1.00 | 40.72 | C |
| ATOM | 1478 | OE1 | GLN | A | 419 | 3.516 | −19.302 | 1.856 | 1.00 | 41.81 | O |
| ATOM | 1479 | NE2 | GLN | A | 419 | 5.589 | −19.809 | 2.599 | 1.00 | 41.17 | N |
| ATOM | 1480 | C | GLN | A | 419 | 2.312 | −22.290 | −1.880 | 1.00 | 38.95 | C |
| ATOM | 1481 | O | GLN | A | 419 | 1.772 | −23.257 | −1.364 | 1.00 | 38.96 | O |
| ATOM | 1482 | N | GLY | A | 420 | 1.716 | −21.520 | −2.776 | 1.00 | 38.60 | N |
| ATOM | 1483 | CA | GLY | A | 420 | 0.329 | −21.659 | −3.178 | 1.00 | 38.25 | C |
| ATOM | 1484 | C | GLY | A | 420 | −0.468 | −20.510 | −2.592 | 1.00 | 38.16 | C |
| ATOM | 1485 | O | GLY | A | 420 | −1.695 | −20.598 | −2.503 | 1.00 | 38.43 | O |
| ATOM | 1486 | N | ASN | A | 421 | 0.212 | −19.431 | −2.155 | 1.00 | 37.46 | N |
| ATOM | 1487 | CA | ASN | A | 421 | −0.531 | −18.310 | −1.572 | 1.00 | 36.31 | C |
| ATOM | 1488 | CB | ASN | A | 421 | 0.435 | −17.236 | −0.996 | 1.00 | 35.81 | C |
| ATOM | 1489 | CG | ASN | A | 421 | 1.202 | −17.717 | 0.233 | 1.00 | 34.95 | C |
| ATOM | 1490 | OD1 | ASN | A | 421 | 0.606 | −17.967 | 1.289 | 1.00 | 32.91 | O |
| ATOM | 1491 | ND2 | ASN | A | 421 | 2.503 | −17.865 | 0.091 | 1.00 | 35.46 | N |
| ATOM | 1492 | C | ASN | A | 421 | −1.502 | −17.698 | −2.609 | 1.00 | 35.82 | C |
| ATOM | 1493 | O | ASN | A | 421 | −1.119 | −17.361 | −3.722 | 1.00 | 35.87 | O |
| ATOM | 1494 | N | VAL | A | 422 | −2.748 | −17.548 | −2.221 | 1.00 | 35.65 | N |
| ATOM | 1495 | CA | VAL | A | 422 | −3.836 | −16.992 | −3.035 | 1.00 | 34.53 | C |
| ATOM | 1496 | CB | VAL | A | 422 | −5.184 | −17.260 | −2.373 | 1.00 | 34.22 | C |
| ATOM | 1497 | CG1 | VAL | A | 422 | −5.468 | −18.754 | −2.321 | 1.00 | 33.79 | C |
| ATOM | 1498 | CG2 | VAL | A | 422 | −5.224 | −16.659 | −0.976 | 1.00 | 34.42 | C |
| ATOM | 1499 | C | VAL | A | 422 | −3.678 | −15.465 | −3.228 | 1.00 | 33.92 | C |
| ATOM | 1500 | O | VAL | A | 422 | −3.582 | −14.753 | −2.239 | 1.00 | 33.64 | O |
| ATOM | 1501 | N | PHE | A | 423 | −3.645 | −14.932 | −4.441 | 1.00 | 33.57 | N |
| ATOM | 1502 | CA | PHE | A | 423 | −3.520 | −13.487 | −4.569 | 1.00 | 33.58 | C |
| ATOM | 1503 | CB | PHE | A | 423 | −2.251 | −13.095 | −5.320 | 1.00 | 33.30 | C |
| ATOM | 1504 | CG | PHE | A | 423 | −0.995 | −13.198 | −4.488 | 1.00 | 32.25 | C |
| ATOM | 1505 | CD1 | PHE | A | 423 | −0.167 | −14.307 | −4.612 | 1.00 | 30.68 | C |
| ATOM | 1506 | CE1 | PHE | A | 423 | 0.988 | −14.415 | −3.875 | 1.00 | 30.81 | C |
| ATOM | 1507 | CZ | PHE | A | 423 | 1.318 | −13.424 | −2.979 | 1.00 | 31.71 | C |
| ATOM | 1508 | CE2 | PHE | A | 423 | 0.507 | −12.309 | −2.844 | 1.00 | 32.18 | C |
| ATOM | 1509 | CD2 | PHE | A | 423 | −0.646 | −12.196 | −3.600 | 1.00 | 31.87 | C |
| ATOM | 1510 | C | PHE | A | 423 | −4.750 | −13.010 | −5.275 | 1.00 | 33.84 | C |
| ATOM | 1511 | O | PHE | A | 423 | −5.294 | −13.736 | −6.095 | 1.00 | 33.51 | O |
| ATOM | 1512 | N | SER | A | 424 | −5.204 | −11.806 | −4.965 | 1.00 | 34.16 | N |
| ATOM | 1513 | CA | SER | A | 424 | −6.425 | −11.325 | −5.616 | 1.00 | 34.95 | C |
| ATOM | 1514 | CB | SER | A | 424 | −7.644 | −11.597 | −4.726 | 1.00 | 35.66 | C |
| ATOM | 1515 | OG | SER | A | 424 | −7.770 | −10.621 | −3.707 | 1.00 | 37.54 | O |
| ATOM | 1516 | C | SER | A | 424 | −6.380 | −9.850 | −6.010 | 1.00 | 34.95 | C |
| ATOM | 1517 | O | SER | A | 424 | −6.069 | −8.954 | −5.218 | 1.00 | 34.73 | O |
| ATOM | 1518 | N | CYS | A | 425 | −6.714 | −9.636 | −7.276 | 1.00 | 34.63 | N |
| ATOM | 1519 | CA | CYS | A | 425 | −6.779 | −8.328 | −7.853 | 1.00 | 34.76 | C |
| ATOM | 1520 | CB | CYS | A | 425 | −6.326 | −8.402 | −9.325 | 1.00 | 35.21 | C |
| ATOM | 1521 | SG | CYS | A | 425 | −6.731 | −6.918 | −10.261 | 1.00 | 35.90 | S |
| ATOM | 1522 | C | CYS | A | 425 | −8.217 | −7.801 | −7.757 | 1.00 | 35.04 | C |
| ATOM | 1523 | O | CYS | A | 425 | −9.151 | −8.378 | −8.348 | 1.00 | 35.28 | O |
| ATOM | 1524 | N | SER | A | 426 | −8.425 | −6.717 | −7.014 | 1.00 | 34.74 | N |
| ATOM | 1525 | CA | SER | A | 426 | −9.773 | −6.169 | −6.934 | 1.00 | 35.18 | C |
| ATOM | 1526 | CB | SER | A | 426 | −10.261 | −6.039 | −5.481 | 1.00 | 36.21 | C |
| ATOM | 1527 | OG | SER | A | 426 | −9.553 | −5.082 | −4.710 | 1.00 | 39.07 | O |
| ATOM | 1528 | C | SER | A | 426 | −9.935 | −4.882 | −7.728 | 1.00 | 34.72 | C |
| ATOM | 1529 | O | SER | A | 426 | −9.047 | −4.009 | −7.690 | 1.00 | 35.21 | O |
| ATOM | 1530 | N | VAL | A | 427 | −11.039 | −4.779 | −8.479 | 1.00 | 33.64 | N |
| ATOM | 1531 | CA | VAL | A | 427 | −11.257 | −3.628 | −9.355 | 1.00 | 32.81 | C |
| ATOM | 1532 | CB | VAL | A | 427 | −11.326 | −4.068 | −10.818 | 1.00 | 32.37 | C |
| ATOM | 1533 | CG1 | VAL | A | 427 | −11.656 | −2.928 | −11.700 | 1.00 | 30.36 | C |
| ATOM | 1534 | CG2 | VAL | A | 427 | −10.022 | −4.709 | −11.237 | 1.00 | 32.20 | C |
| ATOM | 1535 | C | VAL | A | 427 | −12.536 | −2.889 | −8.958 | 1.00 | 33.50 | C |
| ATOM | 1536 | O | VAL | A | 427 | −13.584 | −3.510 | −8.753 | 1.00 | 33.41 | O |
| ATOM | 1537 | N | MET | A | 428 | −12.446 | −1.568 | −8.830 | 1.00 | 33.81 | N |
| ATOM | 1538 | CA | MET | A | 428 | −13.634 | −0.744 | −8.705 | 1.00 | 35.56 | C |
| ATOM | 1539 | CB | MET | A | 428 | −13.538 | 0.142 | −7.485 | 1.00 | 35.27 | C |
| ATOM | 1540 | CG | MET | A | 428 | −13.437 | −0.631 | −6.214 | 1.00 | 37.00 | C |
| ATOM | 1541 | SD | MET | A | 428 | −12.845 | 0.496 | −4.934 | 1.00 | 41.23 | S |
| ATOM | 1542 | CE | MET | A | 428 | −14.422 | 0.795 | −4.117 | 1.00 | 42.19 | C |
| ATOM | 1543 | C | MET | A | 428 | −13.880 | 0.113 | −9.941 | 1.00 | 34.67 | C |
| ATOM | 1544 | O | MET | A | 428 | −12.962 | 0.760 | −10.469 | 1.00 | 36.06 | O |
| ATOM | 1545 | N | HIS | A | 429 | −15.122 | 0.107 | −10.396 | 1.00 | 33.88 | N |
| ATOM | 1546 | CA | HIS | A | 429 | −15.573 | 0.809 | −11.617 | 1.00 | 33.85 | C |
| ATOM | 1547 | CB | HIS | A | 429 | −15.255 | −0.002 | −12.881 | 1.00 | 33.02 | C |
| ATOM | 1548 | CG | HIS | A | 429 | −15.540 | 0.737 | −14.129 | 1.00 | 32.56 | C |
| ATOM | 1549 | ND1 | HIS | A | 429 | −16.748 | 0.625 | −14.794 | 1.00 | 33.60 | N |
| ATOM | 1550 | CE1 | HIS | A | 429 | −16.718 | 1.403 | −15.864 | 1.00 | 32.64 | C |
| ATOM | 1551 | NE2 | HIS | A | 429 | −15.557 | 2.044 | −15.886 | 1.00 | 32.14 | N |
| ATOM | 1552 | CD2 | HIS | A | 429 | −14.808 | 1.655 | −14.804 | 1.00 | 28.69 | C |
| ATOM | 1553 | C | HIS | A | 429 | −17.068 | 0.912 | −11.511 | 1.00 | 33.43 | C |
| ATOM | 1554 | O | HIS | A | 429 | −17.687 | 0.004 | −10.956 | 1.00 | 33.89 | O |
| ATOM | 1555 | N | GLU | A | 430 | −17.651 | 1.973 | −12.046 | 1.00 | 32.66 | N |
| ATOM | 1556 | CA | GLU | A | 430 | −19.092 | 2.184 | −11.905 | 1.00 | 32.40 | C |
| ATOM | 1557 | CB | GLU | A | 430 | −19.489 | 3.506 | −12.536 | 1.00 | 33.44 | C |
| ATOM | 1558 | CG | GLU | A | 430 | −19.098 | 3.639 | −14.013 | 1.00 | 33.54 | C |
| ATOM | 1559 | CD | GLU | A | 430 | −19.794 | 4.790 | −14.615 | 1.00 | 33.97 | C |
| ATOM | 1560 | OE1 | GLU | A | 430 | −19.185 | 5.863 | −14.564 | 1.00 | 34.43 | O |
| ATOM | 1561 | OE2 | GLU | A | 430 | −20.954 | 4.648 | −15.079 | 1.00 | 34.85 | O |
| ATOM | 1562 | C | GLU | A | 430 | −19.938 | 1.129 | −12.554 | 1.00 | 32.19 | C |
| ATOM | 1563 | O | GLU | A | 430 | −21.051 | 0.830 | −12.069 | 1.00 | 32.51 | O |
| ATOM | 1564 | N | ALA | A | 431 | −19.418 | 0.570 | −13.650 | 1.00 | 31.61 | N |
| ATOM | 1565 | CA | ALA | A | 431 | −20.199 | −0.358 | −14.481 | 1.00 | 31.22 | C |
| ATOM | 1566 | CB | ALA | A | 431 | −19.680 | −0.348 | −15.918 | 1.00 | 30.81 | C |
| ATOM | 1567 | C | ALA | A | 431 | −20.316 | −1.800 | −13.929 | 1.00 | 31.04 | C |
| ATOM | 1568 | O | ALA | A | 431 | −21.197 | −2.578 | −14.365 | 1.00 | 31.99 | O |
| ATOM | 1569 | N | LEU | A | 432 | −19.457 | −2.137 | −12.965 | 1.00 | 29.96 | N |
| ATOM | 1570 | CA | LEU | A | 432 | −19.488 | −3.420 | −12.262 | 1.00 | 29.10 | C |
| ATOM | 1571 | CB | LEU | A | 432 | −18.173 | −3.636 | −11.522 | 1.00 | 28.58 | C |
| ATOM | 1572 | CG | LEU | A | 432 | −17.019 | −4.151 | −12.402 | 1.00 | 27.01 | C |
| ATOM | 1573 | CD1 | LEU | A | 432 | −15.641 | −3.799 | −11.761 | 1.00 | 24.42 | C |
| ATOM | 1574 | CD2 | LEU | A | 432 | −17.171 | −5.673 | −12.698 | 1.00 | 23.43 | C |
| ATOM | 1575 | C | LEU | A | 432 | −20.643 | −3.466 | −11.283 | 1.00 | 29.15 | C |
| ATOM | 1576 | O | LEU | A | 432 | −21.017 | −2.470 | −10.710 | 1.00 | 29.57 | O |
| ATOM | 1577 | N | HIS | A | 433 | −21.217 | −4.637 | −11.086 | 1.00 | 29.79 | N |
| ATOM | 1578 | CA | HIS | A | 433 | −22.273 | −4.803 | −10.089 | 1.00 | 29.18 | C |
| ATOM | 1579 | CB | HIS | A | 433 | −22.615 | −6.294 | −9.963 | 1.00 | 28.44 | C |
| ATOM | 1580 | CG | HIS | A | 433 | −23.794 | −6.577 | −9.073 | 1.00 | 29.78 | C |
| ATOM | 1581 | ND1 | HIS | A | 433 | −23.674 | −7.215 | −7.850 | 1.00 | 27.55 | N |
| ATOM | 1582 | CE1 | HIS | A | 433 | −24.865 | −7.322 | −7.300 | 1.00 | 29.74 | C |
| ATOM | 1583 | NE2 | HIS | A | 433 | −25.755 | −6.774 | −8.114 | 1.00 | 32.52 | N |
| ATOM | 1584 | CD2 | HIS | A | 433 | −25.114 | −6.297 | −9.229 | 1.00 | 30.71 | C |
| ATOM | 1585 | C | HIS | A | 433 | −21.798 | −4.248 | −8.736 | 1.00 | 28.96 | C |
| ATOM | 1586 | O | HIS | A | 433 | −20.776 | −4.723 | −8.176 | 1.00 | 28.82 | O |
| ATOM | 1587 | N | ASN | A | 434 | −22.538 | −3.275 | −8.196 | 1.00 | 28.68 | N |
| ATOM | 1588 | CA | ASN | A | 434 | −22.163 | −2.650 | −6.923 | 1.00 | 28.20 | C |
| ATOM | 1589 | CB | ASN | A | 434 | −22.005 | −3.691 | −5.834 | 1.00 | 27.88 | C |
| ATOM | 1590 | CG | ASN | A | 434 | −23.320 | −4.074 | −5.156 | 1.00 | 28.35 | C |
| ATOM | 1591 | OD1 | ASN | A | 434 | −23.335 | −4.853 | −4.196 | 1.00 | 27.24 | O |
| ATOM | 1592 | ND2 | ASN | A | 434 | −24.408 | −3.517 | −5.629 | 1.00 | 27.59 | N |
| ATOM | 1593 | C | ASN | A | 434 | −20.816 | −1.961 | −7.074 | 1.00 | 29.27 | C |
| ATOM | 1594 | O | ASN | A | 434 | −20.185 | −1.667 | −6.056 | 1.00 | 29.25 | O |
| ATOM | 1595 | N | HIS | A | 435 | −20.368 | −1.736 | −8.335 | 1.00 | 29.29 | N |
| ATOM | 1596 | CA | HIS | A | 435 | −19.111 | −1.033 | −8.675 | 1.00 | 29.76 | C |
| ATOM | 1597 | CB | HIS | A | 435 | −19.142 | 0.398 | −8.128 | 1.00 | 29.37 | C |
| ATOM | 1598 | CG | HIS | A | 435 | −20.517 | 0.972 | −8.056 | 1.00 | 30.48 | C |
| ATOM | 1599 | ND1 | HIS | A | 435 | −21.277 | 1.218 | −9.179 | 1.00 | 30.30 | N |
| ATOM | 1600 | CE1 | HIS | A | 435 | −22.446 | 1.706 | −8.812 | 1.00 | 32.52 | C |
| ATOM | 1601 | NE2 | HIS | A | 435 | −22.479 | 1.769 | −7.491 | 1.00 | 35.29 | N |
| ATOM | 1602 | CD2 | HIS | A | 435 | −21.284 | 1.318 | −6.993 | 1.00 | 33.54 | C |
| ATOM | 1603 | C | HIS | A | 435 | −17.811 | −1.739 | −8.219 | 1.00 | 30.44 | C |
| ATOM | 1604 | O | HIS | A | 435 | −16.768 | −1.100 | −8.122 | 1.00 | 29.51 | O |
| ATOM | 1605 | N | TYR | A | 436 | −17.904 | −3.036 | −7.904 | 1.00 | 31.51 | N |
| ATOM | 1606 | CA | TYR | A | 436 | −16.788 | −3.873 | −7.422 | 1.00 | 32.46 | C |
| ATOM | 1607 | CB | TYR | A | 436 | −16.929 | −4.123 | −5.909 | 1.00 | 32.34 | C |
| ATOM | 1608 | CG | TYR | A | 436 | −15.741 | −4.794 | −5.270 | 1.00 | 31.43 | C |
| ATOM | 1609 | CD1 | TYR | A | 436 | −15.755 | −6.164 | −4.999 | 1.00 | 32.40 | C |
| ATOM | 1610 | CE1 | TYR | A | 436 | −14.660 | −6.806 | −4.452 | 1.00 | 32.27 | C |
| ATOM | 1611 | CZ | TYR | A | 436 | −13.529 | −6.068 | −4.147 | 1.00 | 32.67 | C |
| ATOM | 1612 | OH | TYR | A | 436 | −12.424 | −6.688 | −3.566 | 1.00 | 32.73 | O |
| ATOM | 1613 | CE2 | TYR | A | 436 | −13.505 | −4.698 | −4.410 | 1.00 | 31.47 | C |
| ATOM | 1614 | CD2 | TYR | A | 436 | −14.605 | −4.075 | −4.962 | 1.00 | 29.15 | C |
| ATOM | 1615 | C | TYR | A | 436 | −16.770 | −5.231 | −8.145 | 1.00 | 33.21 | C |
| ATOM | 1616 | O | TYR | A | 436 | −17.805 | −5.790 | −8.471 | 1.00 | 33.93 | O |
| ATOM | 1617 | N | THR | A | 437 | −15.586 | −5.725 | −8.427 | 1.00 | 33.91 | N |
| ATOM | 1618 | CA | THR | A | 437 | −15.375 | −7.132 | −8.709 | 1.00 | 34.50 | C |
| ATOM | 1619 | CB | THR | A | 437 | −15.402 | −7.452 | −10.222 | 1.00 | 34.95 | C |
| ATOM | 1620 | OG1 | THR | A | 437 | −15.649 | −8.853 | −10.397 | 1.00 | 31.80 | O |
| ATOM | 1621 | CG2 | THR | A | 437 | −14.034 | −7.031 | −10.927 | 1.00 | 34.25 | C |
| ATOM | 1622 | C | THR | A | 437 | −14.000 | −7.466 | −8.135 | 1.00 | 34.88 | C |
| ATOM | 1623 | O | THR | A | 437 | −13.307 | −6.579 | −7.625 | 1.00 | 34.91 | O |
| ATOM | 1624 | N | GLN | A | 438 | −13.598 | −8.726 | −8.245 | 1.00 | 35.49 | N |
| ATOM | 1625 | CA | GLN | A | 438 | −12.325 | −9.201 | −7.675 | 1.00 | 36.09 | C |
| ATOM | 1626 | CB | GLN | A | 438 | −12.506 | −9.472 | −6.147 | 1.00 | 36.09 | C |
| ATOM | 1627 | CG | GLN | A | 438 | −11.215 | −9.722 | −5.296 | 1.00 | 36.92 | C |
| ATOM | 1628 | CD | GLN | A | 438 | −11.492 | −9.814 | −3.738 | 1.00 | 37.75 | C |
| ATOM | 1629 | OE1 | GLN | A | 438 | −10.632 | −9.474 | −2.917 | 1.00 | 40.41 | O |
| ATOM | 1630 | NE2 | GLN | A | 438 | −12.675 | −10.273 | −3.362 | 1.00 | 37.92 | N |
| ATOM | 1631 | C | GLN | A | 438 | −11.932 | −10.473 | −8.449 | 1.00 | 35.29 | C |
| ATOM | 1632 | O | GLN | A | 438 | −12.760 | −11.344 | −8.618 | 1.00 | 34.59 | O |
| ATOM | 1633 | N | LYS | A | 439 | −10.703 | −10.566 | −8.956 | 1.00 | 35.18 | N |
| ATOM | 1634 | CA | LYS | A | 439 | −10.221 | −11.850 | −9.496 | 1.00 | 35.39 | C |
| ATOM | 1635 | CB | LYS | A | 439 | −10.054 | −11.831 | −11.015 | 1.00 | 35.18 | C |
| ATOM | 1636 | CG | LYS | A | 439 | −11.055 | −10.935 | −11.748 | 1.00 | 36.91 | C |
| ATOM | 1637 | CD | LYS | A | 439 | −12.472 | −11.529 | −11.871 | 1.00 | 37.54 | C |
| ATOM | 1638 | CE | LYS | A | 439 | −12.438 | −12.800 | −12.718 | 1.00 | 40.06 | C |
| ATOM | 1639 | NZ | LYS | A | 439 | −13.754 | −13.538 | −12.788 | 1.00 | 39.20 | N |
| ATOM | 1640 | C | LYS | A | 439 | −8.957 | −12.334 | −8.785 | 1.00 | 35.64 | C |
| ATOM | 1641 | O | LYS | A | 439 | −8.004 | −11.570 | −8.588 | 1.00 | 35.65 | O |
| ATOM | 1642 | N | SER | A | 440 | −9.015 | −13.600 | −8.369 | 1.00 | 36.04 | N |
| ATOM | 1643 | CA | SER | A | 440 | −7.969 | −14.339 | −7.650 | 1.00 | 36.77 | C |
| ATOM | 1644 | CB | SER | A | 440 | −8.599 | −15.343 | −6.626 | 1.00 | 36.99 | C |
| ATOM | 1645 | OG | SER | A | 440 | −8.776 | −14.852 | −5.281 | 1.00 | 36.13 | O |
| ATOM | 1646 | C | SER | A | 440 | −7.101 | −15.138 | −8.637 | 1.00 | 37.22 | C |
| ATOM | 1647 | O | SER | A | 440 | −7.577 | −15.641 | −9.666 | 1.00 | 37.52 | O |
| ATOM | 1648 | N | LEU | A | 441 | −5.835 | −15.285 | −8.283 | 1.00 | 37.31 | N |
| ATOM | 1649 | CA | LEU | A | 441 | −4.879 | −15.994 | −9.082 | 1.00 | 37.72 | C |
| ATOM | 1650 | CB | LEU | A | 441 | −4.015 | −14.945 | −9.776 | 1.00 | 38.01 | C |
| ATOM | 1651 | CG | LEU | A | 441 | −2.903 | −15.284 | −10.750 | 1.00 | 37.80 | C |
| ATOM | 1652 | CD1 | LEU | A | 441 | −3.478 | −16.039 | −11.951 | 1.00 | 39.99 | C |
| ATOM | 1653 | CD2 | LEU | A | 441 | −2.184 | −14.020 | −11.163 | 1.00 | 36.66 | C |
| ATOM | 1654 | C | LEU | A | 441 | −4.066 | −16.784 | −8.068 | 1.00 | 38.89 | C |
| ATOM | 1655 | O | LEU | A | 441 | −3.660 | −16.201 | −7.064 | 1.00 | 39.18 | O |
| ATOM | 1656 | N | SER | A | 442 | −3.858 | −18.094 | −8.298 | 1.00 | 39.91 | N |
| ATOM | 1657 | CA | SER | A | 442 | −3.091 | −19.009 | −7.382 | 1.00 | 41.21 | C |
| ATOM | 1658 | CB | SER | A | 442 | −4.041 | −19.912 | −6.529 | 1.00 | 41.24 | C |
| ATOM | 1659 | OG | SER | A | 442 | −5.163 | −19.213 | −5.962 | 1.00 | 41.41 | O |
| ATOM | 1660 | C | SER | A | 442 | −2.097 | −19.919 | −8.175 | 1.00 | 41.94 | C |
| ATOM | 1661 | O | SER | A | 442 | −2.285 | −20.110 | −9.381 | 1.00 | 42.72 | O |
| ATOM | 1662 | N | LEU | A | 443 | −1.062 | −20.486 | −7.531 | 1.00 | 41.93 | N |
| ATOM | 1663 | CA | LEU | A | 443 | −0.203 | −21.478 | −8.219 | 1.00 | 41.88 | C |
| ATOM | 1664 | CB | LEU | A | 443 | 0.999 | −21.886 | −7.362 | 1.00 | 41.57 | C |
| ATOM | 1665 | CG | LEU | A | 443 | 2.019 | −22.772 | −8.093 | 1.00 | 40.82 | C |
| ATOM | 1666 | CD1 | LEU | A | 443 | 2.613 | −22.098 | −9.312 | 1.00 | 39.70 | C |
| ATOM | 1667 | CD2 | LEU | A | 443 | 3.124 | −23.228 | −7.162 | 1.00 | 41.60 | C |
| ATOM | 1668 | C | LEU | A | 443 | −0.993 | −22.724 | −8.677 | 1.00 | 42.41 | C |
| ATOM | 1669 | O | LEU | A | 443 | −1.729 | −23.332 | −7.887 | 1.00 | 42.74 | O |
| ATOM | 1670 | N | SER | A | 444 | −0.839 | −23.109 | −9.945 | 1.00 | 42.72 | N |
| ATOM | 1671 | CA | SER | A | 444 | −1.763 | −24.087 | −10.559 | 1.00 | 42.90 | C |
| ATOM | 1672 | CB | SER | A | 444 | −2.143 | −23.637 | −11.988 | 1.00 | 43.35 | C |
| ATOM | 1673 | OG | SER | A | 444 | −2.922 | −22.436 | −11.989 | 1.00 | 40.96 | O |
| ATOM | 1674 | C | SER | A | 444 | −1.288 | −25.547 | −10.545 | 1.00 | 42.89 | C |
| ATOM | 1675 | O | SER | A | 444 | −1.163 | −26.170 | −9.485 | 1.00 | 42.84 | O |
| ATOM | 1676 | C1 | NAG | C | 1 | −1.487 | 33.784 | −5.963 | 1.00 | 65.70 | C |
| ATOM | 1677 | C2 | NAG | C | 1 | −1.520 | 33.605 | −7.489 | 1.00 | 70.42 | C |
| ATOM | 1678 | N2 | NAG | C | 1 | −1.903 | 34.844 | −8.184 | 1.00 | 74.29 | N |
| ATOM | 1679 | C7 | NAG | C | 1 | −1.176 | 35.558 | −9.089 | 1.00 | 75.78 | C |
| ATOM | 1680 | O7 | NAG | C | 1 | −0.318 | 35.077 | −9.839 | 1.00 | 76.92 | O |
| ATOM | 1681 | C8 | NAG | C | 1 | −1.459 | 37.040 | −9.207 | 1.00 | 74.31 | C |
| ATOM | 1682 | C3 | NAG | C | 1 | −2.564 | 32.527 | −7.800 | 1.00 | 70.46 | C |
| ATOM | 1683 | O3 | NAG | C | 1 | −2.649 | 32.316 | −9.198 | 1.00 | 72.00 | O |
| ATOM | 1684 | C4 | NAG | C | 1 | −2.378 | 31.222 | −7.007 | 1.00 | 70.47 | C |
| ATOM | 1685 | O4 | NAG | C | 1 | −3.495 | 30.359 | −7.111 | 1.00 | 69.64 | O |
| ATOM | 1686 | C5 | NAG | C | 1 | −2.284 | 31.528 | −5.519 | 1.00 | 71.24 | C |
| ATOM | 1687 | C6 | NAG | C | 1 | −2.013 | 30.186 | −4.827 | 1.00 | 74.78 | C |
| ATOM | 1688 | O6 | NAG | C | 1 | −1.441 | 30.199 | −3.536 | 1.00 | 79.55 | O |
| ATOM | 1689 | O5 | NAG | C | 1 | −1.281 | 32.520 | −5.331 | 1.00 | 69.12 | O |
| ATOM | 1690 | C1 | NAG | C | 2 | −3.378 | 29.399 | −8.171 | 1.00 | 69.66 | C |
| ATOM | 1691 | C2 | NAG | C | 2 | −4.079 | 28.126 | −7.725 | 1.00 | 69.13 | C |
| ATOM | 1692 | N2 | NAG | C | 2 | −3.502 | 27.609 | −6.497 | 1.00 | 67.28 | N |
| ATOM | 1693 | C7 | NAG | C | 2 | −4.242 | 27.295 | −5.420 | 1.00 | 64.10 | C |
| ATOM | 1694 | O7 | NAG | C | 2 | −5.472 | 27.271 | −5.432 | 1.00 | 62.51 | O |
| ATOM | 1695 | C8 | NAG | C | 2 | −3.517 | 26.966 | −4.140 | 1.00 | 61.62 | C |
| ATOM | 1696 | C3 | NAG | C | 2 | −3.973 | 27.065 | −8.807 | 1.00 | 71.50 | C |
| ATOM | 1697 | O3 | NAG | C | 2 | −4.966 | 26.117 | −8.490 | 1.00 | 73.51 | O |
| ATOM | 1698 | C4 | NAG | C | 2 | −4.278 | 27.619 | −10.205 | 1.00 | 72.05 | C |
| ATOM | 1699 | O4 | NAG | C | 2 | −3.835 | 26.722 | −11.214 | 1.00 | 73.09 | O |
| ATOM | 1700 | C5 | NAG | C | 2 | −3.663 | 28.998 | −10.451 | 1.00 | 70.37 | C |
| ATOM | 1701 | C6 | NAG | C | 2 | −4.294 | 29.619 | −11.679 | 1.00 | 70.89 | C |
| ATOM | 1702 | O6 | NAG | C | 2 | −3.704 | 30.878 | −11.890 | 1.00 | 71.87 | O |
| ATOM | 1703 | O5 | NAG | C | 2 | −3.948 | 29.856 | −9.377 | 1.00 | 68.94 | O |
| ATOM | 1704 | C1 | BMA | C | 3 | −4.874 | 25.808 | −11.599 | 1.00 | 72.33 | C |
| ATOM | 1705 | C2 | BMA | C | 3 | −4.874 | 25.711 | −13.110 | 1.00 | 72.77 | C |
| ATOM | 1706 | O2 | BMA | C | 3 | −3.499 | 25.605 | −13.416 | 1.00 | 73.22 | O |
| ATOM | 1707 | C3 | BMA | C | 3 | −5.663 | 24.500 | −13.667 | 1.00 | 74.41 | C |
| ATOM | 1708 | O3 | BMA | C | 3 | −5.404 | 24.151 | −15.032 | 1.00 | 77.63 | O |
| ATOM | 1709 | C4 | BMA | C | 3 | −5.444 | 23.236 | −12.875 | 1.00 | 72.39 | C |
| ATOM | 1710 | O4 | BMA | C | 3 | −6.551 | 22.390 | −13.158 | 1.00 | 71.13 | O |
| ATOM | 1711 | C5 | BMA | C | 3 | −5.474 | 23.526 | −11.384 | 1.00 | 71.54 | C |
| ATOM | 1712 | C6 | BMA | C | 3 | −5.088 | 22.270 | −10.641 | 1.00 | 70.02 | C |
| ATOM | 1713 | O6 | BMA | C | 3 | −5.364 | 22.552 | −9.293 | 1.00 | 67.41 | O |
| ATOM | 1714 | O5 | BMA | C | 3 | −4.557 | 24.551 | −11.029 | 1.00 | 70.70 | O |
| ATOM | 1715 | C1 | MAN | C | 4 | −6.477 | 24.718 | −15.818 | 1.00 | 86.16 | C |
| ATOM | 1716 | C2 | MAN | C | 4 | −6.543 | 24.110 | −17.210 | 1.00 | 91.93 | C |
| ATOM | 1717 | O2 | MAN | C | 4 | −7.724 | 24.606 | −17.831 | 1.00 | 99.47 | O |
| ATOM | 1718 | C3 | MAN | C | 4 | −5.340 | 24.543 | −18.073 | 1.00 | 91.93 | C |
| ATOM | 1719 | O3 | MAN | C | 4 | −5.768 | 24.665 | −19.416 | 1.00 | 92.15 | O |
| ATOM | 1720 | C4 | MAN | C | 4 | −4.620 | 25.860 | −17.689 | 1.00 | 90.76 | C |
| ATOM | 1721 | O4 | MAN | C | 4 | −3.241 | 25.572 | −17.512 | 1.00 | 89.98 | O |
| ATOM | 1722 | C5 | MAN | C | 4 | −5.171 | 26.612 | −16.447 | 1.00 | 90.31 | C |
| ATOM | 1723 | C6 | MAN | C | 4 | −5.203 | 28.154 | −16.584 | 1.00 | 90.79 | C |
| ATOM | 1724 | O6 | MAN | C | 4 | −4.863 | 28.831 | −15.378 | 1.00 | 88.69 | O |
| ATOM | 1725 | O5 | MAN | C | 4 | −6.452 | 26.117 | −16.046 | 1.00 | 86.83 | O |
| ATOM | 1726 | C1 | NAG | C | 5 | −8.982 | 24.383 | −17.104 | 1.00 | 105.34 | C |
| ATOM | 1727 | C2 | NAG | C | 5 | −10.157 | 24.775 | −18.023 | 1.00 | 107.42 | C |
| ATOM | 1728 | N2 | NAG | C | 5 | −11.436 | 24.969 | −17.305 | 1.00 | 107.95 | N |
| ATOM | 1729 | C7 | NAG | C | 5 | −12.285 | 25.988 | −17.532 | 1.00 | 107.70 | C |
| ATOM | 1730 | O7 | NAG | C | 5 | −11.987 | 27.171 | −17.344 | 1.00 | 106.84 | O |
| ATOM | 1731 | C8 | NAG | C | 5 | −13.660 | 25.636 | −18.040 | 1.00 | 107.76 | C |
| ATOM | 1732 | C3 | NAG | C | 5 | −10.210 | 23.764 | −19.191 | 1.00 | 108.18 | C |
| ATOM | 1733 | O3 | NAG | C | 5 | −9.230 | 24.141 | −20.153 | 1.00 | 108.45 | O |
| ATOM | 1734 | C4 | NAG | C | 5 | −9.929 | 22.292 | −18.802 | 1.00 | 107.62 | C |
| ATOM | 1735 | O4 | NAG | C | 5 | −10.946 | 21.436 | −19.281 | 1.00 | 105.68 | O |
| ATOM | 1736 | C5 | NAG | C | 5 | −9.735 | 22.010 | −17.299 | 1.00 | 107.94 | C |
| ATOM | 1737 | C6 | NAG | C | 5 | −8.926 | 20.715 | −17.119 | 1.00 | 108.14 | C |
| ATOM | 1738 | O6 | NAG | C | 5 | −9.803 | 19.682 | −16.718 | 1.00 | 106.50 | O |
| ATOM | 1739 | O5 | NAG | C | 5 | −9.148 | 23.075 | −16.530 | 1.00 | 107.29 | O |
| ATOM | 1740 | C1 | MAN | C | 7 | −4.860 | 21.485 | −8.509 | 1.00 | 70.53 | C |
| ATOM | 1741 | C2 | MAN | C | 7 | −5.141 | 21.853 | −7.051 | 1.00 | 74.25 | C |
| ATOM | 1742 | O2 | MAN | C | 7 | −5.212 | 20.675 | −6.278 | 1.00 | 73.87 | O |
| ATOM | 1743 | C3 | MAN | C | 7 | −4.076 | 22.812 | −6.453 | 1.00 | 75.33 | C |
| ATOM | 1744 | O3 | MAN | C | 7 | −4.216 | 22.920 | −5.037 | 1.00 | 76.92 | O |
| ATOM | 1745 | C4 | MAN | C | 7 | −2.656 | 22.361 | −6.825 | 1.00 | 73.70 | C |
| ATOM | 1746 | O4 | MAN | C | 7 | −1.738 | 23.278 | −6.286 | 1.00 | 74.65 | O |
| ATOM | 1747 | C5 | MAN | C | 7 | −2.596 | 22.269 | −8.355 | 1.00 | 72.51 | C |
| ATOM | 1748 | C6 | MAN | C | 7 | −1.214 | 21.985 | −8.942 | 1.00 | 70.71 | C |
| ATOM | 1749 | O6 | MAN | C | 7 | −0.819 | 20.769 | −8.357 | 1.00 | 67.04 | O |
| ATOM | 1750 | O5 | MAN | C | 7 | −3.492 | 21.237 | −8.750 | 1.00 | 71.62 | O |
| ATOM | 1751 | C1 | NAG | C | 8 | −6.551 | 20.176 | −6.136 | 1.00 | 72.98 | C |
| ATOM | 1752 | C2 | NAG | C | 8 | −6.330 | 18.860 | −5.426 | 1.00 | 73.86 | C |
| ATOM | 1753 | N2 | NAG | C | 8 | −5.146 | 18.176 | −5.938 | 1.00 | 75.74 | N |
| ATOM | 1754 | C7 | NAG | C | 8 | −3.928 | 18.420 | −5.423 | 1.00 | 75.48 | C |
| ATOM | 1755 | O7 | NAG | C | 8 | −3.707 | 19.198 | −4.459 | 1.00 | 74.47 | O |
| ATOM | 1756 | C8 | NAG | C | 8 | −2.819 | 17.676 | −6.122 | 1.00 | 74.17 | C |
| ATOM | 1757 | C3 | NAG | C | 8 | −7.608 | 18.060 | −5.539 | 1.00 | 72.64 | C |
| ATOM | 1758 | O3 | NAG | C | 8 | −7.361 | 16.773 | −4.983 | 1.00 | 73.15 | O |
| ATOM | 1759 | C4 | NAG | C | 8 | −8.684 | 18.856 | −4.794 | 1.00 | 69.95 | C |
| ATOM | 1760 | O4 | NAG | C | 8 | −9.957 | 18.284 | −5.021 | 1.00 | 70.64 | O |
| ATOM | 1761 | C5 | NAG | C | 8 | −8.725 | 20.328 | −5.216 | 1.00 | 69.23 | C |
| ATOM | 1762 | C6 | NAG | C | 8 | −9.565 | 21.136 | −4.242 | 1.00 | 66.36 | C |
| ATOM | 1763 | O6 | NAG | C | 8 | −8.888 | 21.214 | −3.014 | 1.00 | 65.11 | O |
| ATOM | 1764 | O5 | NAG | C | 8 | −7.440 | 20.928 | −5.333 | 1.00 | 69.51 | O |
| ATOM | 1765 | C1 | GAL | C | 9 | −10.427 | 17.625 | −3.816 | 1.00 | 70.09 | C |
| ATOM | 1766 | C2 | GAL | C | 9 | −11.695 | 16.771 | −4.039 | 1.00 | 67.64 | C |
| ATOM | 1767 | O2 | GAL | C | 9 | −12.733 | 17.452 | −4.729 | 1.00 | 64.56 | O |
| ATOM | 1768 | C3 | GAL | C | 9 | −12.166 | 16.245 | −2.668 | 1.00 | 68.80 | C |
| ATOM | 1769 | O3 | GAL | C | 9 | −13.177 | 15.272 | −2.838 | 1.00 | 64.14 | O |
| ATOM | 1770 | C4 | GAL | C | 9 | −10.974 | 15.713 | −1.803 | 1.00 | 72.48 | C |
| ATOM | 1771 | O4 | GAL | C | 9 | −10.467 | 14.454 | −2.255 | 1.00 | 74.26 | O |
| ATOM | 1772 | C5 | GAL | C | 9 | −9.819 | 16.728 | −1.772 | 1.00 | 72.02 | C |
| ATOM | 1773 | C6 | GAL | C | 9 | −8.621 | 16.343 | −0.924 | 1.00 | 74.91 | C |
| ATOM | 1774 | O6 | GAL | C | 9 | −9.059 | 16.255 | 0.423 | 1.00 | 81.18 | O |
| ATOM | 1775 | O5 | GAL | C | 9 | −9.408 | 16.898 | −3.101 | 1.00 | 70.21 | O |
| ATOM | 1776 | C1 | FUC | C | 11 | −0.243 | 29.382 | −3.519 | 1.00 | 84.74 | C |
| ATOM | 1777 | C2 | FUC | C | 11 | 1.029 | 30.270 | −3.436 | 1.00 | 87.16 | C |
| ATOM | 1778 | O2 | FUC | C | 11 | 1.010 | 31.167 | −2.325 | 1.00 | 87.00 | O |
| ATOM | 1779 | C3 | FUC | C | 11 | 2.293 | 29.373 | −3.412 | 1.00 | 88.83 | C |
| ATOM | 1780 | O3 | FUC | C | 11 | 3.485 | 30.141 | −3.434 | 1.00 | 89.75 | O |
| ATOM | 1781 | C4 | FUC | C | 11 | 2.267 | 28.292 | −4.520 | 1.00 | 87.40 | C |
| ATOM | 1782 | O4 | FUC | C | 11 | 2.344 | 28.915 | −5.786 | 1.00 | 85.12 | O |
| ATOM | 1783 | C5 | FUC | C | 11 | 0.956 | 27.492 | −4.362 | 1.00 | 87.17 | C |
| ATOM | 1784 | C6 | FUC | C | 11 | 0.858 | 26.206 | −5.192 | 1.00 | 86.02 | C |
| ATOM | 1785 | O5 | FUC | C | 11 | −0.141 | 28.392 | −4.571 | 1.00 | 87.00 | O |
| ATOM | 1786 | ZN | ZN | I | 1 | −23.927 | 2.563 | −6.294 | 1.00 | 53.50 | ZN |
| ATOM | 1787 | ZN | ZN | I | 2 | −27.285 | −6.524 | −6.617 | 0.50 | 82.97 | ZN |
| ATOM | 1788 | ZN | ZN | I | 3 | −24.670 | 21.331 | −18.634 | 0.50 | 66.38 | ZN |
| ATOM | 1789 | ZN | ZN | I | 4 | −21.826 | −7.068 | −13.742 | 1.00 | 55.82 | O |
| ATOM | 1790 | OW | WAT | W | 1 | 1.745 | 2.912 | −5.310 | 1.00 | 42.67 | O |
| ATOM | 1791 | OW | WAT | W | 2 | −25.206 | −1.824 | −8.886 | 1.00 | 48.03 | O |
| ATOM | 1792 | OW | WAT | W | 3 | −26.222 | 17.362 | −1.284 | 1.00 | 56.50 | O |
| ATOM | 1793 | OW | WAT | W | 4 | −23.816 | 0.876 | −4.347 | 1.00 | 40.49 | O |
| ATOM | 1794 | OW | WAT | W | 5 | −6.478 | 35.996 | −13.098 | 1.00 | 57.30 | O |
| ATOM | 1795 | OW | WAT | W | 6 | −23.234 | −5.632 | −13.594 | 1.00 | 20.00 | O |
| ATOM | 1796 | OW | WAT | W | 7 | −4.287 | −11.875 | 7.514 | 1.00 | 53.40 | O |
| ATOM | 1797 | OW | WAT | W | 8 | 4.844 | −4.257 | −9.188 | 1.00 | 35.28 | O |
| ATOM | 1798 | OW | WAT | W | 9 | −8.110 | 35.966 | −14.975 | 1.00 | 54.77 | O |
| ATOM | 1799 | OW | WAT | W | 10 | −14.550 | 13.402 | −2.556 | 1.00 | 38.28 | O |
| ATOM | 1800 | OW | WAT | W | 11 | −15.505 | 4.284 | −12.606 | 1.00 | 52.56 | O |
| ATOM | 1801 | OW | WAT | W | 12 | −25.360 | 3.290 | −8.202 | 1.00 | 49.22 | O |
| ATOM | 1802 | OW | WAT | W | 13 | −23.459 | 9.865 | −20.021 | 1.00 | 44.19 | O |
| ATOM | 1803 | OW | WAT | W | 14 | −25.107 | 8.483 | −21.335 | 1.00 | 30.57 | O |
| ATOM | 1804 | OW | WAT | W | 15 | −27.666 | 2.042 | −22.066 | 1.00 | 50.32 | O |
| ATOM | 1805 | OW | WAT | W | 16 | −30.285 | 16.042 | −5.140 | 1.00 | 51.39 | O |
| ATOM | 1806 | OW | WAT | W | 17 | −12.486 | 19.490 | −0.850 | 1.00 | 45.83 | O |
| ATOM | 1806 | OW | WAT | W | 18 | −17.836 | 32.100 | −17.680 | 1.00 | 46.89 | O |
| ATOM | 1806 | OW | WAT | W | 19 | −23.010 | 22.771 | −18.199 | 1.00 | 47.98 | O |
| ATOM | 1806 | OW | WAT | W | 20 | −33.656 | 12.489 | −7.950 | 1.00 | 48.11 | O |
| ATOM | 1806 | OW | WAT | W | 21 | −32.128 | 8.390 | −6.655 | 1.00 | 51.33 | O |
| ATOM | 1806 | OW | WAT | W | 22 | −10.738 | 16.672 | 3.005 | 1.00 | 52.21 | O |
| ATOM | 1806 | OW | WAT | W | 23 | −10.891 | 12.599 | 0.649 | 1.00 | 47.76 | O |
| ATOM | 1806 | OW | WAT | W | 24 | −13.467 | 9.066 | −19.677 | 1.00 | 41.28 | O |

TABLE 6
Structural properties of various human IgG and IgG/Fc molecules.

| | | Reso- | | Distance^{a} | | Sugar | | | |
|---|---|---|---|---|---|---|---|---|---|
| | Space | lution | | P329/P329^{a} | V323/V323 | Distance^{b} | | C_{H}2/C_{H}3 Angle^{a} | |
| PDBID | Group | (Å) | State | (Å) | (Å) | (Å) | Chains | L443-Q342-P329 (°) | F423-E430-V323 (°) |
| 1E4K^{c} | P6₅22 | 3.2 | Fc bound to CD 16 | 30.3 | 39.5 | 5.2 | A, B | A (114.6), B (124.7) | A (118.0), B (127.9) |
| 1H3T^{d} | P2₁2₁2₁1 | 2.4 | Free Fc | 22 | 34.5 | 7.6 | A, B | A (116.8), B (112.0) | A (120.3), B (117.9) |
| 1H3U^{d} | P2₁2₁2₁ | 2.4 | Free Fc | 24.2 | 34.7 | 3.3 | A, B | A (117.4), B (112.5) | A (120.8), B (117.9) |
| 1H3V^{d} | P2₁2₁2₁ | 2.4 | Free Fc | 26.9 | 36.1 | 4.1 | A, B | A (118.5), B (115.0) | A (122.7), B (119.4) |
| 1H3W^{d} | C222₁ | 2.85 | Free Fc | 33.8 | 41.3 | 11.8 | M | M (119.4) | M (124.4) |
| 1H3X^{d} | P2₁2₁2₁ | 2.44 | Free Fc | 22.6 | 34.9 | 3.3 | A, B | A (117.0), B (112.0) | A (121.4), B (119.5) |
| 1H3Y^{d} | P6₁22 | 4.1 | Free Fc | 29.6 | 36.2 | 2.9 | A, B | A (117.8), B (122.9) | A (125.3), B (128.7) |
| 1FC1^{e} | P2₁2₁2₁ | 2.9 | Free Fc | 26.8 | 36.8 | 3.2 | A, B | A (116.7), B (115.2) | A (122.4), B (119.8) |
| 1FC2^{e} | P3₁21 | 2.8 | Fc bound to Pt. A | 27.7 | 35.1 | 3.3 | D | D (117.2) | D (120.7) |
| | | | fragment | | | | | | |
| 1T83^{f} | P2₁2₁2₁, | 3 | Fc bound to CD 16 | 31.3 | 39.6 | 8.4 | A, B | A (119.8), B (117.5) | A (121.4), B (121.4) |
| 1T89^{f} | P6₅22 | 3.5 | Fc bound to CD 16 | 30.6 | 39.2 | 7.1 | A, B | A (122.5), B (114.7) | A (128.4), B (118.7) |
| 2GJ7^{g} | P4₃2₁2 | 5 | Fc bound to gE-gl | 31.9 | 40.9 | 6.4 | A, B | A (117.2), B (121.1) | A (121.7), B (123.0) |
| 1HZH^{h} | H32 | 2.7 | Free IgG | 23.9 | 35.4 | 3.2 | H, K | H (111.8), K (112.5) | H (116.2), K (117.1) |
| 2DTQⁱ | P2₁2₁2₁ | 2 | Free Fc | 25.5 | 34.7 | 2.8 | A, B | A (117.2), B (113.4) | A (121.0), B (118.6) |
| 2DTSⁱ | P2₁2₁2₁ | 2.2 | Free Fc | 24.2 | 34.1 | 2.5 | A, B | A (117.0), B (113.7) | A (120.6), B (118.3) |
| 2J6E^{j} | C2 | 3 | Fc bound to RF61 | 22.1 | 32.6 | 3.8 | A, B | A (115.1), B (110.3) | A (120.3), B (113.4) |
| 1MC0^{k} | C222₁ | 3.2 | Free IgG, hinge | 9.5 | 24.3 | 3.1 | H | H (106.1) | H (114.4) |
| | | | deleted | | | | | | |
| 10QO | P2₁2₁2 | 2.3 | Fc bound to Pt. A | 24.3 | 31.0 | 2.8 | A, B | A (114.7), B (116.2) | A (119.1), B (118.0) |
| | | | fragment | | | | | | |
| 10QX | P2₁2₁2₁ | 2.6 | Fc bound to Pt. A | 24.7 | 30.8 | 2.6 | A, B | A (122.5), B (113.7) | A (120.0), B (116.6) |
| | | | fragment | | | | | | |
| 1FCC^{l} | P4₃2₁2 | 3.2 | Fc bound to Pt. G | 34.7 | 40.6 | N/A | A, B | A (118.1), B (118.1) | A (122.8), B (122.8) |
| | | | fragment | | | | | | |
| 1DN2^{m} | P2₁ | 2.7 | Fc bound to peptide | 31.9 | 36.6 | 6.4 | A, B | A (117.2), B (121.1) | A (121.7), B (123.0) |
| 1L6Xⁿ | C222₁ | 1.65 | Fc bound to Pt. A | 26.7 | 37.0 | 2.4 | A | A (115.2) | A (118.8) |
| | | | fragment | | | | | | |
| 2IWG^{o} | P6₁ | 2.35 | Fc bound to TRIM21 | 45.2 | 47.6 | 7.7 | A, D | A (121.7), B (121.7) | A (125.4), D (125.5) |
| 1ADQ^{p} | C2 | 3.15 | Fc bound to IgM Fab | 20.7 | 32.7 | N/A | A | A (114.3) | A (118.8) |
| 2QL1^{qo} | C222₁ | 2.53 | Free Fc | 39.1 | 43.6 | 6.9 | A | A (124.2) | A (129.0) |
| 3DO3 | P2₁2₁2₁ | | Free Fc | 23.50 | 35.10 | | | 118.43 | 122.23 |
| 3DNK | P2₁2₁2₁ | | Free deglycosylated | 27.60 | 37.97 | | | 115.23 | 117.71 |

^{a}Angles and interchain distances were measured as described in the Example section
^{b}Sugar distances correspond to the closest interchain distance between oxygen atoms of each carbohydrate chain. No carbohydrates were described for 1FCC and 1ADQ. Fc/3M (current work).
^{c}Sondermann el al. 2000, Nature 406, 267-273
^{d}Krapp et al. 2003, J. Mol. Biol. 325: 979-989
^{e}Deisenhofer, 1981, Biochemistry 20: 2361-2370
^{f}Radaev et al. 2001, J. Biol. Chem. 276: 16469-16477
^{g}Sprague et al. 2006, PLoS Biol. 4: e148
^{h}Saphire et al. 2001, Science 293: 1155-1159
ⁱMatsumiya et al. 2007, Mol. Biol. 368:767-779
^{j}Duquerroy et al. 2007, J. Mol. Biol. 368: 1321-1331
^{k}Guddat et al. 1993, Proc. Natl. Acad. Sci. U.S.A. 90: 4271-4275
^{l}Sauer-Eriksson et al. 1995, Structure 3: 265-278
^{m}DeLano et al. 2000, The PyMOL Molecular Graphics System, DeLano Scientific, Palo Alto, CA, USA, Available at www.pymol.org.
ⁿIdusogie et al. 2000, J. Immunol. 164: 4178-4184
^{o}James et al. 2007; Proc. Natl. Acad. Sci. U.S.A. 104: 6200-6205
^{p}Corper et al. 1997, Nat. Struct. Biol. 4: 374-381
^{q}Fc/3M (the present application)

TABLE 7
Dissociation constants for the binding of unmutated human Fc
and Fc/3M to human CD16(V158)^{a}.

| Molecule | K_{D}-CD16(nM) |
|---|---|
| Unmutated human Fc | 157 ± 0.7 |
| Fc/3M | 5 ± 1.4 |

^{a}Affinity measurements were carried out by BlAcore as described in Materials and Methods. Errors were estimated as the standard deviations of 2 independent experiments for each interacting pair.

1. A crystal comprising a human IgG Fc variant, wherein the human IgG Fc variant comprises the high effector function amino acid residues 239D, 330L and 332E, as numbered by the EU index as set forth in Kabat, and has an increased binding affinity for an FcγR compared to a wild type human IgG Fc region.

2-3. (canceled)

4. The crystal of claim 1, wherein the human IgG Fc variant comprises the amino acid sequence of SEQ ID NO:1.

5-8. (canceled)

9. The crystal of claim 1, which is characterized by an orthorhombic unit cell of a=49.87±0.2 Å, b=147.49±0.2 Å, and c=74.32 ±0.2 Å and a space group of C222₁.

10-44. (canceled)

45. A method of identifying a compound that binds a human IgG or a human IgG Fc region, comprising using a three-dimensional structural representation of a human IgG Fc variant comprising the effector function amino acid residues 239D, 330L and 332E, as numbered by the EU index as set forth in Kabat, and has an increased binding affinity for a FcγR compared to a wild type human IgG Fc region not comprising the high effector function amino acid residues, or portion thereof, to computationally screen a candidate compound for an ability to bind the human IgG or the human IgG Fc region.

46-47. (canceled)

48. The method of claim 45, wherein the human IgG Fc variant comprises the amino acid sequence of SEQ ID NO:1.

49. The method of claim 45, wherein the three-dimensional structural representation of the human IgG Fc variant is visually inspected to identify a candidate compound.

50. The method of claim 45, wherein the computational screen comprises the steps of:
(a) synthesizing the candidate compound; and
(b) screening the candidate compound for an ability to bind a human IgG or a human IgG Fc region.

51. The method of claim 45, wherein the method further comprises comparing a three-dimensional structural representation of a wild type human IgG Fc region with that of the human IgG Fc variant.

52-95. (canceled)

96. A recombinant polypeptide comprising a human IgG Fc region that comprises one or more amino acid residue deletions compared to a wild type human IgG Fc region, wherein the Fc region comprises a deletion of amino acid residues 295 and 296; or a deletion of amino acid residues 294, 295 and 296; or a deletion of amino acid residues 294, 295, 296, 298 and 299 as numbered by the EU index as set forth in Kabat.

97. The recombinant polypeptide of claim 96, comprising SEQ ID NO:8, 9, or 10.

98. (canceled)

99. The recombinant polypeptide of claim 96, further comprising the substitution of at least one amino acid residue selected from the group consisting of 300S and 301T as numbered by the EU index as set forth in Kabat.

100. The recombinant polypeptide of claim 96, wherein the Fc region comprises the deletion of amino acid residues 294, 295, 296, 298 and 299 and further comprises the amino acid substitutions 300S and 301T as numbered by the EU index as set forth in Kabat.

101. (canceled)

102. The recombinant polypeptide of claim 96, wherein the recombinant polypeptide has a reduced binding affinity for at least one FcγRs as compared to a comparable peptide comprising a wild type human IgG Fc region.

103. The recombinant polypeptide of claim 102, wherein the FcγR is selected from the group consisting of FcγRI, FcγRIIA, FcγRIIB and FcγRIIIA

104. The recombinant polypeptide of claim 97, wherein the recombinant polypeptide has a reduced binding affinity for at least one FcγRs as compared to a comparable peptide comprising a wild type human IgG Fc region.

105. The recombinant polypeptide of claim 104, wherein the FcγR is selected from the group consisting of FcγRI, FcγRIIA, FcγRIIB and FcγRIIIA.

106. The recombinant polypeptide of claim 99, wherein the recombinant polypeptide has a reduced binding affinity for at least one FcγRs as compared to a comparable peptide comprising a wild type human IgG Fc region.

107. The recombinant polypeptide of claim 106, wherein the FcγR is selected from the group consisting of FcγRI, FcγRIIA, FcγRIIB and FcγRIIIA.

108. The recombinant polypeptide of claim 100, wherein the recombinant polypeptide has a reduced binding affinity for at least one FcγRs as compared to a comparable peptide comprising a wild type human IgG Fc region.

109. The recombinant polypeptide of claim 108, wherein the FcγR is selected from the group consisting of FcγRI, FcγRIIA, FcγRIIB and FcγRIIIA.
